SEQUENCE LISTING (55 sequences)

SEQ ID NO: 1 - DNA, 30738 bp, Human coronavirus
  ref <308>: DATABASE ACCESSION NUMBER: AY391777.1
  ref <309>: DATABASE ENTRY DATE: 2005-01-14
  ref <313>: RELEVANT RESIDUES IN SEQ ID NO: (1)..(30738)
gattgtgagcgatttgcgtgcgtgcatcccgcttcactgatctcttgttagatctttttgtaatctaaactttataaaaacatccactccctgtaatctatgcttgtgggcgtagatttttcatagtggtgtttatattcatttctgctgttaacagctttcagccagggacgtgttgtatcctaggcagtggcccgcccataggtcacaatgtcgaagatcaacaaatacggtctcgaactacactgggctccagaatttccatggatgtttgaggacgcagaggagaagttggataaccctagtagttcagaggtggatatgatttgctccaccactgcgcaaaagctggaaacagacggaatttgtcctgaaaatcatgtgatggtggattgtcgccgacttcttaaacaagagtgttgtgtgcagtctagcctaatacgtgaaattgttatgaatgcaagtccatatgatttggaggtgctacttcaagatgctttgcagtcccgtgaagcagttttggttacaacccccttaggtatgtctttagaggcatgctatgtgagaggttgtaatcctaaaggatggaccatgggtttgtttcggcgtagaagtgtgtgtaacactggtcgttgcactgttaataagcatgtggcctatcagttatatatgattgatcctgcaggtgtctgtcttggtgcaggtcaattcgtgggttgggtcatacccttagcctttatgcctgtgcaatcccggaaatttattgttccatgggttatgtacttgcgtaagcgtggcgaaaagggtgcttacaataaagatcatggacgtggcggttttggacatgtttatgattttaaagttgaagatgcttatgaccaggtgcatgatgagcctaagggtaagttttctaagaaggcttatgctttaattagagggtatcgtggtgttaaaccacttctctatgtagaccagtatggttgtgattatactggtagtcttgcagatggcttagaggcttatgctgataagacattgcaagaaatgaaggcattatttcctacttggagtcaggaactcctttttgatgtaattgtggcatggcatgttgtgcgtgatccacgttatgttatgagattgcagagtgctgctactatacgtagtgttgcatatgttgctaatcctactgaagacttgtgtgatggttctgttgttataaaagaacctgtgcatgtttatgcagatgactctattattttacgtcaatataatttagttgacattatgagtcatttttatatggaggcagatacagttgtaaatgctttttatggtgttgctttgaaagattgcggttttgttatgcagtttggttacattgattgcgaacaagactcgtgtgattttaaaggttggattcctggtaacatgatagatggttttgcttgcaccacttgtggtcatgtttatgaagtaggtgatttgatagcacaatcttcaggtgttttgcctgttaaccctgtattgcatactaagagtgcagcaggctatggtggttttggttgtaaagattcttttactctgtatggccaaactgtagtttattttggaggttgtgtgtattggagtccagcacgtaatatatggattcctatattaaaatcctctgttaagtcatatgacagtttggtttatactggagttttaggttgcaaggctattgtaaaggaaacaaatctcatttgcaaagctttgtaccttgattatgttcaacacaagtgtggcaatttacaccaacgggagttgctaggtgtttcagatgtgtggcataaacaattgctattaaatagaggtgtttataaacctctgttagagaatattgattattttaatatgcggcgcgctaaatttagtttagaaacttttactgtttgtgcagatggctttatgccttttcttttagatgatttagttccacgcgcatattatttggcagtaagtggtcaagcattttgtgattatgcagataaactttgccatgccgttgtgtctaagagtaaagagttacttgatgtgtctctggattctttaggtgcagctatacattatttgaattctaagattgttgatttggctcaacattttagtgattttggaacaagtttcgtttctaaaattgttcatttctttaagacttttactactagcactgctcttgcatttgcatgggttttatttcatgttttgcatggtgcttatatagtagtggagagtgatatatattttgttaaaaacattcctcgttatgctagtgctgttgcacaagcatttcagagtgttgctaaagttgtactggactctttaagagttacttttattgatggcctttcttgttttaagattggacgtagaagaatttgtctttcaggcagaaaaatttatgaagttgagcgtggcttgttacattcatcccaattgccattagatgtttatgatttaaccatgcctagtcaagttcagaaagccaagcaaaaacctatttatttaaaaggttctggttctgatttttcattagcggatagtgtagttgaagttgttacaacttcacttacaccatgtggttattctgaaccacctaaagttgcagataaaatttgcattgtggataatgtttatatggccaaggctggtgacaaatattaccctgttgtggttgatgatcatgttggactcttggatcaagcatggagagttccttgtgctggaaggcgtgttacatttaaggaacagcctacagtaaaggagattataagcatgcctaagattattaaggttttttatgagcttgacaacgattttaatactattttaaatactgcgtgtggagtgtttgaagtggatgatactgttgatatggaggaattttatgctgtggtgattgatgccatagaagagaaactttctccatgtaaggagcttgaaggtgtaggtgctaaagttagtgcctttttacagaaattagaggataatcccctatttttatttgatgaggctggcgaggaagttcttgctcctaaattgtattgtgcctttacagctcctgaagatgatgactttcttgaggaaagtgatgttgaagaagatgatgtagaaggtgaggaaactgatttaactgtcacaagtgctggacagccttgtgttgctagtgaacaggaggagtcttctgaagtcttagaggacactttggatgatggtccaagtgtggagacatctgattcacaagttgaagaagatgtagaaatgtcggattttgttgatcttgaatctgtgattcaggattatgaaaatgtttgttttgagttttatactacagagccagaatttgttaaagttttgggtctgtatgtgcctaaagcaactcgcaacaattgctggttgcgatcagttttggcagtgatgcagaaattgccctgtcaatttaaagataaaaatttgcaggatctttgggtgttatacaagcaacagtatagtcagttgtttgttgataccttggttaataagatacctgctaatattgtacttccacaaggtggttatgttgctgattttgcatattggtttttaaccttatgtgattggcagtgtgttgcatactggaaatgcattaaatgtgatttagctcttaagcttaaaggcttggatgctatgttcttttatggtgatgttgtttcacatatatgcaagtgtggtgagtctatggtacttattgatgttgatgtgccatttacagcccactttgctcttaaagataagttgttttgtgcatttattactaagcgtattgtgtataaagcagcttgtgttgtggatgttaatgatagtcattctatggctgttgttgatggtaaacaaattgatgatcatcgtatcactagtattactagtgataagtttgattttattattgggcatggtatgtcattttcaatgactacttttgaaattgcccaattgtatggttcttgtataacacctaatgtgtgttttgttaaaggtgatataattaaagtatctaagcttgttaaagcagaagttgttgtaaaccctgctaatggccatatggcacatggtggtggtgttgcaaaagctattgcagtagcagctggacagcagtttgttaaagagactaccgatatggttaagtctaaaggagtttgtgctactggagattgttatgtctctacagggggcaaattatgtaaaactgtgcttaatgttgttggacctgatgcgagaacacagggtaaacaaagttatgtattgttagagcgtgtttataaacatcttaacaactatgactgtgttgttacaactttgatctcagctggtatatttagtgtgccttctgatgtgtctttaacatatctacttggtactgctaagaaacaagttgttcttgttagcaataatcaagaggattttgatcttatttctaagtgtcagataactgctgttgagggcactaagaaattggcagcgcgtctttcttttaatgttggacgttccattgtttacgaaacagatgctaataagttgattttaatcaatgacgttgcatttgtttcgacatttaatgttttacaggatgttttatccttaagacatgatatagcacttgatgatgatgcacgaaccttcgttcagagcaatgttgatgttgtacctgagggttggcgtgttgtcaataagttttatcaaattaatggtgttagaaccgttaagtattttgagtgtactggaggcatagatatatgcagccaggataaagtttttggttatgtacagcagggtatttttaataaggctactgttgctcaaattaaagccttgtttttggataaagtggacatcttgctaactgttgatggtgttaatttcactaataggtttgtgcctgttggtgaaagttttggtaagagtctaggaaatgtgttttgtgatggagttaatgtcacgaagcataagtgtgatataaattataaaggtaaagtctttttccagtttgataatctttctagtgaagatttaaaggctgtaagaagttcctttaattttgatcagaaggaattgcttgcctattacaacatgcttgttaattgttttaagtggcaggttgttgttaatggtaagtatttcacttttaagcaagctaataacaattgttttgttaatgtttcttgcttaatgctccagagtttgcatctgacatttaaaattgttcaatggcaagaggcatggcttgaatttcgttctggccgccctgctagatttgtagctttggttttggccaaaggtgggtttaaatttggagatcctgctgattctagagatttcttgcgtgttgtgtttagtcaagttgatttgactggggcaatatgtgattttgaaattgcatgtaaatgtggtgtaaagcaggaacagcgtactggtctggacgctgttatgcattttggtacattgagtcgtgaagatcttgagattggttataccgtggactgttcttgcggtaaaaagctaattcattgtgtacgatttgatgtaccatttttaatttgcagtaatacacctgctagtgtaaaattacctaagggtgtaggaagtgcaaatatttttataggtgataaggttggtcattatgttcatgttaagtgtgaacaatcttatcagctttatgatgcttctaatgttaagaaggttacagatgttactggcaagttgtcagattgtctgtatcttaaaaatttgaaacaaacttttaaatcggtgttaaccacctattatttggatgatgttaagaaaattgagtataaacctgacttgtcacaatattattgtgacggaggtaagtattatactcagcgtattattaaagcccaatttaaaacattcgagaaagtagatggtgtgtatactaattttaaattgataggacacaccgtctgtgacagtcttaatgctaagttgggttttgatagctctaaagagtttgttgaatataagattactgagtggccaacagctacaggtgatgtggtgttggctactgatgatttgtatgttaagagatatgagaggggttgtattacttttggtaaacctgttatatggttaagccatgagaaagcttccctcaattctttaacatattttaatagaccttcattggttgatgataataaatttgatgttttaaaagtggatgatgttgacgatggtggtgacagctcagagagtggtgccaaagaaaccaaagaaatcaacattattaagttaagtggtgttaaaaaaccatttaaggttgaagatagtgtcattgttaatgatgatactagtgaaaccaaatatgttaagagtttgtctattgttgatgtgtatgatatgtggcttacaggttgtaagtatgttgttagaactgctaatgctttgagcagagcagttaacgtacctacaatacgtaagtttataaaatttggtatgactcttgttagtataccaattgatttgttaaatttaagagagattaagcctgctgttaatgtggttaaagctgtgcgaaataaaatttctgtatgctttaattttattaaatggctttttgtcttattatttggctggattaaaatatccgctgataataaagtaatctacaccacagaaattgcatcaaagcttacgtgtaagcttgtagctttagcttttaaaaatgcatttttgacatttaagtggagtatggttgctagaggtgcttgcattatagcgactatatttctattgtggtttaattttatatatgccaatgtaatttttagtgatttttatttgcctaaaatcggtttcttgccgacttttgttggtaagattgcacagtggattaagaacacttttagtcttgtaactatttgtgatctatattccatgcaggatgtgggttttaagaatcagtattgtaatggaagtattgcatgtcagttctgcttggcaggatttgatatgttagataattataaagccattgatgtagtacagtatgaagctgataggagagcatttgttgattatacaggtgtgttaaagattgtcattgaattgatagttagttacgccctgtatacggcatggttttatccattgtttgcccttatcagtattcagatcttgaccacttggctgcctgagctttttatgcttagtacattacattggagttttaggttgctggtggctttagctaatatgttaccagcacatgtgtttatgaggttttatattattattgcctcttttattaagctctttagcttgtttaggcatgttgcctatggttgtagtaaatctggttgtttgttttgttacaagaggaatcgtagtctacgtgttaaatgtagtactatcgttggtggcatgatacgctattacgatgttatggctaatggtggcactggcttttgttcaaaacatcaatggaattgcattgattgtgattcttataaaccaggtaatacttttattactgttgaggccgctcttgatctatctaaggaattgaaacggcccattcagcctacagatgttgcttatcatacggttactgatgttaagcaagttggttgttctatgcgcttgttctatgatcgtgatggacagcgcacatatgatgatgttaatgctagtttgtttgtggattatagtaatttgctacattctaaggttaagagtgtgcctaatatgcatgttgtggtagtggaaaatgatgctgataaagccaattttctgaatgctgctgtattttatgcacagtctttgtttagacctattttaatggttgataaaaatctgataactactgctaacactggtacgtctgttacagaaactatgtttgatgtttatgtggatacatttttgtctatgtttgatgtggataaaaagagtcttaatgctttaatagcaactgcgcattcttctataaaacagggtacgcagatttataaagttttggatacctttttaagctgtgctcgtaaaagttgttctattgattcagatgttgatactaagtgtttagctgattctgtcatgtctgctgtatcggcaggtcttgaattgacggatgaaagttgtaataacttggtgccaacatatttgaagagtgacaacattgtggcagctgatttaggtgttctgattcaaaattctgcaaagcatgtgcagggtaatgttgctaaaatagctggtgtttcctgtatatggtctgtggatgcttttaatcagtttagttctgatttccagcataaattgaagaaagcatgttgtaaaactggtttgaaactgaagcttacttataataagcagatggctaatgtctctgttttaactacaccctttagtcttaaagggggtgcagtttttagttattttgtttatgtgtgttttgtgttgagtttggtctgttttattggactgtggtgcttaatgcccacttacacagtacacaaatcagattttcagcttcccgtttatgccagttataaagttttagataatggtgttattagagatgttagcgttgaagatgtttgtttcgctaacaaatttgaacaatttgatcaatggtatgagtctacatttggtctaagttattatagtaacagtatggcttgtcccattgttgttgctgtaatagatcaggattttggctctacagtgtttaatgtccctaccaaagtgttacgatatggttatcatgtgttgcactttattacacatgcactttctgctgatggagtgcagtgttatacgccacatagtcaaatatcgtattctaatttttatgctagtggctgtgtgctttcctctgcttgcactatgtttacaatggccgatggtagtccacaaccttattgttatacagaggggcttatgcaaaatgcttctctgtatagttcattggtacctcacgtgcggtataatcttgctaatgctaaaggttttatccgttttccagaagtgttgcgagaagggcttgtacgtatcgtgcgtactcgttctatgtcgtattgcagagttggattatgtgaggaagctgatgagggtatatgctttaattttaatggttcttgggtgcttaataatgattattatagatcattgcctgggaccttttgtggtagagatgtttttgatttaatttatcagctatttaaaggtttagcacagcctgtggattttttggcattgactgctagttccattgctggtgctatactcgctgtaattgttgttttggtgttttattacctaataaagcttaaacgtgcttttggtgattacaccagtgttgtttttgttaacgtgattgtgtggtgtgtaaattttatgatgctttttgtgtttcaagtttaccccatactttcttgtgtatatgctatttgttatttttatgccacgctttatttcccttcggagataagtgtgataatgcacttacaatggctagttatgtatggcactattatgcctttatggttttgtttgctatatatagctgttgttgtttcaaatcatgctttttgggtattttcttactgcagaaagcttggtacttctgttcgtagtgatggtacatttgaagaaatggctctcactacttttatgattacaaaagattcttattgtaagcttaagaattctttgtctgatgttgcttttaatagatatttgagtttgtataataaatataggtattacagcggtaaaatggatactgctgcatatagggaggctgcttgctctcagttggctaaagcaatggacacatttaccaataataatggtagtgatgtgctttaccaaccgcctactgcttccgtctcaacttcattcttgcaatctggtattgtgaaaatggtaaatcctacttctaaggtagaaccatgtgttgtcagtgttacctatggtaatatgacattgaatggtttatggttggatgacaaggtctactgtcccagacatgtaatatgttctgcttcagatatgactaatccagattatacaaatttgttgtgtagagtaacatcaagtgattttactgtattgtttgatcgtctaagccttacagtgatgtcttatcaaatgcggggttgtatgcttgttcttacagtgaccctgcaaaattctcgtacgccaaaatatacatttggtgtggttaaacctggtgagacttttactgttttagctgcttataacggcaaaccacaaggagcctttcatgtaactatgcgtagtagttataccattaagggttcctttttatgcggatcttgtggatctgttggttatgtaataatgggtgattgtgttaaatttgtttatatgcatcaattggagcttagtactggttgtcatactggtactgacttcaatggggatttttatggtccttataaggatgctcaggttgttcagttgctcattcaggattatatacaatctgttaattttgtagcatggctttatgctgctatacttaacaattgtaattggtttgtacaaagtgataagtgttctgtagaagattttaatgtgtgggctctgtccaatggatttagccaagttaaatctgaccttgttatagatgctttagcttctatgactggtgtgtctttggaaacactgttggctgctattaagcgtcttaagaatggtttccaaggacgtcagattatgggtagttgctcttttgaggatgaattgacacctagcgatgtttatcaacaactcgctggtatcaagttacaatcaaaacgcactagattgtttaaaggcactgtttgttggattatggcttctacatttttgtttagttgcataattacagcatttgtgaaatggactatgtttatgtatgtaactactaatatgtttagtattacgttttgtgcactttgtgttataagtttggccatgttgttggttaagcataagcatctttatttgactatgtatataactcctgtgctttttacactgttgtataacaactatttggttgtgtacaagcatacatttagaggctatgtctatgcatggctatcatattatgttccatcagttgagtacacttatactgatgaagttatttatggcatgttattgcttgtaggaatggtctttgttacattacgtagcattaaccatgatttgttttcttttataatgtttgttggtcgtttgatttctgttttctctttgtggtacaagggttctaacttagaggaagaaattcttcttatgttggcttccctttttggtacttacacatggacaacagttttatctatggctgtagcaaaggttattgctaagtgggttgctgtgaatgtcttgtatttcacagatatacctcaaattaagatagtgcttttgtgctatttgtttattggttatattattagctgttattggggcttgttttccttgatgaacagtttgtttagaatgcctttgggtgtttataattataaaatttcagtacaggaattaagatatatgaatgctaatggattgcgccctcctaagaatagttttgaagcccttatgcttaattttaagctgttgggtattggaggtgttccaatcattgaagtatctcaatttcaatcaaaattgactgatgtcaaatgtgctaatgtcgtcttgcttaattgcttgcaacatttgcatgttgcttctaattctaagttgtggcattattgtagcactttgcacaatgaaatacttgccacttcggatctgagtgttgcttttgaaaagcttgctcagttattaattgttttgtttgctaatccagctgctgtggatagcaagtgcctgactagtattgaagaagtttgcgatgattacgcaaaggacaatactgttttgcaggctttacagagtgaatttgttaatatggctagcttcgttgaatatgaagttgctaagaaaaatcttgatgaggcgcgttttagtggttctgctaatcaacagcagttaaaacagctagagaaagcctgtaatattgctaaatctgcttatgaacgcgaccgtgctgtagcaaaaaagttggagcgtatggctgatttggctctcactaatatgtataaagaagctagaattaatgataagaagagtaaggttgtttctgccttgcaaactatgctttttagtatggtgcgtaagttagataatcaagctctgaattcaatattagataacgctgtgaagggttgtgtaccattgaatgcaataccttcattggcagcaaatactctgaatataattgtaccagataaaagtgtttatgaccaggtagttgataatgtctatgttacctatgcgggtaatgtatggcagattcaaactatccaggattcagatggtacaaataagcagttgaatgagatatctgatgattgtaactggccactagttattattgcaaatcggtataatgaggtatctgctactgttttgcaaaataatgaattaatgcctgctaagttgaaaattcaggttgttaatagtggtccagatcagacttgtaatacacctactcaatgttactataataatagtaacaatgggaagattgtttatgctatacttagtgatgttgatggtcttaagtatacaaaaattcttaaagatgatggcaattttgttgttttggagttagatcctccttgtaaatttactgttcaagatgctaaaggtcttaaaattaagtacctttattttgtaaaaggttgtaacacactagcaagaggctgggttgttggtacaatttcttctacagttagattgcaagctggaactgctactgaatatgcttccaactcatctatattgtctttatgtgcgttttctgtagatcctaagaaaacgtatttagattttatacaacaaggaggaacacctattgccaattgtgttaaaatgttgtgtgaccatgctggtaccggtatggccattactgttaaacccgatgctaccactagtcaggattcatatggtggtgcgtctgtttgtatatattgccgcgcacgagttgaacacccagatgttgatgggttgtgcaaattacgcggcaagtttgtacaagtgcctgtaggtataaaagatcctgtgtcttatgttttgacacatgatgtttgtcgagtttgtggattttggcgggatggaagttgttcatgtgttagcactgacactactgttcaatcaaaagatactaattttttaaacgggttcggggtacgagtgtagatgcccgtctcgtaccctgcgccagtggtttatctactgatgtacaattaagggcatttgatatttacaatgctagtgttgctggcattggtttacatttaaaagttaattgttgccgttttcagcgtgttgatgagaacggtgataaattagatcagttctttgttgttaagaggacagatctgactatatataatagagagatgaaatgctatgagcgtgtaaaagattgtaagtttgtggctgaacacgatttctttacatttgatgtagaaggtagtcgtgtgccacacattgtacgcaaggatttaacaaagtatactatgttggatctttgctatgcattgcgacattttgatcgcaatgattgcatgctgctttgtgacattctctctatatatgctggttgtgaacaatcctactttactaagaaggattggtatgattttgttgaaaatcctgatattattaatgtgtataaaaagctaggacctatttttaatagagccctagttagcgctactgagtttgcggacaaattggtggaggtaggcttagtaggcgttttaacacttgataatcaagatttaaatggtaaatggtatgattttggtgactatgttattgcagccccaggatgtggtgttgctatagcagattcttattattcttatatcatgcctatgctgaccatgtgtcatgcattggattgcgaattgtatgtgaataatgcttatagactatttgatcttgtacagtatgattttactgattacaagcttgaattgtttaataagtattttaagcactggagtatgccatatcatcctaacactgttgattgtcaggatgatcggtgtattatacattgtgctaattttaacatactttttagtatggttttacctaatacatgttttgggcctcttgttaggcaaatttttgtggatggtgtgccttttgttgtttcaattggctaccattataaagaacttggtattgtgatgaatatggatgtggatacacatcgttatcgcttgtctttaaaagacttgcttttatatgctgctgatccagctttgcatgtagcttctgctagtgcattgtatgatttacgcacttgctgttttagtgttgccgctataacaagcggtgtaaaatttcaaacagttaaacctggtaattttaatcaggatttttatgattttgttttaagtaaaggcctgcttaaagagggtagctcagttgatctgaagcactttttctttacacaggatggtaatgctgctattactgattataattattataagtataatttgcccaccatggtggacattaagcagttgttgtttgttttggaagttgtttataagtattttgagatttatgatggtgggtgtataccggcatcacaagtcattgttaataattatgataagagtgctggctatccatttaacaaatttggaaaagccaggctctattatgaagcattatcatttgaggaacaggatgaaatttacgcttatactaagcgtaatgtcctgccaacacttactcaaatgaatttgaaatatgctattagtgctaagaatagagcccgcactgttgctggtgtttccatacttagtactatgactggcagaatgtttcatcaaaaatgtttgaaaagtatagcagctacacgtggtgttcctgtagttataggcaccactaaattttatggtggctgggatgatatgttacgccgccttattaaagatgttgacaatcctgtacttatgggttgggattatcctaagtgtgatcgtgctatgccaaacctactacgtattgttagtagtttggtattagcccgaaaacatgagacatgttgttcgcaaagcgataggttttatcgacttgcgaatgaatgcgcacaagttttgagtgaaattgttatgtgtggtggctgttattatgttaagcctggtggcactagtagtggtgatgcaactactgcttttgctaattcagtctttaacatatgtcaagctgtttcagccaatgtatgtgccttaatgtcatgcaatggcaataagattgaagatcttagtatacgtgctcttcagaagcgcttatactcacatgtgtatagaagtgataaggttgattcaacctttgtcacagaatattatgaatttttaaataagcattttagtatgatgattttgagtgatgatggggttgtgtgttataattctgattatgcgtccaaagggtatattgctaatataagtgcctttcaacaggtattatattatcaaaataacgtttttatgtcagaatccaaatgttgggttgaacatgacataaataatggacctcatgaattctgttcacaacacacaatgcttgtaaagatggatggtgacgatgtctaccttccatatcctaatcctagtcgtatattaggagctggatgttttgtagatgatttgttaaagactgatagtgttcttttaatagaacgatttgtaagtcttgcaatagatgcttatccacttgtgtatcatgaaaatgaagaataccaaaaggtttttcgtgtttatttggcgtatataaagaagttgtacaatgacctgggtaatcagatcttggatagctacagtgttattttaagtacttgtgatggacaaaagttcactgatgagtccttttacaagaacatgtatttaagaagtgcagttatgcagagtgttggagcttgcgtggtctgctcttctcaaacatcattacgttgtggcagttgcatcagaaagcctcttctttgctgcaagtgttgttatgatcatgttatggcgactgatcataaatatgtcttgagtgtttcaccatatgtgtgtaatgcaccaggatgtgatgtaaatgatgttaccaaattgtatctaggtggtatgtcatattattgtgaagaccataagccacaatattcattcaagttggtaatgaatggtctggtttttggtctatataaacaatcttgtacaggatctccgtacatagacgattttaatcgtatagctagttgtaaatggaccgatgtggatgattacatactagctaatgaatgtacagagcgcttgaaattgtttgctgcagaaacgcaaaaggcaaccgaggaagcctttaagcagagttatgcatcagcaacaatacaagagattgttagtgagcgcgaattgattctctcttgggagattggaaaagttaagccaccacttaataaaaattatgtttttactggctaccattttactaaaaatggtaagacagttttaggtgagtatgtttttgataagagtgagttgactaatggtgtgtattatcgcgccacaaccacttataagctatctgtaggagatgtttttgttttaacctctcattcagtagctaatttaagtgctcctacgcttgttccgcaggagaattatagtagtattagatttgctagtgtttatagtgtgcttgagacgtttcagaacaatgttgttaattatcaacacattggtatgaaacgttactgcaccgtgcaaggacctcctggtacagggaagtcacatcttgctattggtcttgctgtattctattgtacagcacgtgttgtatacacagcggccagccatgcagctgttgacgcattgtgtgaaaaagcatataaatttttgaatataaatgattgcactcgtattgttccggccaaggtcagggtggagtgctatgataagtttaaaattaatgacaccactcgtaagtatgtgtttactaccataaatgcattacctgagatggtgactgatattgttgttgtagatgaagttagtatgcttaccaattatgagctttctgttattaatgctcgtattcgcgctaagcattatgtttatattggtgatcctgctcaattgccagcaccacgtgtgttattgagcaagggtacacttgaacctaaatattttaacactgttactaagctcatgtgttgcttagggccagacatttttcttggtacatgttatagatgtcctaaggaaatcgttgatacagtgtccgccttggtttatgaaaataagcttaaggctaagaatgagagtagttcattgtgttttaaggtctattataagggcgttacaacacatgaaagttctagtgctgtaaatatgcagcagatttatttgattaataagtttttgaaggctaaccctttgtggcataaagctgtttttattagcccatataatagtcagaactttgcagctaagcgtgttttgggtttacaaacccaaaccgtggattctgctcaaggttctgaatatgattatgttatatattcacagactgcagaaacagcgcattctgtaaatgttaatcgcttcaatgttgctattactcgagccaagaaaggtattctttgtgttatgagtaatatgcagttgtttgaagcattacagtttactacattgaccttagataaagtgccacaggccgtcgaaactaaagttcaatgtagtactaatttatttaaagattgtagcaagagttatagcggttatcacccagctcatgctccttcatttttggcagtagatgacaaatataaggcaactggcgatttagccgtgtgtcttggtattggtgattctgctgttacatattcaagattaatatcactcatgggttttaaattggatgttacccttgatgggtattgtaagctttttataactaaagaagaagctgttaaacgcgtgcgtgcctgggttggctttgatgctgaaggtgctcatgccacgcgtgatagcattgggacaaatttcccacttcaattaggattttccacaggaattgattttgttgtggaagccactggtttgtttgctgatagagatggttacagctttaaaaaggctgtggcgaaagctcctcctggtgaacaatttaagcacctcatccctttgatgacgagaggtcatcgctgggatgttgttagacctagaatagtacaaatgtttgcagatcatttaattgatctgtctgattgtgttgtgctagttacatgggcagccaactttgagctcacttgtctccgctactttgcaaaagtagggcgtgagatttcttgtaatgtatgcactaaacgtgccacagtttacaattctagaactggttactatggttgttggcgccatagtgttacatgtgattacttgtataatccacttattgttgatattcaacagtggggatatattggttctttatcaagtaatcatgatttatattgtagtgtccataaaggagcacatgttgcttcctctgatgctataatgacacggtgtttggccgtttatgattgcttttgcaataatattaattggaatgtggagtatcccatcatttcaaatgagttaagtattaatacctcttgtagggtcttgcagcgtgtgattcttaaagctgccatgctctgcaacagatatactttgtgttatgatattggcaacccaaaagcgattgcctgtgtcaaagattttgattttaagttctatgatgcccaaccaattgttaagtctgttaagactcttttgtattcttttgaggcacataaggactcttttaaagacggtttgtgtatgttttggaactgtaatgtggataagtatccaccgaatgcagttgtatgtagatttgacactagagtgttgaataatttaaatcttcctggctgtaatggaggtagtttgtatgttaataaacatgcattccacactaaaccctttgctagggcagcctttgagcatttgaagcctatgccattcttctattattcagatacgccttgtgtgtatatggatggcatggatgctaagcaggttgattatgtacctttgaaatctgccacgtgcatcacaagatgcaatttaggtggtgcagtttgtttaaaacatgctgaagagtatcgtgagtacttagagtcttacaatacagctactacagcaggttttactttttgggtctataagacatttgatttttataatttgtggaatacgttcaccaagctacaaagcttggagaatgttgtatataatttagtcaagactggtcattatacaggacaggctggtgaaatgccttgtgccattataaatgataaagttgtggctaagatcgataaggaggatgttgtcatttttattaataatacaacataccctactaatgtggccgttgaattatttgccaagcgcagtgttcgacaccacccagagcttaagctctttagaaatttaaatatagacgtgtgttggaagcacgtcatttgggattatgctagagaaagtatattttgcagtaatacctatggtgtctgcatgtatacagatttaaagttcattgataaattgaatgtcctttttgatggtcgtgataatggtgctcttgaagcttttaaacgttctaataatggcgtttacatttccacgacaaaagttaagagtctttcgatgataagaggtccaccgcgtgctgaattaaatggcgtagtggtggacaaggttggagacactgattgtgtgttttattttgctgtgcgtaaagaaggtcaggatgtcatcttcagccaattcgacagcctgggagtcagctctaaccagagcccacaaggtaatctggggagtaatggtaaacccggtaatgtcggtggtaatgatgctctgtcaatctctactatctttacacaaagccgtgttattagctcttttacatgtcgtactgatatggaaaaagattttatagctttagatcaagatgtgtttattcagaagtatggtttggaggactatgcctttgaacacattgtttatggtaacttcaaccagaagattattggtggtttgcatttgttaataggcttgtaccgaagacagcaaacttccaatctggttgttcaggagtttgtttcatatgactccagcatacactcttattttatcactgacgagaagagtggtggtagtaagagtgtttgcactgttatagatattttgttggatgattttgtggctcttgttaagtcacttaatcttaattgtgtgagtaaggttgttaatgttaatgttgattttaaagattttcagtttatgctttggtgtaacgatgagaaagttatgactttctatcctcgtttgcaagctgcatctgactggaagcctggttattctatgcctgtattatataagtatttgaattctccaatggaaagagttagtctctggaattatgggaagccagttactttgcctacaggctgtatgatgaatgttgctaagtatactcagttatgtcaatatctgaatactacaacattagctgtacctgttaatatgcgagttttgcatttaggtgcaggttcagaaaaaggagtagcaccgggttctgcagttcttaggcagtggttgcctgctggtactattcttgtagataacgatttatacccatttgttagtgacagtgtcgctacatattttggggattgtataactttaccctttgattgtcaatgggatttgataatttctgatatgtatgaccctattactaagaacataggggagtacaatgtgagtaaagatggtttctttacatacatttgtcatatgattcgagacaagttagctctgggtggcagtgttgctataaaaataacagagttttcttggaatgcagaattatataagttaatggggtattttgcattttggactgtgttttgcacaaatgcaaatgcttcttctagtgaaggatttttaattggcataaattatttgtgtaagcccaaggttgagatagatggaaatgttatgcatgccaattatttgttttggagaaattccacagtttggaacgggggtgcttatagcctgtttgatatggctaaattcccgcttaagttggctggtactgccgtaataaatttaagagcagaccagattaatgatatggtttattcccttcttgaaaagggtaaactacttattagagatacaaataaagaagttttcgttggtgacagtttggttaatgtaatctaaactttaaaaatggctgtcgcttatgcagacaagcctaatcattttatcaattttccacttacccattttcagggttttgtgttaaattataaaggtttacaatttcaaattctcgatgaaggagtggattgtaaaatacaaacagcgccacacattagtcttactatgctggacatacagcctgaagactataaaagtgttgatgtcgctattcaagaagttattgatgatatgcattggggtgatggttttcagattaaatttgagaatcctcacatcctaggaagatgcatagttttagatgttaaaggtgtagaagaattgcatgacgatttagttaattacattcgtgataaaggttgtgttgctgaccaatccaggaaatggattggccattgcaccatagctcaactcacggatgcagcactgtccattaaggaaaatgttgattttataaacagcatgcaattcaattataaaatcaccatcaacccctcatcaccggctagacttgaaatagttaagctcggtgctgaaaagaaagatggtttttatgaaaccatagttagtcactggatgggaattcgttttgaatacacatcacccactgataagctagctatgattatgggttattgttgtttagatgtggtacgtaaagagctagaagaaggcgatcttcccgagaatgatgatgatgcttggtttaagctatcgtaccattatgaaaacaattcttggttcttccgacatgtctacaggaaaagttttcatttccgtaaggcttgtcaaaatttagattgtaattgtttggggttttatgaatcttcagttgaagaatattaaactcagtgaaaatgtttttgcttcctagatttattctagttagctgcataattggtagcttaggtttttacaaccctcctaccaatgttgtttcgcatgtaaatggagattggtttttatttggtgacagtcgttcagattgtaatcatattgttaatatcaacccccataattattcttatatggaccttaatcctgttctgtgtgattctggtaaaatatcatctaaagctggcaactccatttttaggagttttcactttaccgatttttataattacacaggcgaaggtcaacaaattattttttatgagggtgttaattttacgccttatcatgcctttaaatgcaaccgttctggtagtaatgatatttggatgcagaataaaggcttgttttatactcaggtttataagaatatggctgtgtatcgcagccttacttttgttaatgtaccatatgtttataatggctccgcacaagctacagctctttgtaaatctggtagtttagtccttaataaccctgcatatatagctcctcaagctaactctggggattattattataaggttgaagctgatttttatttgtcaggttgtgacgagtatatcgtaccactttgtatttttaacggcaagtttttgtcgaatacaaagtattatgatgatagtcaatattattttaataaagacactggtgttatttatggtctcaattctacagaaaccattaccactggttttgatcttaattgttattatttagttttaccctctggtaattatttagccatttcaaatgagctattgttaactgttcctacgaaagcaatctgtcttaataagcgtaaggattttacgcctgtacaggttgttgattcgcggtggaacaatgccaggcagtctgataacatgacggcggttgcttgtcaacctccgtactgttattttcgtaattctactaccaactatgttggtgtttatgatattaatcatggagatgctggttttactagcatacttagtggtttgttatataattcaccttgtttttcgcagcaaggcgtttttaggtatgataatgttagcagtgtctggcctctctacccctatggcagatgtcccactgctgctgatattaatatccctgatttacccatttgtgtgtatgatccgctaccagttattttgcttggcattcttttgggcgttgcgattgtaattattgtagttttgttgttatattttatggtggataatgttactaggctgcatgatgcttagaccataatctaaacatgtttttgatacttttaatttccttaccaacggcttttgctgttataggagatttaaagtgtacttcagatactagttatattaatgataaagacaccggtcctcctcctataagtactgatactgttgatgttactaatggtttgggtacttattatgttttagatcgtgtgtatttaaatactacgttgtttcttaatggttattaccctacttcaggttccacatatcgtaatatggcactgaagggaagtgtactattgagcagactatggtttaaaccaccatttctttctgattttattaatggtatttttgctaaggtcaaaaataccaaggttattaaagatcgtgtaatgtatagtgagttccctgctataactataggtagtacttttgtaaatacatcctatagtgtggtagtacaaccacgtacaatcaattcaacacaggatggttataataaattacaaggtcttttagaggtctctgtttgccagtataatatgtgcgagtacccacaaacgatttgtcatcctaacctgggtaatcatcgcaaagaactatggcatttggatacaggtgttgtttcctgtttatataagcgtaatttcacatatgatgtgaatgctgattatttgtattttcatttttatcaagaaggtggtactttttatgcatattttacagacactggtgttgttactaagtttttgtttaatgtttatttaggcatggcgctttcacactattatgtcatgcctctgacttgtaatagtaaggttaagaatggttttactttagaatattgggttacacctctcacttctagacaatatttactcgctttcaatcaagatggtattatttttaatgctgttgattgtatgagtgattttatgagtgagattaagtgtaaaacacaatctatagcaccacctactggtgtttatgaattaaacggttacactgttcagccaatcgcagatgtttaccgacgtaaacttaatcttcccaattgcaatatagaagcttggcttaatgataagtcggtgccctctccattaaattgggaacgtaagacattttcaaattgtaattttaatatgagcagcctgatgtcttttattcaggcagactcatttacttgtaataatattgatgctgctaagatatatggtatgtgtttttccagcataactatagataagtttgctatacccaatggcaggaaggttgacctacaattgggtaatttgggctatttgcagtcatttaactatagaattgatactactgcaacaagttgtcagttgtattataatttacctgctgctaatgtttctgttagcaggtttaatccttctacttggaataagagatttggttttatagaagattctgtttttaagcctcgacctgcaggtgttcttactaatcatgatgtagtttatgcacaacactgtttcaaagctcctaaaaatttctgtccgtgtaaattgaatggttcgtgtgtaggtagtggtcctggtaaaaataatggtataggcacttgtcctgcaggtactaattatttaacttgtgataatttgtgcactcctgatcctattacatttaaagctacaggtacttataagtgcccccaaactaaatctttagttggcataggtgagcactgttcgggtcttgctgttaaaagtgattattgtggaggcaattcttgtacttgccgaccacaagcatttttgggttggtctgcagactcttgtttacaaggagacaagtgtaatatttttgctaattttattttgcatgatgttaatagtggtcttacttgttctactgatttacaaaaagctaacacagacataattcttggtgtttgtgttaattatgacctctatggtattttaggccaaggcatttttgttgaggttaatgcgacttattataatagttggcagaaccttttatatgattctaatggtaatctctacggttttagagactacataacaaacagaacttttatgattcgtagttgctatagcggtcgtgtttctgcggcctttcacgctaactcttccgaaccagcattgctatttcggaatattaaatgcaactacgtttttaataatagtcttacacgacagctgcaacccattaactattttgatagttatcttggttgtgttgtcaatgcttataatagtactgctatttctgttcaaacatgtgatctcacagtaggtagtggttactgtgtggattactctaaaaacagacgaagtcgtggagcgattaccactggttatcggtttactaattttgagccatttactgttaattcagtaaacgatagtttagaacctgtaggtggtttgtatgaaattcaaataccttcagagtttactataggtaatatggaggagtttattcaaacaagctctcctaaagttactattgattgtgctgcatttgtctgtggtgattatgcagcatgtaaatcacagttggttgaatatggtagtttctgtgataacattaatgccatactcacagaagtaaatgaactacttgacactacacagttgcaagtagctaatagtttaatgaatggtgttactcttagcactaagcttaaagatggcgttaatttcaatgtagacgacatcaatttttcccctgtattaggttgtctaggcagcgaatgtagtaaagcttccagtagatctgctatagaggatttactttttgataaagtaaagttatctgatgtcggttttgttgaggcttataataattgtacaggaggtgccgaaattagggacctcatttgtgtgcaaagttataaaggcatcaaagtgttgcctccactgctctcagaaaatcagatcagtggatacactttggctgccacctctgctagtctatttcctctttggacagcagcagcaggtgtaccattttatttaaatgttcagtatcgcattaatgggcttggtgtcaccatggatgtgctaagtcaaaatcaaaagcttattgctaatgcatttaacaatgccctttatgctattcaggaagggttcgatgcaactaattctgctttagttaaaattcaagctgttgttaatgcaaatgctgaagctcttaataacttattgcaacaactctctaatagatttggtgctataagtgcttctttacaagaaattctatctagacttgatgctcttgaagcggaagctcagatagatagacttattaatggtcgtcttaccgctcttaatgcttatgtttctcaacagcttagtgattctacactggtaaaatttagtgcagcacaagctatggagaaggttaatgaatgtgtcaaaagccaatcatctaggataaatttctgtggtaatggtaatcatattatatcattagtgcagaatgctccatatggtttgtattttatccactttagttatgtccctactaagtatgtcacagcgagggttagtcctggtctgtgcattgctggtgatagaggtatagctcctaagagtggttattttgttaatgtaaataatacttggatgtacactggtagtggttactactaccctgaacctataactgaaaataatgttgttgttatgagtacctgcgctgttaattatactaaagcgccgtatgtaatgctgaacacttcaatacccaaccttcctgattttaaggaagagttggatcaatggtttaaaaatcaaacatcagtggcaccagatttgtcacttgattatataaatgttacattcttggacctacaagttgaaatgaataggttacaggaggcaataaaagtcttaaatcagagctacatcaatctcaaggacattggtacatatgaatattatgtaaaatggccttggtatgtatggcttttaatctgccttgctggtgtagctatgcttgttttactattcttcatatgctgttgtacaggatgtgggactagttgttttaagaaatgtggtggttgttgtgatgattatactggataccaggagttagtaatcaaaacttcacatgacgactaagttcgtctttgattcattgcactgatctcttgttagatctttttgcaatctagcatttgttaaagttcttaaggccacgccctattaatggacatttggagacctgagaagaaatatctccgttatattaacggttttaatgtctcagaattagaagatgcttgttttaaatttaactatcaatttcctaaagtaggatattgtagagttcctagtcatgcttggtgccgtaatcaaggtagattttgtgctacattcactctttatggtaaatccaaacattatgataaatattttggagtaataaatggtttcacagcattcgctaatactgtagaggatgctgttaacaaactggttttcttagctgttgactttattacctggcgcagacaggagttaaatgtttatggctgatgcttatcttgcagacactgtgtggtatgtggggcaaataatttttatagttgccatttgtttattggttacaatagttgtagtggcatttttggcaacttttaaattgtgtattcaactttgcggtatgtgtaataccttagtactgtccccttctatttatgtgtttaatagaggtaggcagttttatgagttttacaatgatgtaaaaccaccagtccttgatgtggatgacgtttaggtaatccaaacattatgagtagtaaaactacaccagcaccagtttatatctggactgctgatgaagctattaaattcctaaaggaatggaatttttctttgggtattatactactttttattacaatcatattgcaatttggatatacaagtcgcagtatgtttgtttatgttattaagatgattattttgtggcttatgtggccccttactataatcttaactattttcaattgcgtatacgcattgaataatgtgtatcttggcctttctatagtttttaccatagtggccattattatgtggattgtgtattttgtgaatagtatcaggttgtttattagaactggaagtttttggagtttcaacccagaaacaaacaacttgatgtgtatagatatgaaaggaacaatgtatgttaggccgataattgaggactatcatactctgacggtcacaataatacgcggccatctttacattcaaggtataaaactaggtactggctattctttggcagatttgccagcttatatgactgttgctaaggttacacacctgtgcacatataagcgtggttttcttgacaggataagcgatactagtggttttgctgtttatgttaagtccaaagtcggtaattaccgactgccatcaacccaaaagggttctggcatggacaccgcattgttgagaaataatatctaaattttaaggatgtcttttactcctggtaagcaatccagtagtagagcgtcctctggaaatcgttctggtaatggcatcctcaagtgggccgatcagtccgaccagtttagaaatgttcaaaccaggggtagaagagctcaacccaagcaaactgctacctctcagcaaccatcaggagggaatgttgtaccctactattcttggttctctggaattactcagtttcaaaagggaaaggagtttgagtttgtagaaggacaaggtgtgcctattgcaccaggagtcccagctactgaagctaaggggtactggtacagacacaacagacgttcttttaaaacagccgatggcaaccagcgtcaactgctgccacgatggtatttttactatctgggaacaggaccgcatgctaaagaccagtacggcaccgatattgacggagtctactgggtcgctagcaaccaggctgatgtcaataccccggctgacattgtcgatcgggacccaagtagcgatgaggctattccgactaggtttccgcctggcacggtactccctcagggttactatattgaaggctcaggaaggtctgctcctaattccagatctacttcgcgcacatccagcagagcctctagtgcaggatcgcgtagtagagccaattctggcaatagaacccctacctctggtgtaacacctgacatggctgatcaaattgctagtcttgttctggcaaaacttggcaaggatgccactaaacctcagcaagtaactaagcatactgccaaagaagtcagacagaaaattttgaataagccccgccagaagaggagccccaataaacaatgcactgttcagcagtgttttggtaagagaggccctaatcagaattttggtggtggagaaatgttaaaacttggaactagtgacccacagttccccattcttgcagaactcgcacccacagctggtgcgtttttctttggatcaagattagagttggccaaagtgcagaatttatctgggaatcctgacgagccccagaaggatgtttatgaattgcgctataacggcgcaattaggtttgacagtacactttcaggttttgagaccataatgaaggtgctgaatgagaatttgaatgcctatcaacaacaagatggtatgatgaatatgagtccaaaaccacagcgtcagcgtggtcataagaatggacaaggagaaaatgataatataagtgttgcagtgcccaaaagccgcgtgcagcaaaataagagtagagagttgactgcagaggacatcagccttcttaagaagatggatgagccctatactgaagacacctcagaaatataagagaatgaaccttatgtcggcatctggtggtaacccctcgcagaaaagtcgagataaggcactctctatcagaatggatgtcttgctgctataatagatagagaaggttatagcagactatagattaattagttgaaagttttgtgttgtaatgtatagtgttggagaaagtgaaagacttgcggaagtaattgccgacaagtgcccaagggaagagccagcatgttaagttaccacccagtaattagtaaatgaatgaagttaattatggccaattggaagaatcac

SEQ ID NO: 2 - DNA, 29934 bp, Human coronavirus
  ref <308>: DATABASE ACCESSION NUMBER: KF430201.1
  ref <309>: DATABASE ENTRY DATE: 2014-09-26
  ref <313>: RELEVANT RESIDUES IN SEQ ID NO: (1)..(29934)
gattgacgttcgtaccgtctatcagcttacgatctcttgtcagatctcattaaatctaaactttttaaacaagattccctgttatccatgcttgtgagtgtggtttaatcataatcttgtattttactttccacacttttcatctctctgccagtgacgtgttggttgtcctcagcgtccctcccataggtcgcaatgattaaaaccagcaaatacggtctcggcttcaagtgggcgccagaatttcgttggctgcttccggatgcagcggaggagttggctagtcctatgaagtcagatgagggtgggttatgcccctctactggtcaagcgatggaaagtgttggattcgtttatgataatcatgtgaagatagattgtcgctgcattcttggacaagaatggcatgtgcagtcaaatcttatccgtgatatttttgttcatgaagatctacatgttgtagaagttctaactaaaacagccgtaaagtccggtacggcaattttaattaaatcacctttgcatagcttgggtggttttcctaaagggtatgttatgggcttgttccgttcatacaagactaaacgttatgttgtacatcatctttctatgactacatctactactaattttggtgaagattttttgggttggattgtaccttttggttttatgccatcttatgttcacaaatggtttcaattctgtaggttgtatattgaagagagtgatttaataatttcaaattttaaatttgatgattatgattttagtgtagaagatgtttatgctgaggttcatgctgagcctaaaggtaaatattcacaaaaagcttatgctttacttagacaatatcgtggtattaaacccgtactttttgtagaccagtatggttgtgactattctggtaaattagcagattgtcttcaagcttatggtcattattctttgcaagatatgagacaaaagcagtctgtatggcttgccaattgtgactttgatattgtagtggcttggcatgtagttcgtgattcacgatttgttatgcgcctgcagactatagctactatttgtggtattaaatatgttgcacaacctacagaagatgtagtagatggagatgtagttatacgtgaacctgtacatttattatctgctgatgcaatagttttaaagcttcctagtttgatgaaagttatgactcatatggatgatttttctattaaatctatatataatgttgatttgtgtgattgtggttttgttatgcagtatggttatgtagattgttttaatgataattgtgatttttatggttgggtttcaggtaatatgatggatggtttttcttgtccattgtgttgtacagtttatgactctagcgaagttaaagcccaatcatctggtgttattcctgaaaatcctgtgttatttactaatagtactgatactgttaaccatgattcttttaatttgtatggttattctgtcacaccatttggttcttgtatatattggtcgccgcgtcctggattgtggattcctataattaaatcttcagtcaagtcttatgatgatttggtttattcaggtgtagtaggttgtaaatctattgttaaagaaactgctcttattactcatgcactttacttagattatgttcaatgtaagtgtggtaatcttgaacaaaatcatattcttggcgttaataattcttggtgtaggcaactgttgcttaatagaggtgattataatatgcttctaaaaaatattgacttgtttgttaagcgtcgtgctgattttgcttgcaagtttgcagtttgtggagatggttttgtaccttttttactagatggtttaattccccgtagttattatctaattcagagtggtattttctttacatctttgatgtctcaattttcacaagaagtttctgatatgtgtttaaaaatgtgtattttgtttatggacagagtttcagttgctacattttatatagagcattatgttaataggttggttactcaatttaagttattgggtactacacttgttaataaaatggttaattggtttaataccatgttagatgctagtgcacctgctacaggctggcttctttaccaattattgaatggtctttttgtagtatctcaagccaactttaattttgttgctttaatacctgattatgctaaaattttagttaataaattttacactttttttaagttattattagagtgtgttacagttgatgttttaaaagatatgcctgttcttaaaactattaatggtttagtttgtattgtaggcaataagttttataacgttagtacagggttaattcctggttttgttttaccatgtaatgcacaggaacaacaaatttatttttttgaaggcgttgcagaatctgttatagtagaagatgatgttattgagaatgtcaaatcttctttatcatcttatgagtattgtcaaccacctaaatctgtagaaaaaatttgtattatagataatatgtacatgggtaagtgtggtgataaatttttccctattgtcatgaatgataaaaatatttgtcttttagatcaggcttggcgttttccatgtgcaggtagaaaagttaattttaacgagaaacctgttgttatggagattccgtctttgatgacagttaaggttatgtttgatttagattctacttttgatgatattttaggtaaagtttgttcagaatttgaagtagaaaagggtgttactgtagatgattttgttgctgttgtttgtgatgctatagagaatgctttaaactcttgtaaagagcatccagtggttggttatcaagttcgtgcatttttaaataaacttaatgataatgttgtttatttatttgatgaggctggtgatgaagcaatggcctctcgtatgtattgtacttttgctattgaggatgttgaagacgttatcagtagtgaagctgtcgaagatactattgatggtgtcgttgaagacactattaatgacgatgaagatgttgttactggtgacaatgacgatgaagatgttgttactggtgacaatgacgatgaagatgttgttactggtgacaatgacgatgaagatgttgttactggtgacaatgacgatgaagatgttgttactggtgacaatgacgatgaagatgttgttactggtgacaatgacgatgaagatgttgttactggtgacaatgacgatgaagatgttgttactggtgacaatgacgatgaagatgttgttactggtgacaatgacgatgaagatgttgttactggtgacaatgacgatgaagatgttgttactggtgacaatgacgatgaagatgttgttactggtgacaatgacgatgaagatgttgttactggtgacaatgacgatgaagatgttgttactggtgacaatgacgatgaagatgttgttactggtgacaatgacgatgaagacaataaagatgaagagattgttactggtgacaatgatgaccaaattgttgttactggtgatgatgtagatgatattgaaagtatttatgactttgatacttataaagctcttttagtttttaatgatgtctataatgatgctttgtttgttagttatggttctagtgttgaaacagaaacatattttaaagttaatggtttatggtcacctactattacacatactaactgttggttgcgttctgtgttacttgtaatgcagaaattaccttttaagtttaaggatttagctattgaaaatatgtggttatcttataaggtgggttataatcaaagttttgttgattatttactgaccactattcctaaagctattgttttgcctcaaggtggttttgtagctgattttgcttattggtttttaaaccagtttgatattaatgcgtatgctaattggtgttgtttaaaatgtggtttttcttttgatttaaatggtttggatgctttgtttttttatggagatattgtgtctcatgtttgtaagtgtggacataatatgactctaatagcagcggacttaccttgtacattacatttttcattatttgatgacaatttttgtgctttttgcacccctaaaaaaatttttattgctgcatgtgctgtggatgtaaacgtttgtcattctgtagctgttataggtgatgaacaaatagatggtaagtttgttactaaatttagtggtgataaatttgattttatagtaggttatggaatgtcatttagtatgtcttcttttgagttagctcaattgtatggtttgtgtataacacctaatgtatgttttgttaaaggtgatattataaatgttgctagacttgttaaagctgatgttattgttaatcctgctaatgggcatatgctccatggtggtggagttgcaaaagctatagctgtagctgcaggtaaaaaattttctaaagaaactgctgctatggttaaatctaaaggtgtttgccaagtaggagattgttatgtttctaccggtggtaaattatgtaaaacaattcttaatattgtaggccctgatgctagacaagatggaagacaatcttatgttttgttagcacgtgcttataagcatcttaataattatgattgttgtttgtctactctcatatcggctggtatatttagtgttcctgctgatgtgtcattaacttaccttctaggtgttgttgataaacaagttatccttgttagtaataataaagaagattttgatattattcaaaaatgtcaaattacttcagttgttggtactaaagcattggctgttagattaactgctaatgtaggccgtgttattaaatttgagacagatgcatacaaactttttttgagtggtgatgattgttttgtttcaaattcttctgttatacaagaagttttattgcttcgtcatgatatacaattgaataatgacgttcgtgattatttgttgtctaagatgactagtcttcctaaagattggcgtcttatcaataaatttgatgttattaacggtgttaaaactgttaagtattttgagtgtcctaattctatttatatatgtagtcagggtaaagactttggttatgtatgtgatggttctttttataaagcaactgttaatcaagtttgtgttttattagctaagaagatagatgttttgcttactgtagatggtgttaattttaaatctatttctcttactgtaggtgaagtttttggtaaaatacttggtaatgttttctgtgatggcattgatgttactaagttaaagtgtagtgatttttatgccgataaaattttatatcagtatgaaaatttgtctttagctgatatttctgctgtacaaagttcatttgggtttgatcagcaacaattgcttgcttattataattttttaacagtatgtaaatggtctgtagttgttaacggtccatttttttcttttgaacagtctcataataattgttatgtgaatgtagcttgtcttatgttgcagcatattaatcttaaatttaataaatggcagtggcaggaagcatggtatgaatttcgtgctggcagaccacatagattagttgctcttgttttagctaaaggtcattttaaatttgatgaaccatcagatgctactgattttattcgtgttgttttgaaacaagctgatttatcaggtgcaatttgtgaattagaacttatttgtgattgtggtattaaacaagaaagtcgtgttggtgttgatgctgttatgcattttggtacattagcaaagactgatctttttaatggttataagattggctgtaattgtgcaggtagaattgtccattgtactaaattgaatgtaccatttttgatttgttctaatactcctctgagtaaggatttacctgatgatgttgtcgcagctaacatgtttatgggtgtaggtgtaggccattatacacatttgaaatgtggttcaccttaccaacattatgatgcttgtagtgttaaaaaatacacaggtgttagtggttgtttaactgactgcttgtatcttaaaaatttaacccagacttttacatctatgttgactaattattttttggatgatgttgaaatggttgcttataaccctgatctttcacaatattattgtgataatggtaagtattatacaaaacctattataaaggctcagtttaaaccatttgctaaagttgacggtgtttatactaactttaagttagttggacatgatatttgtgctcaattgaatgataagttaggttttaatgtagatttgccgtttgttgagtacaaagtaacagtctggcctgtagctactggtgatgttgttttggcatctgatgatttatatgtgaaacgttattttaaaggatgtgaaacttttggtaagcctgttatttggttttgtcatgatgaagcatcattgaattctcttacttattttaataaacctagttttaaatctgaaaatagatatagtgttttgtctgttgattctgtatctgaggagtcacaaggtaatgtggttactcctgttatggaatcgcagattagtactaaagaggttaagttaaagggtgttagaaagactgttaaaatagaagatgctattattgttaatgatgaaaatagttctattaaggttgttaaaagtttatctttagttgatgtttgggatatgtatttgacaggttgtgattatgttgtttgggttgctaatgaattgtcacgcctagttaaatcaccaacagttagggaatatatacgatatggtattaaacctattactatacctatagatttgttatgtttaagagatgataatcaaactcttttagtccctaaaatttttaaagcaagagctatagaattttatggttttttgaagtggttgtttatttatgtttttagtttattacattttacaaatgataaaaccattttttatactacagaaatagcttctaagtttacttttaatttgttttgtttggctcttaaaaatgcttttcagacatttagatggagtatatttataaaaggttttcttgttgtagccactgtgtttttgttttggtttaattttttgtatataaatgttatttttagtgacttttatcttcctaatattagtgtttttcctatttttgtgggaagaattgttatgtggataaaggctacttttggtttggttacaatttgtgatttttattctaagttaggtgtaggttttacaagtcatttttgtaatggtagttttatatgtgaattgtgtcattctggttttgatatgttggatacatatgcagctatagattttgttcagtatgaagtagatagacgtgttttatttgattatgttagtttagtcaaattaattgttgaactcgttattggttattcattatatacagtatggttttatccattattttgtcttattggtttacaattatttactacatggttgcctgatttgtttatgttagaaactatgcattggttgattagatttattgtatttgtagctaatatgttacctgcttttgtcttgttgcggttttatatagttgttactgctatgtataaagtagttggttttattaggcatattgtctatggttgtaataaagctggttgtttattttgttataaacgaaattgtagtgttcgtgttaagtgtagtactattgttggtggtgtaattcgttattatgatattactgctaatggtggtactggtttttgtgttaaacatcaatggaattgttttaattgccattcttttaaaccaggtaacacttttataactgtagaagctgctatagaactttctaaagagcttaaacgacctgtaaatccaactgatgcttcacattatgtagttactgatattaagcaagttggttgtatgatgcgtttgttctatgatagagatggacagcgtgtctacgatgatgttgatgctagtttatttgtagatattaataatctgttacattctaaagttaaagttgttcctaatttgtatgtagttgtagtagagagtgatgctgatagagctaattttctgaatgctgttgtgttttatgcacaatcattgtataggcctatattacttgtagacaaaaagttaattactacagcttgtaatggtatctctgtaacccagactatgtttgatgtttatgttgatacttttatgtctcattttgatgttgatagaaagagttttaataattttgttaatattgctcatgcttctcttagagagggtgtgcaattagaaaaggttttagatacttttgtgggatgtgtacgtaaatgttgttccattgattcagatgttgaaacaagatttattactaaatctatgatatctgcagtagctgctggtttggaatttactgatgaaaattataacaatttggtacctacatatttaaagagtgataatattgtagctgctgatttaggtgttcttatacagaatggtgctaagcatgtacagggtaatgttgctaaggcagctaatatttcttgtatatggtttattgatgcttttaatcaacttactgctgatttacagcataaattaaaaaaagcatgtgttaaaactggcttgaagttaaaattgacttttaataagcaagaggcaagtgtccctattcttacaacacccttttcacttaaaggaggtgttgtattgagtaatttgttatatatattattttttgttagtttaatctgttttatattattgtgggctttattgcctacatatagtgtttataagtctgatattcatttgcctgcttatgctagttttaaagttattgataatggtgttgttagagatatttcagttaatgatttatgttttgctaataaatttttccaatttgatcaatggtatgagtccacttttgggtctgtttactatcataattctatggattgccctattgtagtggcagttatggatgaagatatcggttctactatgtttaatgttcctactaaagttttgagacatggctttcatgttttacattttttaacttatgcatttgctagtgatagtgttcagtgctatacaccacatattcagatttcttataatgatttttatgctagtggttgtgttttatcatctttgtgtactatgtttaaaagaggtgatggtacaccacatccttattgttattcagatggtgttatgaagaatgcttctttgtatacatctttggttccacatacacgttatagccttgctaattctaatggttttataagatttcctgatgttattagtgaaggtattgtacgtattgtaagaacgcgctctatgacttattgtagagtgggtgcatgtgaatacgccgaagagggtatatgttttaattttaatagttcctgggttttgaataatgattattatagaagtatgcctggaactttttgtggtagagatctttttgatttgttttatcaattttttagtagtttaattcgtcctatagatttcttttctcttactgctagttctatttttggagctatattggctatagttgttgtcttggttttttattatttaataaaacttaagcgtgcttttggagattatactagtgttgtagttataaatgttgttgtttggtgtattaattttcttatgctttttgtttttcaagtttatcctatttgtgcatgtgtttatgcttgtttttatttttatgtaacattgtattttccttctgaaattagtgtaattatgcatttgcaatggattgttatgtatggtgctataatgcctttttggttttgtgtcacatatgtagctatggttattgcaaaccatgttttatggttattttcatattgtaggaaaattggtgttaatgtatgtagtgatagtacatttgaagaaacatctcttactacttttatgattactaaagattcttattgtagattaaagaattctgtttctgatgttgcctacaatagatatttgagtttgtataataagtatcgttactatagtggtaaaatggatactgctgcctatagagaagcggcgtgttctcagttagctaaagctatggaaacatttaatcacaataatggtaatgatgtcttataccaacctcctacagcatctgtttctacatcttttttgcaatcaggtattgtaaagatggtatctcctacgtcaaaaattgaaccttgtattgttagtgttacttatggtagtatgactttgaatggtttatggttagatgacaaagtttattgtcctcgtcatgttatatgttcatcctctaatatgaacgaacctgattattctgccttattgtgtagagttactctaggtgattttactataatgtctggtcggatgagtttaacagttgtgtcttaccagatgcagggctgtcaacttgttttgacagtctctttacaaaatccttatactccaaaatatacttttggtaatgttaaacctggtgaaacttttactgttttagctgcgtataatggccgaccacaaggggcatttcatgttactatgcgtagtagttatactattaaaggttcttttttgtgtgggtcatgtggatctgttggttatgtattaacaggtgatagtgttaagtttgtatatatgcatcaattagagctcagtactggttgtcacactggcactgattttactggtaatttttatggtccatatagagatgctcaagttgtacagttgccagttaaggactatgtccagactgttaatgttattgcttggctctatgcagctatacttaataattgtgcttggtttgtacaaaatgatgtttgttctactgaagattttaatgtttgggctatggcaaatggttttagccaagtaaaagcagatcttgttttagatgctttggcttcaatgacaggtgtttctattgaaaccttattggctgctattaagcgtctatatatgggatttcaaggtcgtcaaatactaggaagttgtacttttgaagatgaattggcaccttctgacgtttatcaacaattggctggtgttaaattgcaatctaaaacaaaaagatttattaaagaaacaatttattggattttgatatctacatttttgtttagttgtataatttctgcatttgttaaatggactatatttatgtatattaatacacatatgattggtgttacattatgtgtactttgttttgttagttttatgatgttactagttaaacataagcatttttatttgactatgtatataattcctgtactctgtaccttgttttatgtaaattatttagttgtttataaggaaggttttagaggttttacttatgtctggctctcatattttgttcctgctgtgaattttacttatgtttatgaagtattttatggttgtattttatgtgtttttgctatttttataactatgcatagtattaatcatgacattttttctttgatgtttttggttggtagaatagttactttaatttctatgtggtattttggttcgaatttagaagaggatgttttgttatttattacagcctttttaggtacttatacatggaccactattttgtcattagctatagcaaaaattgttgctaattggttgtctgttaatatattttattttacagatgtaccttatattaaattgattctcttgagttacttatttatagggtatattttatcttgttattggggatttttctctcttttaaacagtgtttttagaatgcctatgggtgtttataattataaaatttctgttcaagaattgcgttatatgaatgctaatggcttacgtccacctcgtaatagttttgaggctattttgttaaatttaaaactgcttggaataggtggcgtgccagttattgaagtctcccaaattcaatcaaaattgactgatgtgaaatgtgctaatgttgttttgttaaattgtttacagcatttgcatgttgcttctaattctaagttgtggcagtattgtagtgttttacataatgaaatactatctacttcagatttgagtgtagcttttgataagcttgctcaattattgattgttttattcgccaatcctgctgcagttgatactaagtgtcttgcaagtatagatgaagttagcgatgattatgttcaagatagtaccgttttgcaggctttgcaaagtgagtttgtaaatatggctagttttgttgaatatgaagtcgcaaagaaaaatttggctgatgctaaaaatagtggttctgttaatcaacaacagataaaacagttagaaaaagcatgtaatatagctaagtctgtgtatgaacgtgataaagctgtagctcgcaaacttgaacgtatggcagacctagcacttactaacatgtataaagaggctcggattaatgataagaagagtaaagttgtttccgctttgcagacaatgctttttagcatggttcgtaaattggataatcaggctttaaattctattctggataatgctgttaaaggttgtgtacctttgagtgctattccagcattggctgctaatactttaactataataataccagataaacaagtttttgataaagttgttgataatgtttatgttacatatgctggtagtgtatggcatatacagactgttcaagatgctgatggtattaataaacagttaactgatattagtgttgattctaattggcctcttgttatcattgcgaacaggtataatgaagttgctaatgctgttatgcagaataatgagttgatgcctcataaattaaaaatacaagttgttaatagtggttctgatatgaattgtaatattcctactcaatgttattataataatggtagtagtggtagaatagtttatgctgttcttagtgatgttgatggtcttaagtatactaagataatgaaagatgatggaaattgtgttgttttagagcttgatcctccttgtaaattttctatacaagatgttaagggacttaaaattaagtatctttattttattaaaggatgtaacactttagctagagggtgggttgttggtactttatcttcaacaattagattgcaggctggtgttgctactgagtatgcagctaattcttctatactttcattatgtgcattttctgtagatcctaagaaaacttatttagattatatacaacaaggtggtgtacctataattaattgtgttaaaatgctctgtgatcatgctggtactggtatggccattactattaaacctgaggctactattaaccaagattcttatggtggtgcctctgtttgtatttattgccgtgcacgtgtagagcatccagatgtagatggtatatgtaaattacgtggtaaatttgtacaagtccctttgggtataaaagaccctattctttatgtgttaacacatgatgtttgtcaagtctgtggtttttggagagatggcagttgttcctgtgtaggttcaagtgtcgctgttcaatctaaagatttaaattttttaaacgggttcggggtactagtgtgaatgcccggctagtaccctgtgctagtggtttatctactgatgttcaattaagggcatttgatatttgtaataccaatagagctggtataggtttatattataaagtgaattgttgccgttttcagcgtatagatgacgacggtaataaattggataagttctttgttgtcaaaagaactaatttagaagtttataataaagagaaaacttattatgagttgactaaaagttgtggtgttgtggctgaacatgatttctttacatttgatattgatggtagtcgcgtgccacatatagttcgtaggaatctttcaaagtatactatgttagatctttgctatgcattgcgtcattttgatcgtaatgattgttcaatattgtgtgaaattctttgtgagtatgctgattgtaaagaatcctacttttctaagaaagattggtatgattttgttgaaaatcctgatattattaatatatataaaaaattaggccctatttttaatagagctttacttaatactgtcatttttgcagacaccttagttgaagtaggtttagttggtgttttaactttagataaccaagatttgtatggtcaatggtatgattttggtgattttatacaaacagccccaggatttggtgtggcagtcgcagattcttactattcttatatgatgcctatgttgactatgtgtcatgtattagattgtgaattatttgttaatgatagttatagacaattcgatcttgtgcagtatgattttactgattacaagttagagttgtttaataagtattttaagtattggggtatgaagtatcatcctaatactgtggattgtgataatgataggtgtattattcattgtgctaattttaatatactatttagtatggttttacctaatacttgttttggtccccttgttagacaaatttttgtagatggtgtaccgtttgttgtttctattggttaccattacaaagagttaggtgtagttatgaacttagatgttgacacacaccgttatcgtttgtctcttaaagatttacttctttatgcagcagatcctgctatgcatgttgcatctgctagtgctctgcttgatttacgaacttgttgttttagtgtagctgccattacaagtggtataaaatttcaaactgtaaaaccaggtaactttaaccaagacttttacgagtttgttaaaagtaaaggcttgtttaaagagggtagtacagttgatttgaaacattttttctttactcaagatggtaatgctgcaattactgattataattattataagtataatttacctactatggttgatattaagcagttattgtttgtattagaagttgtttataaatattttgaaatttatgatggtggttgtataccagcatcacaagttattgttaataattatgataaaagtgctggttatccatttaataaatttggtaaagccagactttattatgaggcattatcatttgaagaacagaatgaaatttatgcatatactaaacgtaatgttctgcccaccttaactcaaatgaatttaaaatatgctatcagtgctaagaatagagctcgcactgtagcaggtgtttctattcttagtactatgacaggccgaatgttccatcaaaaatgtttgaagagtatagcagctacccgaggtgttcctgttgttataggaaccactaaattttatggtggttgggacgatatgttacgtcatcttataaaggatgttgacaaccctgttcttatgggttgggattatcctaaatgtgatcgtgctatgccaaatattttgcgtattgttagtagtttagttttggcccgcaaacatgaattttgttgttcacatggtgatagattttatcgccttgcgaatgaatgtgctcaagttttgagtgaaatagttatgtgtggcggttgctattatgttaagcctggtggtactagcagtggtgatgcaactactgcttttgctaattctgtttttaatatatgtcaggctgttactgctaacgtttgttctcttatggcctgtaatggccataagattgaagatttaagtatacgcaatttacaaaaacgtttatactctaatgtttatcgtacagattatgttgattatacatttgttaatgagtattatgaatttttatgtaagcattttagtatgatgattttgagtgatgatggtgttgtctgttataactctgattatgctagtaagggttatatagctaatataagtgtttttcaacaagttttgtactatcagaataatgtttttatgtctgaatctaaatgttgggttgaaaatgatattactaatggtcctcatgaattttgttcccaacatactatgttggttaagatagatggtgattatgtttatttaccatatccagatccttctagaattctaggagctggttgttttgttgatgatttattgaagactgacagtgttcttttgatagagcgctttgtaagtctagctatagatgcttaccctttagtatatcatgaaaatgaagaataccaaaaagtctttcgtgtatatttagaatatataaaaaaactgtataatgatcttggtactcagatcttagatagttatagtgttattttaagtacttgtgatggtttaaagtttactgaagaatcattttacaagaatatgtatttaaaaagtgccgtgatgcagagtgtaggtgcatgtgttgtttgttcatcacaaacttctttgcgttgtggcagttgtatacgtaagcctttgttatgttgtaaatgttgttatgaccatgttatggcaactaatcataaatatgttttgagtgtctcaccttacgtttgtaatgcacctaactgtgatgtgagtgatgtcaccaaattatatttgggtggtatgtcttactattgtgaaaaccataaaccccattattcatttaagttagttatgaatggtatggtctttggtttgtataaacaatcttgtacgggttcaccttatatagatgattttaataagatagctagttgtaaatggacagaagttgatgattatgttctggcaaatgagtgtattgaacgtttaaagttatttgctgcagaaactcaaaaggcaactgaagaggcttttaaacaaagctatgcttctgctaccattcaagagattgttagtgatagagaagttattttgtgttgggagacaggtaaagttaaaccaccacttaataaaaattatgttttcacaggctaccattttactagtactggtaagacagttttaggtgagtatgtttttgataaaagtgaattaactaacggtgtgtattaccgcgctacaactacttataaactttctataggtgatgtttttgttttaacatcacattctgtagctagtttaagtgcacctacacttgtcccacaagagaactatgctagtataagattttctagtgtttatagtgttccattggtgtttcaaaataatgttgctaattatcagcacattggaatgaaacgttattgcactgttcaaggtccccctggtacgggaaagtctcatcttgctataggtctagctgtttattactacacagcacgtgtagtttatactgctgctagtcatgctgctgtagatgcattgtgtgaaaaagcttataagtttttaaatattaacgattgtacacgtattattcctgctaaagttcgtgtagattgttatgataagtttaaaattaatgataccacttgtaagtatgtttttaccacaataaatgcattaccagagttggttacagatattgttgttgttgatgaagttagtatgcttactaattatgaattgtctgttataaatgctcgtattaaagctaaacattatgtatatattggagatcctgctcaattacctgcaccacgtgtgctgttgagcaagggttctttagaacctaggcacttcaattctattactaaaataatgtgttgtttaggtcctgatatctttttgggaaattgttataggtgtcctaaagaaattgtagaaactgtttcagcattggtttatgataataaactcaaggctaaaaatgataatagttcattatgttttaaagtatattttaagggacagacaacacatgagagttcaagtgctgtaaatattcaacagatatatctaattagtaaatttttaaaagctaatccagtttggaatagtgctgtttttattagtccttataatagtcagaattatgttgctaagcgtgttttaggtgttcaaacacaaactgtagattctgctcaaggttcggaatatgattacgttatatattcacaaacagcagaaacagcccattctgttaatgttaatcgatttaatgttgccataactagagccaagaagggcattttttgtgttatgagtaatatgcaattatttgaatctcttaattttattactctacctttagataaaattcaaaatcaaactttacctcgtttgcattgcacaactaatctttttaaagattgtagtaaaagttgcttaggttatcatccagcgcatgccccctcatttttagcagttgatgataaatataaggttaatgaaaatttggctgtaaatttaaatatttgtgaacctgttttaacatattctcgtttaatatctcttatgggttttaaattagatttgactcttgatggttattctaaattgtttattactaaagatgaagccattaaacgtgttagaggttgggttggttttgatgttgaaggcgctcatgctactcgcgaaaacattggaacaaactttccactgcaaataggtttttcaactggtgtggattttgtagttgaagctactggcttatttgctgagagagattgttatacttttaaaaaaactgtagctaaagctcctcctggtgaaaaatttaaacatttaataccccttatgtcaaaaggtcaaaagtgggatattgttagaattagaattgttcaaatgttatctgattatcttttagacctttctgatagtgtagtatttattacttggtctgccagttttgaacttacttgtttaaggtattttgctaaattaggcagagagcttaattgtaatgtgtgttctaatcgtgctacatgctacaattctagaactggttattatggttgttggcgccatagttatacttgtgattatgtgtataatccacttattgtagatatacaacagtggggttatacaggttctttaactagtaatcacgatataatttgtaatgtacataaaggtgcacatgttgcgtcagctgatgcaattatgactcgttgtttagcaatctatgattgtttttgtaaatctgttaattggaatttagagtatccaataatttctaatgaggtcagtataaatacatcttgtaggttattgcagcgtgtcatgcttaaagctgccatgctatgtaatagatacaacttatgttatgacataggcaatcctaaaggtttagcttgtgtcaaagattatgaatttaaattttatgatgcttttcctgtagccaagtttgttaaacagttattttatgtctatgatgtgcataaagataattttaaagatggtttatgtatgttttggaattgtaatgttgataaatatccatctaattcaattgtttgtagatttgacactcgagttttaaataaattaaaccttcctggatgtaatggtggtagtttgtatgttaataaacatgcattccatactaatccttttactagaactgtttttgaaaatcttaagcctatgccttttttctattattcagatacgccttgtgtgtacgtagatggtttagaatctaaacaagttgattacgttcctttaagaagcgccacttgtatcacacggtgtaatctaggtggagctgtttgttcaaagcatgctgaagaatattgtaactaccttgagtcttataatatagttactacagcaggctttactttttgggtttataagaattttgatttttataatttatggaacacttttactacgttacagagtttagaaaacgtaatatataacttggttaatgttggtcattatgatggacgtacaggtgaattaccttgtgctattatgaatgacaaagttgttgttaagattaataatgtagatactgttatttttaaaaataatacatcatttcctactaatatagctgttgaattgtttacaaaacgtagtatccggcaccaccctgaacttaagattcttagaaatttgaacattgatatttgttggaagcatgtcctgtgggattatgttaaagatagtttgttttgtagttccacttatggtgtttgtaaatacacagatttgaagttcatcgaaaatttgaatatactttttgatggtcgtgacactggcgctttagaagcttttagaaaagcaagaaatggtgtttttattagtactgaaaaattaagtaggttatcaatgattaaaggtccgcaacgagctgatttaaatggtgtgattgtggataaagttggagaactcaaagttgagttttggttcgctatgagaaaagatggtgacgatgttatcttcagccgaacagacagcctatgctcaagccattactggagcccacaaggtaatctaggtggtaattgcgcgggtaatgtcattggtaatgatgctctaacacgttttactatctttactcagagtcgtgtattgtcaagttttgaacctcgctcagatttagaacgggattttattgatatggatgataatctgtttattgctaaatatggtttagaagactatgcatttgatcatatagtttatggtagttttaaccataaagttataggaggtttgcatttgcttataggcttatttcgtaggctaaaaaaatctaatttgttaattcaagagtttttacagtatgattctagtattcattcatattttattactgatcaggagtgtggtagtagtaagagtgtttgtacagttattgatttattattagatgattttgtttctattgttaagtcattaaatttgagttgtgttagtaaagttgttaatattaatgttgattttaaggattttcaatttatgttgtggtgtaatgataataaaattatgactttttatcctaaaatgcaagccactaatgattggaaacctggctattctatgcctgttttgtataagtatttgaatgttccattagagagagtctctttatggaattatggtaaacctattaatttgcctacaggctgtatgatgaatgttgctaagtacactcaattatgtcagtatttgaatactacaacattagctgttcctgttaatatgcgtgttttacatttaggtgcagggtctgataaagaagtagctccaggttctgctgttttaagacagtggttaccatctggtagtattcttgtagataatgatctaaacccatttgttagcgatagtttagttacttattttggagattgtatgactttaccatttgattgtcattgggatttgataatatctgatatgtatgatcctcttactaaaaatattggtgattataatgtgagtaaggatggtttttttacttacatttgtcatttaattcgtgataaattatctttgggtggtagtgtagctataaaaattacagagttttcttggaatgctgatttatataaattaatgagttgttttgcattttggacagttttttgtactaatgtaaatgcttcttctagtgaagggtttttaataggtataaattacctgggtaaatcttcttttgaaatagatggcaatgttatgcatgctaactatttgttttggagaaatagtacaacatggaatggcggtgcttatagtttatttgatatgactaaattttctttgaaattggctggcactgctgttgttaatttaagaccagatcaattaaatgatttagtttattctcttattgaaagaggtaaattattagttcgcgatacgcgtaaagagatttttgttggtgatagtcttgtaaatacttgttagatctcattaaatctaaactatgttaattatttttttatttttttatttctgttatggttttaatgaacctcttaatgttgtgtctcatttaaaccatgactggtttttatttggtgatagtcgttctgattgtaaccatattaataatttaaaaattaaaaattttgattatttggatattcaccctagtttgtgcaacaatggtaagatttcatctagtgccggtgattctatttttaagagttttcatttcactcgattttataattacactggcgaaggtgatcaaattattttttatgagggtgttaattttaatccttatcatagatttaagtgttttcccaatggtagtaatgatgtatggcttcttaacaaggtaagattttatcgtgccttatattctaatatggccttttttcgttatcttacttttgttgatattccttataatgtttctctttctaagtttaattcttgtaaaagtgatattttatcacttaacaatcctatttttattaattattctaaggaagtttattttactttattaggttgttctctttatttagtaccgctttgcctttttaaatctaactttagtcagtactattataacatagatactggctctgtttatggtttttctaatgttgtttatcctgatttagactgtatttatatttctcttaaaccaggttcttataaagtttccaccactgcaccttttttatccttacctactaaagctctctgttttgataaatctaaacaatttgtacctgtacaggttgttgattctagatggaacaacgagcgtgcctcagatatttctttatctgttgcatgtcaattgccatattgttattttcgcaattcttctgctaattatgttggcaagtatgatattaaccacggtgatagtggttttatttctattttatctggtcttttatataatgtttcttgtatttcatattatggtgtatttctatatgataattttacatccatttggccctattattcttttggtaggtgtcctacatcttctattattaaacatccaatttgtgtttatgattttttgcctattattttacaaggtattttattatgtttagctttactttttgttgtttttctattattcttgttatataacgatagatctcattaaatctaaacatgttattaattatttttattttgcctacaacattagctgttataggtgattttaattgtactaattttgctattaatgataaaaacaccacagttcctcgcataagtgagtatgttgtggatgtttcttatggtttgggtacatattatatacttgatcgtgtttatttaaatactattatattatttactggttatttccctaaatctggtgccaattttagggatctatctttaaaaggtactacatatttgagtactctttggtatcagaaaccctttttatctgattttaataatggtattttttctagagttaagaatactaagttgtatgttaataaaactttgtatagtgagtttagtactatagttataggtagtgtttttattaacaactcttatactattgttgttcaacctcataatggtgttttggagattacagcttgtcaatacactatgtgtgagtatcctcatactatttgtaaatctaaaggtagttctcgtaatgaatcttggcattttgataaatctgaacctttgtgtctgttcaagaaaaattttacttataatgtttctacagatttcttgtattttcatttttatcaagaacgtggcactttttatgcttattatgctgactctggcatgcctactacttttttatttagtttgtatcttggtactcttttatctcattattatgttttgcctttgacttgtaatgctatatcttctaatactgataatgagactttacaatattgggtcacacctttgtctaaacgccaatatcttcttaaatttgacaaccgtggtgttattactaatgctgttgattgttctagtagtttctttagtgagattcaatgtaaaactaaatctttattacctaatactggtgtttatgacttatctggttttactgttaagcctgttgcaactgtacatcgtcgtattcctgatttacctgattgtgacattgataaatggcttaacaattttaatgtaccctcacctcttaattgggaacgtaaaattttttctaattgcaactttaatttgagtactttgcttcgtttagttcatactgattctttttcttgtaataattttgatgaatctaagatatatggtagttgttttaagagtattgttttagataaatttgccatacccaactccagacgatctgatttgcagttgggcagttctggttttctgcaatcttctaattataaaattgacactacttctagttcttgtcaattgtattatagtttgcctgcaattaatgttactattaataattataatccttcttcttggaatagaaggtatggttttaataattttaatttgagctctcatagtgttgtttactcacgttattgtttttctgttaataatactttttgtccttgtgctaaatcttcttttgcttcaagttgcaagagtcataaaccaccttctgcttcctgtcctattggtactaattatcgttcttgtgagagtactactgtactcgaccacactgactggtgtaggtgttcttgtttacctgatcctataactgcttatgaccctaggtcttgttctcaaaaaaagtctctggttggtgttggtgaacattgtgcagggttcggtgttgatgaagaaaagtgtggtgtattggatggatataatgtttcttgtctttgtagtactgatgcctttctaggttggtcttatgacacttgcgtcagtaacaaccgttgtaatattttttctaattttattttaaatggtatcaatagtggtaccacttgttctaatgatttattgcagcctaatactgaagtttatactgatgtttgtgttgattacgacctttatggtattacaggacaaggtatttttaaagaagtttctgctgtttattataatagttggcaaaatcttttgtatgattctaatggcaacattattggttttaaagattttgttactaataaaacatataatattttcccttgttatgcaggaagagtttctgctgcttttcatcaaaatgcttcctctttggctttactttatcgtaatttaaaatgtagctatgttttgaataatatttctttagctactcagccatattttgatagttatcttggttgcgtttttaatgctgataatttaactgattattctgtttcttcttgtgctcttcgcatgggtagtggtttttgtgttgattataactcaccttctttttcctcttcgcgtcgtaaacgtagaagtatttctgcttcttatcggtttgttacttttgaaccctttaatgtcagttttgttaatgacagtattgagtctgtgggtggtctttatgagatcaaaattcccactaactttactatagttggtcaagaggaatttattcaaactaattctcctaaagttactattgattgttctttatttgtctgttctaattatgcagcttgccatgacttattgtcagagtatggcactttttgtgataatattaatagtattttagatgaagttaatggtttacttgatactactcaattgcatgtagctgatactcttatgcaaggtgtcacacttagctccaatcttaatactaatttgcattttgatgttgataatattaattttaaatccctagttggatgtttaggtccacactgcggttcttcttctcgttctttttttgaagatttattgtttgacaaagttaaactttcagatgttggttttgttgaagcttataacaattgtactggtggtagtgaaattagagatcttctttgtgtacaatcctttaatggtattaaagttttgcctcctattttgtctgaatctcaaatttctggttacaccacagccgctactgttgctgctatgtttccaccatggtcagcagcagctggcataccattttctcttaatgtacaatatagaattaatggtttgggtgttactatggatgttcttaataaaaatcaaaagttgatagctactgcttttaataatgctcttctttctattcagaatggttttagtgctaccaactctgcacttgctaaaatacaaagtgttgttaattctaatgctcaagcacttaatagtttgttacagcaattatttaataaatttggtgcaattagttcttctttacaagaaattttatctcgtctcgatgctttagaggctcaggttcagattgataggcttattaatggtcgtttaactgctttaaatgcttatgtctctcaacaacttagtgatatttctcttgtaaaatttggtgctgctttagctatggagaaggttaatgagtgtgttaaaagtcaatctcctcgtattaatttttgtggtaatggtaatcatattttgtcattagttcaaaatgctccttatggtttgttgtttatgcattttagttataaacctatttcttttaaaactgttttagtaagtcctggtttgtgtatatcaggtgatgtaggtattgcacctaaacaagggtattttattaaacacaatgatcattggatgttcactggtagttcttactattatcctgaaccaatttcagataaaaatgttgtttttatgaatacttgttctgttaattttactaaagcgcctcttgtttatttgaatcattctgtaccaaaattgtctgattttgaatctgagttatctcattggtttaaaaatcaaacatccattgcgcctaatttgactttaaatcttcatactattaatgctacttttttagatttgtattatgagatgaatcttattcaagagtctattaagtctttgaataatagttatatcaatcttaaagatataggtacatatgaaatgtatgtaaaatggccttggtatgtttggctactaatttctttttcatttataatattccttgtattgctcttttttatatgttgttgtactggttgtggttctgcatgttttagtaaatgtcataattgttgtgatgagtatggtggtcatcatgattttgttatcaaaacatctcatgatgattagaatctcttgtcagatctcattaaatctaaactttatttatggacgtttggagacctagctacacacattctcttgttattagagaatttggtgttacaaaccttgaagatttgtgtttaaagtataattactgtcaacctattgttggttactgtattgtacctttaaatgtttggtgtcgcaagtttggcaaatttgcatctcactttacattacgtagtcacgatatttcccatagtaataattttggtgttgtaactagttttactacttatggtaatactgtttctgaggctgtgtctagattagttgaatcagcttctgaatttattgtttggcgtgcagaggcacttaataagtatggttgatttatttttcaatgatactgcttggtacataggacagattttagttttagttttattttgtcttatttctttaatctttgttgttgcttttttagcaactattaagctttgtatgcaactttgtggtttttgtaatttctttattatttcaccttcggcttacgtttataaaagaggtatgcagttgtataagtcttatagtgaacaagttataccacccacttcagattatttaatctaaatctaaacattatgaataaatctttttttcctcaatttacttctgatcaagctgttacattcttaaaagaatggaatttctctttgggtgtaatactactttttattactatcatattgcagttcggttatacgagccgtagtatgtttgtttattttatcaagatgattattctttggcttatgtggccattgactatcaccttgactatatttaattgcttttatgctctgaataatgcttttcttgcattttctatagtgtttactattatttctattgttatatggattctttattttgttaatagtattcggctttttattagaactggcagttggtggagttttaatccagagaccaataatcttatgtgtattgatatgaaaggcaagatgtttgttaggccagttattgaggactatcacacattaactgctactgttattcgtggtcatctttatatacagggtgtcaaacttggcactggttatactctttcagatttgcccgtatatgttactgtagctaaggtgcaagtactttgtacctataaacgtgcctttttagataagttagatgttaatagtggttttgctgtttttgttaagtctaaagttggtaactatcgtttaccgtctagtaaacctagtggtatggatactgccttgttaagagcttaaatctaaactattaggatgtcttatactcccggtcattatgctggaagtagaagctcctctggaaatcgttcaggaatcctcaagaaaacttcttgggctgaccaatctgagcgaaattaccaaacctttaatagaggcagaaaaacccaacctaaattcactgtgtctactcaaccacaaggaaatactatcccacattattcctggttctccgggatcactcaatttcaaaaaggtagagactttaaattttcagatggtcaaggagttcccattgctttcggagtacccccttctgaagcaaaaggatattggtatagacacagccggcgttcttttaaaacagctgatggtcaacaaaagcagttgttaccgagatggtatttctactatctcggtaccggcccatatgccaatgcatcctatggtgaatccctcgaaggggtcttctgggttgctaatcaccaagctgacacttctactccctccgatgtttcgtcaagggatcctactactcaagaagctatccctactaggtttccgcctggtacgattttgcctcaaggctattatgttgaaggctcaggaaggtctgcttctaatagtcgaccaggttcacgttctcaatcacgtggacccaatactcgttcattaagtagaagtaattctaattttagacattcagattctatagtaaaacctgatatggctgatgagatcgctaatcttgttttagccaagcttggtaaagattctaaacctcagcaagtcactaagcaaaatgccaaggaaatcaggcataaaattttaactaaacctcgccaaaagcgaactcctaataaacattgtaatgttcaacagtgttttggtaaaagaggaccttctcaaaactttggtaatgctgaaatgttaaagcttggtactaatgatcctcagtttcctattcttgcagaattagctcctacaccaggtgcttttttctttggttctaaattagagttggttaaaagagagtccgaggctgactcacctgttaaagatgtttttgaacttcgttattctggttctattaggtttgatagtactttacctggttttgagacaattatgaaagttcttaaagagaatttagatgcttatgttaattctaatcagaacactgtttctggttcgctgagtcctaaacctcagcgtaaaagaggtgttaaacaatcacctgaattgtttgactctcttaatttaagtgctgatactcagcacatttcaaatgattttactcctgaggatcatagtttacttgctactcttgatgatccttatgtagaagactctgttgcttaatgagaatgaatcctaattcgacactaggtggtaacccctcgctattagtcggaataggacactctctatcagaatgaattcttgctgtaataacagatagagtaggttgttacagactatatattaattagtagaaattttatatttagacatttgattgttagagtagttataaggtttagctgtagtataaacgcctccgggaagagctatcaattgtagtgtttaatatatatattagtatatgattgaaatta

SEQ ID NO: 3 - DNA, 27553 bp, Human coronavirus
  ref <308>: DATABASE ACCESSION NUMBER: AY567487.2
  ref <309>: DATABASE ENTRY DATE: 2004-06-22
  ref <313>: RELEVANT RESIDUES IN SEQ ID NO: (1)..(27553)
cttaaagaatttttctatctatagatagagaattttcttatttagactttgtgtctactcttctcaactaaacgaaatttttctagtgctgtcatttgttatggcagtcctagtgtaattgaaatttcgtcaagtttgtaaactggttaggcaagtgttgtattttctgtgtctaagcactggtgattctgttcactagtgcatacattgatatttaagtggtgttccgtcactgcttattgtggaagcaacgttctgtcgttgtggaaaccaataactgctaaccatgttttacaatcaagtgacacttgctgttgcaagtgattcggaaatttcaggttttggttttgccattccttctgtagccgttcgcacctatagcgaagccgctgcacaaggttttcaggcatgccgttttgttgcttttggcttacaggattgtgtaaccggtattaatgatgatgattatgtcattgcattgactggtactaatcagctctgtgccaaaattttacctttttctgatagaccccttaatttgcgaggttggctcattttttctaacagcaattatgttcttcaggactttgatgttgtttttggccatggtgcaggaagtgtggtttttgtggataagtacatgtgtggttttgatggtaaacctgtgttacctaaaaacatgtgggaatttagggattactttaataataatactgatagtattgttattggtggtgtcacttatcaactagcatgggatgttatacgtaaagacctttcttatgaacagcaaaatgttttagccattgagagcattcattaccttggtactacaggtcatactttgaagtctggttgcaaacttactaatgctaagccgcctaaatattcttctaaggttgttttgagtggtgaatggaatgctgtgtatagggcgtttggttcaccatttattacaaatggtatgtcattgctagatataattgttaaaccagttttctttaatgcttttgttaaatgcaattgtggttctgagagttggagtgttggtgcatgggatggttacttatcttcttgttgtggcacacctgctaagaaactttgtgttgttcctggtaatgtcgttcctggtgatgtgatcatcacctcaactagtgctggttgtggtgttaaatactatgctggcttagttgttaaacatattactaacattactggtgtgtctttatggcgtgttacagctgttcattctgatggaatgtttgtggcatcatcttcttatgatgcactcttgcatagaaattcattagaccctttttgctttgatgttaacactttactttctaatcaattacgtctagcttttcttggtgcttctgttacagaagatgttaaatttgctgctagcactggtgttattgacattagtgctggtatgtttggtctttacgatgacatattgacaaacaataaaccttggtttgtacgcaaagcttctgggctttttgatgcaatctgggatgcttttgttgccgctattaagcttgtaccaactactactggtgttttggttaggtttgttaagtctattgcttcaactgttttaactgtctctaatggtgttattattatgtgtgcagatgttccagatgcttttcaatcagtttatcgcacatttacacaagctatttgtgctgcatttgatttttctttagatgtatttaaaattggtgatgttaaatttaaacgacttggtgattatgttcttactgaaaacgctcttgttcgtttgactactgaagttgttcgtggtgttcgtgatgctcgcataaagaaagccatgtttactaaagtagttgtaggtcctacaactgaagttaagttttctgttattgaacttgccactgttaatttgcgtcttgttgattgtgcacctgtagtttgccctaaaggtaagattgttgttattgctggacaagcttttttctatagtggtggtttttatcgttttatggttgatcctacaactgtattaaatgatcctgtttttactggtgatttattctacactattaagtttagtggttttaagcttgatggttttaaccatcagtttgttactgctagttctgctacagatgccattattgctgttgagctgttgttattggattttaaaactgcagtttttgtgtacacatgtgtggttgatggctgtagtgtcattgttagacgtgatgctacattcgctacacatgtgtgttttaaggactgttataatgtttgggagcaattctgcattgataattgtggtgagccatggtttttgactgattataatgctatcttgcagagtaataaccctcaatgtgctattgttcaagcatcagagtctaaagttttgcttgagaggtttttacctaagtgtcctgaaatactgttgagtattgatgatggccatttatggaatctttttgttgaaaagtttaattttgttacagattggttaaaaactcttaagcttacacttacttctaatggtcttttaggtaattgtgccaaacgttttagacgtgttttggtaaaattgcttgatgtctataatggttttcttgaaactgtctgtagtgtcgcatacactgctggtgtttgcatcaaatattatgctgttaatgttccatatgtagttattagtggttttgtaagtcgtgtaattcgtagagaaaggtgtgacatgacttttccttgtgttagttgtgtcacctttttctatgaatttttagacacttgttttggtgttagtaaacctaatgccattgatgttgaacatttagagcttaaagaaactgtttttgttgaacctaaggatggtggtcaattttttgtttctggtgattatctttggtatgttgtagatgacatttattatccagcttcatgtaatggtgtattgcctgttgcttttacaaaattagctggtggtaaaatatctttttctgatgatgttatagttcatgatgttgaacctacccataaagtcaagctcatatttgagtttgaagatgatgttgttaccagtctttgtaagaagagttttggtaagtccattatttatacaggtgattgggaaggtctacatgaagttcttacatctgcaatgaatgtcattgggcaacatattaagttgccacaattttatatttatgatgaagagggtggttatgatgtttctaaaccagttatgatttcacaatggcctattagtaatgatagtaatggttgtgttgttgaagcgagcactgattttcatcaattagaatgtattgttgatgactctgttagagaagaggttgatataattgaacaaccttttgaagaagttgaacatgtgctctcaattaagcaacctttttctttttcttttagagatgaattgggtgttcgtgttttagatcaatctgataataattgttggattagtaccacacttgtacagttgcaacttacaaagcttttggatgattctattgagatgcaattgtttaaagttggtaaagttgattcaattgtccaaaagtgttatgagttgtctcatttaattagtggttcacttggtgatagtggtaaacttcttagtgaacttcttaaagaaaaatatacatgttctataacttttgagatgtcttgtgattgtggtaaaaagtttgatgatcaggttggttgtttgttttggattatgccttacacaaaactttttcaaaaaggtgagtgttgtatttgtcataaaatgcagacttataagcttgttagtatgaaaggtactggtgtgtttgtacaggatccagcacctattgacattgatgctttccctgtgaaacctatatgttcatctgtatatttaggtgttaagggttctggtcattatcaaacaaatttatacagttttaacaaagctattgatggttttggtgtctttgacattaaaaatagtagtgttaatactgtttgttttgttgatgttgattttcatagtgtagaaatagaagctggtgaagttaaaccttttgctgtatataaaaatgttaaattttatttaggtgatatttcacaccttgtaaactgtgtttcttttgactttgttgtcaatgctgctaatgaaaatctcttgcatggaggcggtgttgcacgtgctattgatattttgactgaaggtcaacttcagtcattatctaaagattacattagtagtaatggtccacttaaggttggagcaggtgttatgttggagtgtgaaaaattcaacgtatttaatgttgttggtccgcgaactggtaaacatgagcattcattacttgttgaagcttataattctattttatttgaaaatggtattccacttatgcctcttcttagttgtggtatttttggtgtaaggattgaaaattctcttaaagctttgtttagttgtgacattaataaaccattgcaagtttttgtttattcttcaaatgaagaacaagctgttcttaagtttttagatggtttagatttaacaccagtcattgatgatgttgatgttgttaaaccttttagagttgaaggtaatttttcattctttgattgtggtgtcaatgccttggatggtgatatttacttattatttactaactctattttaatgttggataaacaaggacaattattggacacaaaacttaatggtattttgcaacaggcagctcttgattatcttgctacagttaaaactgtaccagctggtaatttggttaaactttttgttgagagttgtaccatttatatgtgtgttgtaccatcgataaatgatctttcttttgataaaaatcttggtcgttgtgtgcgtaaacttaatagattgaaaacttgtgttattgccaatgttcctgctattgatgttttgaaaaagcttctttcaagtttgactttaactgttaaatttgttgtagagagtaatgttatggatgttaacgactgttttaagaatgataatgtagttttgaaaattactgaagatggtattaatgttaaagatgttgttgttgagtcttctaagtcacttggtaaacaattgggtgttgtgagtgatggtgttgactcttttgaaggtgttttacctattaatactgatactgtcttatctgtagctccagaagttgactgggttgctttttacggttttgaaaaggcagcactttttgcttctttggatgtaaagccatatggttaccctaatgattttgttggtggttttagagttcttgggaccaccgacaataattgttgggttaatgcaacttgtataattttacagtatcttaagcctacttttaaatctaagggtttaaatgttctttggaacaaatttgttacaggtgatgttggaccttttgttagttttatttattttataactatgtcttcaaagggtcaaaagggtgatgctgaagaggcattatctaaattgtcagagtatttgattagtgattctattgttactcttgaacaatattcaacttgtgacatttgtaaaagtactgtagttgaagttaaaagtgctattgtctgtgctagtgtgcttaaagatggttgtgatgttggtttttgtccacacagacataaattgcgttcacgtgttaagtttgttaatggacgtgttgttattaccaatgttggtgaacctataatttcacaaccttctaagttgcttaatggtattgcttatacaacattttcaggttcttttgataacggtcactatgtagtttatgatgctgctaataatgctgtctatgatggtgctcgtttattttcttcagatttgtctactttagctgttacagctattgttgtagtaggtggttgtgtaacatctaatgttccaacaattgttagtgagaaaatttctgttatggataaacttgatactggtgcacaaaaatttttccaatttggtgattttgttatgaataacattgttctgtttttaacttggttgcttagtatgtttagtcttttacgtacttctattatgaagcatgatattaaagttattgccaaggctcctaaacgtacaggtgttattttgacacgtagttttaagtataacattagatctgctttgtttgttataaagcagaagtggtgtgttattgttactttgtttaagttcttattattattatatgctatttatgcacttgtttttatgattgtgcaatttagtccttttaatagtcttttatgtggtgacattgtaagtggttatgaaaaatccacttttaataaggatatttattgtggtaattctatggtttgtaagatgtgtttgttcagttatcaagagtttaatgatttggatcatactagtcttgtttggaagcacattcgtgatcctatattaatcagtttacaaccatttgttatacttgttattttgttaatttttggtaatatgtatttgcgttttggacttttatattttgttgcacaatttattagtacttttggttctttcttaggctttcatcagaaacagtggtttttacattttgtgccgtttgatgttttatgtaatgagtttttagctacatttattgtctgcaaaatcgttttatttgttagacatattattgttggctgtaataatgctgactgtgtagcttgttctaaaagtgctagacttaaacgtgtaccacttcaaactattattaatggtatgcataaatcattctatgttaatgctaatggtggtacttgtttctgtaataaacataacttcttttgtgttaattgtgattcttttgggcctggtaatacttttattaatggtgatattgcaagagagcttggtaatgttgttaaaacagctgttcaacccacagctcctgcatatgttattattgataaggtagattttgttaatggattttatcgtctttatagtggtgacactttttggcggtatgactttgacattactgaatctaagtatagttgtaaagaggttctgaagaattgtaatgttttagaaaattttattgtttacaataatagtggtagtaacattacacagattaaaaatgcttgtgtttatttttctcaattgttgtgtgaacctataaagttggtaaattcagagttgttgtcaactttatctgttgattttaatggtgttttgcataaggcatatgttgatgttttgtgtaatagtttttttaaggagttaactgctaacatgtccatggctgaatgtaaagctacacttggtttgactgtttctgatgatgattttgtttcagctgttgccaatgcacataggtatgacgttttgctttcagatttgtcatttaataatttttttatttcttatgctaaacctgaagataagttgtccgtttatgacattgcttgttgtatgcgtgccggttctaaggttgttaaccataatgttttaattaaagagtcaatacctattgtttggggtgtcaaggactttaatactctttctcaagaaggtaagaagtaccttgttaaaacaactaaagcaaagggtttgacttttttattaacttttaatgataaccaagcaattacacaagttcctgctactagtatagttgcaaaacagggtgctggttttaaacgtacttataattttctgtggtatgtatgtttatttgttgttgcattgtttattggtgtctcatttattgattatacaaccactgtaactagctttcatggttatgattttaagtacattgagaatggtcagttgaaggtgtttgaagcacctttacactgtgttcgtaatgtttttgataattttaatcaatggcatgaggctaagtttggtgttgttactactaatagtgataaatgtcctatagttgttggtgtttcagagcgtattaatgttgttcctggtgttccaacaaatgtatatttggtaggaaagactcttgtttttacattacaggctgcttttggaaacacaggtgtttgttatgactttgatggtgttaccactagtgataagtgtatttttaattctgcttgtactaggttggaaggtttgggtggtgacaatgtttattgttacaacactgatcttattgaaggttctaaaccttatagtactttacagcccaatgcgtattataagtatgatgctaaaaattatgtacgttttccagaaattttagctagaggttttggcttacgtactattagaactttggctacacgttattgtagagttggtgaatgccgtgactcacataaaggtgtttgttttggttttgataaatggtatgttaatgatggacgtgttgatgacggttacatttgtggtgatggtcttatagaccttcttgttaatgtactctcaatctttagttcatcttttagcgttgtggctatgtctggacatatgttgtttaattttctttttgcagcatttattacatttttgtgctttttagttactaaatttaaacgtgtttttggtgatctttcttatggtgtttttactgttgtttgtgcaactttgattaataacatttcttatgttgttactcaaaatttattttttatgttgctttatgctattttgtattttgtttttactaggacagtgcgttatgcttggatttggcatattgcatacattgttgcatacttcttgttaataccatggtggcttctcacatggtttagttttgctgcatttttagagcttttacctaatgtttttaagttaaaaatctctactcaattgtttgaaggtgataagtttataggtacttttgagagtgctgctgcaggtacatttgttcttgacatgcgttcttatgaaaggctgataaatactatttcacctgagaaacttaagaattatgctgcaagttataataaatataaatattatagtggtagtgctagtgaggctgattatcgttgtgcttgttatgctcatttagccaaggctatgttagattatgcaaaagatcataatgacatgttatattctccacctactattagctacaattccaccttacaatctggtcttaagaagatggcacaaccatctggttgtgttgagagatgtgtggttcgcgtctgttatggtagtactgtgcttaatggagtttggttaggtgacactgttacttgtcctagacatgtcatagcaccatcaaccactgttcttattgattatgatcatgcatatagtactatgcgtttgcataatttttcagtgtctcataatggtgtcttcttgggagttgtcggtgttacaatgcatggttctgtgttgcgtattaaggtttcacaatctaatgtacatacacctaaacatgtttttaaaacgttgaaacctggtgattcttttaatattttagcatgttatgaaggtattgcatctggtgtttttggtgttaatttacgtacaaactttactattaaaggttcttttataaatggagcttgtggttctcctggttataatgttagaaatgatggtactgttgagttttgttatttacaccaaattgagttaggtagtggtgctcatgttggttctgattttactggtagtgtttatggtaattttgatgaccaacctagtttgcaagttgagagtgccaaccttatgctatcagataatgttgttgcctttttgtatgctgctttgttgaatggttgtaggtggtggttgtgttcaactagagttaatgttgatggttttaatgaatgggctatggctaatggttatacaagtgtttctagtgttgagtgctattctattttggcagcaaaaactggtgttagtgttgaacaattgttagcttccattcaacatcttcatgaaggttttggtggtaaaaacatacttggttattctagtttatgtgatgagttcacactagctgaagttgtgaagcagatgtatggtgttaacttgcaaagtggtaaggttatttttggtttaaaaacaatgtttttatttagcgttttcttcacaatgttttgggcagaactctttatttatacaaacactatatggataaaccctgtgatacttacacctatattttgtctacttttgtttttgtcattagttttaactatgtttcttaaacataagtttttgtttttgcaagtatttttattacctactgttattgcaactgctttatataattgtgttttggattattacatagtaaaatttttggctgaccattttaactataatgtttcagtattacaaatggatgttcagggtttagttaatgttttggtctgtttatttgttgtatttttacacacatggcgcttttctaaagaacgttttacacattggtttacatatgtgtgttctcttatagcagttgcttacacttatttttatagtggtgactttttgagtttgcttgttatgtttttatgtgctatatctagtgattggtacattggtgccattgtttttaggttgtcacgtttgattgtatttttttcacctgaaagtgtatttagtgtttttggtgatgtgaaacttactttagttgtttatttaatttgtggttatttagtttgtacttattggggcattttgtattggttcaataggttttttaaatgtactatgggtgtttatgattttaaggtgagtgctgctgaatttaaatacatggttgctaatggacttcatgcaccacatggaccttttgatgcactttggttatcattcaaactacttggtattggtggtgaccgttgtataaaaatttcaactgtccaatccaaactgactgatttgaagtgtactaatgttgtgttattgggttgtttgtctagtatgaacattgcagctaattctagtgaatgggcttattgtgttgatttacacaataagattaatctttgtgatgaccctgaaaaagctcaaagtatgttgttagcactccttgcgttctttctaagtaaacatagtgattttggtcttgatggccttattgattcttattttgataatagtagcacccttcagagtgttgcttcatcatttgttagtatgccatcatatattgcttatgaaaatgctagacaagcttatgaggatgctattgctaatggatcttcttctcaacttattaaacaattgaagcgtgccatgaatatcgcaaagtctgaatttgatcatgagatatctgttcagaagaaaattaatagaatggctgaacaagctgctactcagatgtataaagaagcacgctctgttaatagaaaatctaaagttattagtgctatgcactctttactttttggaatgttaagacgtttggatatgtctagtgttgaaactgttttgaatttagcacgtgatggtgttgtgccattgtcagttatacctgcaacttcagcttctaaactaactattgttagtccagatcttgaatcttattctaagattgtttgtgatggttctgttcattatgctggagttgtttggacacttaatgatgttaaagacaatgatggtagacctgttcatgttaaagagattacaaaggaaaatgttgaaactttgacatggcctcttatccttaattgtgaacgtgttgttaaacttcaaaataatgaaattatgcctggtaaacttaagcaaaaacctatgaaagctgagggtgatggtggtgttttaggtgatggtaatgccttgtataatactgagggtggtaaaacttttatgtacgcttatatttctaataaagctgaccttaaatttgttaagtgggagtatgagggtggttgcaacacaatcgagttagactctccttgtcgatttatggtcgaaacacctaatggtcctcaagtgaagtatttgtattttgttaaaaatttaaataccttacgtagaggtgccgttcttggttttataggtgccacaattcgtctacaagctggtaaacaaactgaattggctgttaattctggacttttaactgcttgtgctttttctgttgatccagcaactacttacttggaagctgttaaacatggtgcaaaacctgtaagtaattgtattaagatgttatctaatggtgctggtaatggtcaagctataacaactagtgtagatgctaacaccaatcaagattcttatggtggagcgtctatttgtttgtattgtcgggcccacgttcctcaccctagtatggatggttactgtaagtttaagggtaaatgtgttcaggttcctattggttgtttggatcctattaggttttgtttagaaaataatgtgtgtaatgtttgtggttgttggttgggacacgggtgtgcttgtgaccgtacaactattcaaagtgttgacatttcttatttaaacgagcaaggggttctagtgcagctcgactagaaccctgcaatggcacggacatcgataagtgtgttcgtgcttttgacatttataataaaaatgtttcattcttgggtaagtgtttgaagatgaactgtgttcgttttaaaaatgctgatcttaaggatggttattttgttataaagaggtgtactaagtcggttatggaacacgagcaatccatgtataacctacttaacttttctggtgctttggctgagcatgatttctttacttggaaagatggcagagtcatttatggtaatgttagtagacataatcttactaaatatactatgatggacttggtctatgctatgcgtaactttgatgaacaaaattgtgatgttctaaaagaagtattagttttaactggttgttgtgacaattcttattttgatagtaagggttggtatgacccagttgaaaatgaagatatacatagagtttatgcatctcttggcaaaattgtagctagagctatgcttaaatgcgttgctctatgcgatgcgatggttgctaaaggtgttgttggtgttttaacattagataaccaagatcttaatggtaacttttatgattttggtgattttgttgttagcttacctaatatgggtgttccctgttgtacatcatattattcttatatgatgcctattatgggtttaactaattgtttagctagtgagtgttttgtcaagagtgatatttttggtagtgattttaaaacttttgatttgcttaagtatgatttcactgaacataaagaaaatttattcaataagtactttaagcattggagttttgattatcatcctaattgtagtgactgttatgatgatatgtgtgttatacattgtgctaattttaatacactatttgccacaactataccaggtactgcttttggtccactatgtcgtaaagtttttatagatggtgttccacttgttacaactgctggttatcattttaagcaattaggtttggtttggaataaagatgttaacacacactcagttaggttgacaattactgaacttttgcaatttgtcaccgacccttccttgataatagcttcttccccagcactcgttgatcaacgcactatttgtttttctgttgcagcattgagtactggtttgacaaatcaagttgttaagccaggtcattttaatgaagagttttataactttcttcgtttaagaggtttctttgatgaaggttctgaacttacattaaaacatttcttcttcgcacagaatggtgatgctgctgttaaagattttgacttttaccgttataataagcctaccattttagatatttgtcaagctagagttacatataagatagtctctcgttattttgacatttatgaaggtggctgtattaaggcatgtgaagttgttgtaacaaatcttaataagagtgctggttggccattaaataagtttggtaaagctagtttgtattatgaatctatatcttatgaagaacaggatgctttgtttgctttgacaaagcgtaatgtcctccctactatgacacagctgaatcttaagtatgctattagtggtaaagaacgtgctagaactgttggtggtgtttctctgttgtctacaatgaccacaagacaataccatcaaaaacatcttaaatccattgttaatacacgcaatgccactgttgttattggtactaccaaattttatggtggttggaataatatgttgcgtactttaattgatggtgttgaaaaccctatgcttatgggttgggattatcccaaatgtgatagagctttgcctaacatgatacgtatgatttcagccatggtgttgggctctaagcatgttaattgttgtactgcaacagataggttttataggcttggtaatgagttggcacaagttttaacagaagttgtttattctaatggtggtttttattttaagccaggtggtacgacttctggtgacgctagtacagcttatgctaattctatttttaacatttttcaagccgtgagttctaacattaacaggttgcttagtgtcccatcagattcatgtaataatgttaatgttagggatctacaacgacgtctgtatgataattgttataggttaactagtgttgaagagtcattcattgatgattattatggttatcttaggaaacatttttcaatgatgattctctctgatgacggtgttgtctgttataacaaggattatgctgagttaggttatatagcagacattagtgcttttaaagccactttgtattaccagaataatgtctttatgagtacttctaaatgttgggttgaagaagatttaactaagggaccacatgagttttgttcccagcatactatgcaaatagttgacaaagatggtacctattatttgccttacccagatcctagtaggatcttgtcagctggtgtttttgttgatgatgttgttaagacagatgctgttgttttgttagaacgttatgtgtctttagctattgatgcataccctctttcaaaacaccctaattccgaatatcgtaaggttttttacgtattacttgattgggttaagcatcttaacaaaaatttgaatgagggtgttcttgaatctttttctgttacacttcttgataatcaagaagataagttttggtgtgaagatttttatgctagtatgtatgaaaattctacaatattgcaagctgctggtttatgtgttgtttgtggttcacaaactgtacttcgttgtggtgattgtctgcgtaagcctatgttgtgcactaaatgcgcatatgatcatgtatttggtaccgaccacaagtttattttggctataacaccgtatgtatgtaatgcatcaggttgtggtgttagtgatgtcaaaaaattgtatcttggtggtttgaattactattgtacaaatcataaaccacagttgtcttttccattatgttcagctggtaatatatttggtttatataaaaattcagcaactggttccttagatgttgaagtttttaataggcttgcaacgtctgattggactgatgttagggactataaacttgctaatgatgttaaagatacacttagactctttgcggctgaaactattaaagctaaagaagagagtgttaagtcttcttatgcttttgcaactcttaaagaggttgttggacctaaagaattgcttcttagttgggaaagtggtaaagttaaaccacctttgaatcgtaattctgttttcacttgttttcaaataagtaaggactcaaaattccaaataggtgagttcatctttgagaaggttgaatatggttctgatactgttacgtataagtctactgtaactactaagttagttcctggtatgatttttgtcttaacatctcacaatgtccaacctttacgtgcaccaactattgcaaaccaagagaagtattctagcatttataaattgcaccctgcttttaatgtcagtgatgcatatgctaatttggttccatattaccaacttattggtaaacaaaagataactacaatacagggtcctcctggtagtggtaagtcacattgttccattggacttggattgtactacccaggtgcgcgtattgtttttgttgcttgtgcccatgctgctgttgattccttatgtgcaaaagctatgactgtttatagcattgataagtgtactaggattatacctgcaagagctcgggttgagtgttatagtggctttaaaccaaataacactagtgcacaatacatatttagcactgttaacgcattacctgagtgtaatgctgatatcgttgttgtagatgaagtttcaatgtgtacaaattatgacctttctgttattaaccagcgtttatcatataaacatattgtttatgttggtgatccacaacaacttcctgcacctagagtaatgattactaaaggtgttatggagcctgttgattataacgttgttactcaacgtatgtgtgctataggccctgatgtttttcttcataaatgttatagatgtcctgctgaaatagttaatacagtttctgaacttgtttatgagaacaagtttgtccctgttaaacctgctagtaaacagtgttttaaagtcttttttaagggtaatgtacaggttgacaatggttctagtattaacagaaagcagcttgaaatagttaagctgtttttagttaaaaatccaagttggagtaaggctgtgtttatttctccttataatagtcagaattatgttgctagtagatttttaggacttcaaattcaaactgttgattcttctcaaggtagtgagtatgattatgtaatctatgcacaaacttctgacactgcacatgcttgcaatgtaaaccgttttaatgttgctataacacgtgctaagaagggtatattttgtgtaatgtgtgataaaactttgtttgattcacttaagttttttgagattaaacatgcagatttacactctagccaggtttgtggcttgtttaaaaattgtacacgcactcctcttaatttaccaccaactcatgcacacactttcttgtcgttgtcagatcagtttaagactacaggtgatttagctgttcaaataggttcaaataacgtttgtacttatgaacatgttatatcatttatgggttttaggtttgatattagtattcctggtagtcatagtttgttttgtacacgtgactttgctattcgtaatgtgcgtggttggttgggtatggatgttgaaagtgctcatgtttgtggcgataacataggtactaatgttcctttacaggttggtttttcaaatggtgttaattttgttgtgcaaactgaaggttgtgtgtctaccaattttggtgatgttattaaacctgtttgtgcaaaatctccaccaggtgaacaatttagacaccttattcctcttttacgtaaaggacaaccttggttaattgttcgtagacgcattgtgcaaatgatatctgattatttgtccaatttgtctgacattcttgtctttgttttgtgggcaggtagtttggaattaactacaatgcgttactttgtaaaaatagggccaattaaatattgttattgtggtaattctgccacttgttataattcagttagtaatgaatattgttgttttaaacatgcattgggttgtgattatgtttacaatccgtatgcttttgatatacaacagtggggttatgttggttccttgagccaaaaccaccacacattctgtaacattcatagaaacgagcatgatgcctctggtgatgctgttatgacacgttgtttggcagtacatgattgttttgtcaaaaatgttgattggactgtaacgtacccctttattgcaaatgagaaatttatcaatggctgtgggcgtaatgtccagggacatgttgttcgtgcagccttgaaattgtataaacctagtgttattcatgacattggtaatcctaaaggtgtacgttgtgctgttactgatgccaaatggtactgttatgacaagcaacctgttaatagtaatgtcaagttgttggattatgattatgcaacccatggtcaacttgatggtctttgtttattctggaattgtaatgttgatatgtatccagaattttcaattgtgtgtcgttttgacacacgtactcgttctgtttttaatttagaaggtgttaatggtggttctctttatgttaacaaacatgcgtttcatacaccagcatatgataaacgtgcttttgttaaattaaaacctatgccctttttttactttgatgacagtgattgtgatgttgtgcaagaacaagttaattatgtaccccttcgcgctagtagttgtgttactcgttgtaatataggtggtgctgtttgttcaaaacatgcaaatttgtatcaaaaatatgttgaggcatataatacatttacacaggcaggttttaacatttgggtaccacatagttttgatgtttataatttgtggcaaatttttattgaaactaatttacaaagtcttgaaaatatagcatttaatgttgtaaaaaaagggtgttttactggtgttgatggtgagttacctgttgcagttgttaacgacaaagtttttgttcgctatggcgatgttgacaacttggtttttacaaataaaacaacattgcctactaatgttgcttttgaattgtttgcaaaacgaaaaatgggtttaacaccaccattgtctattctcaaaaatctcggtgttgttgctacatataaatttgttttatgggattatgaagctgaaagaccttttacctcatatactaagagtgtatgtaaatacactgattttaatgaggatgtttgtgtttgttttgacaatagtattcagggttcgtatgagcgttttacgcttactacgaacgctgttttattttctactgttgtcattaaaaatttaacacctataaagttgaattttggtatgttgaatggtatgccagtttcttctattaagggtgataaaggtgttgaaaaattagttaattggtacatatatgttcgtaaaaatggtcaatttcaagatcactatgatggtttttacactcaaggtaggaatttatcagactttacaccaagaagtgatatggagtatgattttcttaacatggatatgggtgtttttattaataaatatggtcttgaggattttaattttgaacatgttgtatatggtgatgtttcaaaaactacattaggaggtcttcatttgttgatatcacagtttaggcttagtaaaatgggtgttttgaaagctgatgattttgtcactgcttctgacacaactttgaggtgctgtactgttacttatcttaatgaacttagttcaaaagttgtttgtacttatatggatttgttgttggacgactttgttactatactaaagagtttagatcttggtgtaatatctaaagttcatgaagttattatagataataaaccttataggtggatgttgtggtgtaaagataaccacttgtccactttttatccacagttgcagtctgctgaatggaagtgtggttatgctatgccacaaatttataagcttcaacgtatgtgtttggaaccttgtaatttatataattatggtgctggtattaagttgcctagtggtataatgttaaatgttgttaaatacactcagctttgtcaatacctaaatagcactacaatgtgcgtacctcataatatgcgtgttttgcactatggtgctggttctgacaaaggtgtggcacctggtacaactgttttaaaacgttggctaccacccgatgcaataatcattgataatgatatcaatgattatgttagtgatgcagattttagcattacaggtgattgtgctactgtttatcttgaagataagtttgacttacttatttctgatatgtatgatggtagaattaaattttgtgatggtgaaaatgtctctaaagatgggttttttacttatcttaatggtgttattagagaaaaattagctattggtggtagtgttgccattaagattacagaatatagttggaataagtatctttatgaattaatacaaagatttgctttttggactttgttttgcacgtctgttaatacatcctcttcagaagcttttcttattggtattaattatttaggtgactttattcaaggtccttttatagctggtaacactgttcatgctaattatatattttggcgtaattctactattatgtctttgtcatacaattcagttttagatttaagtaagtttgaatgtaaacataaagccactgttgttgttacacttaaagatagtgatgtaaatgatatggttttgagtttgattaagagtggtaggttgttgttacgcaataatggtcgttttggtggttttagtaatcatttagtctcaactaaatgaaacttttcttgattttgcttgttttgcccctggcctcttgctttttcacatgtaatagtaatgctaatctctctatgttacaattaggtgttcctgacaattcttcaactattgttacgggtttattgccaactcattggttttgtgctaatcagagtacatctgtttactcagccaatggtttcttttatattgatgttggtaatcaccgtagtgcttttgcgctccatactggttattatgatgctaatcagtattatatttatgttactaatgaaataggcttaaatgcttctgttactcttaagatttgtaagtttagtagaaacactacttttgattttttaagtaatgcttctagttcttttgactgtatagttaatttgttatttacagaacagttaggtgcgcctttgggcataactatatctggtgaaactgtgcgtctgcatttatataatgtaactcgtactttttatgtgccagcagcttataaacttactaaacttagtgttaaatgttactttaactattcctgtgtttttagtgttgtcaacgccaccgttactgtgaatgtcaccacacataatggccgtgtagttaactacactgtttgtgatgattgtaatggttatactgataacatattttctgttcaacaggatggccgcattcctaatggtttcccttttaataattggtttttgttaactaatggttccacactagtggacggggtctctagactttatcaaccactccgtttaacttgtttatggcctgtacctggtcttaaatcttcaactggttttgtttattttaatgccactggttctgatgttaattgtaacggctatcaacataattctgttgttgatgttatgcgttacaatcttaacttcagtgctaattctttggacaatctcaagagtggtgttatagtttttaaaactttacagtacgatgttttgttttattgtagtaattcttcctcaggtgttcttgacaccacaataccttttggcccgtcctctcaaccttattactgttttataaacagcactatcaacactactcatgttagcacttttgtgggtattttaccacccactgtgcgtgaaattgttgttgctagaactggccagttttatattaatggttttaagtatttcgatttgggtttcatagaagctgtcaattttaatgtcacgactgctagcgccacagatttttggacggttgcatttgctacttttgttgatgttttggttaatgttagtgcaactaacattcaaaacttactttattgcgattctccatttgaaaagttgcagtgtgagcacttgcagtttggattgcaggatggtttttattctgcaaattttcttgatgataatgttttgcctgagacttatgttgcactccccatttattatcaacacacggacataaattttactgcaactgcatcttttggtggttcttgttatgtttgtaaaccacaccaggttaatatatctcttaatggtaacacttcagtgtgtgttagaacatctcatttttcaattaggtatatttataaccgcgttaagagtggttcaccaggtgactcttcatggcacatttatttaaagagtggcacttgtccattttctttttctaagttaaataattttcaaaagttcaagactatttgtttctcaaccgtcgaagtgcctggtagttgtaattttccgcttgaagccacctggcattacacttcttatactattgttggtgctttgtatgttacttggtctgaaggtaattctattactggtgtaccttatcctgtctctggtattcgtgagtttagtaatttagttttaaataattgtaccaaatataatatttatgattatgttggtactggaattatacgttcttcaaaccagtcacttgctggtggtattacatatgtttctaactctggtaatttacttggttttaaaaatgtttccactggtaacatttttattgtgacaccatgtaaccaaccagaccaagtagctgtttatcaacaaagcattattggtgccatgaccgctgttaatgagtctagatatggcttgcaaaacttactacagttacctaacttttattatgttagtaatggtggtaacaattgcactacggccgttatgacttattctaattttggtatttgtgctgatggttctttgattcctgttcgtccgcgtaattctagtgataatggtatttcagccataatcactgctaatttatccattccttctaactggactacttcagttcaagttgagtacctccaaattactagtactccaatagttgttgattgtgctacttatgtgtgtaatggtaaccctcgctgtaagaatctacttaagcagtatacttctgcttgtaaaactattgaagatgccttacgacttagtgctcatttggaaactaatgatgttagtagtatgctaactttcgatagcaatgcttttagtttggctaatgttactagttttggagattataacctttctagtgttttacctcagagaaacattcgttcaagccgtatagcaggacgtagtgctttggaagatttgttgtttagcaaagttgttacatctggtttgggtactgttgatgttgactataagtcttgtactaaaggtctttctattgctgaccttgcttgtgctcagtactacaatggcataatggttttgccaggtgttgctgatgctgaacgtatggccatgtacacaggttctcttataggtggcatggtgctcggaggtcttacatcagcagccgccatacctttttctttggcactgcaagcacgacttaactatgttgctttacaaactgatgtgcttcaagaaaatcagaaaattttggctgcatcatttaataaggctattaataatattgttgcttcttttagtagcgttaatgatgctattacacaaactgcagaggctatacatactgttactattgcacttaataagattcaggatgttgttaatcaacagggtagtgctcttaaccatctcacttcacaattgagacataattttcaggccatttctaattcaattcaggctatttatgaccggcttgattcaattcaagccgatcaacaagttgacagattaattactggacggcttgcagctttgaatgcatttgtttcccaagttttgaataaatatactgaagttcgtggttcaagacgcttagcacagcagaagattaatgaatgtgtcaagtcacaatctaatagatatggtttttgtggcaatggcactcacatcttttcaatcgtcaactcagctccagatggtttgctttttcttcatactgttttgctgccaactgattacaagaatgtaaaggcgtggtctggtatctgtgttgatggcatttatggctatgttctgcgtcaacctaacttggttctttattctgataatggtgtctttcgtgtaacttccagggtcatgtttcaacctcgcttacctgttttgtctgattttgtgcaaatatataattgtaatgttacttttgttaacatatctcgtgttgagttacatactgtcatacctgactacgttgatgttaataaaacattacaagagtttgcacaaaacttaccaaagtatgttaagcctaattttgacttgactccttttaatttaacatatcttaatttgagttctgagttgaagcaactcgaagctaaaactgctagtctttttcaaactactgttgaattacaaggtcttattgatcagattaacagtacatatgttgatttgaagttgcttaataggtttgaaaattatatcaaatggccttggtgggtttggctcattatttctgttgtttttgttgtattgttgagtcttcttgtgttttgttgtctttctacaggttgttgtggttgttgcaattgtttaacttcatcaatgcgaggctgttgtgattgtggttcaactaaacttccttattacgaatttgaaaaggtccacgttcaataatgccttttggtggcctatttcaacttactcttgaaagtactattaataagagtgtggctaatctcaaattaccacctcatgatgttactgtcttgcgtgacaatcttaaacctgttactacacttagtactattactgcttatttgttagttagtttgtttgtcacttactttgctttattcaaacctcttactgctagaggtcgtgttgcttgttttgttttaaaactattgacactatttgtctatgtgcctttattggttctttttggtatgtatcttgacagttttataattttttctacgctgttgtttcgattcatacatgttggctattatgcctatctctataaaaatttttcatttgttttgttcaatgttactaaactatgcttcgtttcaggcaagtgttggtatcttgaacaatcattttatgaaaatcgttttgctgctatttatggtggtgaccactatgtcgttttaggtggtgaaactattacttttgtttcttttgatgacctttatgttgctattagaggttcttgtgaaaagaacctacaacttatgcgtaaggttgacttgtataatggtgctgtcatttacatttttgccgaagagcctgttgttggtatagtctactcttctcaactatacgaagatgttccttcgattaattgatgacaatggtattgtcctcaattccattttatggctccttgttatgatatttttctttgtgttggcaatgacctttattaaactgattcaattgtgttttacttgtcattatttttttagtaggacattatatcaaccagtttataaaatttttcttgcttaccaagattatatgcaaatagcacctgttccagctgaagtactaaatgtctaaactaaacgatgtctaatagtagtgtgcctcttttagaggtttatgtccatttacgtaactggaactttagttggaatttaattctaacgctttttatagttgtgttgcagtatgggcattataagtatagcagacttctttatggtttaaagatgtctgttttatggtgtttatggccacttgttctagctttgtctatttttgactgttttgtcaattttaatgtggactgggtcttttttggttttagtattcttatgtctattattacactttgtttatgggttatgtattttgttaatagtttcagactttggcgccgtgttaaaactttttgggcttttaatcctgaaactaatgcaatcatctctctccaggtttacggacataattattacttaccggtgatggctgcacctacaggtgttacattaacacttcttagtggtgtacttcttgttgatggccataagattgctactcgtgttcaagtgggtcagttgcctaaatatgtaatagttgctacgcctagtaccacaattgtttgtgaccgtgttggtcgctctgttaatgaaacaagccagactggttgggcattctacgtccgtgctaaacatggtgatttttctggtgttgcctctcaggagggtgttttgtcagaaagagagaagttgcttcatttaatctaaactaaacaaaatggctagtgtaaattgggccgatgacagagctgctaggaagaaatttcctcctccttcattttacatgcctcttttggttagttctgataaggcaccatatagggtcattcccaggaatcttgtccctattggtaagggtaataaagatgagcagattggttattggaatgttcaagagcgttggcgtatgcgcagggggcaacgtgttgatttgcctcctaaagttcatttttattacctaggtactggacctcataaggaccttaaattcagacaacgttctgatggtgttgtttgggttgctaaggaaggtgctaaaactgttaataccagtcttggtaatcgcaaacgtaatcagaaacctttggaaccaaagttctctattgctttgcctccagagctctctgttgttgagtttgaggatcgctctaataactcatctcgtgctagcagtcgttcttcaactcgtaacaactcacgagactcttctcgtagcacttcaagacaacagtctcgcactcgttctgattctaaccagtcttcttcagatcttgttgctgctgttactttggctttaaagaacttaggttttgataaccagtcgaagtcacctagttcttctggtacttccactcctaagaaacctaataagcctctttctcaacccagggctgataagccttctcagttgaagaaacctcgttggaagcgtgttcctaccagagaggaaaatgttattcagtgctttggtcctcgtgattttaatcacaatatgggggattcagatcttgttcagaatggtgttgatgccaaaggttttccacagcttgctgaattgattcctaatcaggctgcgttattctttgatagtgaggttagcactgatgaagtgggtgataatgttcagattacctacacctacaaaatgcttgtagctaaggataataagaaccttcctaagttcattgagcagattagtgcttttactaaacccagttctatcaaagaaatgcagtcacaatcatctcatgttgctcagaacacagtacttaatgcttctattccagaatctaaaccattggctgatgatgattcagccattatagaaattgtcaacgaggttttgcattaaattgttttgtaattccagttgaatgtttattattattagttgcaaccccatgcgtttagcgcatgataagggtttagtcttacacacaatggtaggccagtgatagtaaagtgtaagtaatttgctatcatattaacatgtctagaggaaagtcagaactttttctgtttgtgttgttggagtacttaaagatcgcataggcgcgccaacaatggaagagccaacaacatatctaaaaatgttttgtctggtacttgttaatgatattgtttttgatatggatacac

SEQ ID NO: 4 - DNA, 27271 bp, Human coronavirus
  ref <308>: DATABASE ACCESSION NUMBER: MF542265.1
  ref <309>: DATABASE ENTRY DATE: 2017-11-24
  ref <313>: RELEVANT RESIDUES IN SEQ ID NO: (1)..(27271)
gatcttaagtaccttatctatctacagatagaaaagttgctttttagactttgtgtctacttttctcaactaaacgaaatttttgctatggccggcatctttgatgctagagtcgtagtgtaattgaaatttcatttgggttgcaacagtttggaagcaagtgctgtgtgttctagtctaagggtttcgtgttccgtcacgagattccattctacaaacgccttacgcgaggttctgtctcgtgtttgtgtggaagcaaagttttgtctttgtggaaaccagtaactgttcctaatggcctgcaaccgtgtgacacttgccgtagcaagtgattctgaaatttctgcatatggctgttctactattgcgcaagccgtccgccgttatagcgaggccgctagcaatggttttagggcatgccgatttgtttcattagatttgcaggattgcatcgttggcattgcagatgatacatatgttatgggtctgcatggcaatcagacgttgttttgcaacataatgaaattttctgaccgtccttttatgcttcatgggtggttggttttttccaattcaaattaccttttggaggaatttgatgttgtcttcggtaagagaggtggtggtaatgtgacatacactgaccagtatctctgtggcgccgatggcaaacctgttatgagtgaagatttatggcagtttgttgaccatttcggtgagaacgaagaaattgtcatcaatggtcgtacttacgtttgtgcttggcttactaagcgtaagcccttagattacaaacgtcagaacaacctcgccattgaagagattgaatacgtgcatggtgatgctttgcacacactacgcaatggttctgttcttgaaatggctaaggaagtgaagacatctagtaaagttgtgttaagcgatgctcttgacaaactttacaaagtctttggttctcctgttatgacaaatggttccaacatcctagaggcctttactaaacctgtgtttattagtgcattagttcaatgtacttgtggtaccaagtcttggtctgtgggtgattggaccggttttaaatcctcttgttgcaacttgatcagtaataaactgtgtgttgtgcctggtaatgttaaacctggtgatgctgtgattactactcagcaagctggtgctggtgttaagtatttttgtggcatgactcttaagtttgttgcaaatattgaaggtgtctctgtttggagagtgattgctcttcagagtgttgattgctttgttgcttcttccacttttgtagaagaggaacatgttaatagaatggacacattctgcttcaatgtacgcaatagtgttactgatgagtgtcgtctggccatgttgggtgctgaaatgactagtaatgtcagaagacaagttgcttcaggtgtcatagacattagtaccggctggtttgatgtttatgatgacatctttgctgaaagcaaaccatggtttgttcgcaaggctgaagacattttttgcccttgttggtctgctcttgtttctgcacttaaacaacttaaagtcactacaggtgaacttgtgagatttgttaagtctatttgcaattcagctgttgctgtcgtgggtggtactatacaaattctcgctagtgtgcctgagaagtttttgaatgcgtttgacgtgtttgtcacaactattcaaactgtctttgactgtgctgttgaaacttgtactattgccggtaaagcatttgacaaggtttttgactatgtcttgcttgataatgcgcttgtaaaacttgtcaccacaaagcttaagggtgttcgtgaaggtggccttaataaagttaagtatgcaacagttgttgttggttccactgaagaagttaaatcttcacgtgttgaacgtagcaatgctgtacttacaatcgccaacaattattccaaactttctgatgaagggtatactgttgtaattggcgatgtggcgtactttgctagtgacggctacttccgtcttatggccagtccaaatagtgtgttgactactgcagtctataaaccattggttgattttaatgtgaatgttattggtactagacctgaaaaatttccaaccactgtgacttgtgaaaatttagagtctgctgttttgtttgttaatgacaaaatcactgaattccaattggattacagtattgatgtcattgataatgaaataattgtcaaacctaatatcagcttatgtgttccactttatgtgagagactatgttgacaaatgggatgatttttgcagacaatatagtaacgagtcttggtttgaggatgattacaaggcttttatcagtgttttggatatcactgatgctgctgtgaaagctgcagaatctaaagccttcgttgatactattgttccaccttgcccatctattttgaaagttatagatggcggcaaaatatggaatggtgttattaaaaatgttaactatgttagagactggcttaagtcgttgaagttaaatctcacacaacagggtttgcttggaacatgtgcaaagcgttttaaacgttggcttggcattttgctagaggcctataatgcgtttttaaacactgttgtttctactgttaaaattggtggtttgacctttaaaacatatgcttttgataaaacttacattgtgatacgtgatatcgtgtgtaaggttgaaaataaagcagaagcagaatggattgaactttttccacataatgacaggattaagtcctttagtacttttgagagtgcttacatgccaattgcagaccctacacattttgacattgaagaagttgaacttttagatgcagagtttgtagaaccaggctgtggtggtattttgtcagtaatagatgagcacgtcttttataagaaggatggtgtttattatccatcaaatggtactaacattctacctgttgcatttacaaaagccgctggtggtaaagtttcattttctgatgacgttgaagtaaaagacattgaacctgtttacagagtcaagctttgctttgagtttgaagatgaaaaacttgtagatgtttgtgaaaaggcaattggcaagaaaattaaacatgaaggtgactgggatagctttggtaagactattcaatcagcactttctgttgtttcatgctatgtaaatctgcctacttattacatttatgatgaagaaggcggtaatgacttgagtttgcccgttatgttttctgaatggcctctttttgagtcagataaggaagaagatgttcaacaagctcaacaagaagctactcatattgctgaggatgttgttgaccaagttgaagaagtcaatagcatttttgacattgagacagtggatgttaaacatgagatgagtccttttgaaatgccatttgaagagttaaatggtttaaagatacttaaacaattggataacaactgctgggttaactcagttatgttacaaatacaattaactggtatacttgatggtgattatgctatgcagttttttaaaatgggccgagttgccaagatgattgaacgctgctacactgctgagcaatgtatacgtggtgctatgggtgatgttggtttgtgtatgtatagacttcttaaagacttacacactggttttatggttatggattataaatgtagttgtaccagtggtaggcttgaagaatcgggagctgttttgttttgtacgcccactaagaaggcgtttccttatggtacttgtctaaattgtaacgcacctcgcatgtgtacaattaggcagttacaaggtaccataatatttgtgcaacaaaaaccagaacctgttaaccctgtttcttttgttgttaaaccagtctgctcatcaatttttcgtggtgctgtgtcttgtggtcattaccacactaacagctattcacaaaatttgtgtgtggatggttttggtcttaacaagattcagccctggacaaatgatgcacttaatactatttgtattaaagatgcagattataatgcaaaagatgaaatatctgttacaccaattaaaaacacagttgatacaatacctaaggaagaatttgttgttaaagagaagttgaacgccttcctcgttcatggcaatgtagttttctaccaaggtgatgttgataccgttgttaatggtgttgactttgactttattgtaaatgctgctaatgagaaccttgctcatggtggaggacttgccaaagctttagatgtgtacactaaaggtaaacttcaacgtttatccaaagaacacattggattagcgggtaaagtaagagttggtacaggagttatggttgagtgtgatagccttagaatttttaatgttgttggtccacgcaagggtaaacatgaacgtgatttactcgtaaaagcttacaacactattaataacgaacaaggcacacctttaacaccaattttgagttgtggtatttttggtgtcaaactcgaaacttcattggaagttttgcttgatgtttgtaatacaaaagaagttaaagtttttgtttatacagacacagaagtttgtaaggttaaggattttgtgtctggtttagtgaatgttcaaaaagttgcacaacctaaaatagaacaaaaaccattgtccttaattaaagttgcacccaagccttacagggtagatggtaaatttagttactttacagaagacttgttgtgtgtcgctgatgacaaacccattgttttgtttactgactctatgcttactttggatgaccgtggtttggctctagacaatgcacttagtggtgtgcttagtgctgctattaaggattgtgttgacataaataaagctataccttctggtaatcttattaagtttgacatagattctgttgttgtttacatgtgtgttgtgccatccgaaaaggacaaacatttagataataatgttcaacgatgcacacgtaagttgaatagacttatgtgtgatatagtttgtactataccagctgactacatcttgccattggtgttgtctagtttgacttgtaatgtttcttttgtaggtgaacttaaagctgctgaagctaaagttataactataaaggtgacagaggatggtgttaatgttcatgatgtgaccgtgacaacagataagtcatttgaacaacaagttggtgttattgctgacaaggacaaagatctttctggtgcagtacccagtgatcttaacacatctgaattgcttactaaagcaatagatgttgattgggtcgaattttatggctttaaagatgctgttacttttgcaacagttgatcatagtgcttttgcctatgaaagtgctgttgttaatggtattagagtgttaaaaaccagtgataataattgttgggtgaatgctgtttgtattgcactacagtatttgaaacctcattttatttcacaaggtcttgatgctgcgtggaataaatttgttttaggcgatgttgaaatttttgttgcatttgtttactatgttgcaagactagtgaaaggtgacaagggtgatgctgaagacactttgaataagttgtctaagtatcttgttaatgaagctcaagttcaattagaacattatagttcttgtgttgaatgtgatgctaaatttaaaaactctgttacatctatcaattctgctatagtttgtgctagtgtcaaacgtgatggtgtgcaagttggttattgtgtccatggtattaagtactattcacgtgttaaaagtgttagaggtagagctattatagtcagtgtcgaacagcttgaaccgtgtgttcagtctagacttttgagtggtgttgcttatactgctttttctggacctgttgataaaggtcattatactgtttatgacactgcaaagaaatcaatgtatgatggtgatcgttttgttaaacatgatctttctctgctgtctgtcacatcagttgttatggttggtgattatgttgcacctgttagtacagtgaaacctaaaccagtcatttctcaacttgatgaaaatgcacagaagttctttgattttggtgattttttggttcataattttgttatttttttcacatggttattgagtatgtttactttgtgtaaaactgcagtaactacatgtgatgttaaaataatggccaaagcaccacaaaggacgggtgttgttttaaaacgtagtcttaaatataacttaaaagcgtcagcagctgttcttaaatctaagtggtggctgcttgctaagtttacgaaactactgttacttatatatacattgtattcagtagttttgttttgtgtacgttttggaccgtttgagttttgtagtgagactgttaatggttatgctaagtcaaactttgtcaaggatgattactgtgatggttcattgggctgcaagatgtgtctttttggttatcaagagttaagtcaatttagccatttggatgttgtgtggaagcatataacagaccctttgtttagtaatatacaacctttcattgttatggttttgctgcttatatttggtgacaattatttgagatgcttcttgctgtattttgttgctcagatgataagcacagttggtgtttttctaggttataaggaaacaaattggttcttgcactttattccatttgatgttatttgtgatgaactgtttgtcactgttattgttattaaggttatttcttttgtcagacatgtgctttttggttgtgaaaacccagattgcattgcgtgttctaagagtgctagacttaagagattccctgttaacacaattgttaatggtgtgcaacgtttattttatgttaatgcaaatggtggtagtaagttttgtaagaaacatagatttttctgtgttgactgtgactcttatggttatggcagcacgtttataacacccgaagtttctagagaacttggtaacattactaaaacaaatgtgcaaccaacaggtccagcgtatgtcatgattgacaaagtggagtttgaaaatggtttttacaggttgtattccggtgaaacattttggcgttacaactttgatataactgaaagcaagtattcttgcaaagaggtttttaaaaattgtaatgttttggatgatttcatcgtgtttaacaataatgggaccaatgtaacgcaggttaaaaatgctagtgtttacttttcacagttgttgtgtaggcccattaaattagttgacagtgaacttttgtccactttgtcagttgattttaatggtgtcttacacaaggcatacatcgatgtactacgtaatagctttggtaaagatcttaatgctaatatgtctttagccgattgcaagagtgctttaggcctgtctattagtgatcatgaatttactagtgctatttctaatgcacatcgttgtgacgtgttgttatctgatttgtcatttaacaactttgtcagttcgtatgctaaacctgaggaaaaattatcagcttatgacttggcgtgttgtatgcgtgcaggtgctaaggttgttaatgccaatgttctgacaaaggaccaaactcctattgtttggcatgcaaaggattttaacagtctttctgctgaaggtcgtaagtatattgtaaaaactagcaaagctaagggtttgactttcttgttgacaattaatgaaaaccaagctgttacgcaaatacctgcaactagcattgttgctaagcaaggtgctggtgatgctggtcattcattaacatggctgtggttactgtgtggtcttgtgtgtttgattcaattctatttgtgctttttcatgccctattttatgcacgatgtagtgagtagttttgagggttatgattttaagtatatagaaaatggtcagttgaagaattttgaagcgccacttaaatgtgtcagaaacgtttttgaaaactttgaggactggcattatgctaagtttggcttcacacctttaaacaagcaaagctgtcctattgtagttggagtttctgaaattgttaatactgtcgctggcattccatctaatgtgtatcttgttggtaaaactttaatttttacactacaagctgcttttggtaatgctggtgtttgttatgacatttttggagtcacaacacctgaaaagtgcatttttacttctgcttgtactagattagaaggtttgggtggtaacaatgtttattgttataacacagagcttatggaaggttctttgccgtacagttcaatacaagctaatgcatattataaatatgataatggcaattttattaagttgccagaagttattgcacaaggctttggctttagaacagtacgtactattgctaccaaatactgccgcgtaggtgaatgtgttgactccaatgcaggtgtgtgttttggctttgacaagtggtttgttaatgatggacgtgttggcaatggttacgtttgtggtactggtttgtggaaccttgtatttaacatactttccatgttctcatcttcattctctgttgctgcaatgtcaggtcaaattttacttaattgtgcattaggtgcttttgctatcttttgttgttttcttgtgacaaagtttagacgcatgtttggtgacctttctgtaggtgtttgtactgttgttgttgctgttttgcttaacaatgtctcttacattgtaactcagaatttagtaacaatgattgcttatgccatattgtatttctttgctactagaagcttacgctatgcatggatttggtgtgctgcatatttaattgcgtatatttcttttgctccatggtggttgtgtgcttggtactttcttgctatgttgacaggtttgttacctagtttgctgaagcttaaagtttcgacaaatcttttcgaaggtgacaaatttgtaggtacatttgaaagtgctgctgcaggaacatttgtcattgacattcgttcttatgagaaacttgctaatagcatctctccagaaaagttgaaaagttatgctgctagctataatagatacaagtactatagtggtaatgcaaatgaagctgattaccgttgtgcttgttatgcctatttagcaaaagcaatgttggacttttcgcgtgatcataatgacatcttgtacacacctccgactgtcagttatggttctacattacaggctggtttgcgcaaaatggcacaaccatctggctttgtggaaaaatgtgttgtccgtgtctgctatggaaacactgtgttgaatgggttgtggcttggtgatattgtttattgcccacgtcatgttatcgcatctaacacaacttctgctatagattatgatcacgaatatagtattatgcggttgcataatttctctataatatctggtacagcatttcttggtgttgtaggtgctactatgcatggagtaactcttaaaattaaggtttcacagactaacatgcacacacctagacactcttttagaacactaaaatctggtgaaggttttaacatcttagcatgctatgatggttgtgctcagggtgtttttggtgtgaacatgagaactaattggactatccgtggttcatttattaatggtgcgtgtggttctcctggctacaatcttaaaaatggcgaagtggaatttgtttatatgcatcaaattgaactcggaagtggtagtcatgtaggttctagctttgatggtgttatgtatggtggttttgaagaccaacctaatcttcaagttgaatctgcaaaccagatgttaacagttaatgtggttgcatttctttatgctgctatattgaatggttgcacatggtggcttaaaggtgataaattgtctgtggagcattataatgagtgggcacaggctaatggtttcacagctatgaatggtgaagacgctttttccattcttgctgttaaaactggtgtctgtgtggaaagattacttcatgctattcaagttttgaataatggatttggtggtaaacaaattttgggttattctagtctcaatgatgagttcagtattaatgaagttgtcaaacaaatgtttggtgttaacctgcaaagtggtaaaaccactagtatgtttaaatccataagcttgtttgctggtttctttgtcatgttctgggctgaattatttgtttataccaccactatttgggttaaccctggttttcttactccttttatgattttgcttgttgctttgtcactctgtcttacatttgttgttaaacataaggttttgtttttgcaagtgtttttgttgccttcaattattgtggctgctattcaaaactgtgcttgggactaccatattacaaaggtgttggcagagaagtttgattataatgtttctgttatgcaaatggacatccagggttttgttaacatttttatttgtctttttgttgcactgttgcatacttggcgctttgctaaagagcgttgtacacattggtgcacttatttgttctcactcattgctgttttatacactgcattgtatagttatgactacgttagtttgctggttatgctactttgtgcaatttctaatgaatggtatattggtgctatcatttttagaatttgtcgttttggtgttgcatttttaccagtggaatacgtgtcttactttgatggtgttaaaactgtgctgttgttttacatgttgttaggctttgttagctgtatgtactatggtttgttgtactggattaacaggttctgtaagtgcacattaggtgtttatgatttctgtgttagtccagccgaatttaagtatatggttgctaatggtttgaatgcaccaaatggcccttttgatgcactctttctgtcttttaaactaatgggtattggtggtcctagaaccattaaagtctctactgtacagtctaaattgactgatcttaagtgcacaaacgtcgttctaatgggcattttgtctaacatgaacatagcttctaattcaaaggagtgggcatattgtgttgaaatgcacaataaaataaatttgtgtgacgaccctgaaactgctcaagagttattgctggcgttgttggcctttttcttgtctaagcatagtgattttggtcttggtgatcttgtcgattcttattttgagaacgactccattttgcaaagtgttgcatcttcttttgttggtatgccatcttttgttgcatatgaaacagcaagacaagagtatgaaaatgctgttgcaaatggttcctcaccacaaataatcaaacaattgaagaaggctatgaatgttgcaaaagctgagtttgacagggagtcatctgttcaaaagaaaattaacagaatggctgaacaagctgctgcagctatgtacaaagaagcacgtgctgttaatagaaaatcaaaagttgtaagtgcaatgcatagtttactctttggcatgctccgacgtttggacatgtctagtgttgacaatatccttaatatggcacgtaatggtgttgtccctctttccgttatccctgctacttctgcagccaggctcgtcgtcgtagtgccagatcatgattcatttgtgaaaatgatggtagatggttttgtgcactacgctggtgttgtttggacattacaggaagttaaggataatgacggtaagaatgtgcatcttaaagatgttacaaaggaaaaccaggaaatacttgtttggcctctgattttgacttgtgaacgtgtcgttaaattgcagaacaatgaaataatgccaggcaagatgaaggtcaaggccaccaaaggtgaaggtgatggaggcattattagtgaaggtaatgctctatacaacaatgaaggtggacgtgcatttatgtatgcatatgtgactacgaagcctggcatgaagtatgttaaatgggaacatgactctggtgtggttacagttgaattggaaccaccttgcagatttgttatagacacacctactggaccccaaattaagtatctttattttgttaagaatcttaacaatttaaggagaggtgctgttttgggttacattggtgccactgtgagattgcaagctggcaaacagactgagtttgtttcaaactcccatttattaacacattgtgcttttgctgttgacccagctgcagcctaccttgatgctgttaaacaaggcgcaaaacctgttggcaattgtgtaaagatgttgactaatggttctggtagtggtcaggctattactagtaccattgattctaacactacgcaggacacatatggtggcgcgtctgtttgtatttattgcagagcacatgttgcacatccaaccatggacggtttttgtcagtataaaggtaagtgggtacaagtgcctataggtacaaatgaccctataagattttgtcttgaaaatactgtttgtaaagtttgtggttgttggcttaatcatggctgtacatgtgaccggactgctatccaaagttttgatagcagttatttaaacgagtcaggggctctagtgccgctcgactagagccctgtaatggtacagacatagattactgtgtccgtgcatttgacggttacaataaagatgcgtcttttatcggaaaaaatctgaagtccaattgtgtgcgcttcaagaatgtagataaggatgacgcgttctatattgttaaacgttgcattaagtcagttatggaccacgagcagtccatgtataacttacttaaaggctgtaatgctgttgctaagcatgatttctttacttggcatgagggcagaaccatttatggtaatgttagtagacaggatcttactaaatacaccatgatggatttgtgcttcgctctgcgtaactttgatgaaaaagattgtggagtttttaaggagatattggttcttactggttgttgtaatactgattactttgaaatgaagaattggtttgaccccatagaaaatgaggacatacaccgtgtgtatgctgctttaggcaaggtagtcgcaaatgcaatgcttaagtgtgttgctttttgcgacgaaatggtgctcaaaggagttgttggtgttttgaccttagacaaccaagatcttaatgggaatttctatgacttcggtgattttgtattgtgtcctcctggaatgggaataccctactgcacgtcatactatgcttatatgatgcctgttatgggtatgactaattgtttagctagtgagtgctttatgaaaagtgacatctttggtcaagacttcaaaacttttgatttgttgaaatatgatttcacagaacataaggaggttttgtttaacaagtactttaagtattggggacaggattatcatcctgattgtgttgattgccatgacgagatgtgtattttgcattgttcaaattttaacacactcttcgcaaccacaattccaaacacagcttttggacctctgtgcagaaaagtgtttattgatggtgtacccgtagttgctactgctggttaccactttaaacaattaggacttgtgtggaacaaagatgttaatacccattctaccagacttactattactgaactcttacagtttgtgacagatccaacgcttatagttgcgtcatcgcctgccttggtggacaaacgcactgtttgtttttctgtcgctgctttgagtacaggattaacatcccaaacagtaaaacctggccattttaataaggagttttatgacttcttacgttctcaagggtttttcgatgagggttcagaattaacattgaagcatttcttttttacacaaaagggtgatgctgcaattaaagattttgattattatcgttacaacagacctaccattctggatattggacaagctcgcgtagcatatcaagtggcagctcgctattttgactgttacgagggtggctgtattacatctagagaggttgttgttacaaaccttaataaaagcgctggttggccccttaataagtttggtaaagctggtttatattatgagtctattagttatgaggaacaagatgctatttttttattaacaaagcgtaatattctccctactatgactcagttaaatcttaaatatgccatatctggtaaggaacgtgcacgtacagtgggtggcgtctctttattagctactatgactacacgacagtttcatcagaaatgtctaaaatccatagtagctaccagaaatgccactgttgttatcggcactaccaagttttatggcggttgggataatatgttaaagaacctgatggccgatgttgacgatcctaaattgatgggatgggactatcctaagtgcgatagagctatgccctcaatgattcgtatgttgtcggctatgattttaggttctaagcatgtcacatgttgtacggctagtgataaattttatagacttagtaatgagcttgctcaagttttgaccgaggttgtttattcaaatggtgggttttattttaaacctggtggtacaacttctggtgatgcaactacagcctacgccaattctgtctttaaaatatttcaggctgtaagttctaacattaattgcgttttgagcgttaactcgtcaaattgcaataattttaatgttaagaagttacagagacaactctatgataattgctatagaaatagtaatgttgatgaatcttttgtggatgacttttatggttatttgcaaaagcatttttctatgatgattctttctgatgatggtgttgtgtgctacaataaaacttatgctgaacttggttacattgctgatattagtgcttttaaagccactttgtattatcagaatggtgtgtttatgagtacagctaagtgttggactgaggaagatctttctataggacctcatgaattttgctcacagcacactatgcagattgtagatgaaaagggtaagtattatctaccatatccagatcctagccgtattatttctgctggtgtttttgtggatgacgtcactaagactgatgctgtcattcttttggaacgctatgtttctctggctatagatgcatacccattgtctaagcatcctaaacctgagtacaggaaggtgttttacgcattgttagattgggttaaacatctcaacaagactcttaacgaaggtgttttggagtctttttctgttacacttttagatgaacatgagtctaagttttgggatgaaagcttttatgctagtatgtatgagaagtctacagtattacaagctgctggtctttgtgtagtatgtggctctcaaacagttctaagatgcggtgattgtttacgcagacctatgttgtgcactaagtgcgcctatgatcatgtgtttggcactgatcataagttcattttagctattacaccatatgtttgtaacacatctggctgcaacgtaactgacgttacaaaactgtatcttggaggtttgaattattactgtgtagaccacaaaccacatctttcattcccactgtgttcagctggtaatgtctttggtttgtacaaaagttctgctttgggttccatggacattgatgtctttaacaaactttctacctctgattggtctgacattcgcgactacaagctggctaatgatgcaaaagagtcactaaggttgtttgcagctgaaacggtcaaggctaaagaggaaagtgttaagtcatcatacgcttatgctaccctaaaggagattgtaggtcctaaggaacttttgcttttatgggaaagtggaaaagccaaaccaccgttaaaccgtaattctgtttttacatgcttccaaattacaaaagactccaagtttcaagttggtgagtttgtgtttgagaaagtagattatggttctgatacggttacttacaaatccactgctactactaagttagtaccaggtatgttatttattttgacttctcataatgttgctccacttagagcgccaacaatggcaaaccaggagaaatattctaccatttacaagttgcacccatcatttaatgttagtgatgcttatgcaaatcttgtaccttattaccaacttattggcaaacagcgtatcaccacaatacagggtcctcctggtagtggaaaatcgcattgctctattggtattggtgtgtattaccctggagcgaggatcgtgttcactgcttgttctcacgctgctgttgattcgctttgtgcaaaagctgtcacagcctatagtgttgataagtgtacacgtattattcctgcacgtgccagagttgagtgttatagtggttttaaacctaacaacactagtgcacaatacgtgtttagtactgttaatgcgttacctgaagttaatgcagacattgttgtcgtggatgaggtgtctatgtgcactaattatgacttgtctgtgattaaccagcgtatatcatataaacacattgtatatgttggtgatcctcaacagcttccagctcctagagttcttatctctaaaggtgttatggaaccaattgactataatgttgtgacacaacgtatgtgtgctataggacccgatgtctttttacacaagtgttacagatgtcctgctgaaatagttaacactgtttcagagcttgtttatgaaaacaagtttgtacctgtcaaagaagctagtaagcagtgcttcaaaatctttgaacgcggtagtgttcaggtagacaatggctccagtataaataggcgtcaacttgatgttgttaagcgatttatacataaaaactccacatggagcaaggctgtgtttatctcaccttacaatagtcaaaattatgtagctgccaggcttttaggtttacaaactcagacagtggattctgctcaaggtagtgaatatgactatgttatattcgcacagacatcagatactgctcatgcctgtaatgccaatcgttttaacgttgccattactagagcaaagaaaggtattttctgtattatgtctgacagaactttgtttgatgcacttaagttctttgaagttactatgacagatttacagtctgaaagcagttgtggtttgtttaaggattgtgcacgtaaccctattgatttaccaccaagtcatgccactacttatttgtcattgtctgatagatttaagactagtggtgacttggctgttcaaataggtaacaacaatgtttgtacctatgaacatgtgatttcatatatgggtttcaggtttgatgttagcatgcctggtagtcatagtttgttctgtactagagactttgccatgcgtcatgtcagaggttggttaggaatggatgtggaaggtgcacatgtcacaggtgacaatgttggcactaatgtacctctacaagttggtttttccaatggtgttgattttgtagctcaacctgaaggttgtgttttaacaaacactggcagtgttataaaacctgttcgtgctcgtgcacctcctggagaacaatttactcacattgtacctctgttacgcaagggacaaccttggagtgtgttgagaaaacgtattgttcaaatgatagcagattttcttgctggctcatctgatgtattggtgtttgtgctttgggctggcggtttagagttgaccactatgcgttattttgttaagattggagctgttaaacattgccaatgtggtactgttgcaacatgctacaattctgttagtaatgactattgttgctttaaacatgcattgggctgtgattatgtttataacccatatgtcatagatattcaacaatggggttatgttggttcactctccaataatcaccatgcaatttgtaatgttcatagaaatgaacacgttgcttctggtgatgctattatgactagatgtttggctgtgtatgattgctttgttaagaatgtggattggtcaattacctaccctatgatagctaatgaaaatgccataaacaagggcggtcgcactgtgcagagtcatattatgcgtgctgctattaaattgtacaaccctaaagcaatccatgacattggtaatcctaagggtattcgttgtgctgtaactgatgccaagtggtattgttatgacaagaaccctattaattctaatgtgaaaacattggagtacgattacatgacacatggccaaatggaaggcttgtgtttattttggaattgtaatgtggatatgtaccctgaattttcaattgtttgcaggtttgacacacgtacacgatctacattgaacctcgaaggtgtaaatggtggttcattgtatgtcaataaccatgcatttcacactcctgcttatgataaacgtgctatggctaaattgaaaccagcaccgtttttctactatgacgacggttcatgtgaggttgttcacgatcaagttaactatgttcctttgagagccactaattgcattaccaagtgtaatattggtggtgctgtatgttctaagcacgctaatctctatagagcatatgttgagtcatataatatttttactcaagctggttttaatatttgggttcctaccacgtttgattgttataatttgtggcagacattcacagatgttaatttacaaggtttagagaacattgcttttaacgttgttaataaaggttcatttgttggtgctgacggtgaattaccagtagccattagtggtgataaagtgttcgtacgtgatggtaacactgataatttagtctttgttaacaaaacatcactgcctacaaatatagcatttgaactttttgctaagaggaaggttggtttaacaccacctcttagtattctcaaaaaccttggtgttgtcgccacatataagtttgtcttgtgggattatgaagctgagcgtcccttgacaagctttactaagtctgtttgtggttatacagactttgcagaagatgtttgtacttgttacgataatagtatacaaggttcatacgaacgttttactctctcaactaatgctgtgttattctctgctactgctgtgaaaacaggtggtaagagtttgccggctattaaactgaattttggaatgcttaacggtaatgcaattgctactgtcaaatcagaagatggtaacataaaaaatattaactggtttgtttacgtacgcaaagatggtaaacatgttgatcattatgatggtttttatacccaaggtcgtaatttacaagactttttgcctcgcagcacaatggaagaagactttttgaacatggatataggcgtgtttattcaaaagtatggtctagaggatttcaacttcgagcacgttgtgtatggtgatgtttcaaaaactactctaggcggtttacacttgttgatttcacaagtacgtctgagtaaaatgggcatcttaaaggcagaggagtttgtggcagcatctgacataacactcaaatgttgtactgtgacttatcttaatgatcctagttctaagactgtttgtacttacatggatttgttgttggatgattgtgtttctttattgaagtctttggagttgactgttgtatcaaaggttcatgaggtcataattgacaacaaaccatggagatggatgctatggtgtaaagataatgccgttgctacattctatcctcagttgcagagtgcagaatggaaatgcgggtattctatgcctggtatttataagacacaacgtatgtgcttagaaccatgtaatttgtataattatggtgcaggtttgaagttgcccagtggcattatgttcaatgttgttaaatacactcaattgtgtcaatattttaacagtaccacgttatgtgttcctcataatatgagagtgttacacttgggtgctggctctgattatggtgttgcaccaggaactgctgttcttaaaaggtggttgccgcacgacgcaattgttgttgacaatgatgttgttgactatgtgagtgatgctgattttagtgttactggtgattgtgcaaccgtttatttggaagacaagtttgacttgttaatttctgatatgtacgatggtaggacaaaggcaattgatggtgaaaatgtttcaaaagaaggatttttcacttacatcaatggtttcatttgtgaaaaccttgccatcggaggttcaattgctattaaagtaacagagtatagctggaataagaaattgtatgaacttgtacaaagattttctttttggactatgttttgcacttctgttaatacgtcatcatcagaagcctttgttgtcggaattaactatcttggtgatttcgcacaaggaccttttatagatggtaacataatacatgcaaattatgtattttggcgtaactccactgttatgagtttgtcctacaactctgttttagacctgagtaaatttaattgcaaacacaaagctactgttgttgtgcaattaaaggatagtgatattaatgacatggtgcttagtcttgttaggagtggtaagttgcttgtaaggggtaatggcaagtgtttgagttttagtaatcatttagtctcaactaaataaaatgtttgttttacttgttgcatatgccttgttgcatattgctggttgtcaaactacaaatgggacgaacactagtaactctgtttgcaacggctgtgttggtcattcggaaattgtatttgatgttgagagtggtggttatataacctccaacgttgcatttaataattggttcctcttaactaatacctcatctgttgtagatggtgttgtgaggagttttcagcctttgttgcttaattgcttatggtctgtttctggctcgcagtttactactggttttgtctattttaatggtactggcagaggtgcttgtaaaggtttttattcaaatgcttcgtctgatgtcatacgttacaacatcaattttgaagaaaaccttagacgtggaaccattttgtttaaaacatcttatggtgctgttgtgttttattgtaccaacaacactttggtttcaggtgatgctcacataccatctggtacagttttgggcaatttttattgctttgtaaatactactattggcaatgaaactacgtctgcttttgtgggtgcactacctaagacagttcgtgagtttgttatttcacgcacaggacatttttatattaatggctatcgctatttctctttaggtgatgtagaagccgttaatttcaatgtcactaatgctgcaaccactgtttgtactgttgcgttagcttcttatgctgacgttttggttaatgtgtcacaaactgctattgctaatataatttattgcaattctgttattaacagactgagatgtgaccagctgtcctttgatgtaccagatggtttttattccacaagccctattcaacccgttgagttacctatgtctattgtgtcgctacctgtttatcacaaacacacgtttattgtgttgcatgttaaattcgaacatcagcgtggccctggcaagtgctataactgtcgtccttctgttattaatattacactggccaattttaatgaaactaaagggcccttgtgtgttgacacatcacacttcactacccaattcgttgataatgtcaagcttgctaggtggagtgctagtattaacacgggaaattgccctttttcttttggcaaagttaataactttgttaaatttggcagtgtatgtttttcgctaaaggatatacccggtggttgcgcaatgcctataatggctaatttggttaatcataaatctcataatataggctcattgtatgtttcttggagtgatggtgatgtaattactggcgtcccgaaacctgttgagggtgttagttcctttatgaatgttacattgaataaatgtactaaatataacatttatgatgtatctggtgtgggtgttattcgcattagcaatgacacctttcttaatggaattacgtacacatcaacttcaggtaaccttctgggttttaaagatgttactaatggcaccatctactctatcactccttgtaacccaccggatcagcttgttgtctatcagcaagctgttgttggtgctatgttgtctgaaaactttactagttacggcttttctaatgttgtagaaatgccgaaatttttctatgcgtccaatggcacttataattgcacagacgctgttttaacgtattctagttttggcgtttgtgcagatggttctataattgctgttcaaccacgtaatgtttcatatgatagtgtttcagctattgtcacagctaatttgtctataccctccaattggaccacttcggtccaggttgagtatttacaaattacaagtacacctatcgtagttgattgctccacttatgtttgcaatggtaatgtgcgctgtgttgaattgcttaagcagtatacttctgcttgtaaaactattgaagacgccttaagaaatagcgccatgctggagtctgcagatgttagtgagatgctcacttttgacaagaaagcgtttacacttgctaatgttagtagttttggtgactacaaccttagcagtgttatacctagcttgcccagaagtggtagtagagtggctggtcgtagtgccatagaagacatactttttagcaaacttgttacgtctggacttggcactgtggacgcagactacaaaaagtgtactaagggtctttccattgctgacttggcttgtgcccaatattataatggcattatggttttgcctggcgtcgctgatgctgaacgtatggccatgtatacaggttctttaattggtggaattgctttaggaggtcttacatcagccgcttcaataccattttcattagcaattcagtcacgtttaaattatgttgcattgcagactgatgttttacaagaaaatcagagaattcttgctgcatcttttaataaagcaatgaccaacatagtagatgcctttactggtgttaatgatgctattacacaaacttcacaagctctacaaacagttgctactgcacttaataagatccaggatgttgttaatcaacaaggcaactcattgaaccatttaacttctcagttgaggcagaattttcaagccatttctagctctattcaggctatctatgacagacttgatattattcaggctgatcaacaagtagataggctgattactggtagattggctgctctgaatgtattcgtttctcatacattgactaagtacactgaagttcgtgcttccagacagcttgcacaacaaaaagtgaatgagtgtgtcaaatcccagtctaagcggtatggcttctgtggaaatggcacgcacattttctcacttgttaatgctgctcctgaggggcttgtttttctccatactgtcttgctgccgacacaatataaggatgttgaggcgtggtctgggttgtgcgttgatggtataaacggttatgtgttgagacaacctaatcttgctctttacaaagaaggcaattattatagaattacatctcgcataatgtttgaaccacgtattcctaccatagcagattttgtccaaattgaaaattgcaatgtcacatttgttaacatttctcgctctgagttgcaaactattgtgccagagtatattgatgttaataagacgctgcaagaattaagttacaaattgccaaattacactgttccagacctagttgttgaacagtacaaccagactattttgaatttgaccagtgaaattagcacccttgaaaataaatctgcggagcttaattacactgttcaaaaactgcaaactttgatcgacaacataaatagcacattagttgacttaaagtggctcaaccgtgttgagacttatatcaagtggccttggtgggtgtggttgtgcatttcagtcgtgctcatctttgtggtgagtatgttgctattatgttgttgttctactggttgctgcggcttctttagttgttttgcatcttctattagaggttgttgtgaatcaactaaacttccttattacgacgttgaaaagatccacatacagtaatggctctaggtttgttcacattgcattttgtgtctgctgttaatcaatctcttatcaagtcgaaagttagtgctgaagtttcacgacaggttatccaagacgtgaaagatggcactgttaccttcaacttgctagcgtatacactaatgagcctctttgttgtgtattttgctttatttaaagcaagatcacaccgtggcagatctgctcttatagtgtttaaaattttaatccttttcgtttatgtgccattgctgtattggtctcaagcatatattgacgcaacgttgattgctgtaattttgcttggaagatttttccatacagcttggcactgctggctctacaagacatgggatttcattgtcttcaatgtaaccacactttgctatgtgcaaggtaagtgttggtttcttgaaaataaggctctgaaaccattcgtctgtttttacggaggggaccaattcctttacataggtgacagaattgttacttatctctcaactaacgatttgtacgttgctcttagaggacgtattgataaagacctcagcctttctagaaaggttgagttatataacggtgaatgtgtatacttgttttgtgaacacccagctgttggaatagtcaatacagatttcaaattagaaatccactaagatgttccttaagctagtggatgatcatgctttgattgttaatgtactactctggtgtgtggtgcttatagtgatactactagtgtgtattacaataattaaactaattaagctttgtttcacttgccatatgttttgtaatagaacagtttatggccccattaaaaatgtgtaccatatttaccaatcatatatgcacatagaccctttccctaaacgagttattgatttctaaactaaacgacaatgtcaaacgacaattgtacgggtgacattgtcacccatttgaagaattggaattttggttggaatgttattctaaccatattcattgttattcttcagtttggacactataaatactccagattgttttatggtttgaagatgcttgtactgtggcttctttggccacttgtacttgctttgtcaatctttgacacctgggctaattgggactctaattgggcctttgttgcatttagcttccttatggccgtatcaacactcgttatatgggtgatgtactttgcaaatagtttcagacttttccgacgtgctcgaactttttgggcatggaatcctgaggttaatgcaatcactgtcacaaccgtgttgggacagacatactaccaacccattcaacaagctccaacaggcattactgtgaccttgttgagcggcgtgctttacgttgacggacatagattggcttcaggtgttcaggttcataacctacctgaatacatgacagttgccgtgcctagcactactataatttatagtagagtcggaaggtccgtaaattctcaaaatagcacaggctgggttttctacgtacgagtaaaacacggtgatttttctgcagtgagctctcccatgagcaacatgacagaaaacgaaagattgcttcattttttctaaactgaacgaaaagatggctacagtcaaatgggctgatgcatctgaaccacaacgtggtcgtcagggtagaataccttattctctttatagccctttgcttgttgatagtgaccaaccttggaaggtgatacctcgtaatttggtacccgtcaacaagaaagacaaaaataagcttataggctattggaatgttcaaaaacgtttcagaactagaaagggcaaacgggtggatttgtcacccaagttacatttttattatcttggcacaggacctcataaagatgcaaaatttagagagcgtgttgaaggtgttgtctgggttgctgttgatggtgctaaaactgaacctacaggttacggtgttaggcgcaagaattcagaaccagagataccacacttcaatcaaaagctcccaaatggtgttactgttgctgatgaacctgactcccgtgctccttcccgttctcagtcaaggtctcagagtcgcagtcgtgttgaatccaaatctcaatctcggaatccttcaagtgacagaaaccataacagtcaggatgacatcatgaaggcagtcgctgcggctcttaaatctttaggttttgacaagcctcaggaaaaagacaaaaagtcagcgaaaacgggtacccctaagccttctcgtaatcagagtcctgcttcttctcaatctgctgccaagattcttgctcgttctcagagttctgaaacaaaagaacaaaagcatgaaatgcaaaagccacggtggaaaagacagcctaacgatgatgtgacatctaatgtcacacaatgttttggccccagagaccttgaccacaactttggaagtgcaggtgttgtggccaatggtgttaaagctaaaggctatccacaatttgctgagcttgtgccgtctacagctgctatgctttttgatagtcacattgtttccaaagagtcaggcaacactgtggtcttgactttcaccactagagtgactgtgcccaaagaccatccacacttgggtaagtttcttgaggaattaaatgcattcactagagaaatgcaacaacagcctcttcttaaccctagtgcactagaattcaacccatcccaaacttcacctgcaactgttgaaccagtgcgtgatgaagtttctattgaaactgacataattgatgaagtcaactaaacatgctactgtgttgaaattcaggctttagttggaattttgcttttgctcttgtttttattatctttcttttatgaatatatttctttcctctgatctacgtaatggtacgatcagagctgcttttaattaacatgatcccttgctttggcttgacaaggatctagtcttatacacaatggtaagcctgtggtagtaaaggtataagaaatttgctactatgttactgaacctaggtgaacgctagtataactcattacaattgtgctggagtaatcaaagatcgcattgacgagccaacaatggaagagccagtcatttgtcttgagacctatctagttagtaactgctaatggaacggtttcgatatggatacacaaaaaaaaaaaaaaaaaaaaaaaaa

SEQ ID NO: 5 - DNA, 20 bp, Artificial Sequence
  Synthetic Oligonucleotide
gatgtggtggttttggacat

SEQ ID NO: 6 - DNA, 24 bp, Artificial Sequence
  Synthetic Oligonucleotide
cttcttagaaaacttacccttagg

SEQ ID NO: 7 - DNA, 26 bp, Artificial Sequence
  Synthetic Oligonucleotide
agttgaagatgcttatgaccaggtgc

SEQ ID NO: 8 - DNA, 24 bp, Artificial Sequence
  Synthetic Oligonucleotide
agacaatatcgtggtattaaaccc

SEQ ID NO: 9 - DNA, 18 bp, Artificial Sequence
  Synthetic Oligonucleotide
agcttgaagacaatctgc

SEQ ID NO: 10 - DNA, 29 bp, Artificial Sequence
  Synthetic Oligonucleotide
ccagaatagtcacaaccatactggtctac

SEQ ID NO: 11 - DNA, 18 bp, Artificial Sequence
  Synthetic Oligonucleotide
cggaggtcttacatcagc

SEQ ID NO: 12 - DNA, 23 bp, Artificial Sequence
  Synthetic Oligonucleotide
atcagtttgtaaagcaacatagt

SEQ ID NO: 13 - DNA, 22 bp, Artificial Sequence
  Synthetic Oligonucleotide
tctttggcactgcaagcacgac

SEQ ID NO: 14 - DNA, 23 bp, Artificial Sequence
  Synthetic Oligonucleotide
cctggtagtcatagtttgttctg

SEQ ID NO: 15 - DNA, 20 bp, Artificial Sequence
  Synthetic Oligonucleotide
acatccattcctaaccaacc

SEQ ID NO: 16 - DNA, 27 bp, Artificial Sequence
  Synthetic Oligonucleotide
ctagagactttgccatgcgtcatgtca

SEQ ID NO: 17 - DNA, 23 bp, Artificial Sequence
  Synthetic Oligonucleotide
aaaggtatggcggctgctgatgt

SEQ ID NO: 18 - DNA, 35 bp, Artificial Sequence
  Synthetic Oligonucleotide
aataaggctattaataatattgttgcttcttttag

SEQ ID NO: 19 - DNA, 28 bp, Artificial Sequence
  Synthetic Oligonucleotide
acaccagcctgcaatctaattgttgaag

SEQ ID NO: 20 - DNA, 23 bp, Artificial Sequence
  Synthetic Oligonucleotide
agacaatatcgtggtattaaacc

SEQ ID NO: 21 - DNA, 20 bp, Artificial Sequence
  Synthetic Oligonucleotide
aggtcggtactaacatcaag

SEQ ID NO: 22 - DNA, 27 bp, Artificial Sequence
  Synthetic Oligonucleotide
aggtcttacatcagcagccgccatacc

SEQ ID NO: 23 - DNA, 16 bp, Artificial Sequence
  Synthetic Oligonucleotide
agtcgtgcttgcagtg

SEQ ID NO: 24 - DNA, 21 bp, Artificial Sequence
  Synthetic Oligonucleotide
atacagcctgaagactataaa

SEQ ID NO: 25 - DNA, 24 bp, Artificial Sequence
  Synthetic Oligonucleotide
attaaaggatgtaacactttagct

SEQ ID NO: 26 - DNA, 25 bp, Artificial Sequence
  Synthetic Oligonucleotide
caatatcgtggtattaaacccgtac

SEQ ID NO: 27 - DNA, 31 bp, Artificial Sequence
  Synthetic Oligonucleotide
cacaattaattataggtacaccaccttgttg

SEQ ID NO: 28 - DNA, 19 bp, Artificial Sequence
  Synthetic Oligonucleotide
cacgttgtctggaagtttc

SEQ ID NO: 29 - DNA, 19 bp, Artificial Sequence
  Synthetic Oligonucleotide
cacgttgtctggaagtttg

SEQ ID NO: 30 - DNA, 20 bp, Artificial Sequence
  Synthetic Oligonucleotide
caggttctcttataggtggc

SEQ ID NO: 31 - DNA, 20 bp, Artificial Sequence
  Synthetic Oligonucleotide
cagtaccagcatgatcacag

SEQ ID NO: 32 - DNA, 20 bp, Artificial Sequence
  Synthetic Oligonucleotide
cgcgtattattgtgaccgtc

SEQ ID NO: 33 - DNA, 25 bp, Artificial Sequence
  Synthetic Oligonucleotide
cgctattcaagaagttattgatgat

SEQ ID NO: 34 - DNA, 29 bp, Artificial Sequence
  Synthetic Oligonucleotide
cgctattcaagaagttattgatgatatgc

SEQ ID NO: 35 - DNA, 17 bp, Artificial Sequence
  Synthetic Oligonucleotide
cggtgaacacgatcctc

SEQ ID NO: 36 - DNA, 28 bp, Artificial Sequence
  Synthetic Oligonucleotide
ctattggtattggtgtgtattaccctgg

SEQ ID NO: 37 - DNA, 23 bp, Artificial Sequence
  Synthetic Oligonucleotide
gaaaatcagaaaattttggctgc

SEQ ID NO: 38 - DNA, 25 bp, Artificial Sequence
  Synthetic Oligonucleotide
gatgtgtatagatatgaaaggaaca

SEQ ID NO: 39 - DNA, 28 bp, Artificial Sequence
  Synthetic Oligonucleotide
gatgtgtatagatatgaaaggaacaatg

SEQ ID NO: 40 - DNA, 24 bp, Artificial Sequence
  Synthetic Oligonucleotide
gcagctaattcttctatactttca

SEQ ID NO: 41 - DNA, 19 bp, Artificial Sequence
  Synthetic Oligonucleotide
gcgtattattgtgaccgtc

SEQ ID NO: 42 - DNA, 17 bp, Artificial Sequence
  Synthetic Oligonucleotide
gtcctcctggtagtgga

SEQ ID NO: 43 - DNA, 20 bp, Artificial Sequence
  Synthetic Oligonucleotide
gtggtttaacaccacgatac

SEQ ID NO: 44 - DNA, 25 bp, Artificial Sequence
  Synthetic Oligonucleotide
gttctcaaatttaatctgaaaacca

SEQ ID NO: 45 - DNA, 23 bp, Artificial Sequence
  Synthetic Oligonucleotide
taaagcaacatagttaagtcgtg

SEQ ID NO: 46 - DNA, 23 bp, Artificial Sequence
  Synthetic Oligonucleotide
tagatggccgtctgtcgtatcca

SEQ ID NO: 47 - DNA, 28 bp, Artificial Sequence
  Synthetic Oligonucleotide
tatggtagtcctcaattattggcctaac

SEQ ID NO: 48 - DNA, 24 bp, Artificial Sequence
  Synthetic Oligonucleotide
tgaaagtatagaagaattagctgc

SEQ ID NO: 49 - DNA, 23 bp, Artificial Sequence
  Synthetic Oligonucleotide
tgtaatagcatcattaacgctac

SEQ ID NO: 50 - DNA, 18 bp, Artificial Sequence
  Synthetic Oligonucleotide
ttataggtggcatggtgc

SEQ ID NO: 51 - DNA, 21 bp, Artificial Sequence
  Synthetic Oligonucleotide
ttcctaggatgtgagggttct

SEQ ID NO: 52 - DNA, 938 bp, Artificial Sequence
  Synthetic Oligonucleotide
tctatgcttcacggaattctgcaatcccggaaatttattgttccatgggttatgtacttgcgtaagcgtggcgaaaagggtgcttacaataaagatcatggatgtggcggttttggacatgtttatgattttaaagttgaagatgcttatgaccaggtgcatgatgagcctaagggtaagttttctaagaaggcttatgctttaattagagggtatcgtggtgttaaaccacttctctatgtagaccagtatggttgtgattatactggtagtcttgcagatggcttagaggcttatgctgataagacattgcaagaaaaaattctcgatgaaggagtggattgtaaaatacaaacagcgccacacattagtcttactatgctggacatacagcctgaagactataaaagtgttgatgtcgctattcaagaagttattgatgatatgcattggggtgatggttttcagattaaatttgagaatcctcacatcctaggaagatgcatagtttttgatgttaaaggtgtagaagaattgcatgacgatttagttaattacattcgtgataaaggttgtgttgctgaccaatccaggaaatggattggccattgtaccatagtatagtttttaccatagtggccattattatgtggattgtgtattttgtgaatagtatcaggttgtttattagaactggaagtttttggagttttaacccagaaacaaacaacttgatgtgtatagatatgaaaggaacaatgtatgttaggccgataattgaggactatcataccctgacggtcacaataatacgcggtcatctttatattcaaggtataaaactaggtattggctattctttggcagatttgccagcttatatgtctgttgctaaggttacacacctgtgcacatgcggccgctgctacactggatcc

SEQ ID NO: 53 - DNA, 942 bp, Artificial Sequence
  Synthetic Oligonucleotide
tctatgcttcacggaattccaaatggtttcaattctgtaggttgtatattgaagagagtgatttaataatttcaaattttaaatttgatgattatgattttagtgtagaagatgtttatgctgaggttcatgctgagcctaaaggtaaatattcacaaaaagcttatgctttacttagacaatatcgtggtattaaacccgtactttttgtagaccagtatggttgtgactattctggtaaattagcagattgtcttcaagcttatggtcattattctttgcaagatatgagacaaaagcagtctgtatggcttgccaattgtgactttgatattgtagtggcttggcatgtagttcgtgattcacgatttgttatgcgcctgcagactatagctactatttgtggtattaaatatgttctaagataatgaaagatgatggaaattgtgttgttttagagcttgatcctccttgtaaattttctatacaagatgttaagggacttaaaattaagtatctttattttattaaaggatgtaacactttagctagagggtgggttgttggtactttatcttcaacaattagattgcaggctggtgttgctactgagtatgcagctaattcttctatactttcattatgtgcattttctgtagatcctaagaaaacttatttagattatatacaacaaggtggtgtacctataattaattgtgttaaaatgctctgtgatcatgctggtactggtatggccattactattaaacctgaggctactattaaccaagattcttatggtggtgcctctgtttgtatttattgccgtgcacgtgtagagcatccagatgtagatggtatatgtaaattacgtggtaaatttgtacaagtccctttgggtataaaagaccctattcttgcggccgctgctacactggatcc

SEQ ID NO: 54 - DNA, 892 bp, Artificial Sequence
  Synthetic Oligonucleotide
tctatgcttcacggaattcttattttgttataaagaggtgtactaagtcggttatggaacacgagcaatccatgtataacctacttaacttttctggtgctttggctgagcatgatttctttacttggaaagatggcagagtcatttatggtaatgttagtagacataatcttactaaatatactatgatggacttggtctatgctatgcgtaactttgatgaacaaaattgtgatgttctaaaagaagtattagttttaactggttgtgccatgtacacaggttctcttataggtggcatggtgctcggaggtcttacatcagcagccgccatacctttttctttggcactgcaagcacgacttaactatgttgctttacaaactgatgtgcttcaagaaaatcagaaaattttggctgcatcatttaataaggctattaataatattgttgcttcttttagtagcgttaatgatgctattacacaaactgcagaggctatacatactgttactattgcacttaataagattcaggatgttgttaatcaacagggtagtgctcttaaccatctcacttcacaattgagacataattttcaggccatttctaattcaattcaggctatttatgaccggcttgattcaattcaagccgatcaacaagttgacagattaattactggacggcttgcagctttgaatgcatttgtttcccaagttttgaataaatatactgaagttcgtggttcaagacgcttagcacagcagaagattaatgaatgtgtcaagtcacaatctaatagatatggtttttgtggcaatggcactcacatcttttcaatcgtcaactcagctccagatggtttggcggccgctgctacactggatcc

SEQ ID NO: 55 - DNA, 999 bp, Artificial Sequence
  Synthetic Oligonucleotide
tctatgcttcacggaattccttagagcgccaacaatggcaaaccaggagaaatattctaccatttacaagttgcacccatcatttaatgttagtgatgcttatgcaaatcttgtaccttattaccaacttattggcaaacagcgtatcaccacaatacagggtcctcctggtagtggaaaatcgcattgctctattggtattggtgtgtattaccctggagcgaggatcgtgttcactgcttgttctcacgctgctgttgattcgctttgtgcaaaagctgtcacagcctatagtgttgataagtgtacacgtattattcctgcacgtgccagagttgagtgttatagtggttttaaacctaacaacactagtgcacaatacgtgtttattgttaatgtactactctggtgtgtggtgcttatagtgatactactagtgtgtattacaataattaaactaattaagctttgtttcacttgccatatgttttgtaatagaacagtttatggccccattaaaaatgtgtaccatatttaccaatcatatatgcacatagaccctttccctaaacgagttattgatttctaaactaaacgacaatgtcaaacgacaattgtacgggtgacattgtcacccatttgaagaattggaattttggttggaatgttattctaaccatattcattgttattcttcagtttggacactataaatactccagattgttttatggtttgaagatgcttgtactgtggcttctttggccacttgtacttgctttgtcaatctttgacacctgggctaattgggactctaattgggcctttgttgcatttagcttccttatggccgtatcaacactcgttatgtgggtgatgtactttgcaaatagtttcagacttttccgacgtgctcgaactttttgggcatggaatcctgaggttaatgcaatcactgtcacaaccgtgttgggacgcggccgctgctacactggatcc

(19) United States
(12) Patent Application Publication
(10) Pub. No.: US 2023/0082096 A1
(43) Pub. Date: Mar. 16, 2023

(54) COMPOSITIONS AND METHODS FOR DETECTING CORONAVIRUS NUCLEIC ACID

(71) Applicant: GEN-PROBE INCORPORATED, San Diego, CA (US)

(72) Inventors: Joshua Kalani MOBERLY, Poway, CA (US); Patrick Lynn PETERSON, San Marcos, CA (US); Megan Renee ALLEN, San Diego, CA (US)

(21) Appl. No.: 17/787,210

(22) PCT Filed: Dec. 17, 2020
(86) PCT No.: PCT/US2020/065487

§ 371 Date: Jun. 17, 2022

Related U.S. Application Data
(60) Provisional application No. 62/949,946, filed on Dec. 18, 2019.

(51) Int. Cl. C12Q 1/70; C12Q 1/6876

(52) U.S. Cl. CPC C12Q 1/701; C12Q 1/6876; C12Q 2600/16

(57) ABSTRACT

Disclosed are nucleic acid oligomers, including amplification oligomers and detection probes, for amplification and/or detection of human coronavirus OC43,HKU1, NL63, and/or 229E nucleic acid. Also disclosed are methods of nucleic acid amplification and/or detection using the disclosed oligomers, as well as corresponding reaction mixtures and kits.

### CROSS-REFERENCE TO RELATED APPLICATIONS

This application claims the benefit of U.S. Provisional Application No. 62/949,946, filed Dec. 18, 2019, which is incorporated herein by reference.

### SEQUENCE LISTING

The Sequence Listing written in file 552196 SeqListing.txt is 162 kilobytes in size, was created Dec. 8, 2020, and is hereby incorporated by reference.

### BACKGROUND

Coronaviruses are a family of RNA viruses that infect avians and mammals, including humans. Coronaviruses belong to the family Coronaviridae, which has four main sub-groupings, known as alphacoronavirus, betacoronavirus, gammacoronavirus, and deltacoronavirus. Human coronaviruses include alphacoronaviruses 229E and NL63 and betacoronaviruses OC43, HKU1, SARS-CoV (the coronavirus that causes severe acute respiratory syndrome, or SARS), SARS-CoV-2 (the coronavirus that causes COVID-19), and MERS-CoV (the coronavirus that causes Middle East Respiratory Syndrome, or MERS).

Human coronaviruses cause approximately 10-15% of all upper and lower respiratory tract infections. They account for significant hospitalizations of children under 18 years of age, the elderly, and immunocompromised individuals. Four human coronaviruses (229E, HKU1, NL63, and OC43) are associated with a range of respiratory outcomes, including bronchiolitis and pneumonia (see Gaunt et al., Journal of Clinical Microbiology 48:2940-2947, 2010). Up to 10% of acute respiratory diseases are caused by human coronavirus NL63 (NL63) (see Abdul-Rasool and Fielding, The Open Virology Journal 4:76-84, 2010). An important aspect NL63 infection is the co-infection with other human coronaviruses, influenza A, respiratory syncytial virus (RSV), and parainfluenza virus human metapneumovirus (see id). In children, coronaviruses are associated with acute respiratory tract illness, pneumonia, and Croup leading in many cases to hospitalization. In one epidemiological study, out of 1471 hospitalized children (<2 years of age), 207 (14%) were positive for human coronavirus (see Dijkman et al., Journal of Clinical Virology 53:135-139, 2012). In a large-scale survey on 11,661 diagnostic respiratory samples collected in Edinburgh, UK, between 2006 and 2009, 267 (2.30%) were positive for at least one coronavirus, accounting for 8.15% of all virus detections (see Gaunt et al., supra). 11 to 41% of coronaviruses detected were present in samples tested positive for other respiratory viruses (e.g., RSV) (see id).

Several commercial assays based on nucleic acids detection are available for detection of human coronavirus OC43, HKU1, NL63, and 229E in clinical specimens. These include the NxTAG® Respiratory Pathogen Panel (Luminex), the BIOFIRE® FILMARRAY® Respiratory Panel (bioMérieux), and the ePlex® Respiratory Pathogen Panel (GenMark Diagnostics).

### SUMMARY

Embodiment 1. A composition for determining the presence or absence of each of human coronaviruses OC43, HKU1, NL63, and 229E in a sample, said composition comprising: a first amplification oligomer combination comprising first and second human coronavirus OC43 (OC43)-specific amplification oligomers capable of amplifying a target region of an OC43 target nucleic acid, wherein the first OC43-specific amplification oligomer comprises a first OC43-specific target-hybridizing sequence substantially corresponding to the nucleotide sequence of SEQ ID NO:5, SEQ ID NO:38, SEQ ID NO:39, or SEQ ID NO:24, and wherein the second OC43-specific amplification oligomer comprises a second OC43-specific target-hybridizing sequence substantially corresponding to the nucleotide sequence of SEQ ID NO:6, SEQ ID NO:43, SEQ ID NO:41, SEQ ID NO:32, SEQ ID NO:44, or SEQ ID NO:51; a second amplification oligomer combination comprising first and second human coronavirus HKU1 (HKU1)-specific amplification oligomers capable of amplifying a target region of an HKU1 target nucleic acid, wherein the first HKU-specific amplification oligomer comprises a first HKU1-specific target-hybridizing sequence substantially corresponding to the nucleotide sequence of SEQ ID NO:8, SEQ ID NO:20, SEQ ID NO:26, SEQ ID NO:40, or SEQ ID NO:25, and wherein the second HKU1-specific amplification oligomer comprises a second HKU1-specific target-hybridizing sequence substantially corresponding to the nucleotide sequence of SEQ ID NO:9, SEQ ID NO:31, or SEQ ID NO:48; a third amplification oligomer combination comprising first and second human coronavirus NL63 (NL63)-specific amplification oligomers capable of amplifying a target region of an NL63 target nucleic acid, wherein the first NL63-specific amplification oligomer comprises a first NL63-specific target-hybridizing sequence substantially corresponding to the nucleotide sequence of SEQ ID NO:11, SEQ ID NO:37, SEQ ID NO:50, or SEQ ID NO:30, and wherein the second NL63-specific amplification oligomer comprises a second NL63-specific target-hybridizing sequence substantially corresponding to the nucleotide sequence of SEQ ID NO:12, SEQ ID NO:49, SEQ ID NO:45, or SEQ ID NO:23; and a fourth amplification oligomer combination comprising first and second human coronavirus 229E (229E)-specific amplification oligomers capable of amplifying a target region of a 229E target nucleic acid, wherein the first 229E-specific amplification oligomer comprises a first 229E-specific target-hybridizing sequence substantially corresponding to the nucleotide sequence of SEQ ID NO:14 or SEQ ID NO:42, and wherein the second 229E-specific amplification oligomer comprises a second 229E-specific target-hybridizing sequence substantially corresponding to the nucleotide sequence of SEQ ID NO:15 or SEQ ID NO:35.

Embodiment 2. The composition of embodiment 1, further comprising an OC43-specific detection probe oligomer comprising an OC43-specific detection probe target-hybridizing sequence that is from about 15 to about 35 nucleotides in length and is configured to hybridize to a target sequence contained within an OC43 amplicon amplifiable by the first and second OC43-specific amplification oligomers.

Embodiment 3. The composition of embodiment 1 or embodiment 2, wherein the first OC43-specific target-hybridizing sequence is SEQ ID NO:5, SEQ ID NO:38, SEQ ID NO:39, or SEQ ID NO:24, or an RNA equivalent or DNA/RNA chimeric thereof and including from 0-7 nucleotide analogs, and/or wherein the second OC43-specific target-hybridizing sequence is SEQ ID NO:6, SEQ ID NO:43, SEQ ID NO:41, SEQ ID NO:32, SEQ ID NO:44, or SEQ ID NO:51, or an RNA equivalent or DNA/RNA chimeric thereof and including from 0-7 nucleotide analogs.

Embodiment 4. The composition of embodiment 1 or embodiment 2, wherein the first OC43-specific target-hybridizing sequence and the second OC43-specific target-hybridizing sequence are: (a) SEQ ID NO:5 and SEQ ID NO:6; (b) SEQ ID NO:5 and SEQ ID NO:43; (c) SEQ ID NO:38 or SEQ ID NO:39 and SEQ ID NO:41 or SEQ ID NO:32; or (d) SEQ ID NO:24 and SEQ ID NO:44 or SEQ ID NO:51.

Embodiment 5. The composition of embodiment 2, 3, or 4, wherein the OC43-specific detection probe target-hybridizing sequence is SEQ ID NO:7, SEQ ID NO:47, SEQ ID NO:33, or SEQ ID NO:34, or an RNA equivalent or DNA/RNA chimeric thereof and including from 0-7 nucleotide analogs.

Embodiment 6. The composition of any one of embodiments 2 to 5, wherein the OC43-specific detection probe oligomer further comprises a detectable label.

Embodiment 7. The composition of embodiment 6, wherein the detectable label is a fluorescent or chemiluminescent label.

Embodiment 8. The composition of embodiment 6, wherein the detectable label is a fluorescent label and the OC43-specific detection probe oligomer further comprises a non-fluorescent quencher.

Embodiment 9. The composition of any one of embodiments 1 to 8, further comprising an HKU1-specific detection probe oligomer comprising an HKU1-specific detection probe target-hybridizing sequence that is from about 15 to about 35 nucleotides in length and is configured to hybridize to a target sequence contained within an HKU1 amplicon amplifiable by the first and second HKU1-specific amplification oligomers.

Embodiment 10. The composition of any one of embodiments 1 to 9, wherein the first HKU1-specific target-hybridizing sequence is SEQ ID NO:8, SEQ ID NO:20, SEQ ID NO:26, SEQ ID NO:40, or SEQ ID NO:25, or an RNA equivalent or DNA/RNA chimeric thereof and including from 0-7 nucleotide analogs, and/or wherein the second HKU1-specific target-hybridizing sequence is SEQ ID NO:9, SEQ ID NO:31, or SEQ ID NO:48, or an RNA equivalent or DNA/RNA chimeric thereof and including from 0-7 nucleotide analogs.

Embodiment 11. The composition of any one of embodiments 1 to 9, wherein the first HKU1-specific target-hybridizing sequence and the second HKU1-specific target hybridizing sequence are (a) SEQ ID NO:8 and SEQ ID NO:9; (b) SEQ ID NO:20 or SEQ ID NO:26 and SEQ ID NO:9; (c) SEQ ID NO:40 and SEQ ID NO:31; or (d) SEQ ID NO:25 and SEQ ID NO:48.

Embodiment 12. The composition of embodiment 9, 10, or 11, wherein the HKU1-specific detection probe target-hybridizing sequence is SEQ ID NO:10, SEQ ID NO:27, or SEQ ID NO:19, or an RNA equivalent or DNA/RNA chimeric thereof and including from 0-7 nucleotide analogs.

Embodiment 13. The composition of any one of embodiments 9 to 11, wherein the HKU1-specific detection probe oligomer further comprises a detectable label.

Embodiment 14. The composition of embodiment 13, wherein the detectable label is a fluorescent or chemiluminescent label.

Embodiment 15. The composition of embodiment 13, wherein the detectable label is a fluorescent label and the HKU1-specific detection probe oligomer further comprises a non-fluorescent quencher.

Embodiment 16. The composition of any one of embodiments 1 to 15, further comprising an NL63-specific detection probe oligomer comprising an NL63-specific detection probe target-hybridizing sequence that is from about 15 to about 35 nucleotides in length and is configured to hybridize to a target sequence contained within an NL63 amplicon amplifiable by the first and second NL63-specific amplification oligomers.

Embodiment 17. The composition of any one of embodiments 1 to 16, wherein the first NL63-specific target-hybridizing sequence is SEQ ID NO:11, SEQ ID NO:37, SEQ ID NO:50, or SEQ ID NO:30, or an RNA equivalent or DNA/RNA chimeric thereof and including from 0-7 nucleotide analogs, and/or wherein the second NL63-specific target-hybridizing sequence is SEQ ID NO:12, SEQ ID NO:49, SEQ ID NO:45, or SEQ ID NO:23, or an RNA equivalent or DNA/RNA chimeric thereof and including from 0-7 nucleotide analogs.

Embodiment 18. The composition of embodiment 16, wherein the first NL63-specific target-hybridizing sequence and the second NL63-specific target hybridizing sequence are (a) SEQ ID NO:11 and SEQ ID NO:12; (b) SEQ ID NO:37 and SEQ ID NO:49; or (c) SEQ ID NO:30 or SEQ ID NO:50 and SEQ ID NO:23 or SEQ ID NO:45.

Embodiment 19. The composition of embodiment 16, 17, or 18, wherein the NL63-specific detection probe target-hybridizing sequence is SEQ ID NO:13, SEQ ID NO:18, SEQ ID NO:22, or SEQ ID NO:17, or an RNA equivalent or DNA/RNA chimeric thereof and including from 0-7 nucleotide analogs.

Embodiment 20. The composition of any one of embodiments 16 to 19, wherein the NL63-specific detection probe oligomer further comprises a detectable label.

Embodiment 21. The composition of embodiment 20, wherein the detectable label is a fluorescent or chemiluminescent label.

Embodiment 22. The composition of embodiment 20, wherein the detectable label is a fluorescent label and the NL63-specific detection probe oligomer further comprises a non-fluorescent quencher.

Embodiment 23. The composition of any one of embodiments 1 to 22, further comprising a 229E-specific detection probe oligomer comprising a 229E-specific detection probe target-hybridizing sequence that is from about 15 to about 35 nucleotides in length and is configured to hybridize to a target sequence contained within a 229E amplicon amplifiable by the first and second 229E-specific amplification oligomers.

Embodiment 24. The composition of any one of embodiments 1 to 23, wherein the first 229E-specific target-hybridizing sequence is SEQ ID NO:14 or SEQ ID NO:42, or an RNA equivalent or DNA/RNA chimeric thereof and including from 0-7 nucleotide analogs, and/or wherein the second 229E-specific target-hybridizing sequence is SEQ ID NO:15 or SEQ ID NO:35, or an RNA equivalent or DNA/RNA chimeric thereof and including from 0-7 nucleotide analogs.

Embodiment 25. The composition of any one of embodiments 1 to 23, wherein the first 229E-specific target-hybridizing sequence and the second 229E-specific target-hybridizing sequence are (a) SEQ ID NO:14 and SEQ ID NO:15; or (b) SEQ ID NO:42 and SEQ ID NO:35.

Embodiment 26. The composition of embodiment 23, 24, or 25, wherein the 229E-specific detection probe target-hybridizing sequence is SEQ ID NO:16 or SEQ ID NO:36, or an RNA equivalent or DNA/RNA chimeric thereof and including from 0-7 nucleotide analogs.

Embodiment 27. The composition of any one of embodiments 24 to 26, wherein the 229E-specific detection probe oligomer further comprises a detectable label.

Embodiment 28. The composition of embodiment 27, wherein the detectable label is a fluorescent or chemiluminescent label.

Embodiment 29. The composition of embodiment 27, wherein the detectable label is a fluorescent label and the 229E-specific detection probe oligomer further comprises a non-fluorescent quencher.

Embodiment 30. A composition for determining the presence or absence of human coronavirus OC43 (OC43) in a sample, said composition comprising: a first amplification oligomer combination comprising first and second human coronavirus OC43-specific amplification oligomers capable of amplifying a target region of an OC43 target nucleic acid, wherein the first OC43-specific amplification oligomer comprises a first OC43-specific target-hybridizing sequence substantially corresponding to the nucleotide sequence of SEQ ID NO:5, SEQ ID NO:38, SEQ ID NO:39, or SEQ ID NO:24, and wherein the second OC43-specific amplification oligomer comprises a second OC43-specific target-hybridizing sequence substantially corresponding to the nucleotide sequence of SEQ ID NO:6, SEQ ID NO:43, SEQ ID NO:41, SEQ ID NO:32, SEQ ID NO:44, or SEQ ID NO:51.

Embodiment 31. The composition of embodiment 30, further comprising an OC43-specific detection probe oligomer comprising an OC43-specific detection probe target-hybridizing sequence that is from about 15 to about 35 nucleotides in length and is configured to hybridize to a target sequence contained within an OC43 amplicon amplifiable by the first and second OC43-specific amplification oligomers.

Embodiment 32. The composition of embodiment 30 or 31, wherein the first OC43-specific target-hybridizing sequence is SEQ ID NO:5, SEQ ID NO:38, SEQ ID NO:39, or SEQ ID NO:24, or an RNA equivalent or DNA/RNA chimeric thereof and including from 0-7 nucleotide analogs, and/or wherein the second OC43-specific target-hybridizing sequence is SEQ ID NO:6, SEQ ID NO:43, SEQ ID NO:41, SEQ ID NO:32, SEQ ID NO:44, or SEQ ID NO:51, or an RNA equivalent or DNA/RNA chimeric thereof and including from 0-7 nucleotide analogs.

Embodiment 33. The composition of embodiment 30 or 31, wherein the first OC43-specific target-hybridizing sequence and the second OC43-specific target-hybridizing sequence are: (a) SEQ ID NO:5 and SEQ ID NO:6; (b) SEQ ID NO:5 and SEQ ID NO:43; (c) SEQ ID NO:38 or SEQ ID NO:39 and SEQ ID NO:41 or SEQ ID NO:42; (d) or SEQ ID NO:24 and SEQ ID NO:44 or SEQ ID NO:51.

Embodiment 34. The composition of embodiment 31, 32, or 33, wherein the OC43-specific detection probe target-hybridizing sequence is SEQ ID NO:7, SEQ ID NO:47, SEQ ID NO:33, or SEQ ID NO:34, or an RNA equivalent or DNA/RNA chimeric thereof and including from 0-7 nucleotide analogs.

Embodiment 35. The composition of any one of embodiments 31 to 34, wherein the OC43-specific detection probe oligomer further comprises a detectable label.

Embodiment 36. The composition of embodiment 35, wherein the detectable label is a fluorescent or chemiluminescent label.

Embodiment 37. The composition of embodiment 35, wherein the detectable label is a fluorescent label and the OC43-specific detection probe oligomer further comprises a non-fluorescent quencher.

Embodiment 38. The composition of any one of embodiments 30 to 37, further comprising: an amplification oligomer combination capable of amplifying a target region of a human coronavirus HKU1 (HKU1) target nucleic acid; and/or an amplification oligomer combination capable of amplifying a target region of a human coronavirus NL63 (NL63) target nucleic acid; and/or an amplification oligomer combination capable of amplifying a target region of a human coronavirus 229E (229E) target nucleic acid.

Embodiment 39. A composition for determining the presence or absence of human coronavirus HKU1 (HKU1) in a sample, said composition comprising: a first amplification oligomer combination comprising first and second human coronavirus HKU1-specific amplification oligomers capable of amplifying a target region of an HKU1 target nucleic acid, wherein the first HKU-specific amplification oligomer comprises a first HKU1-specific target-hybridizing sequence substantially corresponding to the nucleotide sequence of SEQ ID NO:8, SEQ ID NO:20, SEQ ID NO:26, SEQ ID NO:40, or SEQ ID NO:25, and wherein the second HKU1-specific amplification oligomer comprises a second HKU1-specific target-hybridizing sequence substantially corresponding to the nucleotide sequence of SEQ ID NO:9, SEQ ID NO:31, or SEQ ID NO:48.

Embodiment 40. The composition of embodiment 39, further comprising an HKU1-specific detection probe oligomer comprising an HKU1-specific detection probe target-hybridizing sequence that is from about 15 to about 35 nucleotides in length and is configured to hybridize to a target sequence contained within an HKU1 amplicon amplifiable by the first and second HKU 1-specific amplification oligomers.

Embodiment 41. The composition of embodiment 39 or 40, wherein the first HKU1-specific target-hybridizing sequence is SEQ ID NO:8, SEQ ID NO:20, SEQ ID NO:26, SEQ ID NO:40, or SEQ ID NO:25, or an RNA equivalent or DNA/RNA chimeric thereof and including from 0-7 nucleotide analogs, and/or wherein the second HKU1-specific target-hybridizing sequence is SEQ ID NO:9, SEQ ID NO:31, or SEQ ID NO:48, or an RNA equivalent or DNA/RNA chimeric thereof and including from 0-7 nucleotide analogs.

Embodiment 42. The composition of embodiment 39 or 40, wherein the first HKU1-specific target-hybridizing sequence and the second HKU1-specific target hybridizing sequence are (a) SEQ ID NO:8 and SEQ ID NO:9; (b) SEQ ID NO:20 or SEQ ID NO:26 and SEQ ID NO:9; (c) SEQ ID NO:40 and SEQ ID NO:31; or (c) SEQ ID NO:25 and SEQ ID NO:48.

Embodiment 43. The composition of embodiment 40, 41, or 42, wherein the HKU1-specific detection probe target-hybridizing sequence is SEQ ID NO:10, SEQ ID NO:27, or SEQ ID NO:19, or an RNA equivalent or DNA/RNA chimeric thereof and including from 0-7 nucleotide analogs.

Embodiment 44. The composition of any one of embodiments 40 to 43, wherein the HKU-specific detection probe oligomer further comprises a detectable label.

Embodiment 45. The composition of embodiment 44, wherein the detectable label is a fluorescent or chemiluminescent label.

Embodiment 46. The composition of embodiment 44, wherein the detectable label is a fluorescent label and the HKU1-specific detection probe oligomer further comprises a non-fluorescent quencher.

Embodiment 47. The composition of any one of embodiments 39 to 46, further comprising: an amplification oligomer combination capable of amplifying a target region of a human coronavirus OC43 (OC43) target nucleic acid; and/or an amplification oligomer combination capable of amplifying a target region of a human coronavirus NL63 (NL63) target nucleic acid; and/or an amplification oligomer combination capable of amplifying a target region of a human coronavirus 229E (229E) target nucleic acid.

Embodiment 48. A composition for determining the presence or absence of human coronavirus NL63 (NL63) in a sample, said composition comprising: a first amplification oligomer combination comprising first and second NL63-specific amplification oligomers capable of amplifying a target region of an NL63 target nucleic acid, wherein the first NL63-specific amplification oligomer comprises a first NL63-specific target-hybridizing sequence substantially corresponding to the nucleotide sequence of SEQ ID NO:11, SEQ ID NO:37, SEQ ID NO:50, or SEQ ID NO:30, and wherein the second NL63-specific amplification oligomer comprises a second NL63-specific target-hybridizing sequence substantially corresponding to the nucleotide sequence of SEQ ID NO:12, SEQ ID NO:49, SEQ ID NO:45, or SEQ ID NO:23.

Embodiment 49. The composition of embodiment 48, further comprising an NL63-specific detection probe oligomer comprising an NL63-specific detection probe target-hybridizing sequence that is from about 15 to about 35 nucleotides in length and is configured to hybridize to a target sequence contained within an NL63 amplicon amplifiable by the first and second NL63-specific amplification oligomers.

Embodiment 50. The composition of embodiment 48 or 49, wherein the first NL63-specific target-hybridizing sequence is SEQ ID NO:11, SEQ ID NO:37, SEQ ID NO:50, or SEQ ID NO:30, or an RNA equivalent or DNA/RNA chimeric thereof and including from 0-7 nucleotide analogs, and/or wherein the second NL63-specific target-hybridizing sequence is SEQ ID NO:12, SEQ ID NO:49, SEQ ID NO:45, or SEQ ID NO:23, or an RNA equivalent or DNA/RNA chimeric thereof and including from 0-7 nucleotide analogs.

Embodiment 51. The composition of embodiment 48 or 49, wherein the first NL63-specific target-hybridizing sequence and the second NL63-specific target hybridizing sequence are (a) SEQ ID NO:11 and SEQ ID NO:12; (b) SEQ ID NO:37 and SEQ ID NO:49; or (c) SEQ ID NO:30 or SEQ ID NO:50 and SEQ ID NO:23 or SEQ ID NO:45.

Embodiment 52. The composition of embodiment 49, 50, or 51, wherein the NL63-specific detection probe target-hybridizing sequence is SEQ ID NO:13, SEQ ID NO:18, SEQ ID NO:22, or SEQ ID NO:17, or an RNA equivalent or DNA/RNA chimeric thereof and including from 0-7 nucleotide analogs.

Embodiment 53. The composition of any one of embodiments 49 to 52, wherein the NL63-specific detection probe oligomer further comprises a detectable label.

Embodiment 54. The composition of embodiment 53, wherein the detectable label is a fluorescent or chemiluminescent label.

Embodiment 55. The composition of embodiment 53, wherein the detectable label is a fluorescent label and the NL63-specific detection probe oligomer further comprises a non-fluorescent quencher.

Embodiment 56. The composition of any one of embodiments 48 to 55, further comprising: an amplification oligomer combination capable of amplifying a target region of a human coronavirus OC43 (OC43) target nucleic acid; and/or an amplification oligomer combination capable of amplifying a target region of a human coronavirus HKU1 (HKU1) target nucleic acid; and/or an amplification oligomer combination capable of amplifying a target region of a human coronavirus 229E (229E) target nucleic acid.

Embodiment 57. A composition for determining the presence or absence of human coronavirus 229E (229E) in a sample, said composition comprising: a first amplification oligomer combination comprising first and second 229E-specific amplification oligomers capable of amplifying a target region of a 229E target nucleic acid, wherein the first 229E-specific amplification oligomer comprises a first 229E-specific target-hybridizing sequence substantially corresponding to the nucleotide sequence of SEQ ID NO:14 or SEQ ID NO:42, and wherein the second 229E-specific amplification oligomer comprises a second 229E-specific target-hybridizing sequence substantially corresponding to the nucleotide sequence of SEQ ID NO:15 or SEQ ID NO:35.

Embodiment 58. The composition of embodiment 57, further comprising a 229E-specific detection probe oligomer comprising a 229E-specific detection probe target-hybridizing sequence that is from about 15 to about 35 nucleotides in length and is configured to hybridize to a contained within a 229E amplicon amplifiable by the first and second 229E-specific amplification oligomers.

Embodiment 59. The composition of embodiment 57 or 58, wherein the first 229E-specific target-hybridizing sequence is SEQ ID NO:14 or SEQ ID NO:42, or an RNA equivalent or DNA/RNA chimeric thereof and including from 0-7 nucleotide analogs, and/or wherein the second 229E-specific target-hybridizing sequence is SEQ ID NO:15 or SEQ ID NO:35, or an RNA equivalent or DNA/RNA chimeric thereof and including from 0-7 nucleotide analogs.

Embodiment 60. The composition of embodiment 57 or 58, wherein the first 229E-specific target-hybridizing sequence and the second 229E-specific target-hybridizing sequence are (a) SEQ ID NO:14 and SEQ ID NO:15; or (b) SEQ ID NO:42 and SEQ ID NO:35.

Embodiment 61. The composition of embodiment 58, 59, or 60, wherein the 229E-specific detection probe target-hybridizing sequence is SEQ ID NO:16 or SEQ ID NO:36, or an RNA equivalent or DNA/RNA chimeric thereof and including from 0-7 nucleotide analogs.

Embodiment 62. The composition of any one of embodiments 58 to 61, wherein the 229E-specific detection probe oligomer further comprises a detectable label.

Embodiment 63. The composition of embodiment 62, wherein the detectable label is a fluorescent or chemiluminescent label.

Embodiment 64. The composition of embodiment 62, wherein the detectable label is a fluorescent label and the 229E-specific detection probe oligomer further comprises a non-fluorescent quencher.

Embodiment 65. The composition of any one of embodiments 57 to 64, further comprising: an amplification oligomer combination capable of amplifying a target region of a human coronavirus OC43 (OC43) target nucleic acid; and/or an amplification oligomer combination capable of amplifying a target region of a human coronavirus HKU1 (HKU1) target nucleic acid; and/or an amplification oligomer combination capable of amplifying a target region of a human coronavirus NL63 (NL63) target nucleic acid.

Embodiment 66. A kit for determining the presence or absence of each of human coronaviruses OC43, HKU1, NL63, and 229E in a sample, said kit comprising: a first amplification oligomer combination comprising first and second human coronavirus OC43 (OC43)-specific amplification oligomers capable of amplifying a target region of an OC43 target nucleic acid, wherein the first OC43-specific amplification oligomer comprises a first OC43-specific target-hybridizing sequence substantially corresponding to the nucleotide sequence of SEQ ID NO:5, SEQ ID NO:38, SEQ ID NO:39, or SEQ ID NO:24, and wherein the second OC43-specific amplification oligomer comprises a second OC43-specific target-hybridizing sequence substantially corresponding to the nucleotide sequence of SEQ ID NO:6, SEQ ID NO:43, SEQ ID NO:41, SEQ ID NO:32, SEQ ID NO:44, or SEQ ID NO:51; a second amplification oligomer combination comprising first and second human coronavirus HKU1 (HKU1)-specific amplification oligomers capable of amplifying a target region of an HKU1 target nucleic acid, wherein the first HKU-specific amplification oligomer comprises a first HKU1-specific target-hybridizing sequence substantially corresponding to the nucleotide sequence of SEQ ID NO:8, SEQ ID NO:20, SEQ ID NO:26, SEQ ID NO:40, or SEQ ID NO:25, and wherein the second HKU1-specific amplification oligomer comprises a second HKU1-specific target-hybridizing sequence substantially corresponding to the nucleotide sequence of SEQ ID NO:9, SEQ ID NO:31, or SEQ ID NO:48; a third amplification oligomer combination comprising first and second human coronavirus NL63 (NL63)-specific amplification oligomers capable of amplifying a target region of an NL63 target nucleic acid, wherein the first NL63-specific amplification oligomer comprises a first NL63-specific target-hybridizing sequence substantially corresponding to the nucleotide sequence of SEQ ID NO:11, SEQ ID NO:37, SEQ ID NO:50, or SEQ ID NO:30, and wherein the second NL63-specific amplification oligomer comprises a second NL63-specific target-hybridizing sequence substantially corresponding to the nucleotide sequence of SEQ ID NO:12, SEQ ID NO:49, SEQ ID NO:45, or SEQ ID NO:23; and a fourth amplification oligomer combination comprising first and second human coronavirus 229E (229E)-specific amplification oligomers capable of amplifying a target region of a 229E target nucleic acid, wherein the first 229E-specific amplification oligomer comprises a first 229E-specific target-hybridizing sequence substantially corresponding to the nucleotide sequence of SEQ ID NO:14 or SEQ ID NO:42, and wherein the second 229E-specific amplification oligomer comprises a second 229E-specific target-hybridizing sequence substantially corresponding to the nucleotide sequence of SEQ ID NO:15 or SEQ ID NO:35.

Embodiment 67. The kit of embodiment 66, further comprising an OC43-specific detection probe oligomer comprising an OC43-specific detection probe target-hybridizing sequence that is from about 15 to about 35 nucleotides in length and is configured to hybridize to a target sequence contained within an OC43 amplicon amplifiable by the first and second OC43-specific amplification oligomers.

Embodiment 68. The kit of embodiment 66 or 67, wherein the first OC43-specific target-hybridizing sequence is SEQ ID NO:5, SEQ ID NO:38, SEQ ID NO:39, or SEQ ID NO:24, or an RNA equivalent or DNA/RNA chimeric thereof and including from 0-7 nucleotide analogs, and/or wherein the second OC43-specific target-hybridizing sequence is SEQ ID NO:6, SEQ ID NO:43, SEQ ID NO:41, SEQ ID NO:32, SEQ ID NO:44, or SEQ ID NO:51, or an RNA equivalent or DNA/RNA chimeric thereof and including from 0-7 nucleotide analogs.

Embodiment 69. The kit of embodiment 66 or 67, wherein the first OC43-specific target-hybridizing sequence and the second OC43-specific target-hybridizing sequence are: (a) SEQ ID NO:5 and SEQ ID NO:6; (b) SEQ ID NO:5 and SEQ ID NO:43; (c) SEQ ID NO:38 or SEQ ID NO:39 and SEQ ID NO:41 or SEQ ID NO:42; or (d) SEQ ID NO:24 and SEQ ID NO:44 or SEQ ID NO:51.

Embodiment 70. The kit of embodiment 67, 68, or 69, wherein the OC43-specific detection probe target-hybridizing sequence is SEQ ID NO:7, SEQ ID NO:47, SEQ ID NO:33, or SEQ ID NO:34, or an RNA equivalent or DNA/RNA chimeric thereof and including from 0-7 nucleotide analogs.

Embodiment 71. The kit of any one of embodiments 67 to 70, wherein the OC43-specific detection probe oligomer further comprises a detectable label.

Embodiment 72. The kit of embodiment 71, wherein the detectable label is a fluorescent or chemiluminescent label.

Embodiment 73. The kit of embodiment 71, wherein the detectable label is a fluorescent label and the OC43-specific detection probe oligomer further comprises a non-fluorescent quencher.

Embodiment 74. The kit of any one of embodiments 66 to 73, further comprising an HKU1-specific detection probe oligomer comprising an HKU1-specific detection probe target-hybridizing sequence that is from about 15 to about 35 nucleotides in length and is configured to hybridize to a target sequence contained within an HKU1 amplicon amplifiable by the first and second HKU1-specific amplification oligomers.

Embodiment 75. The kit of any one of embodiments 66 to 74, wherein the first HKU1-specific target-hybridizing sequence is SEQ ID NO:8, SEQ ID NO:20, SEQ ID NO:26, SEQ ID NO:40, or SEQ ID NO:25, or an RNA equivalent or DNA/RNA chimeric thereof and including from 0-7 nucleotide analogs, and/or wherein the second HKU1-specific target-hybridizing sequence is SEQ ID NO:9, SEQ ID NO:31, or SEQ ID NO:48, or an RNA equivalent or DNA/RNA chimeric thereof and including from 0-7 nucleotide analogs.

Embodiment 76. The kit of any one of embodiments 66 to 74, wherein the first HKU1-specific target-hybridizing sequence and the second HKU1-specific target hybridizing sequence are (a) SEQ ID NO:8 and SEQ ID NO:9; (b) SEQ ID NO:20 or SEQ ID NO:26 and SEQ ID NO:9; (c) SEQ ID NO:40 and SEQ ID NO:31; or (d) SEQ ID NO:25 and SEQ ID NO:48.

Embodiment 77. The kit of embodiment 74, 75, or 76, wherein the HKU1-specific detection probe target-hybridizing sequence is SEQ ID NO:10, SEQ ID NO:27, or SEQ ID NO:19, or an RNA equivalent or DNA/RNA chimeric thereof and including from 0-7 nucleotide analogs.

Embodiment 78. The kit of any one of embodiments 74 to 77, wherein the HKU1-specific detection probe oligomer further comprises a detectable label.

Embodiment 79. The kit of embodiment 78, wherein the detectable label is a fluorescent or chemiluminescent label.

Embodiment 80. The kit of embodiment 78, wherein the detectable label is a fluorescent label and the HKU1-specific detection probe oligomer further comprises a non-fluorescent quencher.

Embodiment 81. The kit of any one of embodiments 66 to 80, further comprising an NL63-specific detection probe oligomer comprising an NL63-specific detection probe target-hybridizing sequence that is from about 15 to about 35 nucleotides in length and is configured to hybridize to a target sequence contained within an NL63 amplicon amplifiable by the first and second NL63-specific amplification oligomers.

Embodiment 82. The kit of any one of embodiments 66 to 81, wherein the first NL63-specific target-hybridizing sequence is SEQ ID NO:11, SEQ ID NO:37, SEQ ID NO:50, or SEQ ID NO:30, or an RNA equivalent or DNA/RNA chimeric thereof and including from 0-7 nucleotide analogs, and/or wherein the second NL63-specific target-hybridizing sequence is SEQ ID NO:12, SEQ ID NO:49, SEQ ID NO:45, or SEQ ID NO:23, or an RNA equivalent or DNA/RNA chimeric thereof and including from 0-7 nucleotide analogs.

Embodiment 83. The kit of any one of embodiments 66 to 81, wherein the first NL63-specific target-hybridizing sequence and the second NL63-specific target hybridizing sequence are (a) SEQ ID NO:11 and SEQ ID NO:12; (b) SEQ ID NO:37 and SEQ ID NO:49; or (c) SEQ ID NO:30 or SEQ ID NO:50 and SEQ ID NO:23 or SEQ ID NO:45.

Embodiment 84. The kit of embodiment 81, 82, or 83, wherein the NL63-specific detection probe target-hybridizing sequence is SEQ ID NO:13, SEQ ID NO:18, SEQ ID NO:22, or SEQ ID NO:17, or an RNA equivalent or DNA/RNA chimeric thereof and including from 0-7 nucleotide analogs.

Embodiment 85. The kit of any one of embodiments 81 to 84, wherein the NL63-specific detection probe oligomer further comprises a detectable label.

Embodiment 86. The kit of embodiment 85, wherein the detectable label is a fluorescent or chemiluminescent label.

Embodiment 87. The kit of embodiment 85, wherein the detectable label is a fluorescent label and the NL63-specific detection probe oligomer further comprises a non-fluorescent

Embodiment 88. The kit of any one of embodiments 66 to 87, further comprising a 229E-specific detection probe oligomer comprising a 229E-specific detection probe target-hybridizing sequence that is from about 15 to about 35 nucleotides in length and is configured to hybridize to a target sequence contained within a 229E amplicon amplifiable by the first and second 229E-specific amplification oligomers.

Embodiment 89. The kit of any one of embodiments 66 to 88, wherein the first 229E-specific target-hybridizing sequence is SEQ ID NO:14 or SEQ ID NO:42, or an RNA equivalent or DNA/RNA chimeric thereof and including from 0-7 nucleotide analogs, and/or wherein the second 229E-specific target-hybridizing sequence is SEQ ID NO:15 or SEQ ID NO:35, or an RNA equivalent or DNA/RNA chimeric thereof and including from 0-7 nucleotide analogs.

Embodiment 90. The kit of any one of embodiments 66 to 88, wherein the first 229E-specific target-hybridizing sequence and the second 229E-specific target-hybridizing sequence are (a) SEQ ID NO:14 and SEQ ID NO:15; or (b) SEQ ID NO:42 and SEQ ID NO:35.

Embodiment 91. The kit of embodiment 88, 89, or 90, wherein the 229E-specific detection probe target-hybridizing sequence is SEQ ID NO:16 or SEQ ID NO:36, or an RNA equivalent or DNA/RNA chimeric thereof and including from 0-7 nucleotide analogs.

Embodiment 92. The kit of any one of embodiments 88 to 91, wherein the 229E-specific detection probe oligomer further comprises a detectable label.

Embodiment 93. The kit of embodiment 92, wherein the detectable label is a fluorescent or chemiluminescent label.

Embodiment 94. The kit of embodiment 92, wherein the detectable label is a fluorescent label and the 229E-specific detection probe oligomer further comprises a non-fluorescent quencher.

Embodiment 95. A kit for determining the presence or absence of human coronavirus OC43 (OC43) in a sample, said kit comprising: a first amplification oligomer combination comprising first and second human coronavirus OC43-specific amplification oligomers capable of amplifying a target region of an OC43 target nucleic acid, wherein the first OC43-specific amplification oligomer comprises a first OC43-specific target-hybridizing sequence substantially corresponding to the nucleotide sequence of SEQ ID NO:5, SEQ ID NO:38, SEQ ID NO:39, or SEQ ID NO:24, and wherein the second OC43-specific amplification oligomer comprises a second OC43-specific target-hybridizing sequence substantially corresponding to the nucleotide sequence of SEQ ID NO:6, SEQ ID NO:43, SEQ ID NO:41, SEQ ID NO:32, SEQ ID NO:44, or SEQ ID NO:51.

Embodiment 96. The kit of embodiment 95, further comprising an OC43-specific detection probe oligomer comprising an OC43-specific detection probe target-hybridizing sequence that is from about 15 to about 35 nucleotides in length and is configured to hybridize to a target sequence contained within an OC43 amplicon amplifiable by the first and second OC43-specific amplification oligomers.

Embodiment 97. The kit of embodiment 95 or 96, wherein the first OC43-specific target-hybridizing sequence is SEQ ID NO:5, SEQ ID NO:38, SEQ ID NO:39, or SEQ ID NO:24, or an RNA equivalent or DNA/RNA chimeric thereof and including from 0-7 nucleotide analogs, and/or wherein the second OC43-specific target-hybridizing sequence is SEQ ID NO:6, SEQ ID NO:43, SEQ ID NO:41, SEQ ID NO:32, SEQ ID NO:44, or SEQ ID NO:51, or an RNA equivalent or DNA/RNA chimeric thereof and including from 0-7 nucleotide analogs.

Embodiment 98. The kit of embodiment 95 or 96, wherein the first OC43-specific target-hybridizing sequence and the second OC43-specific target-hybridizing sequence are: (a) SEQ ID NO:5 and SEQ ID NO:6; (b) SEQ ID NO:5 and SEQ ID NO:43; (c) SEQ ID NO:38 or SEQ ID NO:39 and SEQ ID NO:41 or SEQ ID NO:42; or (d) SEQ ID NO:24 and SEQ ID NO:44 or SEQ ID NO:51.

Embodiment 99. The kit of embodiment 96, 97, or 98, wherein the OC43-specific detection probe target-hybridizing sequence is SEQ ID NO:7, SEQ ID NO:47, SEQ ID NO:33, or SEQ ID NO:34, or an RNA equivalent or DNA/RNA chimeric thereof and including from 0-7 nucleotide analogs.

Embodiment 100. The kit of any one of embodiments 96 to 99, wherein the OC43-specific detection probe oligomer further comprises a detectable label.

Embodiment 101. The kit of embodiment 100, wherein the detectable label is a fluorescent or chemiluminescent label.

Embodiment 102. The kit of embodiment 100, wherein the detectable label is a fluorescent label and the OC43-specific detection probe oligomer further comprises a non-fluorescent quencher.

Embodiment 103. The kit of any one of embodiments 95 to 102, further comprising: an amplification oligomer combination capable of amplifying a target region of a human coronavirus HKU1 (HKU1) target nucleic acid; and/or an amplification oligomer combination capable of amplifying a target region of a human coronavirus NL63 (NL63) target nucleic acid; and/or an amplification oligomer combination capable of amplifying a target region of a human coronavirus 229E (229E) target nucleic acid.

Embodiment 104. A kit for determining the presence or absence of human coronavirus HKU1 (HKU1) in a sample, said kit comprising: a first amplification oligomer combination comprising first and second human coronavirus HKU1-specific amplification oligomers capable of amplifying a target region of an HKU1 target nucleic acid, wherein the first HKU-specific amplification oligomer comprises a first HKU1-specific target-hybridizing sequence substantially corresponding to the nucleotide sequence of SEQ ID NO:8, SEQ ID NO:20, SEQ ID NO:26, SEQ ID NO:40, or SEQ ID NO:25, and wherein the second HKU1-specific amplification oligomer comprises a second HKU1-specific target-hybridizing sequence substantially corresponding to the nucleotide sequence of SEQ ID NO:9, SEQ ID NO:31, or SEQ ID NO:48.

Embodiment 105. The kit of embodiment 104, further comprising an HKU1-specific detection probe oligomer comprising an HKU1-specific detection probe target-hybridizing sequence that is from about 15 to about 35 nucleotides in length and is configured to hybridize to a target sequence contained within an HKU1 amplicon amplifiable by the first and second HKU1-specific amplification oligomers.

Embodiment 106. The kit of embodiment 104 or 105, wherein the first HKU1-specific target-hybridizing sequence is SEQ ID NO:8, SEQ ID NO:20, SEQ ID NO:26, SEQ ID NO:40, or SEQ ID NO:25, or an RNA equivalent or DNA/RNA chimeric thereof and including from 0-7 nucleotide analogs, and/or wherein the second HKU1-specific target-hybridizing sequence is SEQ ID NO:9, SEQ ID NO:31, or SEQ ID NO:48, or an RNA equivalent or DNA/RNA chimeric thereof and including from 0-7 nucleotide analogs.

Embodiment 107. The kit of embodiment 104 or 105, wherein the first HKU1-specific target-hybridizing sequence and the second HKU1-specific target hybridizing sequence are (a) SEQ ID NO:8 and SEQ ID NO:9; (b) SEQ ID NO:20 or SEQ ID NO:26 and SEQ ID NO:9; (c) SEQ ID NO:40 and SEQ ID NO:31; or (d) SEQ ID NO:25 and SEQ ID NO:48.

Embodiment 108. The kit of embodiment 105, 106 or 107, wherein the HKU1-specific detection probe target-hybridizing sequence is SEQ ID NO:10, SEQ ID NO:27, or SEQ ID NO:19, or an RNA equivalent or DNA/RNA chimeric thereof and including from 0-7 nucleotide analogs.

Embodiment 109. The kit of any one of embodiments 105 to 108, wherein the HKU-specific detection probe oligomer further comprises a detectable label.

Embodiment 110. The kit of embodiment 109, wherein the detectable label is a fluorescent or chemiluminescent label.

Embodiment 111. The kit of embodiment 109, wherein the detectable label is a fluorescent label and the HKU1-specific detection probe oligomer further comprises a non-fluorescent quencher.

Embodiment 112. The kit of any one of embodiments 104 to 111, further comprising: an amplification oligomer combination capable of amplifying a target region of a human coronavirus OC43 (OC43) target nucleic acid; and/or an amplification oligomer combination capable of amplifying a target region of a human coronavirus NL63 (NL63) target nucleic acid; and/or an amplification oligomer combination capable of amplifying a target region of a human coronavirus 229E (229E) target nucleic acid.

Embodiment 113. A kit for determining the presence or absence of human coronavirus NL63 (NL63) in a sample, said kit comprising: a first amplification oligomer combination comprising first and second NL63-specific amplification oligomers capable of amplifying a target region of an NL63 target nucleic acid, wherein the first NL63-specific amplification oligomer comprises a first NL63-specific target-hybridizing sequence substantially corresponding to the nucleotide sequence of SEQ ID NO:11, SEQ ID NO:37, SEQ ID NO:50, or SEQ ID NO:30, and wherein the second NL63-specific amplification oligomer comprises a second NL63-specific target-hybridizing sequence substantially corresponding to the nucleotide sequence of SEQ ID NO:12, SEQ ID NO:49, SEQ ID NO:45, or SEQ ID NO:23.

Embodiment 114. The kit of embodiment 113, further comprising an NL63-specific detection probe oligomer comprising an NL63-specific detection probe target-hybridizing sequence that is from about 15 to about 35 nucleotides in length and is configured to hybridize to a target sequence contained within an NL63 amplicon amplifiable by the first and second NL63-specific amplification oligomers.

Embodiment 115. The kit of embodiment 113 or 114, wherein the first NL63-specific target-hybridizing sequence is SEQ ID NO:11, SEQ ID NO:37, SEQ ID NO:50, or SEQ ID NO:30, or an RNA equivalent or DNA/RNA chimeric thereof and including from 0-7 nucleotide analogs, and/or wherein the second NL63-specific target-hybridizing sequence is SEQ ID NO:12, SEQ ID NO:49, SEQ ID NO:45, or SEQ ID NO:23, or an RNA equivalent or DNA/RNA chimeric thereof and including from 0-7 nucleotide analogs.

Embodiment 116. The kit of embodiment 113 or 114, wherein the first NL63-specific target-hybridizing sequence and the second NL63-specific target hybridizing sequence are (a) SEQ ID NO:11 and SEQ ID NO:12; (b) SEQ ID NO:37 and SEQ ID NO:49; or (c) SEQ ID NO:30 or SEQ ID NO:50 and SEQ ID NO:23 or SEQ ID NO:45.

Embodiment 117. The kit of embodiment 114, 115, or 116, wherein the NL63-specific detection probe target-hybridizing sequence is SEQ ID NO:13, SEQ ID NO:18, SEQ ID NO:22, or SEQ ID NO:17, or an RNA equivalent or DNA/RNA chimeric thereof and including from 0-7 nucleotide analogs.

Embodiment 118. The kit of any one of embodiments 114 to 117, wherein the NL63-specific detection probe oligomer further comprises a detectable label.

Embodiment 119. The kit of embodiment 118, wherein the detectable label is a fluorescent or chemiluminescent label.

Embodiment 120. The kit of embodiment 118, wherein the detectable label is a fluorescent label and the NL63-specific detection probe oligomer further comprises a non-fluorescent quencher.

Embodiment 121. The kit of any one of embodiments 113 to 120, further comprising: an amplification oligomer combination capable of amplifying a target region of a human coronavirus OC43 (OC43) target nucleic acid; and/or an amplification oligomer combination capable of amplifying a target region of a human coronavirus HKU1 (HKU1) target nucleic acid; and/or an amplification oligomer combination capable of amplifying a target region of a human coronavirus 229E (229E) target nucleic acid.

Embodiment 122. A kit for determining the presence or absence of each of human coronavirus 229E (229E) in a sample, said kit comprising: a first amplification oligomer combination comprising first and second 229E-specific amplification oligomers capable of amplifying a target region of a 229E target nucleic acid, wherein the first 229E-specific amplification oligomer comprises a first 229E-specific target-hybridizing sequence substantially corresponding to the nucleotide sequence of SEQ ID NO:14 or SEQ ID NO:42, and wherein the second 229E-specific amplification oligomer comprises a second 229E-specific target-hybridizing sequence substantially corresponding to the nucleotide sequence of SEQ ID NO:15 or SEQ ID NO:35.

Embodiment 123. The kit of embodiment 122, further comprising a 229E-specific detection probe oligomer comprising a 229E-specific detection probe target-hybridizing sequence that is from about 15 to about 35 nucleotides in length and is configured to hybridize to a target sequence contained within a 229E amplicon amplifiable by the first and second 229E-specific amplification oligomers.

Embodiment 124. The kit of embodiment 122 or 123, wherein the first 229E-specific target-hybridizing sequence is SEQ ID NO:14 or SEQ ID NO:42, or an RNA equivalent or DNA/RNA chimeric thereof and including from 0-7 nucleotide analogs, and/or wherein the second 229E-specific target-hybridizing sequence is SEQ ID NO:15 or SEQ ID NO:35, or an RNA equivalent or DNA/RNA chimeric thereof and including from 0-7 nucleotide analogs.

Embodiment 125. The kit of embodiment 122 or 123, wherein the first 229E-specific target-hybridizing sequence and the second 229E-specific target-hybridizing sequence are (a) SEQ ID NO:14 and SEQ ID NO:15; or (b) SEQ ID NO:42 and SEQ ID NO:35.

Embodiment 126. The kit of embodiment 123, 124, or 125, wherein the 229E-specific detection probe target-hybridizing sequence is SEQ ID NO:16 or SEQ ID NO:36, or an RNA equivalent or DNA/RNA chimeric thereof and including from 0-7 nucleotide analogs.

Embodiment 127. The kit of any one of embodiments 123 to 126, wherein the 229E-specific detection probe oligomer further comprises a detectable label.

Embodiment 128. The kit of embodiment 127, wherein the detectable label is a fluorescent or chemiluminescent label.

Embodiment 129. The kit of embodiment 127, wherein the detectable label is a fluorescent label and the 229E-specific detection probe oligomer further comprises a non-fluorescent quencher.

Embodiment 130. The kit of any one of embodiments 122 to 129, further comprising: an amplification oligomer combination capable of amplifying a target region of a human coronavirus OC43 (OC43) target nucleic acid; and/or an amplification oligomer combination capable of amplifying a target region of a human coronavirus HKU1 (HKU1) target nucleic acid; and/or an amplification oligomer combination capable of amplifying a target region of a human coronavirus NL63 (NL63) target nucleic acid.

Embodiment 131. A method for determining the presence or absence of each of human coronaviruses OC43, HKU1, NL63, and 229E in a sample, said method comprising: (1) contacting a sample suspected of containing at least one of human coronavirus OC43 (OC43), human coronavirus HKU1 (HKU1), human coronavirus NL63 (NL63), and human coronavirus 229E (229E) with first, second, third, and fourth amplification oligomer combinations, wherein (a) a first amplification oligomer combination comprising first and second OC43-specific amplification oligomers capable of amplifying a target region of an OC43 target nucleic acid, wherein the first OC43-specific amplification oligomer comprises a first OC43-specific target-hybridizing sequence substantially corresponding to the nucleotide sequence of SEQ ID NO:5, SEQ ID NO:38, SEQ ID NO:39, or SEQ ID NO:24, and wherein the second OC43-specific amplification oligomer comprises a second OC43-specific target-hybridizing sequence substantially corresponding to the nucleotide sequence of SEQ ID NO:6, SEQ ID NO:43, SEQ ID NO:41, SEQ ID NO:32, SEQ ID NO:44, or SEQ ID NO:51; (b) a second amplification oligomer combination comprising first and second HKU1-specific amplification oligomers capable of amplifying a target region of an HKU1 target nucleic acid, wherein the first HKU-specific amplification oligomer comprises a first HKU1-specific target-hybridizing sequence substantially corresponding to the nucleotide sequence of SEQ ID NO:8, SEQ ID NO:20, SEQ ID NO:26, SEQ ID NO:40, or SEQ ID NO:25, and wherein the second HKU1-specific amplification oligomer comprises a second HKU1-specific target-hybridizing sequence substantially corresponding to the nucleotide sequence of SEQ ID NO:9, SEQ ID NO:31, or SEQ ID NO:48; (c) a third amplification oligomer combination comprising first and second NL63-specific amplification oligomers capable of amplifying a target region of an NL63 target nucleic acid, wherein the first NL63-specific amplification oligomer comprises a first NL63-specific target-hybridizing sequence substantially corresponding to the nucleotide sequence of SEQ ID NO:11, SEQ ID NO:37, SEQ ID NO:50, or SEQ ID NO:30, and wherein the second NL63-specific amplification oligomer comprises a second NL63-specific target-hybridizing sequence substantially corresponding to the nucleotide sequence of SEQ ID NO:12, SEQ ID NO:49, SEQ ID NO:45, or SEQ ID NO:23; and (d) a fourth amplification oligomer combination comprising first and second 229E-specific amplification oligomers capable of amplifying a target region of a 229E target nucleic acid, wherein the first 229E-specific amplification oligomer comprises a first 229E-specific target-hybridizing sequence substantially corresponding to the nucleotide sequence of SEQ ID NO:14 or SEQ ID NO:42, and wherein the second 229E-specific amplification oligomer comprises a second 229E-specific target-hybridizing sequence substantially corresponding to the nucleotide sequence of SEQ ID NO:15 or SEQ ID NO:35; (2) performing an in vitro nucleic acid amplification reaction, wherein any OC43, HKU1, NL63, and/or 229E target nucleic acids, if present in the sample, are used as templates for generating amplicons corresponding to the OC43, HKU1, NL63, and 229E target regions present in the sample; and (3) detecting the presence or absence of the amplicons corresponding to the OC43, HKU1, NL63, and 229E target regions, thereby determining the presence or absence of OC43, HKU1, NL63, and 229E in the sample.

Embodiment 132. The method of embodiment 131, wherein the method is a multiplex method comprising contacting the sample with the first, second, third, and fourth amplification oligomer combinations within the same reaction mixture.

Embodiment 133. The method of embodiment 131 or 132, wherein the detecting step comprises contacting the in vitro nucleic acid amplification reaction with an OC43-specific detection probe oligomer comprising an OC43-specific detection probe target-hybridizing sequence that is from about 15 to about 35 nucleotides in length and is configured to hybridize to a target sequence contained within an OC43 amplicon amplifiable by the first and second OC43-specific amplification oligomers.

Embodiment 134. The method of embodiment 131, 132, or 133, wherein the first OC43-specific target-hybridizing sequence is SEQ ID NO:5, SEQ ID NO:38, SEQ ID NO:39, or SEQ ID NO:24, or an RNA equivalent or DNA/RNA chimeric thereof and including from 0-7 nucleotide analogs, and/or wherein the second OC43-specific target-hybridizing sequence is SEQ ID NO:6, SEQ ID NO:43, SEQ ID NO:41, SEQ ID NO:32, SEQ ID NO:44, or SEQ ID NO:51, or an RNA equivalent or DNA/RNA chimeric thereof and including from 0-7 nucleotide analogs.

Embodiment 135. The method of embodiment 131, 132, or 133, wherein the first OC43-specific target-hybridizing sequence and the second OC43-specific target-hybridizing sequence are: (a) SEQ ID NO:5 and SEQ ID NO:6; (b) SEQ ID NO:5 and SEQ ID NO:43; (c) SEQ ID NO:38 or SEQ ID NO:39 and SEQ ID NO:41 or SEQ ID NO:42; or (d) SEQ ID NO:24 and SEQ ID NO:44 or SEQ ID NO:51.

Embodiment 136. The method of embodiment 133, 134, or 135, wherein the OC43-specific detection probe target-hybridizing sequence is SEQ ID NO:7, SEQ ID NO:47, SEQ ID NO:33, or SEQ ID NO:34, or an RNA equivalent or DNA/RNA chimeric thereof and including from 0-7 nucleotide analogs.

Embodiment 137. The method of any one of embodiments 133 to 136, wherein the OC43-specific detection probe oligomer further comprises a detectable label.

Embodiment 138. The method of embodiment 137, wherein the detectable label is a fluorescent or chemiluminescent label.

Embodiment 139. The method of embodiment 137, wherein the detectable label is a fluorescent label and the OC43-specific detection probe oligomer further comprises a non-fluorescent quencher.

Embodiment 140. The method of any one of embodiments 131 to 139, wherein the detecting step comprises contacting the in vitro nucleic acid amplification reaction with an HKU1-specific detection probe oligomer comprising an HKU1-specific detection probe target-hybridizing sequence that is from about 15 to about 35 nucleotides in length and is configured to hybridize to a target sequence contained within an HKU1 amplicon amplifiable by the first and second HKU 1-specific amplification oligomers.

Embodiment 141. The method of any one of embodiments 131 to 140, wherein the first HKU1-specific target-hybridizing sequence is SEQ ID NO:8, SEQ ID NO:20, SEQ ID NO:26, SEQ ID NO:40, or SEQ ID NO:25, or an RNA equivalent or DNA/RNA chimeric thereof and including from 0-7 nucleotide analogs, and/or wherein the second HKU1-specific target-hybridizing sequence is SEQ ID NO:9, SEQ ID NO:31, or SEQ ID NO:48, or an RNA equivalent or DNA/RNA chimeric thereof and including from 0-7 nucleotide analogs.

Embodiment 142. The method of any one of embodiments 131 to 140, wherein the first HKU1-specific target-hybridizing sequence and the second HKU1-specific target hybridizing sequence are (a) SEQ ID NO:8 and SEQ ID NO:9; (b) SEQ ID NO:20 or SEQ ID NO:26 and SEQ ID NO:9; (c) SEQ ID NO:40 and SEQ ID NO:31; or (d) SEQ ID NO:25 and SEQ ID NO:48.

Embodiment 143. The method of embodiment 140, 141, or 142, wherein the HKU1-specific detection probe target-hybridizing sequence is SEQ ID NO:10, SEQ ID NO:27, or SEQ ID NO:19, or an RNA equivalent or DNA/RNA chimeric thereof and including from 0-7 nucleotide analogs.

Embodiment 144. The method of any one of embodiments 140-143, wherein the HKU1-specific detection probe oligomer further comprises a detectable label.

Embodiment 145. The method of embodiment 144, wherein the detectable label is a fluorescent or chemiluminescent label.

Embodiment 146. The method of embodiment 144, wherein the detectable label is a fluorescent label and the HKU1-specific detection probe oligomer further comprises a non-fluorescent quencher.

Embodiment 147. The method of any one of embodiments 131 to 146, wherein the detecting step comprises contacting the in vitro nucleic acid amplification reaction with an NL63-specific detection probe oligomer comprising an NL63-specific detection probe target-hybridizing sequence that is from about 15 to about 35 nucleotides in length and is configured to hybridize to a target sequence contained within an NL63 amplicon amplifiable by the first and second NL63-specific amplification oligomers.

Embodiment 148. The method of any one of embodiments 131 to 147, wherein the first NL63-specific target-hybridizing sequence is SEQ ID NO:11, SEQ ID NO:37, SEQ ID NO:50, or SEQ ID NO:30, or an RNA equivalent or DNA/RNA chimeric thereof and including from 0-7 nucleotide analogs, and/or wherein the second NL63-specific target-hybridizing sequence is SEQ ID NO:12, SEQ ID NO:49, SEQ ID NO:45, or SEQ ID NO:23, or an RNA equivalent or DNA/RNA chimeric thereof and including from 0-7 nucleotide analogs.

Embodiment 149. The method of any one of embodiments 131 to 147, wherein the first NL63-specific target-hybridizing sequence and the second NL63-specific target hybridizing sequence are (a) SEQ ID NO:11 and SEQ ID NO:12; (b) SEQ ID NO:37 and SEQ ID NO:49; or (c) SEQ ID NO:30 or SEQ ID NO:50 and SEQ ID NO:23 or SEQ ID NO:45.

Embodiment 150. The method of embodiment 147, 148, or 149, wherein the NL63-specific detection probe target-hybridizing sequence is SEQ ID NO:13, SEQ ID NO:18, SEQ ID NO:22, or SEQ ID NO:17, or an RNA equivalent or DNA/RNA chimeric thereof and including from 0-7 nucleotide analogs.

Embodiment 151. The method of any one of embodiments 147 to 150, wherein the NL63-specific detection probe oligomer further comprises a detectable label.

Embodiment 152. The method of embodiment 151, wherein the detectable label is a fluorescent or chemiluminescent label.

Embodiment 153. The method of embodiment 151, wherein the detectable label is a fluorescent label and the NL63-specific detection probe oligomer further comprises a non-fluorescent quencher.

Embodiment 154. The method of any one of embodiments 131 to 153, wherein the detecting step comprises contacting the in vitro nucleic acid amplification reaction with a 229E-specific detection probe oligomer comprising a 229E-specific detection probe target-hybridizing sequence that is from about 15 to about 35 nucleotides in length and is configured to hybridize to a target sequence contained within a 229E amplicon amplifiable by the first and second 229E-specific amplification oligomers.

Embodiment 155. The method of any one of embodiments 131 to 154, wherein the first 229E-specific target-hybridizing sequence is SEQ ID NO:14 or SEQ ID NO:42, or an RNA equivalent or DNA/RNA chimeric thereof and including from 0-7 nucleotide analogs, and/or wherein the second 229E-specific target-hybridizing sequence is SEQ ID NO:15 or SEQ ID NO:35, or an RNA equivalent or DNA/RNA chimeric thereof and including from 0-7 nucleotide analogs.

Embodiment 156. The method of any one of embodiments 131 to 154, wherein the first 229E-specific target-hybridizing sequence and the second 229E-specific target-hybridizing sequence are (a) SEQ ID NO:14 and SEQ ID NO:15; or (b) SEQ ID NO:42 and SEQ ID NO:35.

Embodiment 157. The method of embodiment 154, 155, or 156, wherein the 229E-specific detection probe target-hybridizing sequence is SEQ ID NO:16 or SEQ ID NO:36, or an RNA equivalent or DNA/RNA chimeric thereof and including from 0-7 nucleotide analogs.

Embodiment 158. The method of any one of embodiments 154 to 157, wherein the 229E-specific detection probe oligomer further comprises a detectable label.

Embodiment 159. The method of embodiment 158, wherein the detectable label is a fluorescent or chemiluminescent label.

Embodiment 160. The method of embodiment 158, wherein the detectable label is a fluorescent label and the 229E-specific detection probe oligomer further comprises a non-fluorescent quencher.

Embodiment 161. The method of any one of embodiments 133 to 160, wherein the detecting step is performed in real time.

Embodiment 162. The method of any one of embodiments 131 to 161, wherein the in vitro nucleic acid amplification reaction is a PCR amplification reaction.

Embodiment 163. The method of embodiment 132, wherein the detecting step comprises contacting the in vitro nucleic acid amplification reaction with (i) an OC43-specific detection probe oligomer comprising an OC43-specific detection probe target-hybridizing sequence that is from about 15 to about 35 nucleotides in length and is configured to hybridize to a target sequence contained within an OC43 amplicon amplifiable by the first and second OC43-specific amplification oligomers, (ii) an HKU1-specific detection probe oligomer comprising an HKU1-specific detection probe target-hybridizing sequence that is from about 15 to about 35 nucleotides in length and is configured to hybridize to a target sequence contained within an HKU1 amplicon amplifiable by the first and second HKU1-specific amplification oligomers, (iii) an NL63-specific detection probe oligomer comprising an NL63-specific detection probe target-hybridizing sequence that is from about 15 to about 35 nucleotides in length and is configured to hybridize to a target sequence contained within an NL63 amplicon amplifiable by the first and second NL63-specific amplification oligomers, and (iv) 229E-specific detection probe oligomer comprising a 229E-specific detection probe target-hybridizing sequence that is from about 15 to about 35 nucleotides in length and is configured to hybridize to a target sequence contained within a 229E amplicon amplifiable by the first and second 229E-specific amplification oligomers, wherein each of said OC43-specific, HKU1-specific, NL63-specific, and 229E-specific detection probe oligomers comprises a fluorescent label and a non-fluorescent quencher.

Embodiment 164. The method of embodiment 163, wherein the in vitro nucleic acid amplification reaction is a real-time PCR amplification reaction.

Embodiment 165. A method for determining the presence or absence of human coronavirus OC43 (OC43) in a sample, said method comprising: (1) contacting a sample suspected of containing OC43 with an amplification oligomer combination comprising first and second OC43-specific amplification oligomers capable of amplifying a target region of an OC43 target nucleic acid, wherein the first OC43-specific amplification oligomer comprises a first OC43-specific target-hybridizing sequence substantially corresponding to the nucleotide sequence of SEQ ID NO:5, SEQ ID NO:38, SEQ ID NO:39, or SEQ ID NO:24, and wherein the second OC43-specific amplification oligomer comprises a second OC43-specific target-hybridizing sequence substantially corresponding to the nucleotide sequence of SEQ ID NO:6, SEQ ID NO:43, SEQ ID NO:41, SEQ ID NO:32, SEQ ID NO:44, or SEQ ID NO:51; (2) performing an in vitro nucleic acid amplification reaction, wherein OC43 target nucleic acid, if present in the sample, is used as a template for generating an amplicon corresponding to the OC43 target region; and (3) detecting the presence or absence of the amplicon, thereby determining the presence or absence of OC43 in the sample.

Embodiment 166. The method of embodiment 165, wherein the detecting step comprises contacting the in vitro nucleic acid amplification reaction with an OC43-specific detection probe oligomer comprising an OC43-specific detection probe target-hybridizing sequence that is from about 15 to about 35 nucleotides in length and is configured to hybridize to a target sequence contained within an OC43 amplicon amplifiable by the first and second OC43-specific amplification oligomers.

Embodiment 167. The method of embodiment 165 or 166, wherein the first OC43-specific target-hybridizing sequence is SEQ ID NO:5, SEQ ID NO:38, SEQ ID NO:39, or SEQ ID NO:24, or an RNA equivalent or DNA/RNA chimeric thereof and including from 0-7 nucleotide analogs, and/or wherein the second OC43-specific target-hybridizing sequence is SEQ ID NO:6, SEQ ID NO:43, SEQ ID NO:41, SEQ ID NO:32, SEQ ID NO:44, or SEQ ID NO:51, or an RNA equivalent or DNA/RNA chimeric thereof and including from 0-7 nucleotide analogs.

Embodiment 168. The method of embodiment 165 or 166, wherein the first OC43-specific target-hybridizing sequence and the second OC43-specific target-hybridizing sequence are: (a) SEQ ID NO:5 and SEQ ID NO:6; (b) SEQ ID NO:5 and SEQ ID NO:43; (c) SEQ ID NO:38 or SEQ ID NO:39 and SEQ ID NO:41 or SEQ ID NO:42; or (d) SEQ ID NO:24 and SEQ ID NO:44 or SEQ ID NO:51.

Embodiment 169. The method of embodiment 166, 167, or 168, wherein the OC43-specific detection probe target-hybridizing sequence is SEQ ID NO:7, SEQ ID NO:47, SEQ ID NO:33, or SEQ ID NO:34, or an RNA equivalent or DNA/RNA chimeric thereof and including from 0-7 nucleotide analogs.

Embodiment 170. The method of any one of embodiments 166-169, wherein the OC43-specific detection probe oligomer further comprises a detectable label.

Embodiment 171. The method of embodiment 170, wherein the detectable label is a fluorescent or chemiluminescent label.

Embodiment 172. The method of embodiment 170, wherein the detectable label is a fluorescent label and the OC43-specific detection probe oligomer further comprises a non-fluorescent quencher.

Embodiment 173. The method of any one of embodiments 165 to 172, wherein the detecting step is performed in real time.

Embodiment 174. The method of any one of embodiments 165 to 173, wherein the in vitro nucleic acid amplification reaction is a PCR amplification reaction.

Embodiment 175. The method of embodiment 172, wherein the in vitro nucleic acid amplification reaction is a real-time PCR amplification reaction.

Embodiment 176. The method of any one of embodiments 165 to 175, further comprising (i) contacting the sample with an amplification oligomer combination capable of amplifying a target region of a human coronavirus HKU1 (HKU1) target nucleic acid, wherein, at the amplification step, any HKU1 target nucleic acid, if present in the sample, is used as a template for generating an amplicon corresponding to the HKU1 target region, and wherein the detecting step comprises detecting the presence or absence of the amplicon corresponding to the HKU1 target region; and/or (ii) contacting the sample with an amplification oligomer combination capable of amplifying a target region of a human coronavirus NL63 (NL63) target nucleic acid, wherein, at the amplification step, any NL63 target nucleic acid, if present in the sample, is used as a template for generating an amplicon corresponding to the NL63 target region, and wherein the detecting step comprises detecting the presence or absence of the amplicon corresponding to the NL63 target region; and/or (iii) contacting the sample with an amplification oligomer combination capable of amplifying a target region of a human coronavirus 229E (229E) target nucleic acid, wherein, at the amplification step, any 229E target nucleic acid, if present in the sample, is used as a template for generating an amplicon corresponding to the 229E target region, and wherein the detecting step comprises detecting the presence or absence of the amplicon corresponding to the 229E target region.

Embodiment 177. A method for determining the presence or absence of human coronavirus HKU1 (HKU1) in a sample, said method comprising: (1) contacting a sample suspected of containing HKU1 with an amplification oligomer combination comprising first and second HKU1-specific amplification oligomers capable of amplifying a target region of an HKU1 target nucleic acid, wherein the first HKU1-specific amplification oligomer comprises a first HKU1-specific target-hybridizing sequence substantially corresponding to the nucleotide sequence of SEQ ID NO:8, SEQ ID NO:20, SEQ ID NO:26, SEQ ID NO:40, or SEQ ID NO:25, and wherein the second HKU1-specific amplification oligomer comprises a second HKU1-specific target-hybridizing sequence substantially corresponding to the nucleotide sequence of SEQ ID NO:9, SEQ ID NO:31, or SEQ ID NO:48; (2) performing an in vitro nucleic acid amplification reaction, wherein any HKU1 target nucleic acid, if present in the sample, is used as a template for generating an amplicon corresponding to the HKU1 target region; and (3) detecting the presence or absence of the amplicon, thereby determining the presence or absence of HKU1 in the sample.

Embodiment 178. The method of embodiment 177, wherein the detecting step comprises contacting the in vitro nucleic acid amplification reaction with an HKU1-specific detection probe oligomer comprising an HKU1-specific detection probe target-hybridizing sequence that is from about 15 to about 35 nucleotides in length and is configured to hybridize to a target sequence contained within an HKU1 amplicon amplifiable by the first and second HKU1-specific amplification oligomers.

Embodiment 179. The method of embodiment 177 or 178, wherein the first HKU1-specific target-hybridizing sequence is SEQ ID NO:8, SEQ ID NO:20, SEQ ID NO:26, SEQ ID NO:40, or SEQ ID NO:25, or an RNA equivalent or DNA/RNA chimeric thereof and including from 0-7 nucleotide analogs, and/or wherein the second HKU1-specific target-hybridizing sequence is SEQ ID NO:9, SEQ ID NO:31, or SEQ ID NO:48, or an RNA equivalent or DNA/RNA chimeric thereof and including from 0-7 nucleotide analogs.

Embodiment 180. The method of embodiment177 or 178, wherein the first HKU1-specific target-hybridizing sequence and the second HKU 1-specific target hybridizing sequence are: (a) SEQ ID NO:8 and SEQ ID NO:9; (b) SEQ ID NO:20 or SEQ ID NO:26 and SEQ ID NO:9; (c) SEQ ID NO:40 and SEQ ID NO:31; or (d) SEQ ID NO:25 and SEQ ID NO:48.

Embodiment 181. The method of embodiment 178, 179 or 180, wherein the HKU1-specific detection probe target-hybridizing sequence is SEQ ID NO:10, SEQ ID NO:27, or SEQ ID NO:19, or an RNA equivalent or DNA/RNA chimeric thereof and including from 0-7 nucleotide analogs.

Embodiment 182. The method of any one of embodiments 178-181, wherein the HKU1-specific detection probe oligomer further comprises a detectable label.

Embodiment 183. The method of embodiment 182, wherein the detectable label is a fluorescent or chemiluminescent label.

Embodiment 184. The method of embodiment 182, wherein the detectable label is a fluorescent label and the HKU1-specific detection probe oligomer further comprises a non-fluorescent quencher.

Embodiment 185. The method of any one of embodiments 177 to 184, wherein the detecting step is performed in real time.

Embodiment 186. The method of any one of embodiments 177 to 185, wherein the in vitro nucleic acid amplification reaction is a PCR amplification reaction.

Embodiment 187. The method of embodiment 184, wherein the in vitro nucleic acid amplification reaction is a real-time PCR amplification reaction.

Embodiment 188. The method of any one of embodiments 177 to 187, further comprising: (i) contacting the sample with an amplification oligomer combination capable of amplifying a target region of a human coronavirus OC43 (OC43) target nucleic acid, wherein, at the amplification step, any OC43 target nucleic acid, if present in the sample, is used as a template for generating an amplicon corresponding to the OC43 target region, and wherein the detecting step comprises detecting the presence or absence of the amplicon corresponding to the OC43 target region; and/or (ii) contacting the sample with an amplification oligomer combination capable of amplifying a target region of a human coronavirus NL63 (NL63) target nucleic acid, wherein, at the amplification step, any NL63 target nucleic acid, if present in the sample, is used as a template for generating an amplicon corresponding to the NL63 target region, and wherein the detecting step comprises detecting the presence or absence of the amplicon corresponding to the NL63 target region; and/or (iii) contacting the sample with an amplification oligomer combination capable of amplifying a target region of a human coronavirus 229E (229E) target nucleic acid, wherein, at the amplification step, any 229E target nucleic acid, if present in the sample, is used as a template for generating an amplicon corresponding to the 229E target region, and wherein the detecting step comprises detecting the presence or absence of the amplicon corresponding to the 229E target region.

Embodiment 189. A method for determining the presence or absence of human coronavirus NL63 (NL63) in a sample, said method comprising: (1) contacting a sample suspected of containing NL63 with an amplification oligomer combination comprising first and second NL63-specific amplification oligomers capable of amplifying a target region of an NL63 target nucleic acid, wherein the first NL63-specific amplification oligomer comprises a first NL63-specific target-hybridizing sequence substantially corresponding to the nucleotide sequence of SEQ ID NO:11, SEQ ID NO:37, SEQ ID NO:50, or SEQ ID NO:30, and wherein the second NL63-specific amplification oligomer comprises a second NL63-specific target-hybridizing sequence substantially corresponding to the nucleotide sequence of SEQ ID NO:12, SEQ ID NO:49, SEQ ID NO:45, or SEQ ID NO:23; (2) performing an in vitro nucleic acid amplification reaction, wherein any NL63 target nucleic acid, if present in the sample, is used as a template for generating an amplicon corresponding to the NL63 target region; and (3) detecting the presence or absence of the amplicon, thereby determining the presence or absence of NL63 in the sample.

Embodiment 190. The method of embodiment 189, wherein the detecting step comprises contacting the in vitro nucleic acid amplification reaction with an NL63-specific detection probe oligomer comprising an NL63-specific detection probe target-hybridizing sequence that is from about 15 to about 35 nucleotides in length and is configured to hybridize to a target sequence contained within an NL63 amplicon amplifiable by the first and second NL63-specific amplification oligomers.

Embodiment 191. The method of embodiment 189 or 190, wherein the first NL63-specific target-hybridizing sequence is SEQ ID NO:11, SEQ ID NO:37, SEQ ID NO:50, or SEQ ID NO:30, or an RNA equivalent or DNA/RNA chimeric thereof and including from 0-7 nucleotide analogs, and/or wherein the second NL63 -specific target-hybridizing sequence is SEQ ID NO:12, SEQ ID NO:49, SEQ ID NO:45, or SEQ ID NO:23, or an RNA equivalent or DNA/RNA chimeric thereof and including from 0-7 nucleotide analogs.

Embodiment 192. The method of embodiment 189 or 190, wherein the first NL63-specific target-hybridizing sequence and the second NL63-specific target hybridizing sequence are (a) SEQ ID NO:11 and SEQ ID NO:12; (b) SEQ ID NO:37 and SEQ ID NO:49; or (c) SEQ ID NO:30 or SEQ ID NO:50 and SEQ ID NO:23 or SEQ ID NO:45.

Embodiment 193. The method of embodiment 190, 191, or 192, wherein the NL63-specific detection probe target-hybridizing sequence is SEQ ID NO:13, SEQ ID NO:18, SEQ ID NO:22, or SEQ ID NO:17, or an RNA equivalent or DNA/RNA chimeric thereof and including from 0-7 nucleotide analogs.

Embodiment 194. The method of any one of embodiments 190-193, wherein the NL63-specific detection probe oligomer further comprises a detectable label.

Embodiment 195. The method of embodiment 194, wherein the detectable label is a fluorescent or chemiluminescent label.

Embodiment 196. The method of embodiment 194, wherein the detectable label is a fluorescent label and the NL63-specific detection probe oligomer further comprises a non-fluorescent quencher.

Embodiment 197. The method of any one of embodiments 189 to 196, wherein the detecting step is performed in real time.

Embodiment 198. The method of any one of embodiments 189 to 197, wherein the in vitro nucleic acid amplification reaction is a PCR amplification reaction.

Embodiment 199. The method of embodiment 196, wherein the in vitro nucleic acid amplification reaction is a real-time PCR amplification reaction.

Embodiment 200. The method of any one of embodiments 189 to 199, further comprising: (i) contacting the sample with an amplification oligomer combination capable of amplifying a target region of a human coronavirus OC43 (OC43) target nucleic acid, wherein, at the amplification step, any OC43 target nucleic acid, if present in the sample, is used as a template for generating an amplicon corresponding to the OC43 target region, and wherein the detecting step comprises detecting the presence or absence of the amplicon corresponding to the OC43 target region; and/or (ii) contacting the sample with an amplification oligomer combination capable of amplifying a target region of a human coronavirus HKU1 (HKU1) target nucleic acid, wherein, at the amplification step, any HKU1 target nucleic acid, if present in the sample, is used as a template for generating an amplicon corresponding to the HKU1 target region, and wherein the detecting step comprises detecting the presence or absence of the amplicon corresponding to the HKU1 target region; and/or (iii) contacting the sample with an amplification oligomer combination capable of amplifying a target region of a human coronavirus 229E (229E) target nucleic acid, wherein, at the amplification step, any 229E target nucleic acid, if present in the sample, is used as a template for generating an amplicon corresponding to the 229E target region, and wherein the detecting step comprises detecting the presence or absence of the amplicon corresponding to the 229E target region.

Embodiment 201. A method for determining the presence or absence of human coronavirus 229E (229E) in a sample, said method comprising: (1) contacting a sample suspected of containing 229E with an amplification oligomer combination comprising first and second 229E-specific amplification oligomers capable of amplifying a target region of a 229E target nucleic acid, wherein the first 229E-specific amplification oligomer comprises a first 229E-specific target-hybridizing sequence substantially corresponding to the nucleotide sequence of SEQ ID NO:14 or SEQ ID NO:42, and wherein the second 229E-specific amplification oligomer comprises a second 229E-specific target-hybridizing sequence substantially corresponding to the nucleotide sequence of SEQ ID NO:15 or SEQ ID NO:35; (2) performing an in vitro nucleic acid amplification reaction, wherein any 229E target nucleic acid, if present in the sample, is used as a template for generating an amplicon corresponding to the 229E target region; and (3) detecting the presence or absence of the amplicon, thereby determining the presence or absence of 229E in the sample.

Embodiment 202. The method of embodiment 201, wherein the detecting step comprises contacting the in vitro nucleic acid amplification reaction with a 229E-specific detection probe oligomer comprising a 229E-specific detection probe target-hybridizing sequence that is from about 15 to about 35 nucleotides in length and is configured to hybridize to a target sequence contained within a 229E amplicon amplifiable by the first and second 229E-specific amplification oligomers.

Embodiment 203. The method of embodiment 201 or 202, wherein the first 229E-specific target-hybridizing sequence is SEQ ID NO:14 or SEQ ID NO:42, or an RNA equivalent or DNA/RNA chimeric thereof and including from 0-7 nucleotide analogs, and/or wherein the second 229E-specific target-hybridizing sequence is SEQ ID NO:15 or SEQ ID NO:35, or an RNA equivalent or DNA/RNA chimeric thereof and including from 0-7 nucleotide analogs.

Embodiment 204. The method of embodiment 201 or 202, wherein the first 229E-specific target-hybridizing sequence and the second 229E-specific target-hybridizing sequence are (a) SEQ ID NO:14 and SEQ ID NO:15; or (b) SEQ ID NO:42 and SEQ ID NO:35.

Embodiment 205. The method of embodiment 202, 203, or 204, wherein the 229E-specific detection probe target-hybridizing sequence is SEQ ID NO:16 or SEQ ID NO:36, or an RNA equivalent or DNA/RNA chimeric thereof and including from 0-7 nucleotide analogs.

Embodiment 206. The method of any one of embodiments 202-205, wherein the 229E-specific detection probe oligomer further comprises a detectable label.

Embodiment 207. The method of embodiment 206, wherein the detectable label is a fluorescent or chemiluminescent label.

Embodiment 208. The method of embodiment 206, wherein the detectable label is a fluorescent label and the 229E-specific detection probe oligomer further comprises a non-fluorescent quencher.

Embodiment 209. The method of any one of embodiments 201 to 208, wherein the detecting step is performed in real time.

Embodiment 210. The method of any one of embodiments 201 to 209, wherein the in vitro nucleic acid amplification reaction is a PCR amplification reaction.

Embodiment 211. The method of embodiment 208, wherein the in vitro nucleic acid amplification reaction is a real-time PCR amplification reaction.

Embodiment 212. The method of any one of embodiments 201 to 211, further comprising: (i) contacting the sample with an amplification oligomer combination capable of amplifying a target region of a human coronavirus OC43 (OC43) target nucleic acid, wherein, at the amplification step, any OC43 target nucleic acid, if present in the sample, is used as a template for generating an amplicon corresponding to the OC43 target region, and wherein the detecting step comprises detecting the presence or absence of the amplicon corresponding to the OC43 target region; and/or (ii) contacting the sample with an amplification oligomer combination capable of amplifying a target region of a human coronavirus HKU1 (HKU1) target nucleic acid, wherein, at the amplification step, any HKU1 target nucleic acid, if present in the sample, is used as a template for generating an amplicon corresponding to the HKU1 target region, and wherein the detecting step comprises detecting the presence or absence of the amplicon corresponding to the HKU1 target region; and/or (iii) contacting the sample with an amplification oligomer combination capable of amplifying a target region of a human coronavirus NL63 (NL63) target nucleic acid, wherein, at the amplification step, any NL63 target nucleic acid, if present in the sample, is used as a template for generating an amplicon corresponding to the NL63 target region, and wherein the detecting step comprises detecting the presence or absence of the amplicon corresponding to the NL63 target region.

Embodiment 213. A detection probe oligomer comprising: a human coronavirus OC43 (OC43)-specific detection probe target-hybridizing sequence that is from about 15 to about 35 nucleotides in length and is configured to hybridize to a target sequence contained within an OC43 amplicon amplifiable by an amplification oligomer combination comprising first and second OC43-specific amplification oligomers capable of amplifying a target region of an OC43 target nucleic acid, wherein the first OC43-specific amplification oligomer comprises a first OC43-specific target-hybridizing sequence substantially corresponding to the nucleotide sequence of SEQ ID NO:5, SEQ ID NO:38, SEQ ID NO:39, or SEQ ID NO:24, and wherein the second OC43-specific amplification oligomer comprises a second OC43-specific target-hybridizing sequence substantially corresponding to the nucleotide sequence of SEQ ID NO:6, SEQ ID NO:43, SEQ ID NO:41, SEQ ID NO:32, SEQ ID NO:44, or SEQ ID NO:51.

Embodiment 214. The detection probe oligomer of embodiment 213, wherein the OC43-specific detection probe target-hybridizing sequence is SEQ ID NO:7, SEQ ID NO:47, SEQ ID NO:33, or SEQ ID NO:34, or an RNA equivalent or DNA/RNA chimeric thereof and including from 0-7 nucleotide analogs.

Embodiment 215. A detection probe oligomer comprising: a human coronavirus HKU1 (HKU1)-specific detection probe target-hybridizing sequence that is from about 15 to about 35 nucleotides in length and is configured to hybridize to a target sequence contained within an HKU1 amplicon amplifiable by an amplification oligomer combination comprising first and second HKU1-specific amplification oligomers capable of amplifying a target region of an HKU1 target nucleic acid, wherein the first HKU1-specific amplification oligomer comprises a first HKU1-specific target-hybridizing sequence substantially corresponding to the nucleotide sequence of SEQ ID NO:8, SEQ ID NO:20, SEQ ID NO:26, SEQ ID NO:40, or SEQ ID NO:25, and wherein the second HKU1-specific amplification oligomer comprises a second HKU1-specific target-hybridizing sequence substantially corresponding to the nucleotide sequence of SEQ ID NO:9, SEQ ID NO:31, or SEQ ID NO:48.

Embodiment 216. The detection probe oligomer of embodiment 215, wherein the HKU1-specific detection probe target-hybridizing sequence is SEQ ID NO:10, SEQ ID NO:27, or SEQ ID NO:19, or an RNA equivalent or DNA/RNA chimeric thereof and including from 0-7 nucleotide analogs.

Embodiment 217. A detection probe oligomer comprising: a human coronavirus NL63 (NL63)-specific detection probe target-hybridizing sequence that is from about 15 to about 35 nucleotides in length and is configured to hybridize to a target sequence contained within an NL63 amplicon amplifiable by an amplification oligomer combination comprising first and second NL63-specific amplification oligomers capable of amplifying a target region of an NL63 target nucleic acid, wherein the first NL63-specific amplification oligomer comprises a first NL63-specific target-hybridizing sequence substantially corresponding to the nucleotide sequence of SEQ ID NO:11, SEQ ID NO:37, SEQ ID NO:50, or SEQ ID NO:30, and wherein the second NL63-specific amplification oligomer comprises a second NL63-specific target-hybridizing sequence substantially corresponding to the nucleotide sequence of SEQ ID NO:12, SEQ ID NO:49, SEQ ID NO:45, or SEQ ID NO:23.

Embodiment 218. The detection probe oligomer of embodiment 217, wherein the NL63-specific detection probe target-hybridizing sequence is SEQ ID NO:13, SEQ ID NO:18, SEQ ID NO:22, or SEQ ID NO:17, or an RNA equivalent or DNA/RNA chimeric thereof and including from 0-7 nucleotide analogs.

Embodiment 219. A detection probe oligomer comprising: a human coronavirus 229E (229E)-specific detection probe target-hybridizing sequence that is from about 15 to about 35 nucleotides in length and is configured to hybridize to a target sequence contained within a 229E amplicon amplifiable by an amplification oligomer combination comprising first and second 229E-specific amplification oligomers capable of amplifying a target region of a 229E target nucleic acid, wherein the first 229E-specific amplification oligomer comprises a first 229E-specific target-hybridizing sequence substantially corresponding to the nucleotide sequence of SEQ ID NO:14 or SEQ ID NO:42, and wherein the second 229E-specific amplification oligomer comprises a second 229E-specific target-hybridizing sequence substantially corresponding to the nucleotide sequence of SEQ ID NO:15 or SEQ ID NO:35.

Embodiment 220. The detection probe oligomer of embodiment 219, wherein the 229E-specific detection probe target-hybridizing sequence is SEQ ID NO:16 or SEQ ID NO:36, or an RNA equivalent or DNA/RNA chimeric thereof and including from 0-7 nucleotide analogs.

Embodiment 221. The detection probe oligomer of any one of embodiments 213 to 220, wherein the detection probe oligomer further comprises a detectable label.

Embodiment 222. The detection probe oligomer of embodiment 221, wherein the detectable label is a fluorescent or chemiluminescent label.

Embodiment 222.1. The detection probe of embodiment 222, wherein the detectable label is a fluorescent and the detection probe oligomer further comprises a non-fluorescent quencher.

Embodiment 223. A composition comprising: (a) a human coronavirus OC43 (OC43)-specific detection probe oligomer comprising an OC43-specific detection probe target-hybridizing sequence that is from about 15 to about 35 nucleotides in length and is configured to hybridize to a target sequence contained within an OC43 amplicon amplifiable by an amplification oligomer combination comprising first and second OC43-specific amplification oligomers capable of amplifying a target region of an OC43 target nucleic acid, wherein the first OC43-specific amplification oligomer comprises a first OC43-specific target-hybridizing sequence substantially corresponding to the nucleotide sequence of SEQ ID NO:5, SEQ ID NO:38, SEQ ID NO:39, or SEQ ID NO:24, and wherein the second OC43-specific amplification oligomer comprises a second OC43-specific target-hybridizing sequence substantially corresponding to the nucleotide sequence of SEQ ID NO:6, SEQ ID NO:43, SEQ ID NO:41, SEQ ID NO:32, SEQ ID NO:44, or SEQ ID NO:51; and/or (b) a human coronavirus HKU1 (HKU1)-specific detection probe oligomer comprising an HKU 1-specific detection probe target-hybridizing sequence that is from about 15 to about 35 nucleotides in length and is configured to hybridize to a target sequence contained within an HKU1 amplicon amplifiable by an amplification oligomer combination comprising first and second HKU1-specific amplification oligomers capable of amplifying a target region of an HKU1 target nucleic acid, wherein the first HKU 1-specific amplification oligomer comprises a first HKU1-specific target-hybridizing sequence substantially corresponding to the nucleotide sequence of SEQ ID NO:8, SEQ ID NO:20, SEQ ID NO:26, SEQ ID NO:40, or SEQ ID NO:25, and wherein the second HKU1-specific amplification oligomer comprises a second HKU1-specific target-hybridizing sequence substantially corresponding to the nucleotide sequence of SEQ ID NO:9, SEQ ID NO:31, or SEQ ID NO:48; and/or (c) a human coronavirus NL63 (NL63)-specific detection probe oligomer comprising an NL63-specific detection probe target-hybridizing sequence that is from about 15 to about 35 nucleotides in length and is configured to hybridize to a target sequence contained within an NL63 amplicon amplifiable by an amplification oligomer combination comprising first and second NL63-specific amplification oligomers capable of amplifying a target region of an NL63 target nucleic acid, wherein the first NL63-specific amplification oligomer comprises a first NL63-specific target-hybridizing sequence substantially corresponding to the nucleotide sequence of SEQ ID NO:11, SEQ ID NO:37, SEQ ID NO:50, or SEQ ID NO:30, and wherein the second NL63-specific amplification oligomer comprises a second NL63-specific target-hybridizing sequence substantially corresponding to the nucleotide sequence of SEQ ID NO:12, SEQ ID NO:49, SEQ ID NO:45, or SEQ ID NO:23; and/or (d) a human coronavirus 229E (229E)-specific detection probe oligomer comprising a 229E-specific detection probe target-hybridizing sequence that is from about 15 to about 35 nucleotides in length and is configured to hybridize to a target sequence contained within a 229E amplicon amplifiable by an amplification oligomer combination comprising first and second 229E-specific amplification oligomers capable of amplifying a target region of a 229E target nucleic acid, wherein the first 229E-specific amplification oligomer comprises a first 229E-specific target-hybridizing sequence substantially corresponding to the nucleotide sequence of SEQ ID NO:14 or SEQ ID NO:42, and wherein the second 229E-specific amplification oligomer comprises a second 229E-specific target-hybridizing sequence substantially corresponding to the nucleotide sequence of SEQ ID NO:15 or SEQ ID NO:35.

Embodiment 224. The composition of embodiment 223, wherein said composition comprises the OC43-specific detection probe oligomer, the HKU1-specific detection probe oligomer, the NL63-specific detection probe oligomer, and the 229E-specific detection probe oligomer.

Embodiment 225. The composition of embodiment 223 or 224, wherein the OC43-specific detection probe target-hybridizing sequence is SEQ ID NO:7, SEQ ID NO:47, SEQ ID NO:33, or SEQ ID NO:34, or an RNA equivalent or DNA/RNA chimeric thereof and including from 0-7 nucleotide analogs; and/or the HKU1-specific detection probe target-hybridizing sequence is SEQ ID NO:10, SEQ ID NO:27, or SEQ ID NO:19, or an RNA equivalent or DNA/RNA chimeric thereof and including from 0-7 nucleotide analogs; and/or the NL63-specific detection probe target-hybridizing sequence is SEQ ID NO:13, SEQ ID NO:18, SEQ ID NO:22, or SEQ ID NO:17, or an RNA equivalent or DNA/RNA chimeric thereof and including from 0-7 nucleotide analogs; and/or the 229E-specific detection probe target-hybridizing sequence is SEQ ID NO:16 or SEQ ID N0:36, or an RNA equivalent or DNA/RNA chimeric thereof and including from 0-7 nucleotide analogs.

Embodiment 226. The composition of any one of embodiments 223 to 225, wherein one or more of the OC43-specific detection probe oligomer, the HKU1-specific detection probe oligomer, the NL63-specific detection probe oligomer, and the 229E-specific detection probe oligomer further comprises a detectable label.

Embodiment 227. The composition of embodiment 226, wherein the detectable label is a fluorescent or chemiluminescent label.

Embodiments 227.1. The composition of embodiment 223, wherein the composition comprises a human coronavirus OC43 (OC43)-specific detection probe oligomer and a human coronavirus HKU1 (HKU1)-specific detection probe oligomer and a human coronavirus NL63 (NL63)-specific detection probe oligomer and a human coronavirus 229E (229E)-specific detection probe oligomer, wherein each detection probe contains a detectable label.

Embodiment 228. The composition of embodiment 227 or 227.1, wherein the detectable label is different for each of the OC43 -specific detection probe oligomer, the HKU1-specific detection probe oligomer, the NL63-specific detection probe oligomer, and the 229E-specific detection probe oligomer.

Embodiment 229. A composition for determining the presence or absence of each of human coronaviruses OC43, HKU1, NL63, and 229E in a sample, said composition comprising: a first OC43-specific amplification oligomer comprising SEQ ID NO:5, a second OC43-specific amplification oligomer comprising SEQ ID NO:6, a first HKU1-specific amplification oligomer comprising SEQ ID NO:8, a second HKU1-specific amplification oligomer comprising SEQ ID NO:9, a first NL63-specific amplification oligomer comprising SEQ ID NO:11, a second NL63-specific amplification oligomer comprising SEQ ID NO:12; a first 229E-specific amplification oligomer comprising SEQ ID NO:14, and a second 229E-specific amplification oligomer comprising SEQ ID NO:15.

Embodiment 230. A composition for determining the presence or absence of each of human coronaviruses OC43, HKU1, NL63, and 229E in a sample, said composition comprising: a first OC43-specific amplification oligomer having a nucleotide sequence consisting of SEQ ID NO:5, a second OC43-specific amplification oligomer having a nucleotide sequence consisting of SEQ ID NO:6, a first HKU1-specific amplification oligomer having a nucleotide sequence consisting of SEQ ID NO:8, a second HKU1-specific amplification oligomer having a nucleotide sequence consisting of SEQ ID NO:9, a first NL63-specific amplification oligomer having a nucleotide sequence consisting of SEQ ID NO:11, a second NL63-specific amplification oligomer having a nucleotide sequence consisting of SEQ ID NO:12; a first 229E-specific amplification oligomer having a nucleotide sequence consisting of SEQ ID NO:14, and a second 229E-specific amplification oligomer having a nucleotide sequence consisting of SEQ ID NO:15.

Embodiment 231. The composition of embodiment 229 or 230, further comprising: an OC43-specific detection probe oligomer comprising the nucleobase sequence of SEQ ID NO:7, an HKU1-specific detection probe oligomer comprising the nucleobase sequence of SEQ ID NO:10, an NL63-specific detection probe oligomer comprising the nucleobase sequence of SEQ ID NO:13, and an 229E-specific detection probe oligomer comprising the nucleobase sequence of SEQ ID NO:16.

Embodiment 232. A kit for determining the presence or absence of each of human coronaviruses OC43, HKU1, NL63, and 229E in a sample, said composition comprising: a first OC43-specific amplification oligomer comprising SEQ ID NO:5, a second OC43-specific amplification oligomer comprising SEQ ID NO:6, a first HKU1-specific amplification oligomer comprising SEQ ID NO:8, a second HKU1-specific amplification oligomer comprising SEQ ID NO:9, a first NL63-specific amplification oligomer comprising SEQ ID NO:11, a second NL63-specific amplification oligomer comprising SEQ ID NO:12; a first 229E-specific amplification oligomer comprising SEQ ID NO:14, and a second 229E-specific amplification oligomer comprising SEQ ID NO:15.

Embodiment 233. A kit for determining the presence or absence of each of human coronaviruses OC43, HKU1, NL63, and 229E in a sample, said composition comprising: a first OC43-specific amplification oligomer having a nucleotide sequence consisting of SEQ ID NO:5, a second OC43-specific amplification oligomer having a nucleotide sequence consisting of SEQ ID NO:6, a first HKU1-specific amplification oligomer having a nucleotide sequence consisting of SEQ ID NO:8, a second HKU1-specific amplification oligomer having a nucleotide sequence consisting of SEQ ID NO:9, a first NL63-specific amplification oligomer having a nucleotide sequence consisting of SEQ ID NO:11, a second NL63-specific amplification oligomer having a nucleotide sequence consisting of SEQ ID NO:12; a first 229E-specific amplification oligomer having a nucleotide sequence consisting of SEQ ID NO:14, and a second 229E-specific amplification oligomer having a nucleotide sequence consisting of SEQ ID NO:15.

Embodiment 234. The kit of embodiment 232 or 233, further comprising: an OC43-specific detection probe oligomer comprising the nucleobase sequence of SEQ ID NO:7, an HKU1-specific detection probe oligomer comprising the nucleobase sequence of SEQ ID NO:10, an NL63-specific detection probe oligomer comprising the nucleobase sequence of SEQ ID NO:13, and an 229E-specific detection probe oligomer comprising the nucleobase sequence of SEQ ID NO:16.

Embodiment 235. A method for determining the presence or absence of each of human coronaviruses OC43, HKU1, NL63, and 229E in a sample, said method comprising: (a) contacting a sample suspected of containing at least one of human coronavirus OC43 (OC43), human coronavirus HKU1 (HKU1), human coronavirus NL63 (NL63), and human coronavirus 229E (229E) with a first OC43-specific amplification oligomer comprising SEQ ID NO:5, a second OC43-specific amplification oligomer comprising SEQ ID NO:6, a first HKU1-specific amplification oligomer comprising SEQ ID NO:8, a second HKU1-specific amplification oligomer comprising SEQ ID NO:9, a first NL63-specific amplification oligomer comprising SEQ ID NO:11, a second NL63-specific amplification oligomer comprising SEQ ID NO:12; a first 229E-specific amplification oligomer comprising SEQ ID NO:14, and a second 229E-specific amplification oligomer comprising SEQ ID NO:15; and (b) performing an in vitro nucleic acid amplification reaction, wherein any OC43, HKU1, NL63, and/or 229E target nucleic acids, if present in the sample, are used as templates for generating amplicons corresponding to the OC43, HKU1, NL63, and 229E target regions present in the sample; and (3) detecting the presence or absence of the amplicons corresponding to the OC43, HKU1, NL63, and 229E target regions, thereby determining the presence or absence of OC43, HKU1, NL63, and 229E in the sample.

Embodiment 236. The method of embodiment 235, wherein the detecting step comprises contacting the in vitro nucleic acid amplification reaction with an OC43-specific detection probe oligomer comprising the nucleobase sequence of SEQ ID NO:7, an HKU1-specific detection probe oligomer comprising the nucleobase sequence of SEQ ID NO:10, an NL63-specific detection probe oligomer comprising the nucleobase sequence of SEQ ID NO:13, and an 229E-specific detection probe oligomer comprising the nucleobase sequence of SEQ ID NO:16.

In some embodiments, aqueous formulations for the detection of nucleic acid from one or more of human coronaviruses OC43, HKU1, NL63, and 229E are described. The aqueous formulations comprise (1) one or more of an OC43-specific detection probe oligomer, an HKU1-specific detection probe oligomer, and NL63-specific detection probe oligomer, and a 229E-specific detection probe oligomer as above and (2) an organic buffer. In some embodiments, the aqueous formulation further includes one or more components selected from a surfactant (e.g., polyethylene glycol mono [4-(1,1,3,3-tetramethylbutyl) phenyl] ether, polysorbate 20, or a combination thereof), a DNA polymerase enzyme, a reverse transcriptase enzyme, at least one amplification oligomer, and a bulking agent (e.g., trehalose, raffinose, or a combination thereof). In certain variations comprising a surfactant, the surfactant is a non-linear surfactant such as, for example, polysorbate 20. In some embodiments, the aqueous formulation contains inorganic salt at a concentration of 4 mM or less. In related aspects, there are provided reaction mixtures for the detection of one or more of human coronaviruses OC43, HKU1, NL63, and 229E comprising an aqueous formulation as above.

In some embodiments, dried formulations for the detection of nucleic acid from one or more of human coronaviruses OC43, HKU1, NL63, and 229E are described. The dried formulations comprise: (1) one or more of an OC43-specific detection probe oligomer, an HKU1-specific detection probe oligomer, and NL63-specific detection probe oligomer, and a 229E-specific detection probe oligomer as above and (2) a bulking agent. In some embodiments, the bulking agent is trehalose, raffinose, or a combination thereof. In some embodiments, the dried formulation further includes one or more components selected from an inorganic salt, a DNA polymerase enzyme, a reverse transcriptase enzyme, at least one amplification oligomer, and a surfactant (e.g., polyethylene glycol mono [4-(1,1,3,3-tetramethylbutyl) phenyl] ether, polysorbate 20, or a combination thereof). In some embodiments, the dried formulations further comprise an inorganic salt. The percent mass of the inorganic salt to the mass of the dried formulation is 0.249% or less. In certain variations comprising a surfactant, the surfactant is a non-linear surfactant such as, for example, polysorbate 20. In certain variations, the dried formulation is a lyophilized formulation. In some embodiments, there are provided reaction mixtures for the detection of one or more of human coronaviruses OC43, HKU1, NL63, and 229E, where the reaction mixtures are reconstituted with water and an organic buffer from a dried formulation. In some embodiments, the reaction mixture contains an inorganic salt such as, e.g., magnesium, potassium, or sodium. In some variations, the concentration of the inorganic salt is 4 mM or less.

These and other aspects will become evident upon reference to the following detailed description and the attached drawings.

### FIGURES

FIG. 1. Chart illustrating variations in Ct values for the PPR mixes tested in Example 1.

FIG. 2. Chart illustrating variations in RFUs for the PPR mixes tested in Examples 1.

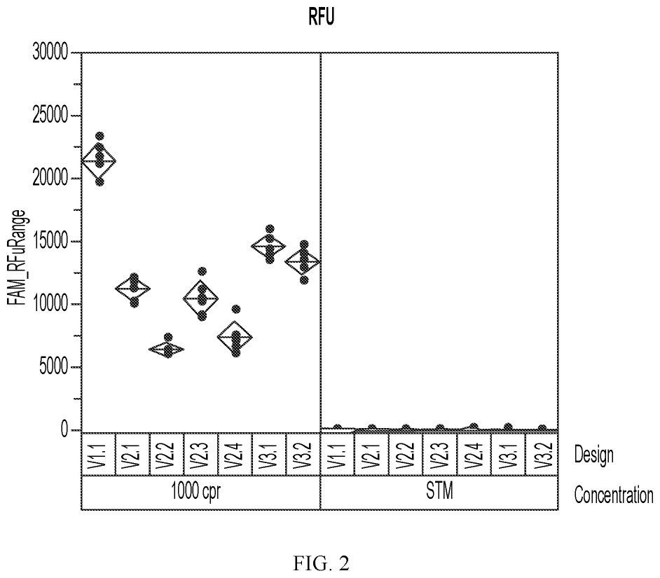

FIG. 3. Chart illustrating variations in Ct values for the PPR mixes tested in Example 2.

FIG. 4. Chart illustrating variations in RFUs for the PPR mixes tested in Example 2.

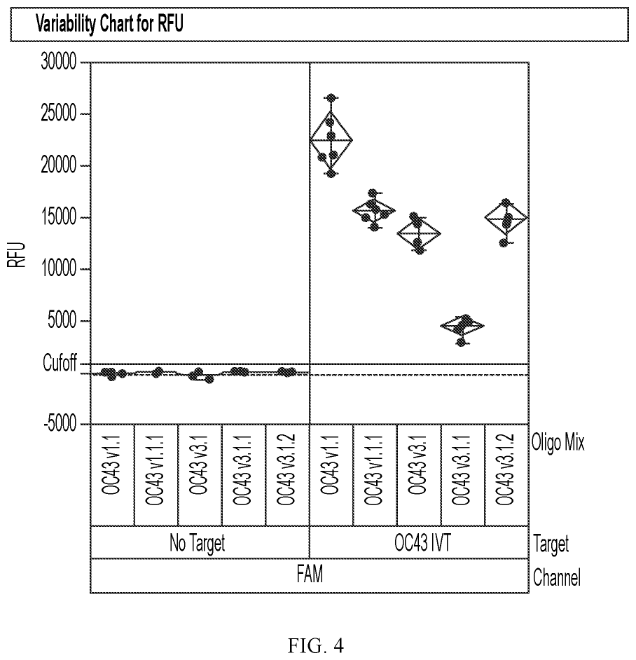

FIG. 5 Chart illustrating variations in Ct values for the PPR mixes tested in Example 3.

FIG. 6. Chart illustrating variations in RFUs for the PPR mixes tested in Example 3.

FIG. 7. Chart illustrating background RFUs for the PPR mixes tested in Example 3.

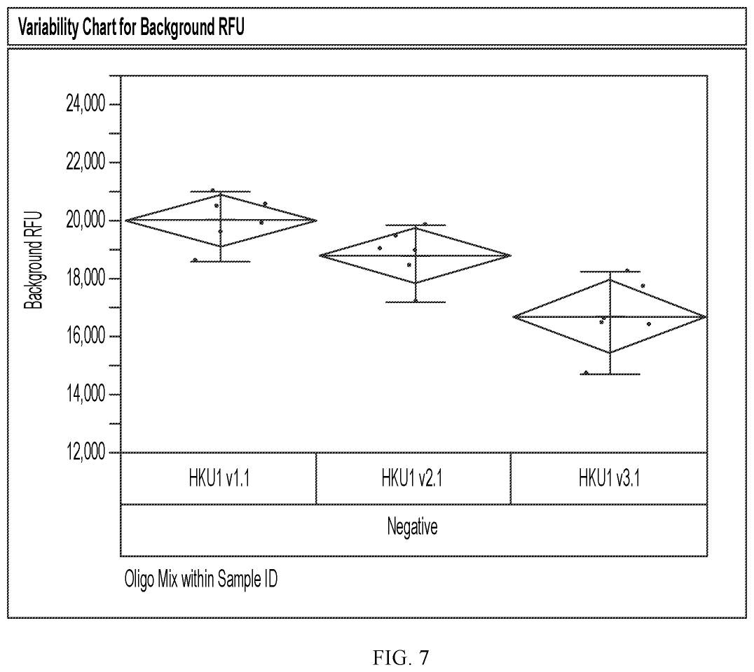

FIG. 8. Chart illustrating variations in Ct values for the NL63 (1000 copies) PPR mixes tested in Example 5.

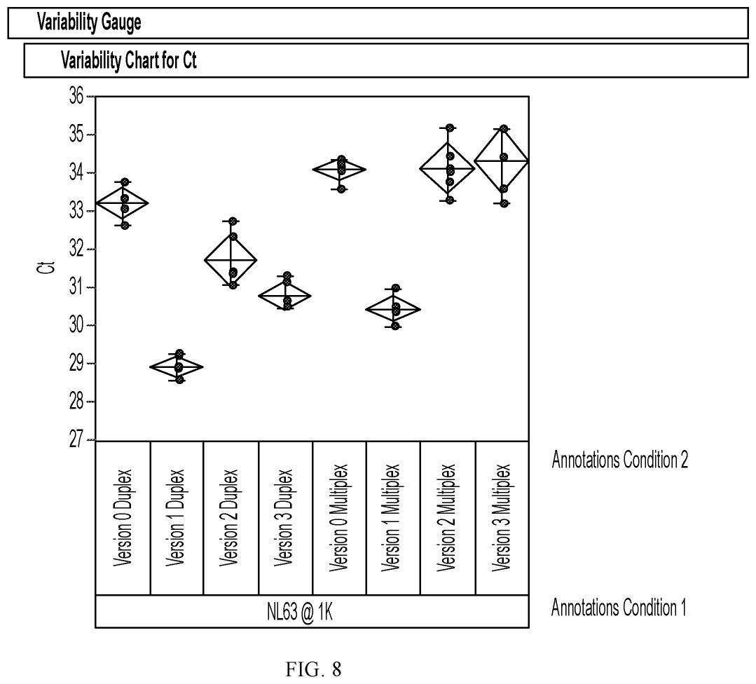

FIG. 9. Chart illustrating variations in RLUs for the NL63 (1000 copies) PPR mixes tested in Example 5.

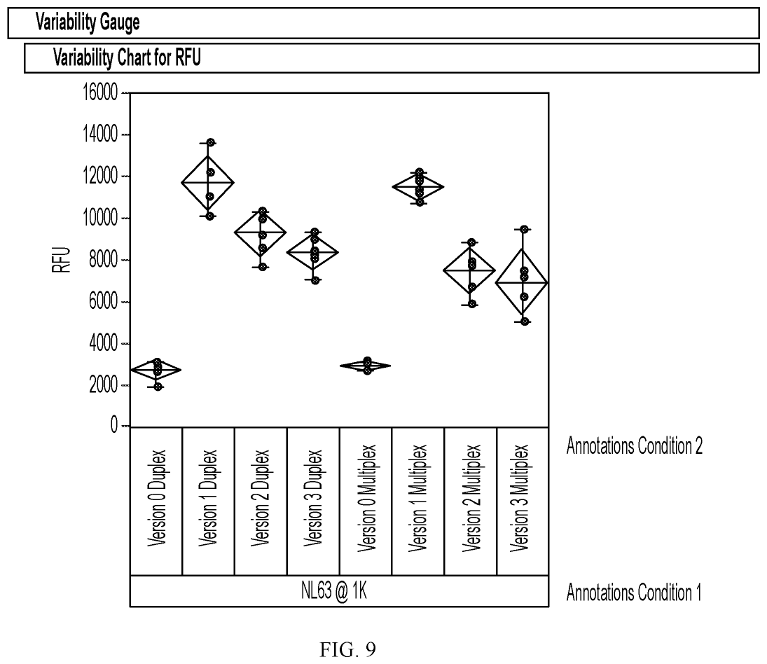

FIG. 10. Chart illustrating variations in Ct values for the OC43 (1000 copies) PPR mixes tested in Example 5.

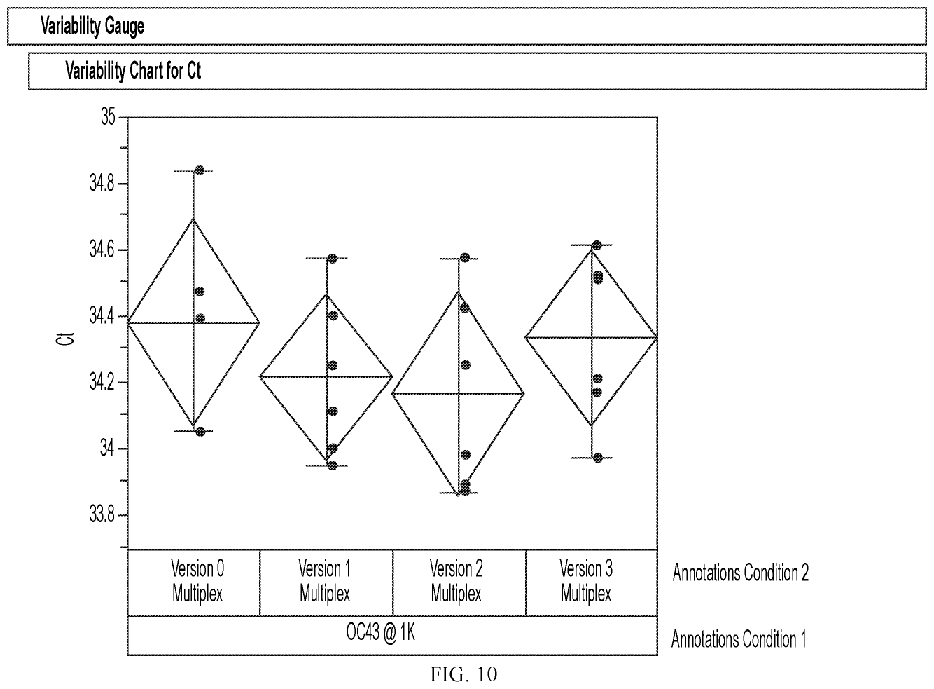

FIG. 11. Chart illustrating variations in RLUs for the OC43 (1000 copies) PPR mixes tested in Example 5.

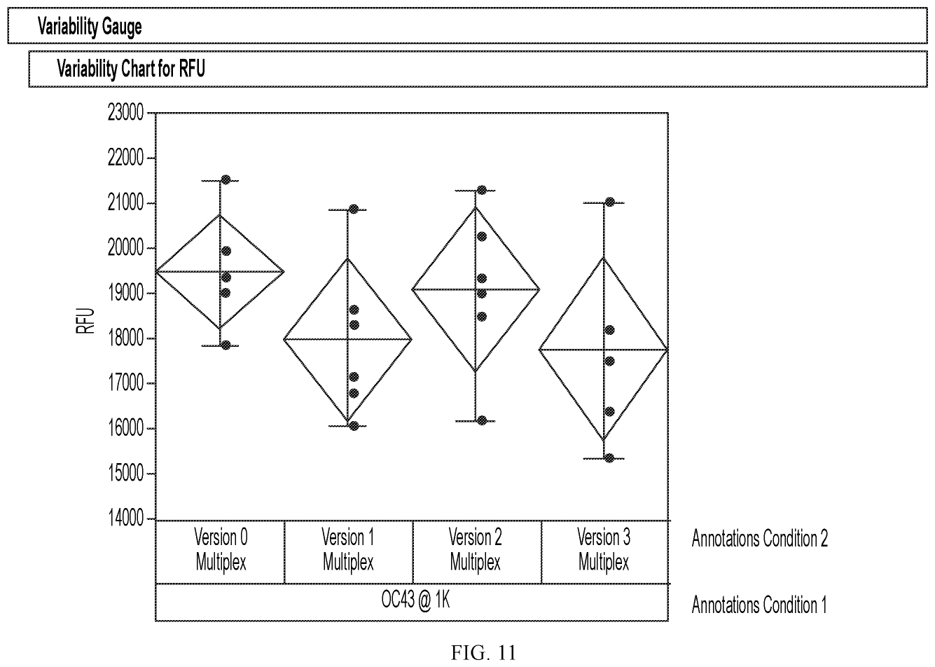

FIG. 12. Chart illustrating variations in Ct values for the HKU1 (1000 copies) PPR mixes tested in Example 5.

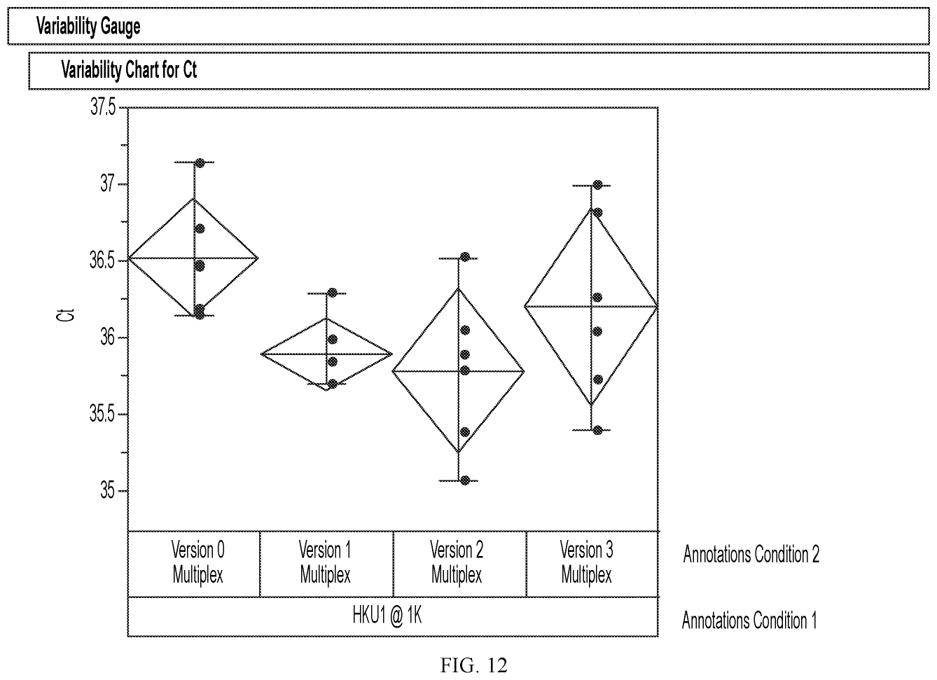

FIG. 13. Chart illustrating variations in RLUs for the HKU1 (1000 copies) PPR mixes tested in Example 5.

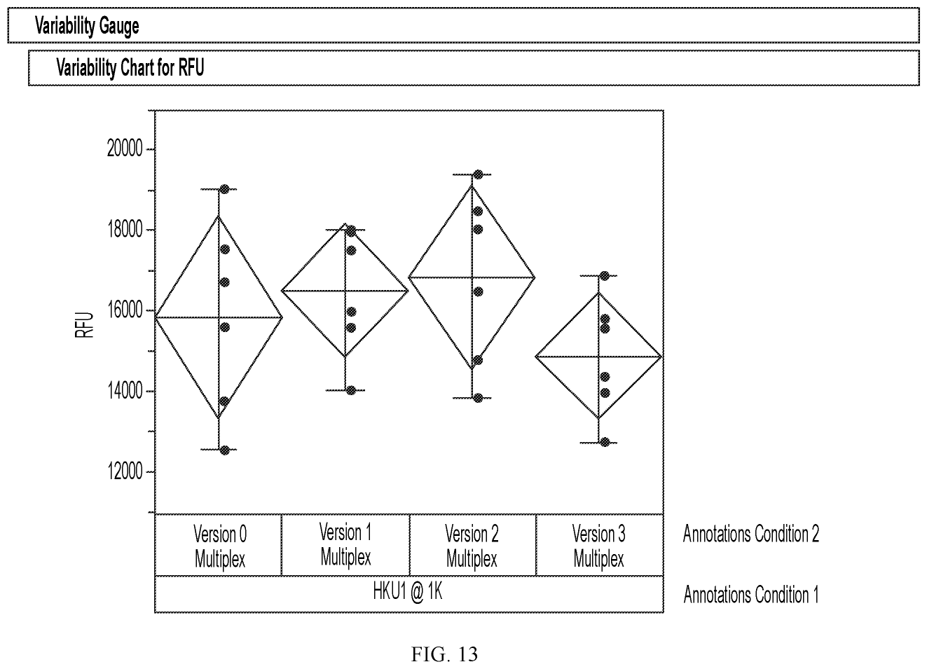

FIG. 14. Chart illustrating variations in Ct values for the 229E (1000 copies) PPR mixes tested in Example 5.

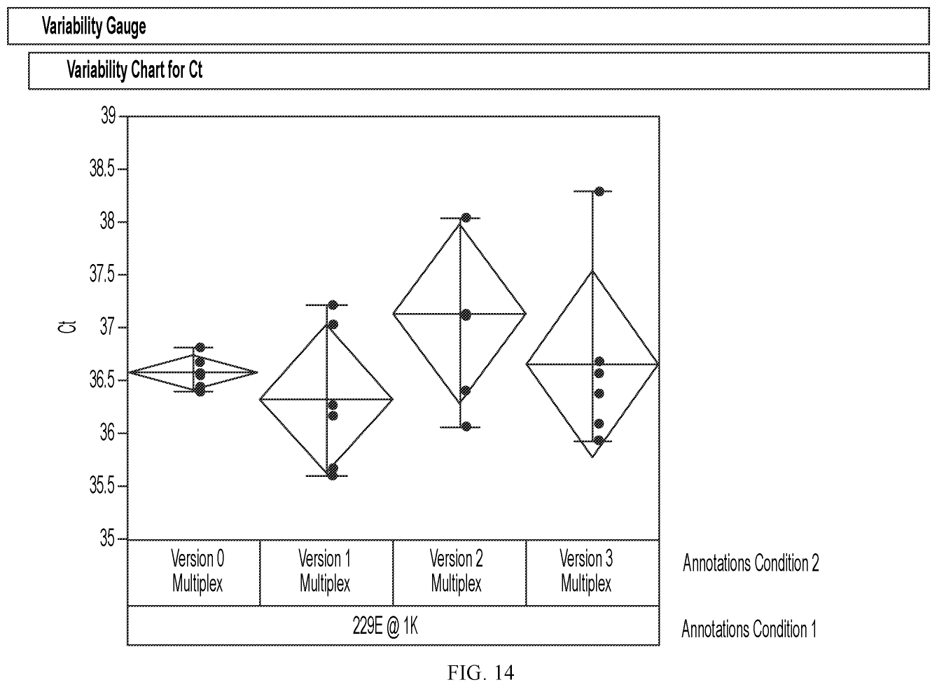

FIG. 15. Chart illustrating variations in RLUs for the 229E (1000 copies) PPR mixes tested in Example 5.

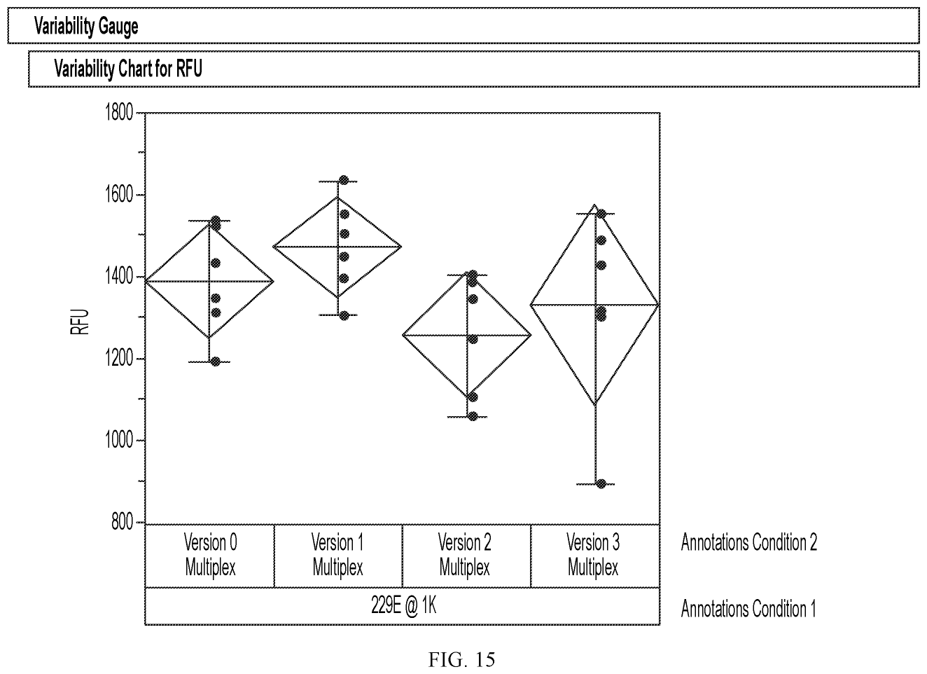

### DEFINITIONS

Unless defined otherwise, all technical and scientific terms used herein have the same meaning as commonly understood by one of ordinary skill in the art pertinent to the methods and compositions described. General definitions may be found in technical books relevant to the art of molecular biology, e.g., Dictionary of Microbiology and Molecular Biology, 2nd ed. (Singleton et al., 1994, John Wiley & Sons, New York, N.Y.) or The Harper Collins Dictionary of Biology (Hale & Marham, 1991, Harper Perennial, New York, N.Y.). As used herein, the following terms and phrases have the meanings ascribed to them unless specified otherwise.

The terms “a,” “an,” and “the” include plural referents, unless the context clearly indicates otherwise. For example, “a nucleic acid” as used herein is understood to represent one or more nucleic acids. As such, the terms “a” (or “an”), “one or more,” and “at least one” can be used interchangeably herein.

It will be appreciated that there is an implied “about” prior to the temperatures, concentrations, times, etc. discussed in the present disclosure, such that slight and insubstantial deviations are within the scope of the present teachings herein. In general, the term “about” indicates insubstantial variation in a quantity of a component of a composition not having any significant effect on the activity or stability of the composition. All ranges are to be interpreted as encompassing the endpoints in the absence of express exclusions such as “not including the endpoints.” One skilled in the art will understand that the recited ranges include the end values, as whole numbers in between the end values, and where practical, rational numbers within the range (e.g., the range 5-10 includes 5, 6, 7, 8, 9, and 10, and where practical, values such as 6.8, 10.35, etc.).

Unless specifically noted, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the embodiments). “Consisting essentially of” means that additional component(s), composition(s) or method step(s) that do not materially change the basic and novel characteristics of the compositions and methods described herein may be included in those compositions or methods. Such characteristics include the ability to detect a coronavirus nucleic acid sequence present in a sample with specificity that distinguishes the coronavirus nucleic acid from other nucleic acids in the sample. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

“Sample” includes any specimen that may contains or suspected of containing any one or more of human coronaviruses OC43, HKU1, NL63, and 229E or components thereof, such as nucleic acids or fragments of nucleic acids. Samples include “biological samples” which include any tissue or material derived from a living or dead human that may contain one or more of human coronaviruses OC43, HKU1, NL63, and 229E or target nucleic acid derived therefrom. Samples include, but are not limited to, swab samples, nasopharyngeal swab, nasal swab, mid-turbinate swab, oropharyngeal swab, throat swab, nasal wash, bronchial wash, nasal aspirate, mucus, tracheal aspirates, brush samples, respiratory tissue or exudates such as bronchoscopy, bronchoalveolar lavage (BAL) or lung biopsy, sputum, saliva, peripheral blood, plasma, serum, lymph node, gastrointestinal tissue, feces, urine, semen or other body fluids or materials. A sample may be treated or processed by sample preparation. A sample may be an individual sample (i.e., a sample derived from a single subject) or a pooled sample (i.e., a sample prepared by pooling a plurality of individual samples).

“Sample preparation” refers to any steps or methods required to prepare a sample for amplification and/or detection. A sample may be treated chemically, physically, and/or mechanically to disrupt tissue, cells, or cellular components to release intracellular components into an aqueous or organic solution which may further contain enzymes, buffers, salts, detergents and the like, which are used to prepare a biological sample for analysis. A sample may also be treated chemically, physically, and/or mechanically to remove cellular components or debris. A sample may be processed by passing the samples over or through a filtering device, centrifugation, or by adherence to a medium, matrix, or support. Sample preparation includes knowns method of concentrating components, such as polynucleotides, from a larger sample volume, such as by filtration from larger volume sample, centrifugation, or by isolation of microbes from a sample by using standard microbiology methods. Sample preparation may also include use of a polynucleotide to specifically or non-specifically capture a target nucleic acid and separate it from other sample components (e.g., as described in U.S. Pat. No. 6,110,678 and International Patent Application Pub. No. WO 2008/016988, each incorporated by reference herein).

“Separating” or “purifying” refers to removal of one or more components of a mixture, such as a sample, from one or more other components in the mixture. Sample components include nucleic acids, cellular fragments, proteins, carbohydrates, lipids, and other compounds. Separating or purifying does not connote any particular degree of purification. In some embodiments, at least 70%, at least 75%, at least 80%, at least 85%, at least 90%, or at least 95%, of the target nucleic acid or amplified product is separated or removed from other components in the mixture.

“Nucleic acid” and “polynucleotide” refer to a multimeric compound comprising nucleotides or nucleotide analogs linked together to form a polynucleotide, including conventional RNA, DNA, mixed RNA-DNA, and polymers containing analogs thereof. A nucleic acid “backbone” may be made up of a variety of linkages, including one or more of sugar-phosphodiester linkages, peptide-nucleic acid bonds (“peptide nucleic acids” or PNA; PCT Publication No. WO 95/32305), phosphorothioate linkages, methylphosphonate linkages, or combinations thereof. Sugar moieties of a nucleic acid may be ribose, deoxyribose, or similar compounds with substitutions, e.g., 2′ methoxy or 2′ halide substitutions. Nitrogenous bases may be conventional bases (A, G, C, T, U), analogs thereof (e.g., inosine, 5 methyl 2′ deoyxcytosine (“5-Me-dC” or “5MeC”), isoguanine; see The Biochemistry of the Nucleic Acids 5-36, Adams et al., ed., 11^{th} ed., 1992, Abraham et al., BioTechniques 43: 617-24, 2007), derivatives of purines or pyrimidines (e.g., N⁴-methyl deoxyguanosine, deaza- or aza-purines, deaza- or aza-pyrimidines, pyrimidine bases with substituent groups at the 5 or 6 position, purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT Publication No. WO 93/13121). Nucleic acids may include one or more “abasic” residues where the backbone includes no nitrogenous base for position(s) of the polymer (U.S. Pat. No. 5,585,481). A nucleic acid may comprise only conventional RNA or DNA sugars, bases and linkages, or may include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). Embodiments of oligomers that may affect stability of a hybridization complex include PNA oligomers, oligomers that include 2′-methoxy or 2′-fluoro substituted RNA, or oligomers that affect the overall charge, charge density, or steric associations of a hybridization complex, including oligomers that contain charged linkages (e.g., phosphorothioates) or neutral groups (e.g., methylphosphonates).

A “nucleotide” as used herein is a subunit of a nucleic acid consisting of a phosphate group, a 5-carbon sugar, and a nitrogenous base (also referred to herein as “nucleobase”). The 5-carbon sugar found in RNA is ribose. In DNA, the 5-carbon sugar is 2′-deoxyribose. The term also includes analogs of such subunits, such as a methoxy group at the 2′ position of the ribose (also referred to herein as “2′-O-Me” or “2′-methoxy”).

By “RNA and DNA equivalents” is meant RNA and DNA molecules having essentially the same complementary base pair hybridization properties. RNA and DNA equivalents have different sugar moieties (i.e., ribose versus deoxyribose) and may differ by the presence of uracil in RNA and thymine in DNA. The differences between RNA and DNA equivalents do not contribute to differences in homology because the equivalents have the same degree of complementarity to a particular sequence. By “DNA/RNA chimeric” is meant a nucleic acid comprising both DNA and RNA nucleotides. Unless the context clearly dictates otherwise, reference to a nucleic acid of human coronavirus OC43 (OC43), human coronavirus HKU1 (HKU1), human coronavirus NL63 (NL63), or human coronavirus 229E (229E) includes OC43, HKU1, NL63, or 229E RNA and DNA equivalents and DNA/RNA chimerics thereof.

A “target nucleic acid” is a nucleic acid comprising a target region or sequence to be amplified and/or detected. Target nucleic acids may be DNA or RNA and may be either single-stranded or double-stranded. A target nucleic acid can be, but is not limited to, a genomic nucleic acid, or a transcribed nucleic acid, such as an mRNA. For a single stranded target nucleic acid, such as a single stranded RNA virus or an mRNA, the target nucleic acid includes the complement thereof. A target nucleic acid can also be a nucleic acid derived from a genomic or transcribed nucleic acid. A target nucleic acid (including where appropriate its complement) contains sequences that hybridize to capture oligonucleotides, primers, and/or probes used to amplify and/or detect the target nucleic acid. The target nucleic acid may include other sequences besides the target sequence which may not be amplified.

The term “target region” or “target sequence” refers to the particular nucleotide sequence of the target nucleic acid that is to be amplified and/or detected. The target region includes the hybridizing sequences to which oligonucleotides (e.g., priming oligonucleotides and/or promoter oligonucleotides) hybridize during an amplification processes (e.g., PCR, TMA). Where the target nucleic acid is originally single-stranded, the term target region will also refer to the sequence complementary to the target sequence as present in the target nucleic acid. Where the target nucleic acid is originally double-stranded, the term target region refers to both the sense (+) and antisense (−) strands.

“Target-hybridizing sequence” or “target-specific sequence” refer to the portion of an oligomer that is configured to hybridize with a target nucleic acid sequence. The target hybridizing region is a contiguous sequence of nucleotides that hybridizes to a complementary contiguous sequence of nucleotides in the target nucleic acid sequence. In some embodiments, the target-hybridizing sequences are configured to specifically hybridize with a target nucleic acid sequence. Target-hybridizing sequences may be 100% complementary to the portion of the target sequence to which they are configured to hybridize, but not necessarily. Target-hybridizing sequences may also include inserted, deleted and/or substituted nucleotide residues relative to a target sequence. Less than 100% complementarity of a target-hybridizing sequence to a target sequence may arise, for example, when the target nucleic acid is a plurality strains within a species. It is understood that other reasons exist for configuring a target-hybridizing sequence to have less than 100% complementarity to a target nucleic acid.

Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

The term “target a sequence,” as used herein in reference to a region of a human coronavirus nucleic acid, refers to a process whereby an oligonucleotide hybridizes to a target sequence in a manner that allows for amplification and/or detection as described herein. In some embodiments, the oligonucleotide is complementary with the targeted human coronavirus nucleic acid sequence and contains no mismatches. In some embodiments, the oligonucleotides described herein are 100% complementary to the corresponding targeted human coronavirus nucleic acid sequence. In some embodiments, the oligonucleotides described herein contain 1, 2, 3, 4, or 5 mismatches with the corresponding targeted human coronavirus nucleic acid sequence In some embodiments, the oligomer specifically hybridizes to the target sequence.

The term “configured to” denotes an actual arrangement of the polynucleotide sequence configuration of a referenced oligonucleotide target-hybridizing sequence. For example, amplification oligomers that are configured to generate a specified amplicon from a target sequence have polynucleotide sequences that hybridize to the target sequence and can be used in an amplification reaction to generate the amplicon. Also, as an example, oligonucleotides that are configured to specifically hybridize to a target sequence have a polynucleotide sequence that specifically hybridizes to the referenced sequence under stringent hybridization conditions.

The term “configured to specifically hybridize to” indicates the target-hybridizing region of an amplification oligonucleotide, detection probe, or other oligonucleotide is designed to have a polynucleotide sequence that can target a sequence of the referenced human coronavirus target region. Such an oligonucleotide is not limited to targeting that sequence only, but is rather useful as a composition, in a kit, or in a method for targeting a human coronavirus target nucleic acid. The oligonucleotide is designed to function as a component of an assay for amplification and/or detection of human coronavirus target nucleic acid from a sample, and therefore is designed to target human coronavirus in the presence of other nucleic acids commonly found in testing samples. “Specifically hybridize to” does not mean exclusively hybridize to, as some small level of hybridization to non-target nucleic acids may occur, as is understood in the art. Rather, “specifically hybridize to” means that the oligonucleotide is configured to function in an assay to primarily hybridize the target so that an accurate detection of target nucleic acid in a sample can be determined.

“Oligomer,” “oligonucleotide,” or “oligo” refers to a nucleic acid of generally less than 1,000 nucleotides (nt), including those in a size range having a lower limit of about 5 nt and an upper limit of about 900 nt. In some embodiments, the oligomers are in a size range of 10-100 nucleobases, 10-90 nucleobases, 10-80 nucleobases, 10-70 nucleobases, 10-60 nucleobases, 17-35 nucleobases. In some embodiments, the oligomers are 17, 18, 19, 20, 21, 22, 23, 24, 25, 26, 27, 28, 29, 30, 31, 32, 33, 34, or 35 nucleobases in length. The oligonucleotide may be DNA and/or RNA and/or analogs thereof. Oligomers may be purified from naturally occurring sources or may be synthesized by using any well-known enzymatic or chemical method, such as, for example, the phosphoramidite synthesis method. The term oligonucleotide does not denote any particular function to the reagent; rather, it is used generically to cover all such reagents described herein. Oligomers may be referred to by a functional name (e.g., capture probe, primer or promoter primer) but those skilled in the art will understand that such terms refer to oligomers.

As used herein, an oligonucleotide “substantially corresponding to” a specified reference nucleic acid sequence means that the oligonucleotide is sufficiently similar to the reference nucleic acid sequence such that the oligonucleotide has similar hybridization properties to the reference nucleic acid sequence in that it would hybridize with the same target nucleic acid sequence under stringent hybridization conditions. One skilled in the art will understand that “substantially corresponding oligonucleotides” can vary from a reference sequence and still hybridize to the same target nucleic acid sequence. It is also understood that a nucleic acid corresponding to another nucleic acid includes the RNA or DNA equivalent thereof as well as DNA/RNA chimerics thereof, and includes the complements thereof, unless the context clearly dictates otherwise. This variation from the nucleic acid may be stated in terms of a percentage of identical bases within the sequence or the percentage of perfectly complementary bases between the probe or primer and its target sequence. In some embodiments, an oligonucleotide is “substantially corresponds” to a reference nucleic acid sequence if these percentage of base identity or complementarity is from 100% to about 80%, from 100% to about 85%, or from 100% to about 90% or from 100% to about 95%. Variation in nucleic acid sequence may also be stated in terms of the number of nucleobase substitutions in a nucleic acid sequence relative to a reference sequence, or the number of mismatches within a sequence relative to a target sequence. In some embodiments, an oligonucleotide “substantially corresponds” to a reference nucleic acid sequence if the number of nucleobase substitutions or mismatches is up to four, up to three, or up to two, or up to one substitution(s) or mismatch(es) (i.e., from zero to four, from zero to three, from zero to two, or from zero to one, inclusive). Similarly, a region of a nucleic acid or amplified nucleic acid can be referred to herein as corresponding to a reference nucleic acid sequence. One skilled in the art will understand the various modifications to the hybridization conditions that might be required at various percentages of complementarity to allow hybridization to a specific target sequence without causing an unacceptable level of non-specific hybridization.

As used herein, the phrase “or its complement, or an RNA equivalent or DNA/RNA chimeric thereof,” with reference to a DNA sequence, includes (in addition to the referenced DNA sequence) the complement of the DNA sequence, an RNA equivalent of the referenced DNA sequence, an RNA equivalent of the complement of the referenced DNA sequence, a DNA/RNA chimeric of the referenced DNA sequence, and a DNA/RNA chimeric of the complement of the referenced DNA sequence. Similarly, the phrase “or its complement, or a DNA equivalent or DNA/RNA chimeric thereof,” with reference to an RNA sequence, includes (in addition to the referenced RNA sequence) the complement of the RNA sequence, a DNA equivalent of the referenced RNA sequence, a DNA equivalent of the complement of the referenced RNA sequence, a DNA/RNA chimeric of the referenced RNA sequence, and a DNA/RNA chimeric of the complement of the referenced RNA sequence. When used in reference to a SEQ ID NO, it is understood that the term “complement thereof” would refer to the reverse complement in the case of primers, probes and the like.

An “amplification oligonucleotide” or “amplification oligomer” is an oligonucleotide that hybridizes to a target nucleic acid, or its complement, and participates in a nucleic acid amplification reaction, e.g., serving as a primer. In some embodiments, amplification oligomers contain at least about 10 contiguous bases that are complementary to a region of the target nucleic acid sequence or its complementary strand. In some embodiments, amplification oligomers contain 10, 11, 12, 13, 14, 15, 16, 17, 18, 19, 20, 21, 22, 23, 24, 25, 26, 27, or 28 contiguous bases, that are complementary to a region of the target nucleic acid sequence or its complementary strand. In some embodiments, amplification oligomers contain 10, 11, 12, 13, 14, 15, 16, 17, 18, 19, 20, 21, 22, 23, 24, 25, 26, 27, or 28 contiguous bases that are at least about 80%, at least about 90% complementary, or 100% complementary to a region of the target nucleic acid sequence or its complementary strand. In some embodiments, the amplification oligomers are about 10 to about 60 bases long. The amplification probes may be DNA, RNA, or combinations thereof (e.g., DNA/RNA chimeric). In some embodiments, the amplification oligomers contain one or more nucleotide analogs (i.e., modified nucleotides). An amplification oligomer may contain 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotide analogs. The nucleotide analogs can be independently selected from the group consisting of: nucleotide having a modified sugar (e.g., 2′-O-Me nucleotide), nucleotide having a modified nucleobase (e.g., 5-methyl cytosine), nucleotide having with modified internucleotide linkage (e.g., phosphorothioate), or combinations thereof.

An amplification oligomer combination comprises at least one first (e.g., forward or reverse) amplification oligomer and at least one second (e.g., reverse) amplification oligomer. An amplification oligomer combination is configured to amplify a target sequence.

A “primer” is an oligomer that hybridizes to a template nucleic acid and has a 3′ end that is extended by polymerization. A primer may be optionally modified, e.g., by including a 5′ region that is non-complementary to the target sequence. Such modification can include functional additions, such as tags, promoters, or other non-target-specific sequences used or useful for manipulating or amplifying the primer or target oligonucleotide.

“Non-target-specific sequence” or “non-target-hybridizing sequence” as used herein refers to a region of an oligomer sequence, wherein said region does not stably hybridize with a target sequence under standard hybridization conditions. Oligomers with non-target-specific sequences include, but are not limited to, promoter primers and molecular beacons.

“Nucleic acid amplification” refers to any in vitro procedure that produces multiple copies of a target nucleic acid sequence, or its complementary sequence, or fragments thereof (i.e., an amplified sequence containing less than the complete target nucleic acid). Examples of nucleic acid amplification procedures include transcription associated methods, such as transcription-mediated amplification (TMA), nucleic acid sequence-based amplification (NASBA) and others (e.g., U.S. Pat. Nos. 5,399,491, 5,554,516, 5,437,990, 5,130,238, 4,868,105, and 5,124,246), replicase-mediated amplification (e.g., U.S. Pat. No. 4,786,600), the polymerase chain reaction (PCR) (e.g., U.S. Pat. Nos. 4,683,195, 4,683,202, and 4,800,159), ligase chain reaction (LCR) (e.g., EP Patent No. 0320308), helicase-dependent amplification (e.g., U.S. Pat. No. 7,282,328), and strand-displacement amplification (SDA) (e.g., U.S. Pat. No. 5,422,252). Amplification may be linear or exponential. PCR amplification uses DNA polymerase, primers, and thermal cycling steps to synthesize multiple copies of the two complementary strands of DNA or cDNA. Particular embodiments herein use PCR, but it will be apparent to persons of ordinary skill in the art that oligomers disclosed herein may be readily used with other amplification methods.

In cyclic amplification methods that detect amplicons in real-time, the term “Threshold cycle” (Ct) is a measure of the emergence time of a signal associated with amplification of target and is generally 10x standard deviation of the normalized reporter signal. Once an amplification reaches the “threshold cycle,” generally there is considered to be a positive amplification product of a sequence to which the probe binds. The identity of the amplification product can then be determined through methods known to one of skill in the art, such as reporter dyes, gel electrophoresis, nucleic acid sequencing, and other such analytical procedures.

By “amplicon” or “amplification product” is meant a nucleic acid molecule generated in a nucleic acid amplification reaction and which is derived from a target nucleic acid. An amplicon may include all or part of a target region sequence. An amplicon or amplification product contains a target nucleic acid sequence that may be of the same or opposite sense as the target nucleic acid.

As used herein, the term “relative fluorescence unit” (“RFU”) is a unit of measurement of fluorescence intensity. RFU varies with the characteristics of the detection means used for the measurement and can be used as a measurement to compare relative intensities between samples and controls.

“Detection probe oligomer,” “detection probe,” or “probe” refers to an oligomer that hybridizes specifically to a target sequence, including an amplified sequence, under conditions that promote nucleic acid hybridization, for detection of the target nucleic acid. Detection may either be direct (i.e., probe hybridized directly to the target) or indirect (i.e., a probe hybridized to an intermediate structure that links the probe to the target). Detection probes may be DNA, RNA, analogs thereof or combinations thereof (e.g., DNA/RNA chimerics), and they may be labeled or unlabeled. In some embodiments, detection probe oligomers contain at least about 10 contiguous bases that are complementary to a region of the target nucleic acid sequence or its complementary strand. In some embodiments, detection probe oligomers contain 22, 23, 24, 25, 26, 27, 28, 29, 30, 31, 32, 33, 34, or 35 contiguous bases, that are complementary to a region of the target nucleic acid sequence or its complementary strand. In some embodiments, detection probe oligomers contain 22, 23, 24, 25, 26, 27, 28, 29, 30, 31, 32, 33, 34, or 35 contiguous bases that are at least about 80%, at least about 90% complementary, or 100% complementary to a region of the target nucleic acid sequence or its complementary strand. In some embodiments, the detection probe oligomers contain one or more nucleotide analogs (i.e., modified nucleotides). An detection probe oligomers may contain 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotide analogs. The nucleotide analogs can be independently selected from the group consisting of: nucleotide having a modified sugar (e.g., 2′-O-Me nucleotide), nucleotide having a modified nucleobase (e.g., 5-methyl cytosine), nucleotide having with modified internucleotide linkage (e.g., phosphorothioate), or combinations thereof. Detection probes may include alternative backbone linkages such as, e.g., 2′-O-methyl linkages. A probe's target sequence generally refers to the specific sequence within a larger sequence which the probe hybridizes specifically. A detection probe may include target-specific sequence(s) and non-target-specific sequence(s). Such non-target-specific sequences can include sequences which will confer a desired secondary or tertiary structure, such as a hairpin structure, which can be used to facilitate detection and/or amplification (see, e.g., U.S. Pat. Nos. 5,118,801, 5,312,728, 6,835,542, and 6,849,412). Probes of a defined sequence may be produced by techniques known to those of ordinary skill in the art, such as by chemical synthesis, and by in vitro or in vivo expression from recombinant nucleic acid molecules.

By “hybridization” or “hybridize” is meant the ability of two completely or partially complementary nucleic acid strands to come together under specified hybridization assay conditions in a parallel or antiparallel orientation to form a stable structure having a double-stranded region. The two constituent strands of this double-stranded structure, sometimes called a hybrid, are held together by hydrogen bonds. Although these hydrogen bonds most commonly form between nucleotides containing the bases adenine and thymine or uracil (A and T or U) or cytosine and guanine (C and G) on single nucleic acid strands, base pairing can also form between bases which are not members of these “canonical” pairs. Non-canonical base pairing is well-known in the art. See, e.g., R. L. P. Adams et al., The Biochemistry of the Nucleic Acids (11th ed. 1992).

By “preferentially hybridize” is meant that under stringent hybridization conditions, an amplification or detection probe oligomer can hybridize to its target nucleic acid to form stable oligomer:target hybrid, but not form a sufficient number of stable oligomer:non-target hybrids. Amplification and detection oligomers that preferentially hybridize to a target nucleic acid are useful to amplify and detect target nucleic acids, but not non-targeted organisms, especially phylogenetically closely related organisms. Thus, the oligomer hybridizes to target nucleic acid to a sufficiently greater extent than to non-target nucleic acid to enable one having ordinary skill in the art to accurately amplify and/or detect the presence (or absence) of nucleic acid derived from the specified target as appropriate. In general, reducing the degree of complementarity between an oligonucleotide sequence and its target sequence will decrease the degree or rate of hybridization of the oligonucleotide to its target region. However, the inclusion of one or more non-complementary nucleosides or nucleobases may facilitate the ability of an oligonucleotide to discriminate against non-target organisms.

Preferential hybridization can be measured using techniques known in the art and described herein, such as in the examples provided below. In some embodiments, there is at least a 10-fold difference between target and non-target hybridization signals in a test sample, at least a 100-fold difference, or at least a 1,000-fold difference. In some embodiments, non-target hybridization signals in a test sample are no more than the background signal level.

By “stringent hybridization conditions,” or “stringent conditions” is meant conditions permitting an oligomer to preferentially hybridize to a target nucleic acid and not to nucleic acid derived from a closely related non-target nucleic acid. While the definition of stringent hybridization conditions does not vary, the actual reaction environment that can be used for stringent hybridization may vary depending upon factors including the GC content and length of the oligomer, the degree of similarity between the oligomer sequence and sequences of non-target nucleic acids that may be present in the test sample, and the target sequence. Hybridization conditions include the temperature and the composition of the hybridization reagents or solutions. Exemplary hybridization assay conditions for amplifying and/or detecting target nucleic acids derived from a human coronavirus with the oligomers of the present disclosure correspond to a temperature of about 60° C. when the salt concentration, such as a monovalent salt, e.g., KCl, is in the range of about 0.6-0.9 M. Other acceptable stringent hybridization conditions are readily ascertained by those having ordinary skill in the art.

By “assay conditions” is meant conditions permitting stable hybridization of an oligonucleotide to a target nucleic acid. Assay conditions do not require preferential hybridization of the oligonucleotide to the target nucleic acid.

“Label” or “detectable label” refers to a moiety or compound joined directly or indirectly to a probe that is detected or leads to a detectable signal. Direct joining may use covalent bonds or non-covalent interactions (e.g., hydrogen bonding, hydrophobic or ionic interactions, and chelate or coordination complex formation) whereas indirect joining may use a bridging moiety or linker (e.g., via an antibody or additional oligonucleotide(s), which amplify a detectable signal. Any detectable moiety may be used, e.g., radionuclide, ligand such as biotin or avidin, enzyme, enzyme substrate, reactive group, chromophore such as a dye or particle (e.g., latex or metal bead) that imparts a detectable color, luminescent compound (e.g., bioluminescent, phosphorescent, or chemiluminescent compound), and fluorescent compound (i.e., fluorophore). Fluorophores may be used in combination with a quencher molecule that absorbs light when in close proximity to the fluorophore to diminish background fluorescence. Methods of synthesizing labels, attaching labels to nucleic acid, and detecting signals from labels are well known (e.g., Sambrook et al., Molecular Cloning, A Laboratory Manual, 2nd ed. (Cold Spring Harbor Laboratory Press, Cold Spring Harbor, N.Y., 1989) at Chapt. 10, and U.S. Pat. Nos. 5,658,737, 5,656,207, 5,547,842, 5,283,174, and 4,581,333, and EP Pat. App. 0 747 706). Particular methods of linking an AE compound to a nucleic acid are known (e.g., U.S. Pat. Nos. 5,585,481 and 5,639,604, see column 10, line 6 to column 11, line 3, and Example 8). Detectably labeled probes include TaqMan™ probes, AE-labeled probes, molecular torches, and molecular beacons. TaqMan™ probes include a fluorophore and quencher labels wherein fluorescence is detected upon enzymatically degrading the probe during amplification in order to release the fluorophore from the presence of the quencher. Molecular torches and beacons exist in open and closed configurations wherein the closed configuration quenches the fluorophore and the open position separates the fluorophore from the quencher to allow fluorescence. Hybridization to target opens the otherwise closed probes. Particular homogeneous detectable labels include chemiluminescent compounds, including acridinium ester (“AE”) compounds, such as standard AE or AE derivatives which are well known (U.S. Pat. Nos. 5,656,207, 5,658,737, and 5,639,604).

A “quencher” is a molecule that absorbs light. Quenchers are commonly used in combination with a light emitting label such as a fluorophore to absorb emitted light when in close proximity to the fluorophore. Quenchers are well-known in the art and include, but are not limited to, Black Hole Quencher™ (or BHQ™, BHQ-1™, or BHQ-2™), Blackberry Quencher, Dabcyl, QSY, and Tamra™ compounds, to name a few.

Sequences are “sufficiently complementary” if they allow stable hybridization of two nucleic acid sequences, e.g., stable hybrids of probe and target sequences, although the sequences need not be completely complementary. That is, a “sufficiently complementary” sequence that hybridizes to another sequence by hydrogen bonding between a subset series of complementary nucleotides by using standard base pairing (e.g., G:C, A:T, or A:U), although the two sequences may contain one or more residues (including abasic positions) that are not complementary so long as the entire sequences in appropriate hybridization conditions to form a stable hybridization complex. Sufficiently complementary sequences may be at least about 80%, at least about 90%, or completely complementary in the sequences that hybridize together. Appropriate hybridization conditions are well-known to those skilled in the art, can be predicted based on sequence composition, or can be determined empirically by using routine testing (e.g., Sambrook et al., Molecular Cloning, A Laboratory Manual, 2^{nd} ed. at §§ 1.90-1.91, 7.37-7.57, 9.47-9.51 and 11.47-11.57, particularly §§ 9.50-9.51, 11.12-11.13, 11.45-11.47 and 11.55-11.57).

A “non-extendable” oligomer includes a blocking moiety at or near its 3′-terminus to prevent extension. A blocking group near the 3′ end is in some embodiments within five residues of the 3′ end and is sufficiently large to limit binding of a polymerase to the oligomer, and other embodiments contain a blocking group covalently attached to the 3′ terminus. Many different chemical groups may be used to block the 3′ end, e.g., alkyl groups, non-nucleotide linkers, alkane-diol dideoxynucleotide residues, and cordycepin. Further examples of blocking moieties include a 3′-deoxy nucleotide (e.g., a 2′,3′-dideoxy nucleotide); a 3′-phosphorylated nucleotide; a fluorophore, quencher, or other label that interferes with extension; an inverted nucleotide (e.g., linked to the preceding nucleotide through a 3′-to-3′ phosphodiester, optionally with an exposed 5′-OH or phosphate, referred to herein as “revC,” “revA,” ‘revG,” etc.); or a protein or peptide joined to the oligonucleotide so as to prevent further extension of a nascent nucleic acid chain by a polymerase. A non-extendable oligonucleotide of the present disclosure may be at least 10 bases in length, and may be up to 15, 20, 25, 30, 35, 40, 50 or more nucleotides in length. Non-extendable oligonucleotides that comprise a detectable label can be used as probes.

Reference, particularly in the embodiments, to “the sequence of SEQ ID NO:X” refers to the nucleobase sequence of the corresponding sequence listing entry and does not require identity of the backbone (e.g., RNA, DNA, or sugar modification such as 2′-O-Me nucleotide) or any nucleobase modifications (e.g., methylation of cytosine residues (“5MeC”)) unless the context clearly dictates otherwise. Reference to “include nucleotide analogs,” or “including from x-y nucleotide analogs” in the context of a reference to a SEQ ID NO: indicates the oligonucleotide may contain nucleotide analogs within the indicated sequence (e.g., nucleotide analog substitution), in addition to the indicated sequence, or both.

The term “non-linear surfactant,” is a surfactant having a branched chain structure. A non-linear surfactant may include one or more ring structures, which may be, for example, in a principal chain and/or in one or more branch chains. Exemplary non-linear surfactants include polysorbate 20, polysorbate 40, polysorbate 60, and digitonin. In certain variations, a non-linear surfactant is non-ionic.

The term “specificity,” in the context of an amplification and/or detection system, is used herein to refer to the characteristic of the system which describes its ability to distinguish between target and non-target sequences dependent on sequence and assay conditions. In terms of nucleic acid amplification, specificity generally refers to the ratio of the number of specific amplicons produced to the number of side-products (e.g., the signal-to-noise ratio). In terms of detection, specificity generally refers to the ratio of signal produced from target nucleic acids to signal produced from non-target nucleic acids.

The term “sensitivity” is used herein to refer to the precision with which a nucleic acid amplification reaction can be detected or quantitated. The sensitivity of an amplification reaction is generally a measure of the smallest copy number of the target nucleic acid that can be reliably detected in the amplification system, and will depend, for example, on the detection assay being employed, and the specificity of the amplification reaction, e.g., the ratio of specific amplicons to side-products.

### DETAILED DESCRIPTION

Provided herein are compositions, kits, formulations, and methods for amplifying and/or detecting human coronavirus OC43, HKU1, NL63, and/or 229E nucleic acid in a sample. In some embodiments, the samples are biological samples. The compositions, kits, formulations and methods provide oligonucleotide sequences that target the ORF 1a gene sequences of OC43 and HKU1, the spike protein gene sequence of NL63, and the ORF 1b gene sequence of 229E, or their complementary sequences. The described amplification oligonucleotides, which may include primers, promoter primers, blocked oligonucleotides, and promoter provider oligonucleotides, whose functions have been described previously (see, e.g., U.S. Pat. Nos. 4,683,195; 4,683,202; 4,800,159; 5,399,491; 5,554,516; 5,824,518; and 7,374,885; each incorporated by reference herein). The described detection probe oligonucleotides may be used as probes for detecting amplified sequences of human coronavirus OC43, HKU1, NL63, or 229E, or for capture of human coronavirus OC43, HKU1, NL63, or 229E target nucleic acid.

The methods provide for the sensitive and specific detection of human coronavirus OC43, HKU1, NL63, and/or 229E nucleic acids. The methods include performing nucleic acid amplification of an OC43 target region, an HKU1 target region, an NL63 target region, and/or a 229E target region, and optionally detecting the amplified product. Detection may be performed by, for example, specifically hybridizing the amplified product with a nucleic acid detection probe that provides a signal to indicate the presence of OC43, HKU1, NL63, and/or 229E in the sample. The amplification step includes contacting the sample with one or more amplification oligomers specific for a target sequence in an OC43, HKU1, NL63, and/or 229E target nucleic acid to produce an amplified product if OC43, HKU1, NL63, or 229E nucleic acid is present in the sample. Amplification synthesizes additional copies of the target sequence or its complement by using at least one nucleic acid polymerase and an amplification oligomer to produce the copies from a template strand (e.g., by extending the sequence from a primer using the template strand). One embodiment for detecting the amplified product uses a hybridizing step that includes contacting the amplified product with at least one detection probe oligomer specific for a sequence amplified by the selected amplification oligomers, e.g., a sequence contained in the target sequence flanked by a pair of selected amplification oligomers.

In some aspects, the compositions are configured to specifically hybridize to human coronavirus OC43, HKU1, NL63, and/or 229E nucleic acid with minimal cross-reactivity to one or more non-coronavirus pathogens. In some aspects, the compositions are configured to specifically hybridize to OC43, HKU1, NL63, and/or 229E nucleic acid with minimal cross-reactivity to one or more non-coronavirus pathogens listed in Table 25 (see Example 11, infra). In other, non-mutually exclusive aspects, the compositions are configured to specifically hybridize to OC43, HKU1, NL63, and/or 229E nucleic acid with minimal cross-reactivity to a coronavirus pathogen that is not OC43, HKU1, NL63, or 229E. In one aspect, the compositions are part of a multiplex system that further includes components and methods for detecting one of more pathogens that are not human coronavirus OC43, HKU1, NL63, or 229E (e.g., one or more non-coronavirus pathogens such as, for example, one or more of the pathogens listed in Table 25).

In some aspects, there are provided methods for utilizing an oligomer or oligomer combination as described herein. Any method disclosed herein is also to be understood as a disclosure of corresponding uses of materials involved in the method directed to the purpose of the method. Any of the oligomers comprising a human coronavirus OC43, HKU1, NL63, or 229E target-hybridizing sequence and any combinations (e.g., kits and compositions) comprising such an oligomer are to be understood as also disclosed for use in detecting or quantifying OC43, HKU1, NL63, and/or 229E, and for use in the preparation of a composition for detecting or quantifying OC43, HKU1, NL63 and/or 229E.

Broadly speaking, methods may comprise one or more of the following components: target capture, in which OC43, HKU1, NL63, or 229E nucleic acid (e.g., from a sample, such as a clinical sample) is annealed to a capture oligomer; isolation, e.g., washing, to remove material not associated with a capture oligomer; amplification; and amplicon detection, e.g., amplicon quantification, which may be performed in real time with amplification. Certain embodiments involve each of the foregoing steps. Certain embodiments involve exponential amplification, optionally with a preceding linear amplification step. Certain embodiments involve exponential amplification and amplicon detection. Certain embodiments involve any two of the components listed above. Certain embodiments involve any two components listed adjacently above, e.g., washing and amplification, or amplification and detection.

Amplifying a coronavirus target sequence utilizes an in vitro amplification reaction using at least two amplification oligomers that flank a target region to be amplified. Particularly suitable oligomer combinations for amplification of these coronavirus target regions are described herein. In some instances, two, three, or four of the coronavirus OC43, HKU1, NL63 and 229E target regions are to be amplified in a multiplex format. In some instances, one, two, three or four of the coronavirus target regions and an internal control target region are to be amplified in a multiplex format. In some instances, combinations of first and second amplification oligomers for any two of OC43, HKU1, NL63 and 229E target regions are provided. In some instances, combinations of first and second amplification oligomers for any three of OC43, HKU1, NL63 and 229E target regions are provided. In some instances, combinations of first and second amplification oligomers for all four of OC43, HKU1, NL63 and 229E target regions are provided. In some instances, combinations of first and second amplification oligomers for each of the regions to be amplified are provided.

In some embodiments, the methods further include purifying one or more of the human coronavirus OC43, HKU1, NL63, and/or 229E target nucleic acids from other components in the sample, e.g., before an amplification. Such purification may include methods of separating and/or concentrating organisms contained in a sample from other sample components, or removing or degrading non-nucleic acid sample components, e.g., protein, carbohydrate, salt, lipid, etc. In some embodiments, purifying comprises a capture step. Target capture can be used to separate the target nucleic acid(s) from other sample components. Target nucleic acid can be captured specifically or non-specifically. Non-specific target capture methods may involve selective precipitation of nucleic acids from a substantially aqueous mixture, adherence of nucleic acids to a support that is washed to remove other sample components, or other means of physically separating nucleic acids from other sample components a mixture that contains human coronavirus OC43, HKU1, NL63, and/or 229E nucleic acid

Target capture typically occurs in a solution phase mixture that contains one or more capture probe oligomers that hybridize to the OC43, HKU1, NL63, or 229E target sequence under hybridizing conditions. For embodiments comprising a capture probe tail, the OC43-target:capture-probe, HKU1-target:capture-probe, NL63-target:capture-probe, 229E-target:capture-probe hybridization complex is captured by adjusting the hybridization conditions so that the capture probe tail hybridizes to an immobilized probe. Certain embodiments use a particulate solid support, such as paramagnetic beads. In some embodiments, a capture probe tail hybridizes to a complementary nucleic acid linked to a solid support.

Isolation can follow capture, where, for example, the complex on the solid support is separated from other sample components. Isolation can be accomplished by any appropriate technique, e.g., washing a support associated with the OC43, HKU1, NL63, or 229E target-sequence one or more times (e.g., two or three times) to remove other sample components and/or unbound oligomer. In embodiments using a particulate solid support, such as paramagnetic beads, particles associated with the OC43, HKU1, NL63, or 229E target may be suspended in a washing solution and retrieved from the washing solution, in some embodiments by using magnetic attraction. To limit the number of handling steps, the OC43, HKU1, NL63, or 229E target nucleic acid may be amplified by simply mixing the target sequence in the complex on the support with amplification oligomers and proceeding with amplification steps.

Exponentially amplifying a human coronavirus OC43, HKU1, NL63, and/or 229E target sequence utilizes an in vitro amplification reaction using at least two amplification oligomers that flank a target region to be amplified. The amplification reaction can be cycled or isothermal.

A detection step may be performed using any of a variety of known techniques to detect a signal specifically associated with the amplified target sequence, such as, e.g., by hybridizing the amplification product with a labeled detection probe and detecting a signal resulting from the labeled probe (including from label released from the probe following hybridization in some embodiments). In some embodiments, the labeled probe comprises a second moiety, such as a quencher or other moiety that interacts with the first label, as discussed above. The detection step may also provide additional information on the amplified sequence, such as, e.g., all or a portion of its nucleic acid base sequence. Detection may be performed after the amplification reaction is completed or may be performed simultaneously with amplifying the target region, e.g., in real time. In some embodiments, the amplified product is detected near or at the end of the amplification step using a linear detection probe that provides a signal to indicate hybridization of the probe to the amplified product. One example of such detection uses a luminescently labeled probe that hybridizes to target nucleic acid. The luminescent label is then hydrolyzed from non-hybridized probe. Detection is performed by chemiluminescence using a luminometer. (see, e.g., International Patent Application Pub. No. WO 89/002476, incorporated by reference herein). In some embodiments, detection is performed in real-time detection using a detection probe comprising a hairpin probe such as, for example, a molecular beacon, molecular torch, or hybridization switch probe that is labeled with a reporter moiety that is detected when the probe binds to amplified product (e.g., a dual-labeled hairpin probe comprising both a fluorescent label and a quenching moiety). In some embodiments, detected is performed in real-time detection using a detection probe comprising a linear oligomer such as, e.g., an oligomer labeled with both a fluorophore and a quenching moiety (e.g., a TaqMan probe). Such probes may comprise target-hybridizing sequences and non-target-hybridizing sequences. Various forms of such probes have been described previously (see, e.g., U.S. Pat. Nos. 5,210,015; 5,487,972; 5,118,801; 5,312,728; 5,925,517; 6,150,097; 6,849,412; 6,835,542; 6,534,274; and 6,361,945; and US Patent Application Pub. Nos. 20060068417A1 and 20060194240A1; each incorporated by reference herein).

Assays for detection of the human coronavirus OC43, HKU1, NL63, and/or 229E nucleic acid may optionally include a non-OC43/HKU1/NL3/229E internal control (IC) nucleic acid that is amplified and detected in the same assay reaction mixtures by using amplification and detection oligomers specific for the IC sequence. IC nucleic acid sequences can be, e.g., a DNA plasmid, an RNA template sequence (e.g., an in vitro transcript), or a synthetic nucleic acid that is spiked into a sample. Alternatively, the IC nucleic acid sequence may be a cellular component, which may be from exogenous cellular sources or endogenous cellular sources relative to the specimen. The internal control nucleic acid can be co-amplified with the OC43, HKU1, NL63, and/or 229E nucleic acid in the amplification reaction mixtures. The internal control amplification product and the OC43, HKU1, NL63, and/or 229E target sequence amplification product can be detected independently.

In certain embodiments, amplification and detection of a signal from an amplified IC sequence indicates that amplifying and detecting steps of the method were properly performed and any reagents and equipment functioned properly in the assay. Amplification and detection of a control sequence can be useful in instances when no signal is obtained for the intended target OC43, HKU1, NL63, and/or 229E nucleic acid (e.g., samples that test negative for OC43, HKU1, NL63, and/or 229E). An IC may also be used as an internal calibrator for the assay when a quantitative result is desired, i.e., the signal obtained from the IC amplification and detection is used to set a parameter used in an algorithm for quantitating the amount of OC43, HKU1, NL63, and/or 229E nucleic acid in a sample based on the signal obtained for an amplified OC43, HKU1, NL63, and/or 229E target sequence. ICs are also useful for monitoring the integrity of one or more steps in an assay. The primers and probe for the IC target sequence are configured and synthesized by using any well-known method provided that the primers and probe function for amplification of the IC target sequence and detection of the amplified IC sequence using substantially the same assay conditions used to amplify and detect the OC43, HKU1, NL63, and/or 229E target sequence. In some embodiments that include a target capture-based purification step, a target capture probe specific for the IC target is included in the assay in the target capture step so that the IC is treated in the assay in a manner analogous to that for the intended OC43, HKU1, NL63, and/or 229E analyte in all of the assay steps.

Also provided herein are formulations for determining the presence or absence of human coronavirus OC43, HKU1, NL63, and/or 229E in a sample. In some embodiments, a formulation is an aqueous formulation comprising (1) (a) at least two OC43-specific amplification oligomers for amplification of an OC43 target region as described herein, (b) at least two HKU1-specific amplification oligomers for amplification of an HKU1 target region as described herein, (c) at least two NL63-specific amplification oligomers for amplification of an NL63 target region as described herein, and/or (d) at least two 229E-specific amplification oligomers for amplification of a 229E target region as described herein, and (2) an organic buffer. In some embodiments, the aqueous formulation comprises: (a) at least two OC43-specific amplification oligomers for amplification of an OC43 target region as described herein, (b) at least two HKU1-specific amplification oligomers for amplification of an HKU1 target region as described herein, (c) at least two NL63-specific amplification oligomers for amplification of an NL63 target region as described herein, and (d) at least two 229E-specific amplification oligomers for amplification of a 229E target region as described herein. In some embodiments, the aqueous formulation further comprises IC amplification oligomers for amplification of an IC target region. An aqueous formulation for amplification of an OC43, HKU1, NL63, and/or 229E nucleic acid may include one or more additional components such as, e.g., a DNA polymerase enzyme, a reverse transcriptase enzyme, or a detection probe oligomer. In some embodiments, the aqueous formulation further comprises an OC43-specific detection probe oligomer, a HKU1-specific detection probe oligomer, a NL63-specific detection probe oligomer, and/or a detection probe oligomer 229E-specific detection probe oligomer as described herein. In some embodiments, the aqueous formulation further comprises an OC43-specific detection probe oligomer, a HKU1-specific detection probe oligomer, a NL63-specific detection probe oligomer, and a detection probe oligomer 229E-specific detection probe oligomer as described herein. In some embodiments, the aqueous formulation further comprises an IC detection probe oligomer. In some embodiments, a formulation is an aqueous formulation comprising (1) an OC43-specific, HKU1-specific, NL63-specific, and/or 229E-specific detection probe oligomer as described herein and (2) an organic buffer. An aqueous formulation comprising one or more detection probe oligomers may include one or more additional components such as, e.g., a surfactant, a DNA polymerase enzyme, a reverse transcriptase enzyme, or at least one amplification oligomer. Particularly suitable surfactants include, for example, polyethylene glycol mono [4-(1,1,3,3-tetramethylbutyl) phenyl] ether and polyoxyethylene sorbitan fatty acid esters (e.g., polysorbate 20, polysorbate 40, or polysorbate 60). In some embodiments, a surfactant in an aqueous detection probe formulation is a non-linear surfactant such as, for example, a polyoxyethylene sorbitan fatty acid ester (e.g., polysorbate 20, polysorbate 40, or polysorbate 60) or digitonin. An aqueous formulation as above for amplification or detection of OC43, HKU1, NL63, and/or 229E nucleic acid may further include a bulking agent such as, e.g., trehalose, raffinose, or a combination thereof. In some embodiments, an aqueous formulation as above contains at inorganic salt such as, e.g., magnesium, potassium, or sodium; in some such variations, the concentration of the inorganic salt is 4 mM or less. A particularly suitable organic buffer for an aqueous formulation as above is Tris (2-amino-2-(hydroxymethyl)-1,3-propanediol).

In a related aspect, for long-term storage, an aqueous formulation as described herein may be aliquoted into, e.g., vials, ampules, or other containers and dried (e.g., lyophilized) according to procedures known in the art. The dried product typically appears as a powder or cake. The containers are then sealed. Methods of preparing such dried formulations from the aqueous formulation, as well as the dried formulations prepared by such methods, are additional aspects of the instant disclosure. In yet another aspect, there is provided a dried formulation that enables reconstitution into an aqueous formulation as described herein. Dried formulations for amplification or detection of OC43, HKU1, NL63, and/or 229E nucleic acids typically contain, in addition to one or more amplification oligomers and/or detection probes as described herein, a bulking agent such as, e.g., trehalose, raffinose, or a combination thereof. In some embodiments, a dried formulation further comprises an inorganic salt. The percent mass of the inorganic salt is 0.249% or less, 0.222% or less, or 0.195% or less of the total mass of the dried formulation. A dried formulation by made by drying any of the described aqueous formulations using methods known in the art (e.g., lyophilization). Aqueous formulations may be made by reconstitution of any of the described dried formulations. Aqueous formulations are typically made from dried formulations by dissolving the dried formulation in a suitable diluent (e.g., an organic buffer or water) to provide a reconstituted formulation.

Also provided reaction mixtures for determining the presence or absence of an OC43, HKU1, NL63, and/or 229E target nucleic acid in a sample. A reaction mixture in accordance with the present disclosure includes one or both of (a) an oligomer combination as described herein for amplification of an OC43, HKU1, NL63, and/or 229E target nucleic acid and (b) one or more detection probe oligomers as described herein for determining the presence or absence of an OC43, HKU1, NL63, and/or 229E amplification product. In some embodiments, the reaction mixture comprises oligomer combinations as described herein for amplification of an each of OC43, HKU1, NL63, and 229E target nucleic acid detection probe oligomers as described herein for determining the presence or absence of each of OC43, HKU1, NL63, and 229E amplification products. In some embodiments, the reaction mixture may comprise one or more capture probes, e.g., poly-(k) capture probes as described in US 2013/0209992, which is incorporated herein by reference. For an amplification reaction mixture, the reaction mixture will typically include other reagents suitable for performing in vitro amplification such as, e.g., buffers, salt solutions, appropriate nucleotide triphosphates (e.g., dATP, dCTP, dGTP, and dTTP; and/or ATP, CTP, GTP and UTP), and/or enzymes (e.g., a thermostable DNA polymerase, or reverse transcriptase and/or RNA polymerase), and will typically include test sample components, in which an OC43, HKU1, NL63, or 229E target nucleic acid may or may not be present. In some embodiments, a reaction mixture includes amplification oligomers for amplification of a target region of one of OC43, HKU1, NL63, or 229E. In some embodiments, a reaction mixture includes amplification oligomers for amplification of target regions for any two of OC43, HKU1, NL63, and 229E. In some embodiments, a reaction mixture includes amplification oligomers for amplification of target regions for any three of OC43, HKU1, NL63, and 229E. In some embodiments, a reaction mixture includes amplification oligomers for amplification of target regions for each of OC43, HKU1, NL63, and 229E. A reaction mixture can include amplification oligomers for amplification of a single target region in each of OC43, HKU1, NL63, and/ or 229E or two or more target regions in each of OC43, HKU1, NL63, and/ or 229E. In addition, for a reaction mixture that includes a detection probe together with an amplification oligomer combination, selection of amplification oligomers and detection probe oligomers for a reaction mixture are linked by a common target region (i.e., the reaction mixture will include a probe that binds to a sequence amplifiable by an amplification oligomer combination of the reaction mixture). In some embodiments, a reaction mixture comprises an aqueous formulation as described above. In some embodiments, a reaction mixture is reconstituted with water or an organic buffer from a dried formulation as described above.

Also provided are kits for practicing the methods as described herein. A kit in accordance with the present disclosure includes one or both of (a) an oligomer combination as described herein for amplification of an OC43, HKU1, NL63, and/or 229E target nucleic acid and (b) one or more detection probe oligomers as described herein for determining the presence or absence of an OC43, HKU1, NL63, and/or 229E amplification products. The kits may further include one or more optional components such as, for example, capture probes, e.g., poly-(k) capture probes as described in US 2013/0209992. Other reagents that may be present in the kits include reagents suitable for performing in vitro amplification such as, e.g., buffer, organic buffer, salt solution, appropriate nucleotide triphosphates (e.g., dATP, dCTP, dGTP, dTTP; and/or ATP, CTP, GTP and UTP), and/or enzyme (e.g., a thermostable DNA polymerase, or a reverse transcriptase, and/or RNA polymerase). Oligomers as described herein may be packaged in a variety of different embodiments. Those skilled in the art will appreciate that the disclosure embraces many different kit configurations. In some embodiments, a kit includes amplification oligomers for amplification of a target region of one of OC43, HKU1, NL63, or 229E. In some embodiments, a kit includes amplification oligomers for amplification of target regions for any two of OC43, HKU1, NL63, and 229E. In some embodiments, a kit includes amplification oligomers for amplification of target regions for any three of OC43, HKU1, NL63, and 229E. In some embodiments, a kit includes amplification oligomers for amplification of target regions for each of OC43, HKU1, NL63, and 229E. A kit can include amplification oligomers for amplification of a single target region in each of OC43, HKU1, NL63, and/ or 229E or two or more target regions in each of OC43, HKU1, NL63, and/ or 229E. In addition, for a kit that includes a detection probe together with an amplification oligomer combination, selection of amplification oligomers and detection probe oligomers for a kit are linked by a common target region (i.e., the kit will include a probe that binds to a sequence amplifiable by an amplification oligomer combination of the kit). In certain embodiments, the kit further includes a set of instructions for practicing methods in accordance with the present disclosure. Instructions may be associated with a package insert and/or the packaging of the kit or the components thereof.

The compositions, kits, formulations, reaction mixtures, and methods are further illustrated by the following non-limiting examples.

### EXAMPLES

### Example 1

This example describes screening experiments testing several primer and probe combinations for the real-time PCR amplification and detection of coronavirus types OC43. Reactions were generally prepared and performed as presented herein and as follows.

Several primer and probe mixtures (PPR mixes) were prepared in a microfuge tube to include a forward primer, a reverse primer and a dual labeled hydrolysis detection probe. The internal control PPR mix comprised 0.625 μM of each primer and 0.5 μM of the probe, while the coronavirus PPR mixes contained 1.25 μM of each of the primers and the probe. These PPR mixes also contained 150 mM of KCl, 10 mM MgCl₂, and were brought to final volume using 10 mM TRIS. For this experiment, the IC detection probe was labeled with Quasar 705 and Black Hole Quencher 2 and each coronavirus (“CoV”) detection probe was labeled with FAM and Black Hole Quencher 1 (all available from BioSearch Technologies, Inc., Novato, Calif. or Glen Research, Inc., Sterling, Va.). The combinations of primers and probes are recited in Table 1.

TABLE 1

| PPR Mix | Forward Primer | Reverse Primer | Detection Probe |
|---|---|---|---|
| IC PPR Mix | SEQ ID NO: 21 | SEQ ID NO: 29 | SEQ ID NO: 46 |
| OC43 PPR Mix v1.1 | SEQ ID NO: 5 | SEQ ID NO: 43 | SEQ ID NO: 7 |
| OC43 PPR Mix v2.1 | SEQ ID NO: 38 | SEQ ID NO: 41 | SEQ ID NO: 47 |
| OC43 PPR Mix v2.2 | SEQ ID NO: 38 | SEQ ID NO: 32 | SEQ ID NO: 47 |
| OC43 PPR Mix v2.3 | SEQ ID NO: 39 | SEQ ID NO: 41 | SEQ ID NO: 47 |
| OC43 PPR Mix v2.4 | SEQ ID NO: 39 | SEQ ID NO: 32 | SEQ ID NO: 47 |
| OC43 PPR Mix v3.1 | SEQ ID NO: 24 | SEQ ID NO: 44 | SEQ ID NO: 33 |
| OC43 PPR Mix v3.2 | SEQ ID NO: 24 | SEQ ID NO: 51 | SEQ ID NO: 33 |

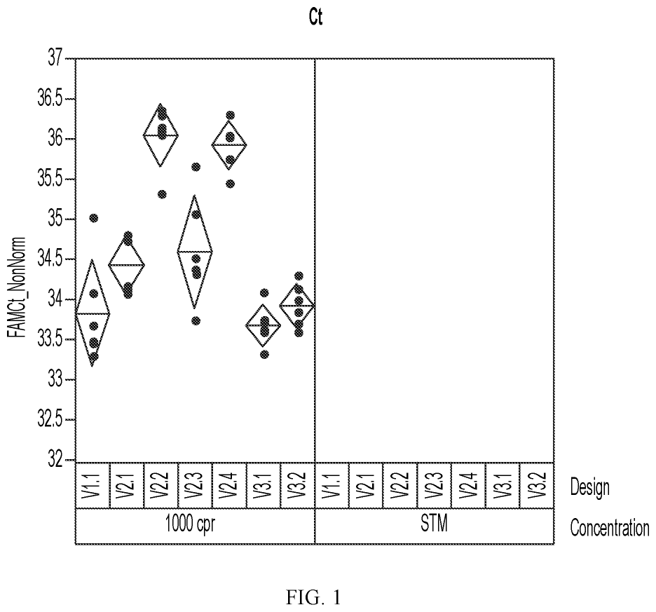

An equal volume of the internal control PPR mix (275 μL) was added each of the coronavirus mixtures (275 μL) to provide 1.25X PPR mixes (550 μL total volume). Each of the 1.25× PPR mixes was then overlaid with 250 μL of oil. A synthetic coronavirus OC43 target nucleic acid (SEQ ID NO:52) was prepared from a stock concentration to provide 1,000 copies per reaction in a 5 μL aliquot (200 copies/μL). Dilutions were made into a sample transport media (containing LLS, EDTA, and sodium phosphate). Amplification and detection reactions were set up at 12 reactions per condition; 6 reactions positive for the target nucleic acid and 6 reactions negative for the target nucleic acid. Negative reactions included sample transport media without the coronavirus target nucleic acid. The reactions were performed using a Panther Fusion system (available from Hologic, Inc., Marlborough Mass.) using its Open Access workflow feature (LDT-RNA-10 mm), and the data was analyzed using the MyAccess Tool and JMP chart analysis. Results are shown in FIGS. 1 & 2.

The resulting amplification curves were evaluated for differences in Ct and RFU signal for the positive samples, and background RFU for the negative samples. Background RFU was negligible in all designs tested in this group. OC43 PPR Mix v1.1 had the highest average RFU signal for positive sample in both the raw and processed curves and was therefore identified as being the design with best sensitivity for OC43 in this group. OC43 PPR Mix v1.1, 0C43 PPR Mix v3.1, and OC43 PPR Mix v3.2 had the fastest Ct performance.

### Example 2

This example describes screening experiments testing the impact of nucleobase modifications and quencher placement on PPR mix performance. Reactions were generally prepared and performed as follows.

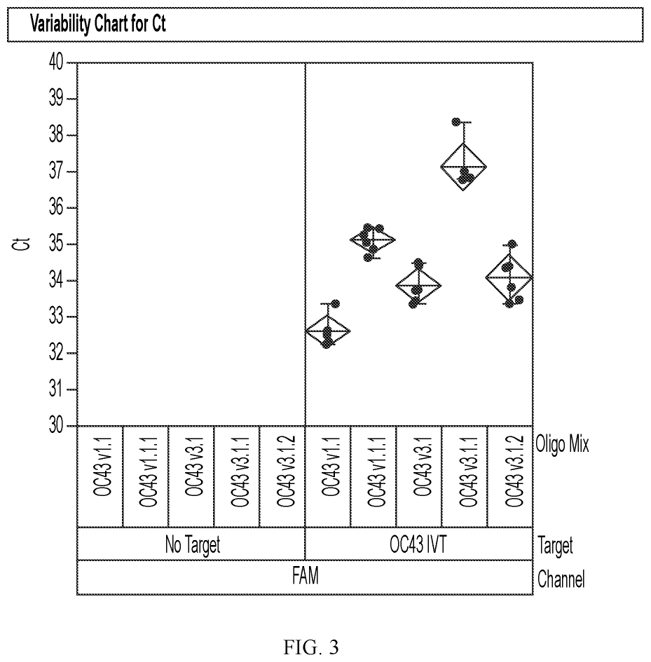

Several PPR mixes were prepared as generally described in Example 1, above. The various PPR mixes included primers and probes comprising nucleotide sequences and further comprising analog nucleotides and varied placement of fluorophores/quenchers, see Table 2. Quasar 705 (Q705); Black Hole Quencher 1 (BHQ-1); Black Hole Quencher 2 (BHQ-2); and revC are available from Biosearch Technologies, Inc., Novato, Calif. 6-Carboxyfluorescein (FAM) is available from Integrated DNA Technologies, Inc., Coralville, Iowa. 5MeC is available from Sigma-Aldrich Corp., St. Louis, Mo. Positive reactions contained 10,000 copies of coronavirus OC43 target nucleic acid (SEQ ID NO:52) per reaction. Negative reactions contained only STM. Results are shown in FIGS. 3 & 4.

TABLE 2

| PPR Mix | Forward Primer | Reverse Primer | Detection Probe |
|---|---|---|---|
| IC PPR Mix | SEQ ID NO: 21 | SEQ ID NO: 29 | SEQ ID NO: 46 |
| | | | 5′ Q705 |
| | | | 3′BHQ-2 |
| OC43 PPR Mix v1.1 | SEQ ID NO: 5 | SEQ ID NO: 43 | SEQ ID NO: 7 |
| | | | 5′ FAM |
| | | | 3′BHQ-l |
| OC43 PPR Mix v1.1.1 | SEQ ID NO: 5 | SEQ ID NO: 43 | SEQ ID NO: 7 |
| | | 5MeC at residues | 5′ FAM |
| | | 10, 12, 13, & 15 | Internal BHQ-1 (residue 13) |
| | | | 3′RevC |
| OC43 PPR Mix v3.1 | SEQ ID NO: 24 | SEQ ID NO: 44 | SEQ ID NO: 33 |
| | | | 5′ FAM |
| | | | 3′BHQ-l |
| OC43 PPR Mix v3.1.1 | SEQ ID NO: 24 | SEQ ID NO: 44 | SEQ ID NO: 33 |
| | 5MeC at residues | 5MeC at residues | 5′FAM |
| | 4, 7, 8, & 15 | 4, 6, 16, 23, & 24 | Internal BHQ-1 (residue 15) |
| | | | 5MeC at residues 3 & 8 |
| | | | 3′RevC |
| OC43 PPR Mix v3.1.2 | SEQ ID NO: 24 | SEQ ID NO: 44 | SEQ ID NO: 34 |
| | 5MeC at residues | 5MeC at residues | 5′ FAM |
| | 4, 7, 8, & 15 | 4, 6, 16, 23, & 24 | Internal BHQ-1 (residue 15) |
| | | | 5MeC at residues 3 & 8 |
| | | | 3′RevC |

Conclusions: OC43 PPR Mix v1.1 shows the fastest Ct performance and highest RFU above background compared to the other systems. OC43 PPR Mix v3.1 and OC43 PPR Mix v3.1.2 both showed about equally fast Ct performance, and OC43 PPR Mix v3.1.2 showed a slightly higher RFU above background than did OC43 PPR Mix v3.1.

### Example 3

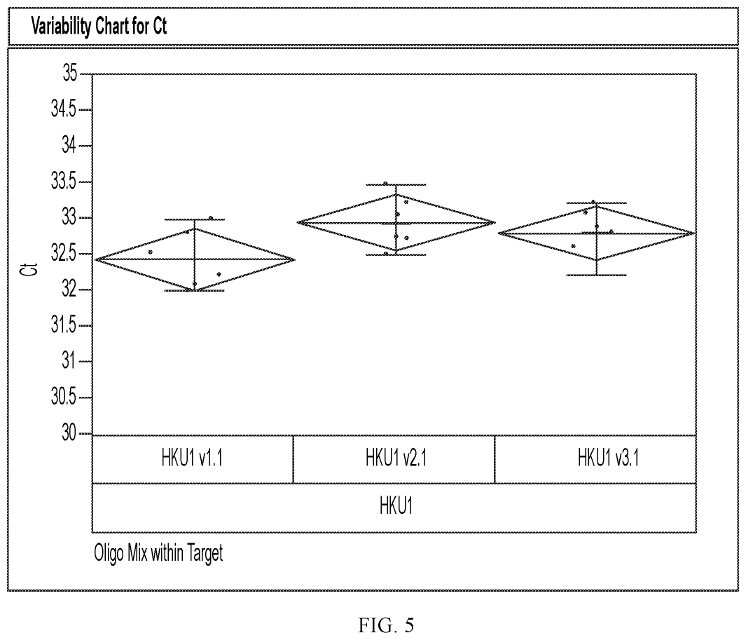

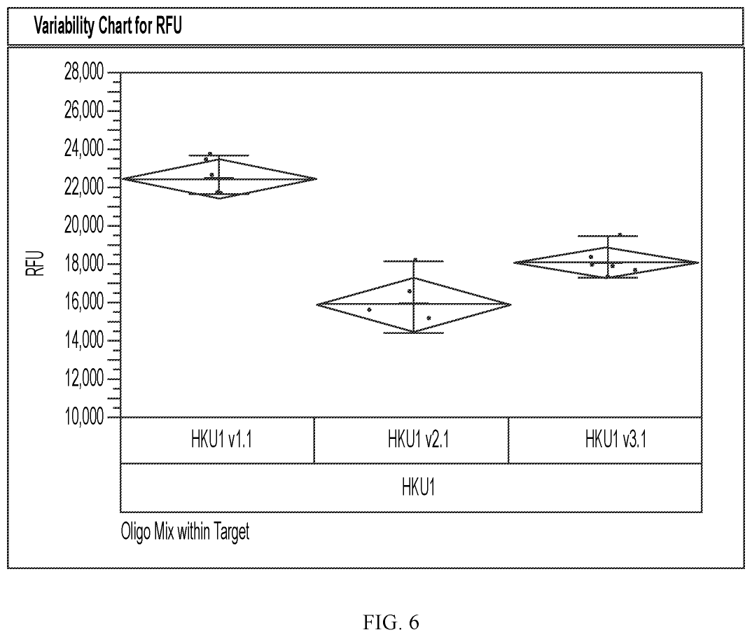

A series of experiments testing several primer and probe combinations for the real-time PCR amplification and detection of coronavirus type HKU 1. Reactions were generally prepared and performed as presented in Example 1. The combinations of primers and probes are recited in Table 3. The IC detection probe was labeled with Quasar 705 and Black Hole Quencher 2 and each CoV detection probe was labeled with FAM and Black Hole Quencher 1 (all available from BioSearch Technologies, Inc. or Glen Research, Inc). Positive reactions contained 1,000 copies of coronavirus HKU1 target nucleic acid (SEQ ID NO:53) per reaction. Negative reactions contained only STM. Results are shown in FIGS. 5, 6 & 7.

For this example, each of the tested HKU1 PPR mixes showed similar Ct performance and RFU, but also showed high background signal for each of the PPR mixes, indicating that the negative wells were possibly contaminated with HKU1 target nucleic acids (HKU1 PPR Mix v1.1 showed 22,630 RFU average RFU for the positive reactions and 20,082 RFU for the negative reactions; HKU1 PPR Mix v2.1 showed 16,064 RFU for the positive reactions and 18,768 for the negative reactions; and HKU1 PPR Mix v3.1 showed 18,239 RFU for the positive reaction and 18,474 for the negative reactions). A new negative control reaction mixture was made for subsequent testing of HKU1 PPR mixes. The HKU1 PPR Mix V1.1 showed a slightly faster Ct and a higher RFU than did the other HKU1 PPR mixes.

TABLE 3

| PPR Mix | Forward Primer | Reverse Primer | Detection Probe |
|---|---|---|---|
| IC PPR Mix | SEQ ID NO: 21 | SEQ ID NO: 29 | SEQ ID NO: 46 |
| HKU1 PPR Mix v1.l | SEQ ID NO: 20 | SEQ ID NO: 9 | SEQ ID NO: 10 |
| HKU1 PPR Mix v2.1 | SEQ ID NO: 40 | SEQ ID NO: 31 | SEQ ID NO: 27 |
| HKU1 PPR Mix v3.1 | SEQ ID NO: 25 | SEQ ID NO: 48 | SEQ ID NO: 19 |

An additional experiment was performed to test a further collection of primer and probe combinations, including PPR mixes wherein the primer and probe sequences further comprised analog nucleotides and/or varied label positions. Reactions were generally prepared and performed as presented in Example 1. The collection of PPR Mix combinations are described in Table 4. Positive reactions contained 10,000 copies of coronavirus HKU1 target nucleic acid (SEQ ID NO:53) per reaction. Negative reactions contained only STM. Results are shown in Tables 5 & 6.

For this additional experiment, the HKU1 PPR Mix v1 system showed faster Cts than did the HKU1 PPR Mix v2 system (˜33.5 vs ˜37) and higher RFUs (>20K vs <15K). Within the HKU1 PPR Mix vl system, v1.1.1 showed tighter Cts and RFUs than did HKU1 PPR Mix v1.1 and HKU1 PPR Mix v1.1.2. Within the HKU1 PPR Mix v2 system, HKU1 PPR Mix 2.1 and HKU1 PPR Mix 2.1.1 showed faster Cts thank did HKU1 PPR Mix 2.1.2 by about half a Ct, but the variability is high across the 3 PPR mixes. HKU1 PPR Mix 2.1.2 showed the lowest RFUs.

TABLE 4

| PPR Mix | Forward Primer | Reverse Primer | Detection Probe |
|---|---|---|---|
| IC PPR Mix | SEQ ID NO: 21 | SEQ ID NO: 29 | SEQ ID NO: 46 |
| | | | 5′ Q705 |
| | | | 3′BHQ-2 |
| HKU1 PPR Mix v1.1 | SEQ ID NO: 20 | SEQ ID NO: 9 | SEQ ID NO: 10 |
| | | | 5′ FAM |
| | | | 3′BHQ-l |
| HKU1 PPR Mix v1.1.1 | SEQ ID NO: 20 | SEQ ID NO: 9 | SEQ ID NO: 10 |
| | | | 5′ FAM |
| | | | Internal BHQ-1 (residue 10) |
| | | | 3′RevC |
| HKU1 PPR Mix v1.1.2 | SEQ ID NO: 20 | SEQ ID NO: 9 | SEQ ID NO: 10 |
| | | | 5′ FAM |
| | | | Internal BHQ-1 (residue 10) |
| | | | 5MeC at residues 2, 11, 13, 16, |
| | | | 17, 21, & 26 |
| | | | 3′RevC |
| HKU1 PPR Mix v2.1 | SEQ ID NO: 40 | SEQ IDNO: 31 | SEQ ID NO: 27 |
| | | | 5′ FAM |
| | | | 3′BHQ-l |
| HKU1 PPR Mix v2.1.1 | SEQ ID NO: 40 | SEQ ID NO: 31 | SEQ ID NO: 27 |
| | 5MeC at residues 2, | 5MeC at residues | 5′ FAM |
| | 5, 11, 14, 19, & 23 | 6, 7, 10, 16 & 18 | Internal BHQ-1 (residue 11) |
| | | | 5MeC at residues 3, 19, 21, 22, |
| | | | 24, & 25 |
| | | | 3′RevC |
| HKU1 PPR Mix v2.1.2 | SEQ ID NO: 40 | SEQ ID NO: 31 | SEQ ID NO: 27 |
| | 5MeC at residues 2, | 5MeC at residues | 5′FAM |
| | 5, 11, 14, 19, & 23 | 6, 7, 10, 16 & 18 | Internal BHQ-1 (residue 11) |
| | | | 3′RevC |

TABLE 5
Fam Channel

| | | | | Mean | | Mean | SD | Mean | |
|---|---|---|---|---|---|---|---|---|---|
| PPR Mix | Target | N | N Pos | CT | SD CT | RFU | RFU | BKG | SD BKG |
| HKU1 v1.1 | Negative | 6 | 0 | | | | | 25810 | 2287 |
| | 1E4 CPR IVT | 6 | 6 | 33.5 | 0.7 | 24986 | 1989 | 26994 | 1371 |
| HKU1 v1.1.1 | Negative | 6 | 0 | | | | | 8168 | 888 |
| | 1E4 CPR IVT | 6 | 6 | 33.7 | 0.1 | 21927 | 971 | 8742 | 436 |
| HKU1 v1.1.2 | Negative | 6 | 0 | | | | | 8144 | 583 |
| | 1E4 CPR IVT | 6 | 6 | 33.4 | 0.6 | 27658 | 2565 | 8380 | 337 |
| HKU1 v2.1 | Negative | 6 | 0 | | | | | 22500 | 1288 |
| | 1E4 CPR IVT | 6 | 6 | 36.8 | 0.5 | 13267 | 1398 | 23093 | 2902 |
| HKU1 v2.1.1 | Negative | 6 | 0 | | | | | 9177 | 1047 |
| | 1E4 CPR IVT | 6 | 6 | 36.4 | 0.8 | 13505 | 1558 | 9535 | 1025 |
| HKU1 v2.1.2 | Negative | 6 | 0 | | | | | 9020 | 641 |
| | 1E4 CPR IVT | 6 | 6 | 37.4 | 0.9 | 8268 | 1638 | 8995 | 343 |

TABLE 6
Quasar 705 Channel

| | | | N | Mean | | Mean | SD | Mean | SD |
|---|---|---|---|---|---|---|---|---|---|
| PPR Mix | Target | N | Pos | CT | SD CT | RFU | RFU | BKG | BKG |
| HKU1 v1.1 | Negative | 6 | 6 | 36.5 | 0.8 | 3572 | 542 | 805 | 90 |
| | 1E4 CPR IVT | 6 | 6 | 36.6 | 0.5 | 3594 | 156 | 857 | 54 |
| HKU1 v1.1.1 | Negative | 6 | 6 | 37.4 | 0.9 | 3034 | 523 | 778 | 95 |
| | 1E4 CPR IVT | 6 | 6 | 37.1 | 0.4 | 3428 | 113 | 845 | 28 |
| HKU1 v1.1.2 | Negative | 6 | 6 | 37.3 | 0.6 | 3290 | 373 | 817 | 71 |
| | 1E4 CPR IVT | 6 | 6 | 36.8 | 0.5 | 3502 | 180 | 874 | 39 |
| HKU1 v2.1 | Negative | 6 | 6 | 37 | 0.5 | 3219 | 197 | 742 | 52 |
| | 1E4 CPR IVT | 6 | 6 | 37 | 0.8 | 3325 | 495 | 775 | 95 |
| HKU1 v2.1.1 | Negative | 6 | 6 | 37.4 | 0.5 | 3119 | 346 | 736 | 90 |
| | 1E4 CPR IVT | 6 | 6 | 36.7 | 0.3 | 3486 | 310 | 796 | 89 |
| HKU1 v2.1.2 | Negative | 6 | 6 | 37.4 | 0.8 | 3113 | 367 | 730 | 51 |
| | 1E4 CPR IVT | 6 | 6 | 37.3 | 0.6 | 3060 | 82 | 756 | 22 |

### Example 4

In this example, several variations on the HKU1 PPR Mix v1.1 were tested. The collection of PPR Mix combinations are described in Table 7. Hexachlorofluorescein (HEX) is available from Integrated DNA Technologies, Inc., Coralville, IA. Reactions were generally prepared and performed as presented in Example 1. Positive reactions contained 100 or 1,000 copies of coronavirus HKU1 target nucleic acid (SEQ ID NO:53) per reaction. Negative reactions contained only STM. Results are shown in Table 8.

In this experiment both HKU1 PPR Mix v1.1.3 and HKU1 PPR Mix v1.1.4 showed very similar Ct performance and RFU output. HKU1 PPR Mix v1.1.3 showed slightly faster CT performance and slightly higher RFU output over the other PPR mixes, but HKU1 PPR Mix v1.1.4 showed a tighter grouping of replicate values. All three PPR mixes showed good sensitivity at 100 copies per reaction.

TABLE 7

| PPR Mix | Forward Primer | Reverse Primer | Detection Probe |
|---|---|---|---|
| IC PPR Mix | SEQ ID NO: 21 | SEQ ID NO: 29 | SEQ ID NO: 46 |
| | | | 5′ Q705 |
| | | | 3′BHQ-2 |
| HKU1 PPR Mix v1.1.2 | SEQ ID NO: 20 | SEQ ID NO: 9 | SEQ ID NO: 10 |
| | | | 5′ HEX |
| | | | Internal BHQ-1 (residue 10) |
| | | | 5MeC at residues 2, 11, 13, |
| | | | 16, 17, 21, & 26 |
| | | | 3′RevC |
| HKU1 PPR Mix v1.1.3 | SEQ ID NO: 8 | SEQ ID NO: 9 | SEQ ID NO: 10 |
| | | | 5′ HEX |
| | | | Internal BHQ-1 (residue 10) |
| | | | 5MeC at residues 2, 11, 13, |
| | | | 16, 17, 21, & 26 |
| | | | 3′RevC |
| HKU1 PPR Mix v 1.1.4 | SEQ ID NO: 26 | SEQ ID NO: 9 | SEQ ID NO: 10 |
| | | | 5′ HEX |
| | | | Internal BHQ-1 (residue 10) |
| | | | 5MeC at residues 2, 11, 13, |
| | | | 16, 17, 21, & 26 |
| | | | 3′RevC |

TABLE 8

| | | | | N | Mean | SD | Mean | SD | | SD |
|---|---|---|---|---|---|---|---|---|---|---|
| Channel | PPR Mix | Target | N | Pos | CT | CT | RFU | RFU | Mean BKG | BKG |
| HEX | HKU1 v1.1.2 | Negative | 6 | 0 | | | | | 3732 | 368 |
| | | HKU1 IVT | 6 | 5 | 37 | 0.7 | 15351 | 2376 | 3915 | 402 |
| | | 100CPR | | | | | | | | |
| | | HKU1 IVT | 6 | 6 | 34.5 | 0.7 | 17656 | 950 | 3557 | 247 |
| | | 1000CPR | | | | | | | | |
| | HKU1 v1.1.3 | Negative | 6 | 0 | | | | | 3732 | 361 |
| | | HKU1 IVT | 6 | 6 | 37.5 | 0.8 | 14155 | 2413 | 3466 | 431 |
| | | 100CPR | | | | | | | | |
| | | HKU1 IVT | 6 | 6 | 34.2 | 0.9 | 19405 | 2670 | 3653 | 445 |
| | | 1000CPR | | | | | | | | |
| | HKU1 v1.1.4 | Negative | 6 | 0 | | | | | 3202 | 260 |
| | | HKU1 IVT | 6 | 6 | 37.6 | 1 | 13551 | 2586 | 3028 | 518 |
| | | 100CPR | | | | | | | | |
| | | HKU1 IVT | 6 | 6 | 34.1 | 0.5 | 18744 | 1786 | 3224 | 324 |
| | | 1000CPR | | | | | | | | |
| Q705 | HKU1 v1.1.2 | Negative | 6 | 6 | 37.3 | 0.3 | 3572 | 290 | 1242 | 120 |
| | | HKU1 IVT | 6 | 6 | 37.6 | 0.6 | 3647 | 353 | 1285 | 121 |
| | | 100CPR | | | | | | | | |
| | | HKU1 IVT | 6 | 6 | 37.5 | 0.9 | 3243 | 155 | 1147 | 78 |
| | | 1000CPR | | | | | | | | |
| | HKU1 v1.1.3 | Negative | 6 | 6 | 37.4 | 0.5 | 3538 | 541 | 1224 | 141 |
| | | HKU1 IVT | 6 | 6 | 38 | 1.3 | 3250 | 601 | 1185 | 143 |
| | | 100CPR | | | | | | | | |
| | | HKU1 IVT | 6 | 6 | 37.6 | 1.2 | 3518 | 611 | 1238 | 179 |
| | | 1000CPR | | | | | | | | |
| | HKU1 v1.1.4 | Negative | 6 | 6 | 37.3 | 0.5 | 3427 | 275 | 1239 | 71 |
| | | HKU1 IVT | 6 | 6 | 37.4 | 0.5 | 3208 | 439 | 1185 | 140 |
| | | 100CPR | | | | | | | | |
| | | HKU1 IVT | 6 | 6 | 37.4 | 0.6 | 3381 | 410 | 1197 | 159 |
| | | 1000CPR | | | | | | | | |

### Example 5

Example 5 describes several multiplex reactions testing primer and probe combinations for the real-time PCR amplification and detection of Coronavirus types OC43, HKU1, 229E, and NL63. The NL63 primer probe combinations are varied in each testing condition. Collections of PPR mixes are described in Tables 9, 10, 11, & 12. For the IC PPR Mix in each collection SEQ ID NO:46 comprised 5′ Q705 and 3′ BHQ-2. For the OC43 PPR Mix in each collection SEQ ID NO:6 comprised 5MeC at residues 4, 13, & 17-19, and SEQ ID NO:7 comprised 5′ FAM, internal BHQ-1 at residue 13, and 3′ revC. For the HKU1 PPR Mix in each collection SEQ ID NO:10 comprised 5′ HEX, internal BHQ-1 at residue 10, and 3′ revC. For the 229E PPR Mix in each collection SEQ ID NO:36 comprised 5′ Quasar 670 (Q670), which is available from Biosearch Technologies, Inc., Novato, Calif., internal BHQ-2 at residue 11, and 3′ revC. For the NL63 PPR Mixes, see Tables 9-12. Ca1610 is available from Biosearch Technologies, Inc., Novato, Calif. Reactions were generally prepared and performed as presented in Example 1.

Positive Reactions for the duplex reactions (only the IC PPR Mix and the NL63 PPR Mix from each of Tables 9-12) contained 1,000 copies of coronavirus NL63 target nucleic acid (SEQ ID NO:54) per reaction. Positive reactions for the full multiplex reactions (each of the PPR Mixes in Tables 9-12) contained 1,000 copies of coronavirus OC43 target nucleic acids (SEQ ID NO:52) per reaction, 1,000 copies of coronavirus HKU1 target nucleic acids (SEQ ID NO:53) per reaction, 1,000 copies of coronavirus NL63 target nucleic acids (SEQ ID NO:54) per reaction, and 1,000 copies of coronavirus 229E target nucleic acids (SEQ ID NO:55) per reaction. Negative reactions contained only STM. Results are shown in FIGS. JEL-3a to JEL-3i.

This experiment showed that the NL63-1 PPR Mix had a better Ct performance (greater than 3 Ct improvement, with is about a 1 log improvement) and higher RFU than did the NL63-PPR Mix v2.1.1. NL63-2 PPR Mix and NL63-3 PPR Mix both showed Ct performance that was roughly equivalent to NL63-PPR Mix v2.1.1, but both showed higher RFU readings compared to NL63-PPR Mix v2.1.1. None of the other target amplification and detections (OC43, HKU1, and 229E) were negatively affected in these multiplex reactions with any of the four NL63 PPR Mixes.

TABLE 9

| PPR Mix | Forward Primer | Reverse Primer | Detection Probe |
|---|---|---|---|
| IC PPR Mix | SEQ ID NO: 21 | SEQ ID NO: 29 | SEQ ID NO: 46 |
| OC43 PPR Mix | SEQ ID NO: 5 | SEQ ID NO: 6 | SEQ ID NO: 7 |
| HKU1 PPR Mix | SEQ ID NO: 8 | SEQ ID NO: 9 | SEQ ID NO: 10 |
| 229E PPR Mix | SEQ ID NO: 42 | SEQ ID NO: 35 | SEQ ID NO: 36 |
| NL63 PPR Mix | SEQ ID NO: 37 | SEQ ID NO: 49 | SEQ ID NO: 18 |
| v2.1.1 | 5MeC at residues | 5MeC at residues | 5′Cal610 |
| | 7 & 20. | 9, 12, 18, & 20 | Internal BHQ-2 |
| | | | at residue 11 |
| | | | 3′RevC |

TABLE 10

| PPR Mix | Forward Primer | Reverse Primer | Detection Probe |
|---|---|---|---|
| IC PPR Mix | SEQ ID NO: 21 | SEQ ID NO: 29 | SEQ ID NO: 46 |
| OC43 PPR Mix | SEQ ID NO: 5 | SEQ ID NO: 6 | SEQ ID NO: 7 |
| HKU1 PPR Mix | SEQ ID NO: 8 | SEQ ID NO: 9 | SEQ ID NO: 10 |
| 229E PPR Mix | SEQ ID NO: 42 | SEQ ID NO: 35 | SEQ ID NO: 36 |
| NL63-1 PPR Mix | SEQ ID NO: 11 | SEQ ID NO: 12 | SEQ ID NO: 13 |
| | | | 5′ Cal610 |
| | | | 5MeC at residues |
| | | | 8, 10, 13, & 17 |
| | | | 3′ BHQ-2 |

TABLE 11

| PPR Mix | Forward Primer | Reverse Primer | Detection Probe |
|---|---|---|---|
| IC PPR Mix | SEQ ID NO: 21 | SEQ ID NO: 29 | SEQ ID NO: 46 |
| OC43 PPR Mix | SEQ ID NO: 5 | SEQ ID NO: 6 | SEQ ID NO: 7 |
| HKU1 PPR Mix | SEQ ID NO: 8 | SEQ ID NO: 9 | SEQ ID NO: 10 |
| 229E PPR Mix | SEQ ID NO: 42 | SEQ ID NO: 35 | SEQ ID NO: 36 |
| NL63-2 PPR Mix | SEQ ID NO: 50 | SEQ ID NO: 45 | SEQ ID NO: 22 |
| | | | 5′ Cal610 |
| | | | 3′ BHQ-2 |

TABLE 12

| PPR Mix | Forward Primer | Reverse Primer | Detection Probe |
|---|---|---|---|
| IC PPR Mix | SEQ ID NO: 21 | SEQ ID NO: 29 | SEQ ID NO: 46 |
| OC43 PPR Mix | SEQ ID NO: 5 | SEQ ID NO: 6 | SEQ ID NO: 7 |
| HKU1 PPR Mix | SEQ ID NO: 8 | SEQ ID NO: 9 | SEQ ID NO: 10 |
| 229E PPR Mix | SEQ ID NO: 42 | SEQ ID NO: 35 | SEQ ID NO: 36 |
| NL63-3 PPR Mix | SEQ ID NO: 30 | SEQ ID NO: 23 | SEQ ID NO: 17 |
| | | | 5′ Cal610 |
| | | | 5MeC at residues |
| | | | 11 & 14 |
| | | | 3′ BHQ-2 |

### Example 6

Examples 7-13 describe studies evaluating the amplification oligonucleotides listed in Table 13 and the detection probes listed in Table 14 for detection of human coronaviruses OC43, HKU1, NL63, and 229E. All detection probes were designed as TaqMan probes (linear hydrolysis probes) utilizing the indicated fluorophore and quencher labels (available from Biosearch Technologies, Inc. and/or Glen Research, Inc.). Except where otherwise indicated, reactions were performed using final primer, probe, and MgCl₂ concentrations of 0.6 μM, 0.4 μM, and 4 μM, respectively. For all studies, KCl was kept constant at 65 mM in the final reaction. All experiments were performed in multiplex with oligonucleotides for detection of an RNA Internal Control. All experiments were performed with a thermocycling program for RNA amplification and detection as listed in Table 15.

TABLE 13
Amplification Oligonucleotides

| Oligo Sequence | Target | Function |
|---|---|---|
| SEQ ID NO: 5 | OC43 | Forward primer |
| SEQ ID NO: 6 | OC43 | Reverse primer |
| SEQ ID NO: 8 | HKU1 | Forward primer |
| SEQ ID NO: 9 | HKU1 | Reverse primer |
| SEQ ID NO: 11 | NL63 | Forward primer |
| SEQ ID NO: 12 | NL63 | Reverse primer |
| SEQ ID NO: 14 | 229E | Forward primer |
| SEQ ID NO: 15 | 229E | Reverse primer |

TABLE 14
Detection Probes

| Oligo Sequence | Target | Fluorophore | Quencher |
|---|---|---|---|
| SEQ ID NO: 7 | OC43 | FAM | BHQ-1 |
| SEQ ID NO: 10 | HKU1 | Cal Fluor Orange 560 | BHQ-1 |
| SEQ ID NO: 13 | NL63 | Cal Fluor Red 610 | BHQ-2 |
| SEQ ID NO: 16 | 229E | Quasar 670 | BHQ-2 |

TABLE 15
RNA PCR Cycling Profile
RNA Profile

| | | | Temp | |
|---|---|---|---|---|
| Stage | Cycles | Step | (° C.) | Time |
| 1 | 1 | 1 | 46 | 8 min |
| 2 | 1 | 1 | 95 | 2 min |
| 3 | 45 | 1 | 95 | 5 sec |
| | | 2 | 60 | 22 sec |

### Example 7: Multiplex Formulation with OC43, HKU1, NL63, 229E, and Internal Control Oligonucleotides

Coronavirus oligonucleotides shown in Tables 13 and 14 were evaluated in a multiplex formulation with internal control oligonucleotides. Control conditions were OC43, HKU1, NL63, and 229E oligonucleotides tested in separate PCR formulations. Samples contained OC43 IVT, HKU1 IVT, NL63 IVT, or 229E IVT (SEQ ID NOs:52-55, respectively) diluted to 1000 copies per reaction in STM with HeLa cells at a final concentration of 1.0×10⁴ cells/mL.

Internal control was detected in all conditions tested. Multiplex formulation results for OC43, HKU1, NL63, and 229E are shown in Table 16. 0C43, HKU1, NL63, and 229E oligonucleotides were assessed based on changes in cycle time (CT), RFU, background, signal to noise, and slope at threshold in the FAM, HEX, ROX, and Quasar 670 channels compared against control conditions. Minimal changes (no more than 1 CT) were observed in the performance of OC43, HKU1, NL63, and 229E primer/probe mixes in a multiplex formulation versus in individual formulations (Control). IC detection was within 1 CT between the control and multiplex formulation for each analyte.

### Example 8: IVT Linearity

OC43, HKU1, NL63, and 229E in vitro transcripts (IVTs) were evaluated for reactivity with a PCR formulation including all primers and probes listed in Tables 13 and 14 and internal control oligonucleotides. OC43 IVT, HKU1 IVT, NL63 IVT, or 229E IVT (SEQ ID NOs:52-55, respectively) was spiked into STM containing HeLa cells (approximately 10,000 cells/mL) at the concentrations indicated in Table 17. The specimens were processed on a Panther Fusion system (Hologic, Inc.) using its wave1 sequence file in which 360 μL of sample was extracted. 5 μL of eluate was used in the final PCR reaction. PCR amplification data are shown in Table 17. 100% detection was seen for OC43, HKU1, NL63, and 229E IVTs in STM at all concentrations tested down to 50 copies/reaction. Linearity plots demonstrated high PCR efficiencies with R² values >0.99 and slopes at −3.4, −3.3, −3.5, and −3.4 for OC43, HKU1, NL63, and 229E. The internal control had 100% detection.

### Example 9: OC43, HKU1, NL63, and 229E Analytical Sensitivity

OC43, HKU1, NL63, and 229E viruses and IVTs (HKU1 virus was not tested because no live virus was available) were evaluated for reactivity with a PCR formulation including all primers and probes listed in Tables 13 and 14 and internal control oligonucleotides. OC43, HKU1 (IVT only), NL63, and 229E IVT (SEQ ID NOs:52-55) or virus in culture fluid was spiked into STM containing HeLa cells (approximately 10,000 cells/mL). All specimens were processed using a Panther Fusion system (Hologic, Inc.) using its Wave 1 sequence file. Data are described in Table 18 for OC43, Table 19 for HKU1, Table 20 for NL63, and Table 21 for 229E. 100% detection was observed for OC43 virus in all dilutions down to 3.55E-03 TCID50/mL, NL63 virus was detected at 100% in all dilutions down to 1.00E-02 TCID50/mL, and 100% detection for 229E virus was observed in all dilutions down to 1.00E-01 TCID50/mL. All IVTs were detected 100% down to 50 copies/reaction except NL63 which had one drop out. Since the average CT for NL63 at 50 copies/reaction was 36.19 and the viral LoD at 1.00E-2 TCID50/mL had an average CT of 36.22, the one drop out appears more like an unexplained error. Internal control was detected at 100%.

### Example 10: Clinical Specimen Detection and Clinical Specificity

Analytical sensitivity and specificity were evaluated using 10 OC43 positive, 10 NL63 positive, 10 229E positive, and 20 negative specimens. No HKU1 positive samples were available. The specimens were nasopharyngeal swab samples. Specimens were procured from TriCore Reference Laboratory (Albuquerque, N. Mex.) and delivered frozen. The specimens were processed according to the relevant clinical matrix workflow for nasopharyngeal swabs. A PCR formulation including primers and probes listed in Tables 13 and 14 and internal control oligonucleotides was used for this study. Table 22 shows there is a 90% agreement with OC43 positive samples, with 1 false negative, and a false positive with the negative NPS samples. Table 23 shows a 90% agreement with NL63 samples, with 1 false negative result, and Table 24 shows a 90% agreement with 229E samples, with 1 false negative result. There was a 97.5% concordance with negative samples.

TABLE 16
Multiplex Formulation Results

| | | | | | | | | AVG | SD | AVG | | |
|---|---|---|---|---|---|---|---|---|---|---|---|---|
| | | | | AVG | SD | AVG | SD | Base- | Base- | Slope at | SD | Signal: |
| Channel | Sample | Oligos | Reactivity* | CT | CT | RFU | RFU | line | line | Threshold | Tslope | Noise |
| FAM | OC43 Control | OC43 | 3/3 | 31.43 | 0.12 | 22630.33 | 1911.65 | 9026.67 | 1263.71 | 576.33 | 42.85 | 2.52 |
| | OC43 | OC43, | 3/3 | 32.42 | 0.14 | 16520.00 | 1424.10 | 7675.00 | 402.00 | 563.30 | 46.30 | 2.15 |
| | Multiplex | HKU1, | | | | | | | | | | |
| | | NL63, | | | | | | | | | | |
| | | 229E | | | | | | | | | | |
| HEX | HKU1 Control | HKU1 | 3/3 | 32.80 | 0.22 | 18507.00 | 1599.56 | 2760.33 | 217.01 | 555.33 | 138.07 | 6.70 |
| | HKU1 | OC43, | 3/3 | 33.38 | 0.16 | 16328.67 | 3295.82 | 2599.67 | 494.06 | 593.67 | 60.05 | 6.27 |
| | Multiplex | HKU1, | | | | | | | | | | |
| | | NL63, | | | | | | | | | | |
| | | 229E | | | | | | | | | | |
| ROX | NL63 Control | NL63 | 3/3 | 32.70 | 0.28 | 9058.67 | 1070.25 | 434.67 | 60.38 | 284.33 | 42.90 | 20.89 |
| | NL63 | OC43, | 3/3 | 33.80 | 0.19 | 6255.33 | 834.02 | 448.67 | 32.75 | 231.33 | 21.39 | 13.90 |
| | Multiplex | HKU1, | | | | | | | | | | |
| | | NL63, | | | | | | | | | | |
| | | 229E | | | | | | | | | | |
| Q760 | 229E Control | 229E | 3/3 | 32.88 | 0.12 | 3017.00 | 307.44 | 1068.67 | 168.83 | 225.00 | 29.51 | 2.84 |
| | 229E | OC43, | 3/3 | 33.10 | 0.19 | 3064.33 | 178.99 | 1145.00 | 7.23 | 235.67 | 25.54 | 2.68 |
| | Multiplex | HKU1, | | | | | | | | | | |
| | | NL63, | | | | | | | | | | |
| | | 229E | | | | | | | | | | |

*Reactivity is defined as an amplification curve crossing a threshold of 500 RFU in the ROX or Q670 channels or 1000 RFU in the FAM or HEX channels.

TABLE 17
OC43, HKU1, NL63, and 229E IVT Linearity Results

| | Conc. | | | | | | | Standard | | Standard |
|---|---|---|---|---|---|---|---|---|---|---|
| | (TCID50/ | | | | | | Average | Deviation | | Deviation |
| | mL or | | | Standard | | Standard | Back- | of Back- | Average | of |
| | copies/ | | Average | Deviation | Average | Deviation | ground | ground | Slop at | Slope at |
| Sample | reaction) | Reactivity | CT | of CT | RFU | of RFU | RFU | RFU | Threshold | Threshold |
| OC43 | 1E6 copies | 6/6 | 22.60 | 0.20 | 21523.00 | 2385.00 | 8554.00 | 796.00 | 576.00 | 56.00 |
| IVT | 1E5 copies | 6/6 | 26.00 | 0.10 | 23160.00 | 1573.00 | 9328.00 | 626.00 | 601.00 | 163.00 |
| | 1E4 copies | 6/6 | 29.50 | 0.30 | 17843.00 | 1736.00 | 8014.00 | 560.00 | 651.00 | 82.00 |
| | 1E3 copies | 6/6 | 33.00 | 0.30 | 17733.00 | 3007.00 | 8064.00 | 938.00 | 626.00 | 112.00 |
| | 1E2 copies | 6/6 | 36.20 | 0.50 | 12286.00 | 2257.00 | 8541.00 | 603.00 | 739.00 | 152.00 |
| HKU1 | 1E6 copies | 6/6 | 22.60 | 0.20 | 21523.00 | 2385.00 | 8554.00 | 796.00 | 576.00 | 56.00 |
| IVT | 1E5 copies | 6/6 | 26.00 | 0.10 | 23160.00 | 1573.00 | 9328.00 | 626.00 | 601.00 | 163.00 |
| | 1E4 copies | 6/6 | 29.50 | 0.30 | 17843.00 | 1736.00 | 8014.00 | 560.00 | 651.00 | 82.00 |
| | 1E3 copies | 6/6 | 33.00 | 0.30 | 17733.00 | 3007.00 | 8064.00 | 938.00 | 626.00 | 112.00 |
| | 1E2 copies | 6/6 | 36.20 | 0.50 | 12286.00 | 2257.00 | 8541.00 | 603.00 | 739.00 | 152.00 |
| NL63 | 1E6 copies | 6/6 | 21.76 | 0.23 | 10709.83 | 1003.87 | 468.83 | 48.09 | 137.50 | 25.41 |
| IVT | 1E5 copies | 6/6 | 25.04 | 0.13 | 9924.67 | 745.16 | 455.00 | 38.98 | 171.00 | 32.00 |
| | 1E4 copies | 6/6 | 28.64 | 0.47 | 8402.17 | 723.29 | 438.33 | 44.37 | 174.33 | 29.72 |
| | 1E3 copies | 6/6 | 32.00 | 0.24 | 7166.33 | 942.51 | 462.00 | 48.20 | 159.17 | 30.49 |
| | 1E2 copies | 6/6 | 35.92 | 0.50 | 3981.33 | 694.72 | 405.33 | 35.78 | 164.17 | 25.65 |
| 229E | 1E6 copies | 6/6 | 22.43 | 0.28 | 2653.50 | 356.11 | 986.33 | 119.39 | 137.50 | 19.61 |
| IVT | 1E5 copies | 6/6 | 25.73 | 0.25 | 2765.67 | 225.24 | 1032.67 | 85.70 | 138.17 | 20.10 |
| | 1E4 copies | 6/6 | 29.16 | 0.21 | 2773.17 | 254.37 | 1090.33 | 102.00 | 151.00 | 20.45 |
| | 1E3 copies | 6/6 | 32.58 | 0.41 | 2690.17 | 216.96 | 1069.17 | 97.77 | 141.33 | 9.48 |
| | 1E2 copies | 6/6 | 35.86 | 0.28 | 2203.67 | 581.46 | 1054.17 | 62.36 | 144.83 | 22.12 |

*Reactivity defined as an amplification curve crossing a threshold of 1000 RFU for OC43 and HKU1, and 500 RFU for NL63 and 229E.

TABLE 18
OC43 Analytical Sensitivity

| | Conc. | | | | | | Average | Standard | | Standard |
|---|---|---|---|---|---|---|---|---|---|---|
| | (TCID50/ | | | Standard | | Standard | Back- | Deviation of | Average | Deviation of |
| | mL or copies/ | | Average | Deviation | Average | Deviation | ground | Background | Slop at | Slope at |
| Sample | reaction) | Reactivity | CT | of CT | RFU | of RFU | RFU | RFU | Threshold | Threshold |
| OC43 | 3.55E+01 | 3/3 | 26.84 | 0.37 | 20771.33 | 2366.45 | 8963.00 | 1191.81 | 544.00 | 83.36 |
| virus | 3.55E+00 | 3/3 | 30.30 | 0.10 | 20424.67 | 825.80 | 9404.00 | 491.94 | 608.33 | 30.66 |
| | 3.55E−01 | 3/3 | 34.45 | 0.79 | 16752.00 | 466.54 | 8774.67 | 1188.16 | 585.33 | 97.29 |
| | 3.55E−02 | 3/3 | 36.77 | 0.61 | 11780.67 | 2791.62 | 9389.33 | 750.27 | 708.33 | 46.69 |
| | 3.55E−03 | 3/3 | 38.88 | 0.58 | 5536.67 | 1873.77 | 8073.00 | 584.61 | 614.00 | 27.50 |
| | 3.55E−04 | 0/3 | 0 | 0 | 189.33 | 17.79 | 9202.67 | 560.16 | 0 | 0 |
| | 3.55E−05 | 0/3 | 0 | 0 | 218.67 | 26.27 | 9380.00 | 1357.49 | 0 | 0 |
| | 3.55E−06 | 0/3 | 0 | 0 | 226.00 | 14.18 | 8094.33 | 180.80 | 0 | 0 |
| | 3.55E−07 | 0/3 | 0 | 0 | 258.00 | 35.16 | 8538.00 | 521.54 | 0 | 0 |
| OC43 | 5000 copies | 12/12 | 30.29 | 0.18 | 18040.92 | 1589.39 | 8426.50 | 586.23 | 678.42 | 70.20 |
| IVT | 500 copies | 12/12 | 33.45 | 0.35 | 16236.08 | 1476.26 | 8712.83 | 803.89 | 618.00 | 94.84 |
| | 50 copies | 12/12 | 36.56 | 0.38 | 13246.00 | 2602.52 | 9133.33 | 707.68 | 660.83 | 127.99 |
| | 5 copies | 6/12 | 38.94 | 0.40 | 5464.33 | 1918.67 | 8562.00 | 866.06 | 638.50 | 102.16 |

TABLE 19
HKU1 Analytical Sensitivity

| | Conc. | | | | | | Average | Standard | | Standard |
|---|---|---|---|---|---|---|---|---|---|---|
| | (TCID50/ | | | Standard | | Standard | Back- | Deviation of | Average | Deviation of |
| | mL or copies/ | | Average | Deviation | Average | Deviation | ground | Background | Slop at | Slope at |
| Sample | reaction) | Reactivity | CT | of CT | RFU | of RFU | RFU | RFU | Threshold | Threshold |
| HKU1 | 5000 copies | 12/12 | 30.30 | 0.25 | 18590.25 | 1889.25 | 2805.92 | 290.04 | 579.33 | 83.59 |
| IVT | 500 copies | 12/12 | 34.44 | 0.53 | 15299.25 | 2252.76 | 2625.42 | 352.59 | 565.08 | 89.77 |
| | 50 copies | 12/12 | 37.01 | 0.88 | 12073.42 | 2813.17 | 2703.58 | 254.25 | 514.08 | 100.78 |
| | 5 copies | 5/12 | 38.84 | 0.22 | 2869.25 | 3393.63 | 2532.50 | 227.27 | 461.40 | 102.99 |

TABLE 20
NL63 Analytical Sensitivity

| | Conc. | | | | | | Average | Standard | | Standard |
|---|---|---|---|---|---|---|---|---|---|---|
| | (TCID50/ | | | Standard | | Standard | Back- | Deviation of | Average | Deviation of |
| | mL or copies/ | | Average | Deviation | Average | Deviation | ground | Background | Slop at | Slope at |
| Sample | reaction) | Reactivity | CT | of CT | RFU | of RFU | RFU | RFU | Threshold | Threshold |
| NL63 | 1.00E+04 | 3/3 | 16.67 | 0.33 | 8981.33 | 1445.84 | 323.67 | 43.84 | 293.00 | 24.27 |
| virus | 1.00E+03 | 3/3 | 19.91 | 0.15 | 9969.00 | 1061.27 | 401.33 | 26.76 | 266.67 | 62.18 |
| | 1.00E+02 | 3/3 | 23.41 | 0.36 | 9163.67 | 940.08 | 408.33 | 34.03 | 287.33 | 49.24 |
| | 1.00E+01 | 3/3 | 26.65 | 0.34 | 8961.33 | 1030.34 | 415.33 | 49.52 | 294.00 | 37.51 |
| | 1.00E+00 | 3/3 | 30.08 | 0.19 | 8341.00 | 390.04 | 456.33 | 32.19 | 289.00 | 56.32 |
| | 1.00E−01 | 3/3 | 33.46 | 0.22 | 6164.00 | 274.44 | 424.67 | 10.02 | 268.00 | 28.00 |
| | 1.00E−02 | 3/3 | 36.22 | 0.21 | 5112.67 | 497.55 | 485.33 | 34.02 | 310.33 | 30.66 |
| | 1.00E−03 | 2/3 | 38.74 | 1.75 | 1970.67 | 1798.42 | 480.67 | 39.43 | 235.50 | 48.79 |
| | 1.00E−04 | 0/3 | 0 | 0 | 51.67 | 4.04 | 467.00 | 37.99 | 0 | 0 |
| NL63 | 5000 copies | 12/12 | 29.35 | 0.20 | 8631.50 | 1021.01 | 462.58 | 38.43 | 167.25 | 18.48 |
| IVT | 500 copies | 12/12 | 33.18 | 0.27 | 5835.67 | 638.62 | 421.17 | 40.66 | 168.75 | 30.33 |
| | 50 copies | 11/12 | 36.19 | 0.49 | 3709.58 | 1363.09 | 446.92 | 38.96 | 187.55 | 19.30 |
| | 5 copies | 3/12 | 38.03 | 0.12 | 714.17 | 1292.39 | 477.00 | 38.69 | 107.55 | 77.40 |

TABLE 21
229E Analytical Sensitivity

| | Conc. | | | | | | Average | Standard | | Standard |
|---|---|---|---|---|---|---|---|---|---|---|
| | (TCID50/ | | | Standard | | Standard | Back- | Deviation of | Average | Deviation of |
| | mL or copies/ | | Average | Deviation | Average | Deviation | ground | Background | Slop at | Slope at |
| Sample | reaction) | Reactivity | CT | of CT | RFU | of RFU | RFU | RFU | Threshold | Threshold |
| 229E virus | 1.00E+04 | 3/3 | 20.75 | 0.05 | 3031.00 | 50.09 | 1056.67 | 14.19 | 213.00 | 4.00 |
| | 1.00E+03 | 3/3 | 24.65 | 0.49 | 2588.67 | 487.72 | 916.00 | 163.02 | 225.67 | 26.31 |
| | 1.00E+02 | 3/3 | 27.47 | 0.40 | 2713.67 | 306.65 | 1055.33 | 140.55 | 229.00 | 38.59 |
| | 1.00E+01 | 3/3 | 30.73 | 0.08 | 2907.67 | 133.06 | 1125.33 | 58.07 | 216.00 | 6.54 |
| | 1.00E+00 | 3/3 | 34.67 | 0.16 | 2598.67 | 81.45 | 1068.00 | 80.52 | 214.00 | 8.89 |
| | 1.00E−01 | 3/3 | 38.12 | 0.34 | 1976.67 | 187.95 | 1072.00 | 33.05 | 221.33 | 16.62 |
| | 1.00E−02 | 2/3 | 40.30 | 0.36 | 1076.33 | 936.10 | 987.33 | 57.84 | 216.00 | 39.60 |
| | 1.00E−03 | 1/3 | 41.82 | 0 | 355.33 | 542.71 | 1041.33 | 132.31 | 153.00 | 0 |
| | 1.00E−04 | 0/3 | 0 | 0 | 37.33 | 4.04 | 1092.67 | 90.94 | 0 | 0 |
| 229E IVT | 5000 copies | 12/12 | 30.03 | 0.20 | 2793.83 | 207.20 | 1090.75 | 91.17 | 148.92 | 19.86 |
| | 500 copies | 12/12 | 33.36 | 0.27 | 2416.25 | 381.19 | 1050.75 | 119.69 | 146.50 | 17.04 |
| | 50 copies | 12/12 | 36.50 | 0.59 | 2016.00 | 422.41 | 1091.17 | 96.43 | 135.83 | 17.51 |
| | 5 copies | 9/12 | 39.23 | 0.37 | 1090.92 | 667.81 | 1029.25 | 77.68 | 126.00 | 16.55 |

TABLE 22
OC43 Clinical Specimen Sensitivity/Specificity
OC43 Assay Clinical Nasopharyngeal Swab Samples

| | | Reference Method | | |
|---|---|---|---|---|
| | | Positive | Negative | Total |
| OC43 | Positive | 9 | 1 | 10 |
| Primers/Probe | Negative | 1 | 39 | 40 |
| | Total | 10 | 40 | 50 |
| | | % agreement | | |
| | Positive | 90.0 | | |
| | Negative | 97.5 | | |

TABLE 23
NL63 Clinical Specimen Sensitivity/Specificity
NL63 Assay Clinical Nasopharyngeal Swab Samples

| | | Reference Method | | |
|---|---|---|---|---|
| | | Positive | Negative | Total |
| NL63 | Positive | 9 | 0 | 9 |
| Primer/Probe | Negative | 1 | 40 | 41 |
| | Total | 10 | 40 | 50 |

| | |
|---|---|
| | % agreement |
| Positive | 90.0 |
| Negative | 100.0 |

TABLE 24
229E Clinical Specimen Sensitivity/Specificity
229E Assay Clinical Nasopharyngeal Swab Samples

| | | Reference Method | | |
|---|---|---|---|---|
| | | Positive | Negative | Total |
| 229E | Positive | 9 | 0 | 9 |
| Primers/Probe | Negative | 1 | 40 | 41 |
| | Total | 10 | 40 | 50 |

| | |
|---|---|
| | % agreement |
| Positive | 90.0 |
| Negative | 100.0 |

### Example 11: Specificity

Forty-four organisms commonly found in nasopharyngeal swabs were prepared in 11 panels by spiking as close as possible (dependent on availability) to 1E6 (1×10⁶) cp/ml into STM. Each panel was evaluated for specificity with a PCR formulation including primers and probes listed in Tables 13 and 14 and internal control oligonucleotides. Panel composition and reactivity results are shown in Table 25. 0% of the organisms in panels 1-11 tested positive for OC43, HKU1, NL63 and 229E. The internal control was detected in all tests. The positive control was positive for OC43, HKU1, NL63, and 229E and IC and the negative control was positive for IC only.

### Example 12: Interference

OC43, HKU1, NL63, and 229E reactivity was evaluated in the presence of 44 organisms from the specificity study. OC43, HKU1, NL63, or 229E IVT (SEQ ID NOs:52-55) was spiked in panels 1-11 from the specificity study at 1000 copies/reaction. Each panel was evaluated for OC43, HKU1, NL63, and 229E performance in the presence of 4-5 commonly found organisms with a PCR formulation including primers and probes listed in Tables 13and 14 and internal control oligonucleotides. Panel composition and reactivity results are shown in Table 26. Results were compared to a positive control consisting of OC43, HKU1, NL63, and 229E at 1000 copies/reaction in STM. OC43, HKU1, NL63, and 229E were detected in 100% of the panels with less than a 1.0 Ct shift when compared to the positive control. The internal control was detected in 100% of the panels. Positive control was positive for OC43, HKU1, NL63, and 229E and IC and the negative control was positive for IC only.

### Example 13: Oligonucleotide Performance Under Different Primers, Probe, and MgCl₂ Concentrations

To evaluate the flexibility of the OC43, HKU1, NL63, and 229E oligonucleotides to function under different assay conditions, different concentrations of primers, probe, and MgCl₂ were tested. Three concentrations of primers (0.4, 0.7, and 1.0 μM), 3 concentrations of the probe (0.2, 0.5, and 0.8 μM), and 3 concentrations of MgCl₂ (2, 4, and 6 mM) were tested in a response surface Box-Behnken design. The experiment was run on a Panther Fusion system (Hologic, Inc.) using its Open Access feature and wave 1 workflow, with all the conditions in different Primer Probe Recon (PPR) buffers, 500 copies/reaction of OC43 IVT or HKU1 IVT (SEQ ID NOs:52 & 53), or 100 copies/reaction of NL63 IVT or 229E IVT (SEQ ID NOs:54 & 55) in sample tubes, and internal control (IC) plasmid present in the IC reagent tube. The results showed that RFU and CT values were robust to different concentrations of MgCl₂, and that the OC43, HKU1, NL63, and 229E ASRs can be used under different assay conditions. The results demonstrate that the OC43, HKU1, NL63, and 229E primer and probe systems functions in a wide range of assay conditions. The CTs are consistent within 1 CT across all oligo concentrations tested and across a wide range of MgCl2 concentrations.

TABLE 25
Specificity Results

| | | | | | Final | | |
|---|---|---|---|---|---|---|---|
| Panel | Organism | Manufacturer | Strain | ATTC/PN# | Concentration | Units | Reactivity |
| 1 | Adenovirus Culture Fluid | ZeptoMetrix | Type 1 | 0810050CF | 1.00E+06 | TCID50/mL | 0/3 |
| | Adenovirus Culture Fluid | ZeptoMetrix | Type 4 | 0810070CF | 1.00E+05 | TCID50/mL | |
| | Adenovirus Culture Fluid | ZeptoMetrix | Type 7A | 0810021CF | 1.00E+05 | TCID50/mL | |
| | Mumps Virus Culture Fluid | ZeptoMetrix | Isolate 1 | 0810079CF | 1.00E+04 | TCID50/mL | |
| 2 | Measles Virus Culture Fluid | ZeptoMetrix | NA | 0810025CF | 1.00E+04 | TCID50/mL | 0/3 |
| | Corynebacterium diphtheriae | ZeptoMetrix | Z116 | CFU/mL | 1.00E+06 | TCID50/mL | |
| | Cytomegalovirus | ZeptoMetrix | AD-169 | 0810003CF | 1.00E+04 | TCID50/mL | |
| | (CMV) Culture Fluid | | | | | | |
| | Epstein-Barr Virus | ZeptoMetrix | B95-8 | 0810008CF | 1.00E+06 | TCID50/mL | |
| | (EBV) Culture Fluid | | | | | | |
| 3 | Varicella Zoster Virus (VZV) | ZeptoMetrix | Strain 82 | 0810167CF | 1.00E+03 | TCID50/mL | 0/3 |
| | Culture Fluid | | | | | | |
| | Herpes Simplex Virus Type 1 | ZeptoMetrix | MacIntyre | 0810005CF | 1.00E+05 | TCID50/mL | |
| | (HSV-1) Culture Fluid | | | | | | |
| | Human Herpes Virus Type | ZeptoMetrix | Z29 | 0810072CF | 1.00E+06 | TCID50/mL | |
| | 6B (HHV-6B) Culture Fluid | | | | | | |
| | Human Herpes Virus Type 7 | ZeptoMetrix | SB | 0810071CF | 1.00E+05 | TCID50/mL | |
| | (HHV-7) Culture Fluid | | | | | | |
| 4 | Human Metapneumovirus (hMPV) | ZeptoMetrix | IA14-2003 | 0810163CF | 1.00E+04 | TCID50/mL | 0/3 |
| | 20 Type A2 Culture Fluid | | | | | | |
| | Human Metapneumovirus (hMPV) | ZeptoMetrix | Peru2-220 | 0810156CF | 1.00E+04 | TCID50/mL | |
| | 3 Type B1 Culture Fluid | | | | | | |
| | Human Metapneumovirus (hMPV) | ZeptoMetrix | Peru1-2002 | 0810157CF | 1.00E+04 | TCID50/mL | |
| | 4 Type B2 Culture Fluid | | | | | | |
| | Human Metapneumovirus (hMPV) | ZeptoMetrix | IA3-2002 | 0810160CF | 1.00E+04 | TCID50/mL | |
| | 9 Type A1 Culture Fluid | | | | | | |
| 5 | Influenza A H3N2 | ZeptoMetrix | Brisbane/10/07 | 0810138CF | 1.00E+03 | TCID50/mL | 0/3 |
| | Virus Culture Fluid | | | | | | |
| | Influenza B Culture Fluid | ZeptoMetrix | B/Florida/02/06 | 0810037CF | 1.00E+03 | TCID50/mL | |
| | Respiratory Syncytial Virus Type A | ZeptoMetrix | 2006 Isolate | 0810040ACF | 1.00E+04 | TCID50/mL | |
| | (RSV-A) Culture Fluid | | | | | | |
| | Respiratory Syncytial Virus Type B | ZeptoMetrix | CH93(18)-18 | 0810040CF | 1.00E+04 | TCID50/mL | |
| | (RSV-B) Culture Fluid | | | | | | |
| 6 | Parainfluenza Virus Culture Fluid | ZeptoMetrix | Type 1 | 0810014CF | 1.00E+04 | TCID50/mL | 0/3 |
| | Parainfluenza Virus Culture Fluid | ZeptoMetrix | Type 2 | 0810015CF | 1.00E+04 | TCID50/mL | |
| | Parainfluenza Virus Culture Fluid | ZeptoMetrix | Type 3 | 0810016CF | 1.00E+06 | TCID50/mL | |
| | Parainfluenza Virus Culture Fluid | ZeptoMetrix | Type 4A | 0810060CF | 1.00E+05 | TCID50/mL | |
| 7 | Rhinovirus Culture Fluid | ZeptoMetrix | Type B14 | 0810284CF | 1.00E+04 | TCID50/mL | 0/3 |
| | Rhinovirus Culture Fluid | ZeptoMetrix | Type 1A | 0810012CFN | 1.00E+04 | TCID50/mL | |
| | Enterovirus Type 68 Culture Fluid | ZeptoMetrix | 2007 Isolate | 0810237CF | 1.00E+04 | TCID50/mL | |
| | Enterovirus Type 71 Culture Fluid | ZeptoMetrix | 200E Isolate | 0810236CF | 1.00E+04 | TCID50/mL | |
| 8 | E. coli | Hologic | GP1914 | 43653 | 1.00E+06 | CFU/mL | 0/3 |
| | H. influenzae | Hologic | GP1915 | 51654 | 1.00E+06 | CFU/mL | |
| | Mycobacterium tuberculosis | ZeptoMetrix | H37Ra-1 | 801660 | 1.00E+06 | CFU/mL | |
| | L. plantarum | Hologic | GP1916 | 8014 | 1.00E+06 | CFU/mL | |
| 9 | L. pneumophila | Hologic | GP0004 | 33152 | 1.00E+06 | CFU/mL | 0/3 |
| | B. pert | Hologic | GP0267 | 8467 | 1.00E+06 | CFU/mL | |
| | M. pneumoniae | Hologic | GP0100 | 29342 | 1.00E+06 | Copies/mL | |
| | M. catarrhalis | Hologic | GP0119 | 25238 | 1.00E+06 | CFU/mL | |
| 10 | N. meningiditis | Hologic | GP0755 | 13077 | 1.00E+06 | CFU/mL | 0/3 |
| | N. mucosa | Hologic | GP0190 | 19696 | 1.00E+06 | CFU/mL | |
| | P. aeruginosa | Hologic | GP1917 | 19142 | 1.00E+06 | CFU/mL | |
| | S. aureus | Hologic | GP0018 | 12600 | 1.00E+06 | CFU/mL | |
| 11 | S. epidermidis | Hologic | GP1918 | 35984 | 1.00E+06 | CFU/mL | 0/3 |
| | S. pneumoniae | Hologic | GP1919 | 49619 | 1.00E+06 | CFU/mL | |
| | S. pyogenes | Hologic | GP1920 | 19615 | 1.00E+06 | CFU/mL | |
| | S. salivarius | Hologic | GP0816 | 13419 | 1.00E+06 | CFU/mL | |

TABLE 26
OC43, HKU1, NL63, and 229E Performance in the Presence of Common Organisms

| | | | | | Final | | |
|---|---|---|---|---|---|---|---|
| Panel | Organism | Manufacturer | Strain | ATTC/PN# | Concentration | Units | Reactivity |
| 1 | Adenovirus Culture Fluid | ZeptoMetrix | Type 1 | 0810050CF | 1.00E+06 | TCID50/mL | 12/12 = |
| | Adenovirus Culture Fluid | ZeptoMetrix | Type 4 | 0810070CF | 1.00E+05 | TCID50/mL | 100% |
| | Adenovirus Culture Fluid | ZeptoMetrix | Type 7A | 0810021CF | 1.00E+05 | TCID50/mL | |
| | Mumps Virus Culture Fluid | ZeptoMetrix | Isolate 1 | 0810079CF | 1.00E+04 | TCID50/mL | |
| 2 | Measles Virus Culture Fluid | ZeptoMetrix | NA | 0810025CF | 1.00E+04 | TCID50/mL | 12/12 = |
| | Corynebacterium diphtheriae | ZeptoMetrix | Z116 | CFU/mL | 1.00E+06 | TCID50/mL | 100% |
| | Cytomegalovirus | ZeptoMetrix | AD-169 | 0810003CF | 1.00E+04 | TCID50/mL | |
| | (CMV) Culture Fluid | | | | | | |
| | Epstein-Barr Virus | ZeptoMetrix | B95-8 | 0810008CF | 1.00E+06 | TCID50/mL | |
| | (EBV) Culture Fluid | | | | | | |
| 3 | Varicella Zoster Virus (VZV) Culture | ZeptoMetrix | Strain 82 | 0810167CF | 1.00E+03 | TCID50/mL | 12/12 = |
| | Fluid | | | | | | 100% |
| | Herpes Simplex Virus Type 1 | ZeptoMetrix | MacIntyre | 0810005CF | 1.00E+05 | TCID50/mL | |
| | (HSV-1) Culture Fluid | | | | | | |
| | Human Herpes Virus Type 6B | ZeptoMetrix | Z29 | 0810072CF | 1.00E+06 | TCID50/mL | |
| | (HHV-6B) Culture Fluid | | | | | | |
| | Human Herpes Virus Type 7 | ZeptoMetrix | SB | 0810071CF | 1.00E+05 | TCID50/mL | |
| | (HHV-7) Culture Fluid | | | | | | |
| 4 | Human Metapneumovirus (hMPV) | ZeptoMetrix | IA14-2003 | 0810163CF | 1.00E+04 | TCID50/mL | 12/12 = |
| | 20 Type A2 Culture Fluid | | | | | | 100% |
| | Human Metapneumovirus (hMPV) 3 | ZeptoMetrix | Peru2-220 | 0810156CF | 1.00E+04 | TCID50/mL | |
| | Type B1 Culture Fluid | | | | | | |
| | Human Metapneumovirus (hMPV) 4 | ZeptoMetrix | Peru1-2002 | 0810157CF | 1.00E+04 | TCID50/mL | |
| | Type B2 Culture Fluid | | | | | | |
| | Human Metapneumovirus (hMPV) 9 | ZeptoMetrix | IA3-2002 | 0810160CF | 1.00E+04 | TCID50/mL | |
| | Type A1 Culture Fluid | | | | | | |
| 5 | Influenza A H3N2 | ZeptoMetrix | Brisbane/10/07 | 0810138CF | 1.00E+03 | TCID50/mL | 12/12 = |
| | Virus Culture Fluid | | | | | | 100% |
| | Influenza B Culture Fluid | ZeptoMetrix | B/Florida/02/06 | 0810037CF | 1.00E+03 | TCID50/mL | |
| | Respiratory Syncytial Virus Type A | ZeptoMetrix | 2006 Isolate | 0810040ACF | 1.00E+04 | TCID50/mL | |
| | (RSV-A) Culture Fluid | | | | | | |
| | Respiratory Syncytial Virus Type B | ZeptoMetrix | CH93(18)-18 | 0810040CF | 1.00E+04 | TCID50/mL | |
| | (RSV-B) Culture Fluid | | | | | | |
| 6 | Parainfluenza Virus Culture Fluid | ZeptoMetrix | Type 1 | 0810014CF | 1.00E+04 | TCID50/mL | 12/12 = |
| | Parainfluenza Virus Culture Fluid | ZeptoMetrix | Type 2 | 0810015CF | 1.00E+04 | TCID50/mL | 100% |
| | Parainfluenza Virus Culture Fluid | ZeptoMetrix | Type 3 | 0810016CF | 1.00E+06 | TCID50/mL | |
| | Parainfluenza Virus Culture Fluid | ZeptoMetrix | Type 4A | 0810060CF | 1.00E+05 | TCID50/mL | |
| 7 | Rhinovirus Culture Fluid | ZeptoMetrix | Type B14 | 0810284CF | 1.00E+04 | TCID50/mL | 12/12 = |
| | Rhinovirus Culture Fluid | ZeptoMetrix | Type 1A | 0810012CFN | 1.00E+04 | TCID50/mL | 100% |
| | Enterovirus Type 68 Culture Fluid | ZeptoMetrix | 2007 Isolate | 0810237CF | 1.00E+04 | TCID50/mL | |
| | Enterovirus Type 71 Culture Fluid | ZeptoMetrix | 200E Isolate | 0810236CF | 1.00E+04 | TCID50/mL | |
| 8 | E. coli | Hologic | GP1914 | 43653 | 1.00E+06 | CFU/mL | 12/12 = |
| | H. influenzae | Hologic | GP1915 | 51654 | 1.00E+06 | CFU/mL | 100% |
| | Mycobacterium tuberculosis | ZeptoMetrix | H37Ra-1 | 801660 | 1.00E+06 | CFU/mL | |
| | L. plantarum | Hologic | GP1916 | 8014 | 1.00E+06 | CFU/mL | |
| 9 | L. pneumophila | Hologic | GP0004 | 33152 | 1.00E+06 | CFU/mL | 12/12 = |
| | B. pert | Hologic | GP0267 | 8467 | 1.00E+06 | CFU/mL | 100% |
| | M. pneumoniae | Hologic | GP0100 | 29342 | 1.00E+06 | Copies/mL | |
| | M. catarrhalis | Hologic | GP0119 | 25238 | 1.00E+06 | CFU/mL | |
| 10 | N. meningiditis | Hologic | GP0755 | 13077 | 1.00E+06 | CFU/mL | 12/12 = |
| | N. mucosa | Hologic | GP0190 | 19696 | 1.00E+06 | CFU/mL | 100% |
| | P. aeruginosa | Hologic | GP1917 | 19142 | 1.00E+06 | CFU/mL | |
| | S. aureus | Hologic | GP0018 | 12600 | 1.00E+06 | CFU/mL | |
| 11 | S. epidermidis | Hologic | GP1918 | 35984 | 1.00E+06 | CFU/mL | 12/12 = |
| | S. pneumoniae | Hologic | GP1919 | 49619 | 1.00E+06 | CFU/mL | 100% |
| | S. pyogenes | Hologic | GP1920 | 19615 | 1.00E+06 | CFU/mL | |
| | S. salivarius | Hologic | GP0816 | 13419 | 1.00E+06 | CFU/mL | |

### SEQUENCES

TABLE 27
Coronavirus Reference Sequences

| SEQ | | |
|---|---|---|
| ID | | |
| NO | Description | Sequence (5′ → 3′) |
| 1 | OC43 | GENBANK ACCESSION NO AY391777.1 |
| 2 | HKU1 | GENBANK ACCESSION NO KF430201.1 |
| 3 | NL63 | GENBANK ACCESSION NO AY567487.2 |
| 4 | 229E | GENBANK ACCESSION NO MF542265.1 |

TABLE 28
Sequences

| SEQ ID | | | |
|---|---|---|---|
| NO | Sequence (5′→3′) | Target | Description |
| 5 | GATGTGGTGGTTTTGGACAT | OC43 | Amplification oligomer |
| 6 | CTTCTTAGAAAACTTACCCTTAGG | OC43 | Amplification oligomer |
| 7 | AGTTGAAGATGCTTATGACCAGGTGC | OC43 | Detection probe |
| 8 | AGACAATATCGTGGTATTAAACCC | HKU1 | Amplification oligomer |
| 9 | AGCTTGAAGACAATCTGC | HKU1 | Amplification oligomer |
| 10 | CCAGAATAGTCACAACCATACTGGTCTAC | HKU1 | Detection probe |
| 11 | CGGAGGTCTTACATCAGC | NL63 | Amplification oligomer |
| 12 | ATCAGTTTGTAAAGCAACATAGT | NL63 | Amplification oligomer |
| 13 | TCTTTGGCACTGCAAGCACGAC | NL63 | Detection probe |
| 14 | CCTGGTAGTCATAGTTTGTTCTG | 229E | Amplification oligomer |
| 15 | ACATCCATTCCTAACCAACC | 229E | Amplification oligomer |
| 16 | CTAGAGACTTTGCCATGCGTCATGTCA | 229E | Detection probe |
| 17 | AAAGGTATGGCGGCTGCTGATGT | NL63 | Detection probe |
| 18 | AATAAGGCTATTAATAATATTGTTGCTTCTTTTAG | NL63 | Detection probe |
| 19 | ACACCAGCCTGCAATCTAATTGTTGAAG | HKU1 | Detection probe |
| 20 | AGACAATATCGTGGTATTAAACC | HKU1 | Amplification oligomer |
| 21 | AGGTCGGTACTAACATCAAG | IC | Amplification oligomer |
| 22 | AGGTCTTACATCAGCAGCCGCCATACC | NL63 | Detection probe |
| 23 | AGTCGTGCTTGCAGTG | NL63 | Amplification oligomer |
| 24 | ATACAGCCTGAAGACTATAAA | OC43 | Amplification oligomer |
| 25 | ATTAAAGGATGTAACACTTTAGCT | HKU1 | Amplification oligomer |
| 26 | CAATATCGTGGTATTAAACCCGTAC | HKU1 | Amplification oligomer |
| 27 | CACAATTAATTATAGGTACACCACCTTGTTG | HKU1 | Detection probe |
| 28 | CACGTTGTCTGGAAGTTTC | | |
| 29 | CACGTTGTCTGGAAGTTTG | IC | Amplification oligomer |
| 30 | CAGGTTCTCTTATAGGTGGC | NL63 | Amplification oligomer |
| 31 | CAGTACCAGCATGATCACAG | HKU1 | Amplification oligomer |
| 32 | CGCGTATTATTGTGACCGTC | OC43 | Amplification oligomer |
| 33 | CGCTATTCAAGAAGTTATTGATGAT | OC43 | Detection probe |
| 34 | CGCTATTCAAGAAGTTATTGATGATATGC | OC43 | Detection probe |
| 35 | CGGTGAACACGATCCTC | 229E | Amplification oligomer |
| 36 | CTATTGGTATTGGTGTGTATTACCCTGG | 229E | Detection probe |
| 37 | GAAAATCAGAAAATTTTGGCTGC | NL63 | Amplification oligomer |
| 38 | GATGTGTATAGATATGAAAGGAACA | OC43 | Amplification oligomer |
| 39 | GATGTGTATAGATATGAAAGGAACAATG | OC43 | Amplification oligomer |
| 40 | GCAGCTAATTCTTCTATACTTTCA | HKUl | Amplification oligomer |
| 41 | GCGTATTATTGTGACCGTC | OC43 | Amplification oligomer |
| 42 | GTCCTCCTGGTAGTGGA | 229E | Amplification oligomer |
| 43 | GTGGTTTAACACCACGATAC | OC43 | Amplification oligomer |
| 44 | GTTCTCAAATTTAATCTGAAAACCA | OC43 | Amplification oligomer |
| 45 | TAAAGCAACATAGTTAAGTCGTG | NL63 | Amplification oligomer |
| 46 | TAGATGGCCGTCTGTCGTATCCA | IC | Detection probe |
| 47 | TATGGTAGTCCTCAATTATTGGCCTAAC | OC43 | Detection probe |
| 48 | TGAAAGTATAGAAGAATTAGCTGC | HKUl | Amplification oligomer |
| 49 | TGTAATAGCATCATTAACGCTAC | NL63 | Amplification oligomer |
| 50 | TTATAGGTGGCATGGTGC | NL63 | Amplification oligomer |
| 51 | TTCCTAGGATGTGAGGGTTCT | OC43 | Amplification oligomer |
| 52 | TCTATGCTTCACGGAATTCTGCAATCCCGGAAATTTATTGTTCCA | OC43 | Synthetic Target |
| | TGGGTTATGTACTTGCGTAAGCGTGGCGAAAAGGGTGCTTACAAT | | |
| | AAAGATCATGGATGTGGCGGTTTTGGACATGTTTATGATTTTAAA | | |
| | GTTGAAGATGCTTATGACCAGGTGCATGATGAGCCTAAGGGTAAG | | |
| | TTTTCTAAGAAGGCTTATGCTTTAATTAGAGGGTATCGTGGTGTT | | |
| | AAACCACTTCTCTATGTAGACCAGTATGGTTGTGATTATACTGGT | | |
| | AGTCTTGCAGATGGCTTAGAGGCTTATGCTGATAAGACATTGCAA | | |
| | GAAAAAATTCTCGATGAAGGAGTGGATTGTAAAATACAAACAGCG | | |
| | CCACACATTAGTCTTACTATGCTGGACATACAGCCTGAAGACTAT | | |
| | AAAAGTGTTGATGTCGCTATTCAAGAAGTTATTGATGATATGCAT | | |
| | TGGGGTGATGGTTTTCAGATTAAATTTGAGAATCCTCACATCCTA | | |
| | GGAAGATGCATAGTTTTTGATGTTAAAGGTGTAGAAGAATTGCAT | | |
| | GACGATTTAGTTAATTACATTCGTGATAAAGGTTGTGTTGCTGAC | | |
| | CAATCCAGGAAATGGATTGGCCATTGTACCATAGTATAGTTTTTA | | |
| | CCATAGTGGCCATTATTATGTGGATTGTGTATTTTGTGAATAGTA | | |
| | TCAGGTTGTTTATTAGAACTGGAAGTTTTTGGAGTTTTAACCCAG | | |
| | AAACAAACAACTTGATGTGTATAGATATGAAAGGAACAATGTATG | | |
| | TTAGGCCGATAATTGAGGACTATCATACCCTGACGGTCACAATAA | | |
| | TACGCGGTCATCTTTATATTCAAGGTATAAAACTAGGTATTGGCT | | |
| | ATTCTTTGGCAGATTTGCCAGCTTATATGTCTGTTGCTAAGGTTA | | |
| | CACACCTGTGCACATGCGGCCGCTGCTACACTGGATCC | | |
| 53 | TCTATGCTTCACGGAATTCCAAATGGTTTCAATTCTGTAGGTTGT | HKU1 | Synthetic Target |
| | ATATTGAAGAGAGTGATTTAATAATTTCAAATTTTAAATTTGATG | | |
| | ATTATGATTTTAGTGTAGAAGATGTTTATGCTGAGGTTCATGCTG | | |
| | AGCCTAAAGGTAAATATTCACAAAAAGCTTATGCTTTACTTAGAC | | |
| | AATATCGTGGTATTAAACCCGTACTTTTTGTAGACCAGTATGGTT | | |
| | GTGACTATTCTGGTAAATTAGCAGATTGTCTTCAAGCTTATGGTC | | |
| | ATTATTCTTTGCAAGATATGAGACAAAAGCAGTCTGTATGGCTTG | | |
| | CCAATTGTGACTTTGATATTGTAGTGGCTTGGCATGTAGTTCGTG | | |
| | ATTCACGATTTGTTATGCGCCTGCAGACTATAGCTACTATTTGTG | | |
| | GTATTAAATATGTTCTAAGATAATGAAAGATGATGGAAATTGTGT | | |
| | TGTTTTAGAGCTTGATCCTCCTTGTAAATTTTCTATACAAGATGT | | |
| | TAAGGGACTTAAAATTAAGTATCTTTATTTTATTAAAGGATGTAA | | |
| | CACTTTAGCTAGAGGGTGGGTTGTTGGTACTTTATCTTCAACAAT | | |
| | TAGATTGCAGGCTGGTGTTGCTACTGAGTATGCAGCTAATTCTTC | | |
| | TATACTTTCATTATGTGCATTTTCTGTAGATCCTAAGAAAACTTA | | |
| | TTTAGATTATATACAACAAGGTGGTGTACCTATAATTAATTGTGT | | |
| | TAAAATGCTCTGTGATCATGCTGGTACTGGTATGGCCATTACTAT | | |
| | TAAACCTGAGGCTACTATTAACCAAGATTCTTATGGTGGTGCCTC | | |
| | TGTTTGTATTTATTGCCGTGCACGTGTAGAGCATCCAGATGTAGA | | |
| | TGGTATATGTAAATTACGTGGTAAATTTGTACAAGTCCCTTTGGG | | |
| | TATAAAAGACCCTATTCTTGCGGCCGCTGCTACACTGGATCC | | |
| 54 | TCTATGCTTCACGGAATTCTTATTTTGTTATAAAGAGGTGTACTA | NL63 | Synthetic Target |
| | AGTCGGTTATGGAACACGAGCAATCCATGTATAACCTACTTAACT | | |
| | TTTCTGGTGCTTTGGCTGAGCATGATTTCTTTACTTGGAAAGATG | | |
| | GCAGAGTCATTTATGGTAATGTTAGTAGACATAATCTTACTAAAT | | |
| | ATACTATGATGGACTTGGTCTATGCTATGCGTAACTTTGATGAAC | | |
| | AAAATTGTGATGTTCTAAAAGAAGTATTAGTTTTAACTGGTTGTG | | |
| | CCATGTACACAGGTTCTCTTATAGGTGGCATGGTGCTCGGAGGTC | | |
| | TTACATCAGCAGCCGCCATACCTTTTTCTTTGGCACTGCAAGCAC | | |
| | GACTTAACTATGTTGCTTTACAAACTGATGTGCTTCAAGAAAATC | | |
| | AGAAAATTTTGGCTGCATCATTTAATAAGGCTATTAATAATATTG | | |
| | TTGCTTCTTTTAGTAGCGTTAATGATGCTATTACACAAACTGCAG | | |
| | AGGCTATACATACTGTTACTATTGCACTTAATAAGATTCAGGATG | | |
| | TTGTTAATCAACAGGGTAGTGCTCTTAACCATCTCACTTCACAAT | | |
| | TGAGACATAATTTTCAGGCCATTTCTAATTCAATTCAGGCTATTT | | |
| | ATGACCGGCTTGATTCAATTCAAGCCGATCAACAAGTTGACAGAT | | |
| | TAATTACTGGACGGCTTGCAGCTTTGAATGCATTTGTTTCCCAAG | | |
| | TTTTGAATAAATATACTGAAGTTCGTGGTTCAAGACGCTTAGCAC | | |
| | AGCAGAAGATTAATGAATGTGTCAAGTCACAATCTAATAGATATG | | |
| | GTTTTTGTGGCAATGGCACTCACATCTTTTCAATCGTCAACTCAG | | |
| | CTCCAGATGGTTTGGCGGCCGCTGCTACACTGGATCC | | |
| 55 | TCTATGCTTCACGGAATTCCTTAGAGCGCCAACAATGGCAAACCA | 229E | Synthetic Target |
| | GGAGAAATATTCTACCATTTACAAGTTGCACCCATCATTTAATGT | | |
| | TAGTGATGCTTATGCAAATCTTGTACCTTATTACCAACTTATTGG | | |
| | CAAACAGCGTATCACCACAATACAGGGTCCTCCTGGTAGTGGAAA | | |
| | ATCGCATTGCTCTATTGGTATTGGTGTGTATTACCCTGGAGCGAG | | |
| | GATCGTGTTCACTGCTTGTTCTCACGCTGCTGTTGATTCGCTTTG | | |
| | TGCAAAAGCTGTCACAGCCTATAGTGTTGATAAGTGTACACGTAT | | |
| | TATTCCTGCACGTGCCAGAGTTGAGTGTTATAGTGGTTTTAAACC | | |
| | TAACAACACTAGTGCACAATACGTGTTTATTGTTAATGTACTACT | | |
| | CTGGTGTGTGGTGCTTATAGTGATACTACTAGTGTGTATTACAAT | | |
| | AATTAAACTAATTAAGCTTTGTTTCACTTGCCATATGTTTTGTAA | | |
| | TAGAACAGTTTATGGCCCCATTAAAAATGTGTACCATATTTACCA | | |
| | ATCATATATGCACATAGACCCTTTCCCTAAACGAGTTATTGATTT | | |
| | CTAAACTAAACGACAATGTCAAACGACAATTGTACGGGTGACATT | | |
| | GTCACCCATTTGAAGAATTGGAATTTTGGTTGGAATGTTATTCTA | | |
| | ACCATATTCATTGTTATTCTTCAGTTTGGACACTATAAATACTCC | | |
| | AGATTGTTTTATGGTTTGAAGATGCTTGTACTGTGGCTTCTTTGG | | |
| | CCACTTGTACTTGCTTTGTCAATCTTTGACACCTGGGCTAATTGG | | |
| | GACTCTAATTGGGCCTTTGTTGCATTTAGCTTCCTTATGGCCGTA | | |
| | TCAACACTCGTTATGTGGGTGATGTACTTTGCAAATAGTTTCAGA | | |
| | CTTTTCCGACGTGCTCGAACTTTTTGGGCATGGAATCCTGAGGTT | | |
| | AATGCAATCACTGTCACAACCGTGTTGGGACGCGGCCGCTGCTAC | | |
| | ACTGGATCC | | |

From the foregoing, it will be appreciated that, although specific embodiments have been described herein for purposes of illustration, various modifications may be made without deviating from the spirit and scope of the disclosure. All publications, patents, and patent applications cited herein are hereby incorporated by reference in their entireties for all purposes.

1. (canceled)

2. (canceled)

3. A composition for determining the presence or absence of human coronavirus OC43 (OC43) in a sample, said composition comprising a first amplification oligomer combination comprising first and second human coronavirus OC43-specific amplification oligomers capable of amplifying a target region of an OC43 target nucleic acid, wherein the first OC43-specific amplification oligomer comprises a first OC43-specific target-hybridizing sequence substantially corresponding to the nucleotide sequence of SEQ ID NO:5, SEQ ID NO:38, SEQ ID NO:39, or SEQ ID NO:24 and including from 0-7 nucleotide analogs, and wherein the second OC43-specific amplification oligomer comprises a second OC43-specific target-hybridizing sequence substantially corresponding to the nucleotide sequence of SEQ ID NO:6, SEQ ID NO:43, SEQ ID NO:41, SEQ ID NO:32, SEQ ID NO:44, or SEQ ID NO:51 and including from 0-7 nucleotide analogs.

4. The composition of claim 3, further comprising an OC43-specific detection probe comprising a target-hybridizing sequence selected from the group consisting of SEQ ID NO:7, SEQ ID NO:47, SEQ ID NO:33, or SEQ ID NO:34, or an RNA equivalent or DNA/RNA chimeric thereof and including from 0-7 nucleotide analogs.

5. The composition of claim 4 further comprising a second amplification oligomer combination for determining the presence or absence of human coronavirus HKU1 (HKU1) in a sample, said second amplification oligomer combination comprising first and second human coronavirus HKU1-specific amplification oligomers capable of amplifying a target region of an HKU1 target nucleic acid, wherein the first HKU-specific amplification oligomer comprises a first HKU1-specific target-hybridizing sequence substantially corresponding to the nucleotide sequence of SEQ ID NO:8, SEQ ID NO:20, SEQ ID NO:26, SEQ ID NO:40, or SEQ ID NO:25 and including from 0-7 nucleotide analogs, and wherein the second HKU1-specific amplification oligomer comprises a second HKU1-specific target-hybridizing sequence substantially corresponding to the nucleotide sequence of SEQ ID NO:9, SEQ ID NO:31, or SEQ ID NO:48 and including from 0-7 nucleotide analogs.

6. The composition of claim 5, further comprising an HKU1-specific detection probe comprising a target-hybridizing sequence selected from the group consisting of SEQ ID NO:10, SEQ ID NO:27, or SEQ ID NO:19, or an RNA equivalent or DNA/RNA chimeric thereof and including from 0-7 nucleotide analogs.

7. The composition of claim 5 further comprising a third amplification oligomer combination for determining the presence or absence of human coronavirus NL63 (NL63) in a sample, said third amplification oligomer combination comprising first and second NL63-specific amplification oligomers capable of amplifying a target region of an NL63 target nucleic acid, wherein the first NL63-specific amplification oligomer comprises a first NL63-specific target-hybridizing sequence substantially corresponding to the nucleotide sequence of SEQ ID NO:11, SEQ ID NO:37, SEQ ID NO:50, or SEQ ID NO:30 and including from 0-7 nucleotide analogs, and wherein the second NL63-specific amplification oligomer comprises a second NL63-specific target-hybridizing sequence substantially corresponding to the nucleotide sequence of SEQ ID NO:12, SEQ ID NO:49, SEQ ID NO:45, or SEQ ID NO:23 and including from 0-7 nucleotide analogs.

8. The composition of claim 7, further comprising an NL63-specific detection probe comprising a target-hybridizing sequence selected from the group consisting of SEQ ID NO:13, SEQ ID NO:18, SEQ ID NO:22, or SEQ ID NO:17, or an RNA equivalent or DNA/RNA chimeric thereof and including from 0-7 nucleotide analogs.

9. The composition of claim 8 further comprising a fourth amplification oligomer combination for determining the presence or absence of human coronavirus 229E (229E) in a sample, said fourth amplification oligomer combination comprising first and second 229E-specific amplification oligomers capable of amplifying a target region of a 229E target nucleic acid, wherein the first 229E-specific amplification oligomer comprises a first 229E-specific target-hybridizing sequence substantially corresponding to the nucleotide sequence of SEQ ID NO:14 or SEQ ID NO:42 and including from 0-7 nucleotide analogs, and wherein the second 229E-specific amplification oligomer comprises a second 229E-specific target-hybridizing sequence substantially corresponding to the nucleotide sequence of SEQ ID NO:15 or SEQ ID NO:35 and including from 0-7 nucleotide analogs.

10. The composition of claim 9, further comprising a 229E-specific detection probe comprising a target-hybridizing sequence selected from the group consisting of SEQ ID NO:16 or SEQ ID NO:36, or an RNA equivalent or DNA/RNA chimeric thereof and including from 0-7 nucleotide analogs.

11. (canceled)

12. (canceled)

13. A kit for determining the presence or absence of human coronavirus OC43 (OC43) in a sample, said kit comprising a first amplification oligomer combination comprising first and second human coronavirus OC43-specific amplification oligomers capable of amplifying a target region of an OC43 target nucleic acid, wherein the first OC43-specific amplification oligomer comprises a first OC43-specific target-hybridizing sequence substantially corresponding to the nucleotide sequence of SEQ ID NO:5, SEQ ID NO:38, SEQ ID NO:39, or SEQ ID NO:24 and including from 0-7 nucleotide analogs, and wherein the second OC43-specific amplification oligomer comprises a second OC43-specific target-hybridizing sequence substantially corresponding to the nucleotide sequence of SEQ ID NO:6, SEQ ID NO:43, SEQ ID NO:41, SEQ ID NO:32, SEQ ID NO:44, or SEQ ID NO:51 and including from 0-7 nucleotide analogs.

14. The kit of claim 13, further comprising an OC43-specific detection probe comprising a target-hybridizing sequence selected from the group consisting of SEQ ID NO:7, SEQ ID NO:47, SEQ ID NO:33, or SEQ ID NO:34, or an RNA equivalent or DNA/RNA chimeric thereof and including from 0-7 nucleotide analogs.

15. The kit of claim 14 further comprising a second amplification oligomer combination for determining the presence or absence of human coronavirus HKU1 (HKU1) in a sample, said second amplification oligomer combination comprising first and second human coronavirus HKU1-specific amplification oligomers capable of amplifying a target region of an HKU1 target nucleic acid, wherein the first HKU-specific amplification oligomer comprises a first HKU1-specific target-hybridizing sequence substantially corresponding to the nucleotide sequence of SEQ ID NO:8, SEQ ID NO:20, SEQ ID NO:26, SEQ ID NO:40, or SEQ ID NO:25 and including from 0-7 nucleotide analogs, and wherein the second HKU1-specific amplification oligomer comprises a second HKU1-specific target-hybridizing sequence substantially corresponding to the nucleotide sequence of SEQ ID NO:9, SEQ ID NO:31, or SEQ ID NO:48 and including from 0-7 nucleotide analogs.

16. The kit of claim 15, further comprising an HKU1-specific detection probe comprising a target-hybridizing sequence selected from the group consisting of SEQ ID NO:10, SEQ ID NO:27, or SEQ ID NO:19, or an RNA equivalent or DNA/RNA chimeric thereof and including from 0-7 nucleotide analogs.

17. The kit of claim 16 further comprising a third amplification oligomer combination for determining the presence or absence of human coronavirus NL63 (NL63) in a sample, said third amplification oligomer combination comprising first and second NL63-specific amplification oligomers capable of amplifying a target region of an NL63 target nucleic acid, wherein the first NL63-specific amplification oligomer comprises a first NL63-specific target-hybridizing sequence substantially corresponding to the nucleotide sequence of SEQ ID NO:11, SEQ ID NO:37, SEQ ID NO:50, or SEQ ID NO:30 and including from 0-7 nucleotide analogs, and wherein the second NL63-specific amplification oligomer comprises a second NL63-specific target-hybridizing sequence substantially corresponding to the nucleotide sequence of SEQ ID NO:12, SEQ ID NO:49, SEQ ID NO:45, or SEQ ID NO:23 and including from 0-7 nucleotide analogs.

18. The kit of claim 17, further comprising an NL63-specific detection probe comprising a target-hybridizing sequence selected from the group consisting of SEQ ID NO:13, SEQ ID NO:18, SEQ ID NO:22, or SEQ ID NO:17, or an RNA equivalent or DNA/RNA chimeric thereof and including from 0-7 nucleotide analogs.

19. The kit of claim 18 further comprising a fourth amplification oligomer combination for determining the presence or absence of each of human coronavirus 229E (229E) in a sample, said fourth first amplification oligomer combination comprising first and second 229E-specific amplification oligomers capable of amplifying a target region of a 229E target nucleic acid, wherein the first 229E-specific amplification oligomer comprises a first 229E-specific target-hybridizing sequence substantially corresponding to the nucleotide sequence of SEQ ID NO:14 or SEQ ID NO:42 and including from 0-7 nucleotide analogs, and wherein the second 229E-specific amplification oligomer comprises a second 229E-specific target-hybridizing sequence substantially corresponding to the nucleotide sequence of SEQ ID NO:15 or SEQ ID NO:35 and including from 0-7 nucleotide analogs.

20. The kit of claim 19, further comprising a 229E-specific detection probe comprising a target-hybridizing sequence selected from the group consisting of SEQ ID NO:16 or SEQ ID NO:36, or an RNA equivalent or DNA/RNA chimeric thereof and including from 0-7 nucleotide analogs

21. A method for determining the presence or absence of each of human coronaviruses OC43, HKU1, NL63, and 229E in a sample, said method comprising:
(a) contacting a sample containing or suspected of containing human coronavirus OC43 (OC43), human coronavirus HKU1 (HKU1), human coronavirus NL63 (NL63), and/or human coronavirus 229E (229E) with first, second, third, and fourth amplification oligomer combinations, wherein
(i) the first amplification oligomer combination comprises first and second OC43-specific amplification oligomers capable of amplifying a target region of an OC43 target nucleic acid, wherein the first OC43-specific amplification oligomer comprises a first OC43-specific target-hybridizing sequence substantially corresponding to the nucleotide sequence of SEQ ID NO:5, SEQ ID NO:38, SEQ ID NO:39, or SEQ ID NO:24 and including from 0-7 nucleotide analogs, and wherein the second OC43-specific amplification oligomer comprises a second OC43-specific target-hybridizing sequence substantially corresponding to the nucleotide sequence of SEQ ID NO:6, SEQ ID NO:43, SEQ ID NO:41, SEQ ID NO:32, SEQ ID NO:44, or SEQ ID NO:51 and including from 0-7 nucleotide analogs;
(ii) the second amplification oligomer combination comprises first and second HKU1-specific amplification oligomers capable of amplifying a target region of an HKU1 target nucleic acid, wherein the first HKU-specific amplification oligomer comprises a first HKU1-specific target-hybridizing sequence substantially corresponding to the nucleotide sequence of SEQ ID NO:8, SEQ ID NO:20, SEQ ID NO:26, SEQ ID NO:40, or SEQ ID NO:25 and including from 0-7 nucleotide analogs, and wherein the second HKU1-specific amplification oligomer comprises a second HKU1-specific target-hybridizing sequence substantially corresponding to the nucleotide sequence of SEQ ID NO:9, SEQ ID NO:31, or SEQ ID NO:48 and including from 0-7 nucleotide analogs;
(iii) the third amplification oligomer combination comprises first and second NL63-specific amplification oligomers capable of amplifying a target region of an NL63 target nucleic acid, wherein the first NL63-specific amplification oligomer comprises a first NL63-specific target-hybridizing sequence substantially corresponding to the nucleotide sequence of SEQ ID NO:11, SEQ ID NO:37, SEQ ID NO:50, or SEQ ID NO:30 and including from 0-7 nucleotide analogs, and wherein the second NL63-specific amplification oligomer comprises a second NL63-specific target-hybridizing sequence substantially corresponding to the nucleotide sequence of SEQ ID NO:12, SEQ ID NO:49, SEQ ID NO:45, or SEQ ID NO:23 and including from 0-7 nucleotide analogs; and
(iv) the fourth amplification oligomer combination comprises first and second 229E-specific amplification oligomers capable of amplifying a target region of a 229E target nucleic acid, wherein the first 229E-specific amplification oligomer comprises a first 229E-specific target-hybridizing sequence substantially corresponding to the nucleotide sequence of SEQ ID NO:14 or SEQ ID NO:42 and including from 0-7 nucleotide analogs, and wherein the second 229E-specific amplification oligomer comprises a second 229E-specific target-hybridizing sequence substantially corresponding to the nucleotide sequence of SEQ ID NO:15 or SEQ ID NO:35 and including from 0-7 nucleotide analogs;
(b) performing an in vitro nucleic acid amplification reaction, wherein any OC43, HKU1, NL63, and/or 229E target nucleic acids, if present in the sample, are used as a template for generating amplicons corresponding the OC43, HKU1, NL63, and/or 229E target regions present in the sample; and
(c) detecting the presence or absence of the amplicons, thereby determining the presence or absence of OC43, HKU1, NL63, and 229E in the sample.

22. The method of claim 21, further comprising one or more of
(a) an OC43-specific detection probe comprising a target-hybridizing sequence selected from the group consisting of SEQ ID NO:7, SEQ ID NO:47, SEQ ID NO:33, or SEQ ID NO:34, or an RNA equivalent or DNA/RNA chimeric thereof and including from 0-7 nucleotide analogs;
(b) an HKU1-specific detection probe comprising a target-hybridizing sequence selected from the group consisting of SEQ ID NO:10, SEQ ID NO:27, or SEQ ID NO:19, or an RNA equivalent or DNA/RNA chimeric thereof and including from 0-7 nucleotide analogs;
(c) an NL63-specific detection probe comprising a target-hybridizing sequence selected from the group consisting of SEQ ID NO:13, SEQ ID NO:18, SEQ ID NO:22, or SEQ ID NO:17, or an RNA equivalent or DNA/RNA chimeric thereof and including from 0-7 nucleotide analogs; and
(d) a 229E-specific detection probe comprising a target-hybridizing sequence selected from the group consisting of SEQ ID NO:16 or SEQ ID NO:36, or an RNA equivalent or DNA/RNA chimeric thereof and including from 0-7 nucleotide analogs.

23-35. (canceled)

36. The composition of claim 10, wherein
a) the first amplification oligomer combination comprises:
(i) the first OC43-specific amplification oligomer comprising the first OC43-specific target-hybridizing sequence substantially corresponding to the nucleotide sequence of SEQ ID NO:5 and the second OC43-specific amplification oligomer comprising the second OC43-specific target-hybridizing sequence substantially corresponding to the nucleotide sequence of SEQ ID NO:6 or SEQ ID NO:43;
(ii) the first OC43-specific amplification oligomer comprising the first OC43-specific target-hybridizing sequence substantially corresponding to the nucleotide sequence of SEQ ID NO:38 or SEQ ID NO:30 and the second OC43-specific amplification oligomer comprising a second OC43-specific target-hybridizing sequence substantially corresponding to the nucleotide sequence of SEQ ID NO:41 or SEQ ID NO:32; or
(iii) the first OC43-specific amplification oligomer comprising the first OC43-specific target-hybridizing sequence substantially corresponding to the nucleotide sequence of SEQ ID NO:24 and the second OC43-specific amplification oligomer comprising the second OC43-specific target-hybridizing sequence substantially corresponding to the nucleotide sequence of SEQ ID NO:44 or SEQ ID NO:51;
(b) the second amplification oligomer combination comprises:
(i) the first HKU1-specific amplification oligomer comprising the first HKU1-specific target-hybridizing sequence substantially corresponding to the nucleotide sequence of SEQ ID NO:8, SEQ ID NO:20, or SEQ ID NO:26 and the second HKU1-specific amplification oligomer comprising the second HKU1-specific target-hybridizing sequence substantially corresponding to the nucleotide sequence of SEQ ID NO:9;
(ii) the first HKU1-specific amplification oligomer comprising the first HKU1-specific target-hybridizing sequence substantially corresponding to the nucleotide sequence of SEQ ID NO:40 and the second HKU1-specific amplification oligomer comprising the second HKU1-specific target-hybridizing sequence substantially corresponding to the nucleotide sequence of SEQ ID NO:31; or
(iii) the first HKU1-specific amplification oligomer comprising the first HKU1-specific target-hybridizing sequence substantially corresponding to the nucleotide sequence of SEQ ID NO:25 and the second HKU1-specific amplification oligomer comprising the second HKU1-specific target-hybridizing sequence substantially corresponding to the nucleotide sequence of SEQ ID NO:48;
(c) the third amplification oligomer combination comprises:
(i) the first NL63-specific amplification oligomer comprising the first NL63-specific target-hybridizing sequence substantially corresponding to the nucleotide sequence of SEQ ID NO:11, and the second NL63-specific amplification oligomer comprising the second NL63-specific target-hybridizing sequence substantially corresponding to the nucleotide sequence of SEQ ID NO:12;
(ii) the first NL63-specific amplification oligomer comprising the first NL63-specific target-hybridizing sequence substantially corresponding to the nucleotide sequence of SEQ ID NO:37, and the second NL63-specific amplification oligomer comprising the second NL63-specific target-hybridizing sequence substantially corresponding to the nucleotide sequence of SEQ ID NO:49; or
(iii) the first NL63-specific amplification oligomer comprising the first NL63-specific target-hybridizing sequence substantially corresponding to the nucleotide sequence of SEQ ID NO:50 or SEQ ID NO:30 and the second NL63-specific amplification oligomer comprising the second NL63-specific target-hybridizing sequence substantially corresponding to the nucleotide sequence of SEQ ID NO:45 or SEQ ID NO:23;
(d) a fourth amplification oligomer combination comprises:
(i) the first 229E-specific amplification oligomer comprising the first 229E-specific target-hybridizing sequence substantially corresponding to the nucleotide sequence of SEQ ID NO:14 and the second 229E-specific amplification oligomer comprising the second 229E-specific target-hybridizing sequence substantially corresponding to the nucleotide sequence of SEQ ID NO:15; or
(ii) the first 229E-specific amplification oligomer comprising the first 229E-specific target-hybridizing sequence substantially corresponding to the nucleotide sequence of SEQ ID NO:42 and the second 229E-specific amplification oligomer comprising the second 229E-specific target-hybridizing sequence substantially corresponding to the nucleotide sequence of SEQ ID NO:35.
